(19) United States
(12) Patent Application Publication
(10) Pub. No.: US 2026/0193669 A1
(43) Pub. Date: Jul. 9, 2026

(54) Co-Expression of Constructs and Polypeptides

(71) Applicant: Nykode Therapeutics ASA, Oslo (NO)

(72) Inventors: Audun Trygge Haugen Bersaas, Oslo (NO); Pierre Dillard, Oslo (NO); Bruno Douradinha, Oslo (NO); Lars-Egil Fallang, Nittedal (NO); Stine Granum, Oslo (NO); Joel Benjamin Heim, Oslo (NO); Gunnstein Norheim, Oslo (NO); Monika Sekelja, Oslo (NO); Flourina Kumar Thakor, Oslo (NO); Inga Winge, Høvik (NO); Louise Bjerkan, Røyse (NO)

(73) Assignee: Nykode Therapeutics ASA, Oslo (NO)

(21) Appl. No.: 19/128,436

(22) PCT Filed: Nov. 9, 2023
(86) PCT No.: PCT/EP2023/081312

§ 371 Date: May 8, 2025

(30) Foreign Application Priority Data
Nov. 9, 2022 (DK) PA202270541
Nov. 9, 2022 (DK) PA202270542
Sep. 18, 2023 (DK) PA202370476

(51) Int. Cl. C12N 15/85; A61K 31/7088; A61K 39/00; A61K 39/35; A61K 48/00; A61P 35/00; C07K 14/00; C12N 15/62

(52) U.S. Cl. CPC C12N 15/85; A61K 31/7088; A61K 39/35; A61K 48/005; A61P 35/00; C07K 14/00; C12N 15/62; A61K 2039/53; A61K 2039/575; C07K 2317/526; C07K 2317/53; C07K 2319/00; C12N 2800/10; C12N 2830/34; C12N 2840/206

(57) ABSTRACT

The present disclosure relates to vectors, such as DNA plasmids, comprising multiple nucleic acid sequences of interest engineered to be co-expressed as separate molecules, pharmaceutical compositions comprising such vectors and the use of such vectors and such pharmaceutical compositions in the treatment or prevention of diseases or in the prophylactic or therapeutic treatment of autoimmune diseases, allergic diseases and graft rejection.

### TECHNICAL FIELD

The present disclosure relates to vectors, such as DNA plasmids, comprising multiple nucleic acid sequences of interest engineered to be co-expressed as separate molecules, pharmaceutical compositions comprising such vectors and the use of such vectors and such pharmaceutical compositions in the treatment or prevention of diseases or in the prophylactic or therapeutic treatment of autoimmune diseases, allergic diseases and graft rejection.

### BACKGROUND

The Vaccibody construct is a dimeric fusion protein consisting of two polypeptides, each comprising a targeting unit, which targets antigen-presenting cells (APCs), a dimerization unit and an antigenic unit, which comprises one or more disease-relevant antigens or parts thereof. In other embodiments, the Vaccibody construct is a multimeric fusion protein consisting of multiple polypeptides, each comprising a targeting unit that targets APCs, a multimerization unit, and an antigenic unit that comprises one or more disease-relevant antigens or parts thereof-see for example WO 2004/076489 A1, WO 2011/161244 A1, WO 2013/092875 A1 or WO 2017/118695 A1. These constructs have shown to be efficient in generating an immune response against the antigens or parts thereof, e.g., epitopes, comprised in the antigenic unit.

The Vaccibody construct may be administered to a subject in the form of a polynucleotide encoding the polypeptide, e.g., a polynucleotide comprised in a vector, such as a DNA plasmid. After administration to host cells, e.g., administration to muscle cells of a subject, a polypeptide is expressed which, due to the multimerization unit, such as dimerization unit, forms a multimeric fusion protein, such as a dimeric protein.

### SUMMARY

While Vaccibodies have been proven as efficient therapeutics, some challenges still remain.

For example, adding at least one large antigenic protein C-terminally in the antigenic unit of a Vaccibody may result in proteins not folding in their native conformation, or a conformation mimicking its native conformation, and thus not activating an antibody response, or inducing incorrect and inefficient immune responses to epitopes not displayed by the native antigen.

Furthermore, it is plausible that the secretion of a protein may be inversely correlated with its size and positively correlated with immunogenicity or immune tolerance, respectively. Large proteins are also known to cause a higher burden on the protein production machinery, thus further reducing the yield. Thus, to ensure a satisfactory protein yield and thus overall immunogenicity or immune tolerance, respectively, the total size of the Vaccibody construct would preferably have to be reduced, or the protein units would have to be produced separately. A similar challenge is posed by any Vaccibody construct with an antigenic unit of significant length.

Additionally, while eliciting both a T- and B-cell response is desirable, some targeting unit of the Vaccibody construct may not be ideal to present folded antigens to B-cells as the mechanism of action to activate B cells may benefit from expression of non-targeted antigens. Specifically, the targeted uptake of Vaccibody molecules by APCs may not be the most efficient mechanism to induce a strong B-cell response as it may reduce the availability of the antigen for presentation to lymph nodes.

It has also been shown that combining T cell epitopes and correctly folded B-cell antigens (i.e., a conformation able to induce functional antibodies) on the same Vaccibody molecule is challenging and may result in reduced protein secretion.

Finally, certain antigens might not be suitable for expression with Vaccibodies, such as large antigens, oligomeric antigens, protein complexes, membrane proteins, proteins that need a native N-terminus or which don't tolerate N-terminal fusion.

Allergic symptoms may be triggered by the recognition of environmental antigens, such as allergens, by IgE antibodies and the subsequent activation of inflammatory cell responses by allergen-IgE immune complexes. Accordingly, it may be desirable to avoid inducing an IgE immune response when treating an allergic disease prophylactically or therapeutically.

The present disclosure addresses the above challenges.

The present disclosure provides vectors, e.g., DNA plasmids, for co-expression of a construct and one or more further polypeptides, wherein the one or more further polypeptides comprise one or more epitopes.

In an aspect, the present disclosure concerns a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more epitopes; and
(b) one or more further nucleic acid sequences encoding one or more further polypeptides, wherein the one or more further polypeptides comprise a further antigenic unit comprising one or more further epitopes, wherein the vector allows for the co-expression of the first polypeptide and the one or more further polypeptides as separate molecules.

While said first polypeptide and said one or more further polypeptides are expressed as separate molecules, in some embodiments, the first polypeptide and at least one further polypeptide interact with each other, as detailed herein.

The present disclosure further provides vectors, e.g., DNA plasmids, for co-expression of a construct and one or more further polypeptides, wherein the one or more further polypeptides comprise one or more allergens, hypoallergenic allergens, self-antigens or alloantigens. The term “hypoallergenic allergen” refers to an allergen that has been engineered to reduce allergenic activity, such a IgE reactivity.

In an aspect, the present disclosure concerns a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more T cell epitopes of an allergen, self-antigen or alloantigen; and
(b) one or more further nucleic acid sequences encoding one or more further polypeptides, wherein the one or more further polypeptides comprise a further antigenic unit comprising one or more allergens, hypoallergenic allergens, self-antigens, alloantigens, derivatives thereof or parts thereof,
wherein the vector allows for the co-expression of the first polypeptide and the one or more further polypeptides as separate molecules.

In an aspect, the present disclosure concerns a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more T cell epitopes of an allergen, self-antigen or alloantigen; and
(b) one or more further nucleic acid sequences encoding one or more further polypeptides, wherein the one or more further polypeptides comprise a further antigenic unit comprising one or more allergens, hypoallergenic allergens, self-antigens or alloantigens,
   wherein the vector allows for the co-expression of the first polypeptide and the one or more further polypeptides as separate molecules.

Such a vector will, once administered to a subject, induce a combined tolerance-inducing immune response wherein:
i) The one or more T cell epitopes of an allergen, self-antigen or alloantigen in the antigenic unit are presented in a tolerance-inducing manner which may lead to T cell deletion and/or anergy and/or induction of regulatory T cells (Tregs) and suppression of memory and effector T cell responses towards the relevant allergen self-antigen or alloantigen, and
ii) The one or more allergens or hypoallergenic allergens, induce an allergen-specific IgG response which inhibits the binding of IgE antibodies to allergens, without inducing an IgE immune response or in the case of self-antigens or alloantigens to broaden the tolerogenic response.

Such a vector is thus suitable for use as a prophylactic or therapeutic treatment of autoimmune diseases, allergic diseases or graft rejection.

In an aspect, the present disclosure concerns a vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more T cell epitopes of an allergen; and
(b) one or more further nucleic acid sequences encoding one or more further polypeptides, wherein the one or more further polypeptides comprise a further antigenic unit comprising one or more allergens or hypoallergenic allergens,
wherein the vector allows for the co-expression of the first polypeptide and the one or more further polypeptides as separate molecules.

Such a vector will, once administered to a subject, induce a combined tolerance-inducing immune response wherein:
i) The one or more T cell epitopes of an allergen in the antigenic unit are presented in a tolerance-inducing manner which may lead to T cell deletion and/or anergy and/or induction of regulatory T cells (Tregs) and suppression of memory and effector T cell response towards the relevant allergen, and
ii) The one or more hypoallergenic allergens induce an allergen-specific IgG response which inhibits the binding of IgE antibodies to allergens, without inducing an IgE immune response.

Such a vector is thus suitable for use as a prophylactic or therapeutic treatment of allergic diseases.

As the tolerance-inducing construct causes downregulation of the disease-specific cells of the immune system causing the immune disease in question, it will not suppress the general immune system. Thus, treatment of the immune disease in question with the construct of the disclosure will therefore not result in increased susceptibility to infections and decreased cancer immunosurveillance. However, bystander suppression of immune cells specific for related disease antigens are expected, due to the release of short-range inhibitory cytokines by cell-to-cell contact with the induced antigen-specific regulatory cells.

The tolerance-inducing construct of the disclosure may be administered in the form of a pharmaceutical composition comprising the construct of the disclosure and a pharmaceutically acceptable carrier, for use in the prophylactic or therapeutic treatment of immune disease such as autoimmune diseases, allergic disease and graft rejection.

In another aspect, the present disclosure concerns a method of producing a vector as defined herein comprising the following steps:
a) transfecting cells in vitro with the vector as defined herein;
b) culturing said cells;
c) optionally, lysing the cells to release the vector from the cells; and
d) collecting and optionally purifying the vector.

In another aspect, the present disclosure concerns a host cell comprising a vector as defined herein.

In another aspect, the present disclosure concerns a vector as defined herein for use as a medicament.

In another aspect, the present disclosure concerns a pharmaceutical composition comprising the vector as defined herein and a pharmaceutically acceptable carrier or diluent.

In another aspect, the present disclosure concerns a method of treating a subject having a disease or being in need of prevention of said disease, the method comprising administering to the subject a vector as defined herein or a pharmaceutical composition as defined herein.

In another aspect, the present disclosure concerns a method of treating a subject having cancer, the method comprising administering to the subject a vector as defined herein or a pharmaceutical composition as defined herein comprising such vector.

In another aspect, the present disclosure concerns a method of treating a subject having an infectious disease or being in need of prevention of an infectious disease, the method comprising administering to the subject a vector as defined herein or a pharmaceutical composition as defined herein comprising such vector.

In another aspect, the present disclosure concerns a method of treating a subject having an allergic disease or being in need of prevention an allergic disease, the method comprising administering to the subject a vector as defined herein or a pharmaceutical composition as defined herein comprising such vector.

In another aspect, the disclosure provides a pharmaceutical composition as defined herein for use in the treatment of autoimmune diseases, allergic diseases or graft rejection.

In another aspect, the disclosure provides a pharmaceutical composition as defined herein for use in the treatment of allergic diseases.

In another aspect, the disclosure provides a pharmaceutical composition as defined herein for use in the treatment of autoimmune diseases.

In another aspect, the disclosure provides a pharmaceutical composition as defined herein for use in the treatment of graft rejection.

### DESCRIPTION OF DRAWINGS

FIG. 1 Co-expression elements for use in the vector of the disclosure (IRES)

Shows an IRES co-expression element for use in the vector of the disclosure, which is inserted between two coding regions. When the mRNA has been produced, two ribosomes (T) are able to start translation at two separate sites on the mRNA and two proteins (A and B) are formed. A and B can for example be a first polypeptide and a further polypeptide.

FIG. 2 Co-expression elements for use in the vector of the disclosure (2A peptide)

Shows a 2A self-cleaving peptide co-expression element for use in the vector of the disclosure, which is inserted between two genes. After transcription, one ribosome translates the mRNA and two proteins (A and B) are formed. Upper part of the figure shows how a fusion protein is formed if a 2A peptide sequence is not part of the coding sequence. Lower part of the figure shows the 2 proteins (A and B) formed when the 2A peptide is part of the sequence. A and B can for example be a first polypeptide and further polypeptide.

FIG. 3 Co-expression elements for use in the vector of the disclosure

FIG. 3a shows two promoters (P), i.e., co-expression elements for use in the vector of the disclosure, which are located before two coding regions. Two mRNAs are produced and two ribosomes (T) can start translation at two different mRNAs and two proteins (A and B) are formed. A and B can for example be a first polypeptide and a further polypeptide comprising a further antigenic unit comprising one or more further epitopes of the disclosure. FIG. 3b shows a bidirectional promoter (P) co-expression element for use in the vector of the disclosure, which is located between two coding regions. Two mRNAs are produced by two RNA polymerases transcribing the DNA in separate directions from the promoter, and two ribosomes (T) are able to start translation at two different mRNAs and two proteins (A and B) are formed. A and B can for example be a first polypeptide and further polypeptide.

FIG. 4 Embodiment of a first polypeptide and/or further polypeptides

FIG. 4 illustrates an embodiment of an immunogenic first polypeptide encoded by the first nucleic acid sequence comprised in the vector of the disclosure (FIGS. 4a and 4b) and of a tolerance-inducing first polypeptide encoded by the first nucleic acid sequence comprised in the vector of the disclosure (FIGS. 4c and 4d). The first polypeptide has an N-terminal start and a C-terminal end (illustrated in FIG. 4). The elements and units of the first polypeptide—a targeting unit (TU), a multimerization unit, such as, in this FIG. 4, a dimerization unit (DimU), and an antigenic unit—may be arranged in the first polypeptide such that the antigenic unit is located at the C-terminal end of the first polypeptide (FIGS. 4a and 4c) or at the N-terminal start of the first polypeptide (FIGS. 4b and 4d). Preferably, the antigenic unit is located at the C-terminal end of the first polypeptide.

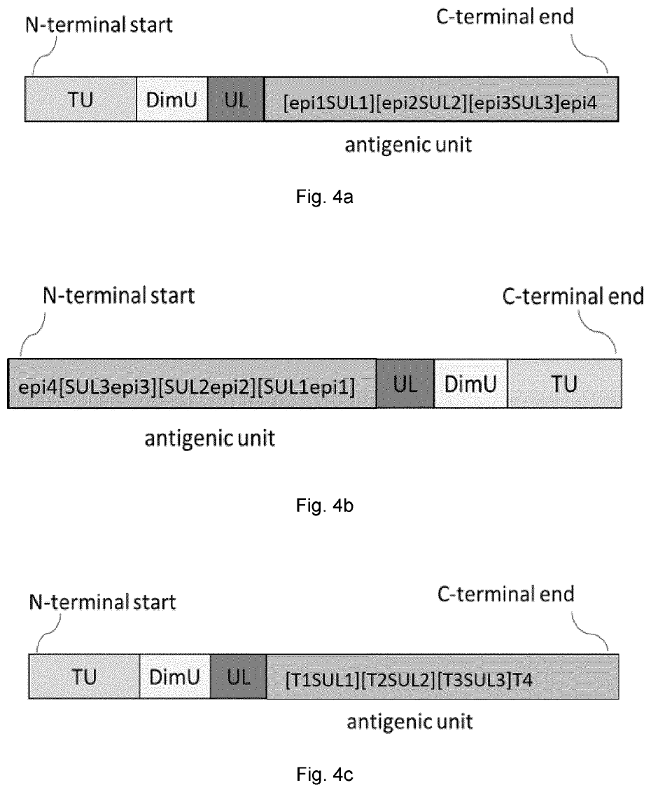

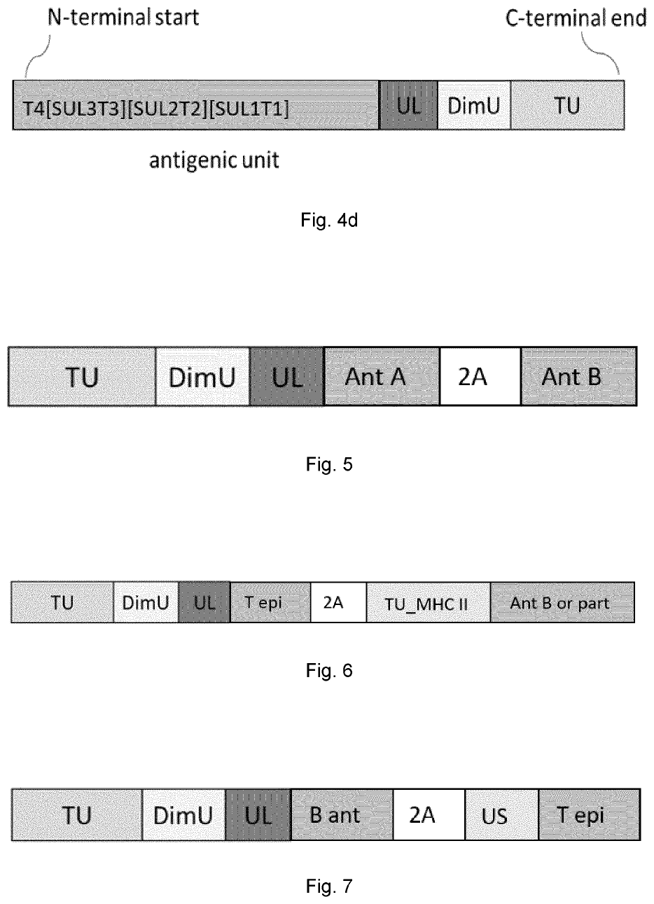

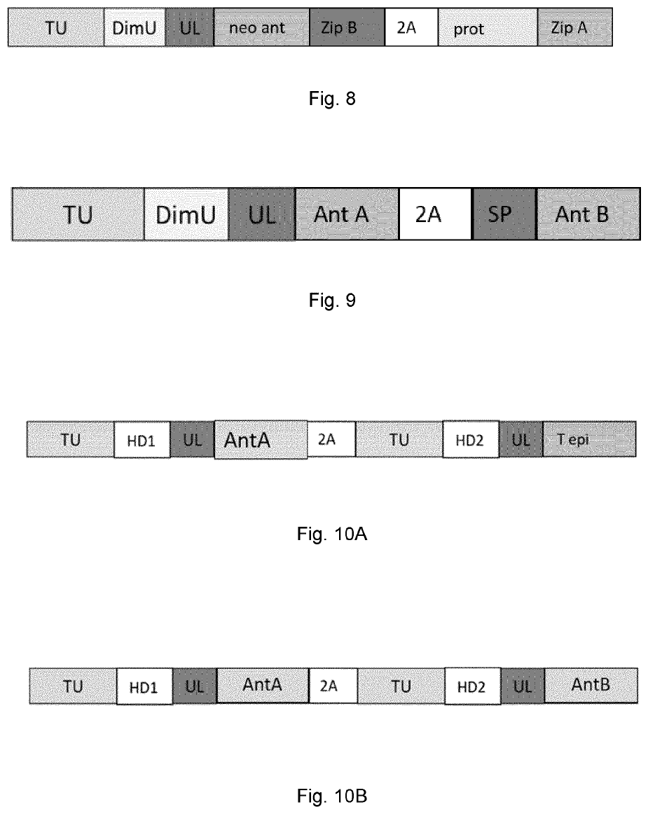

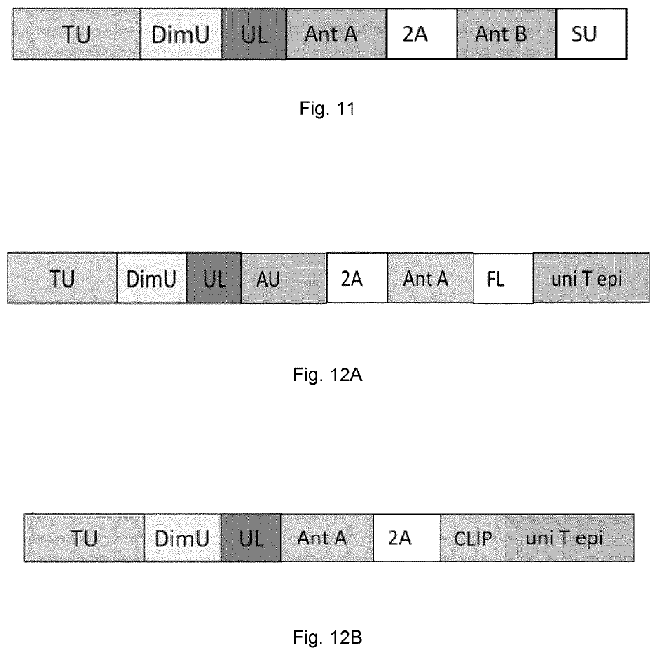

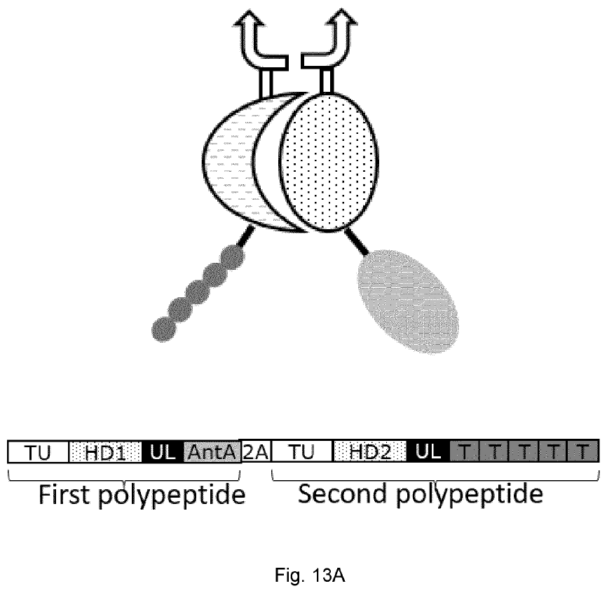

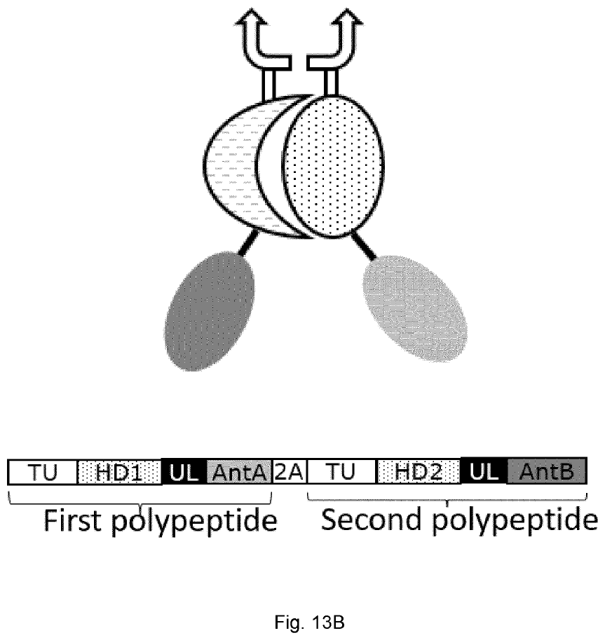

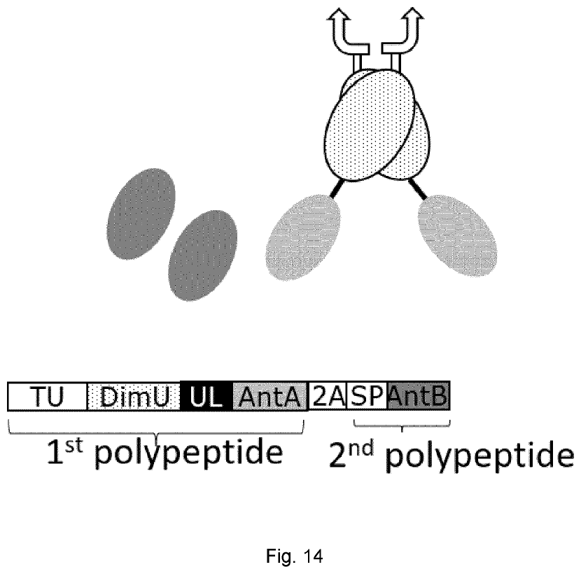

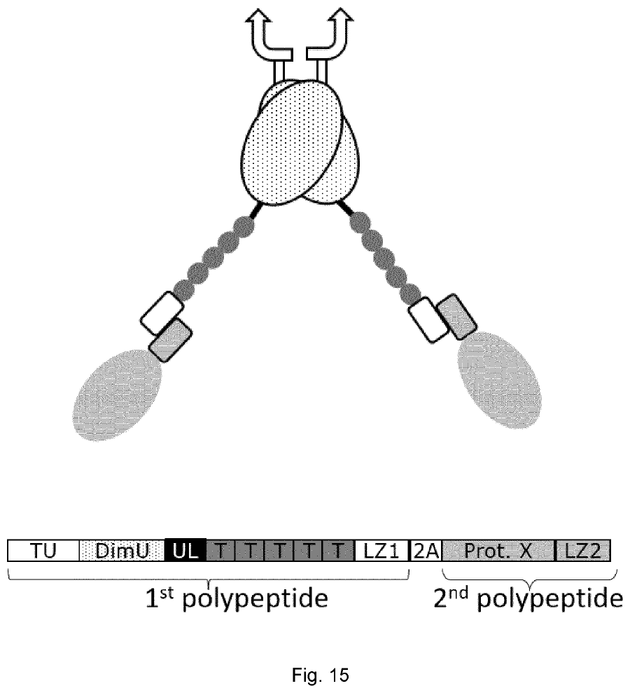

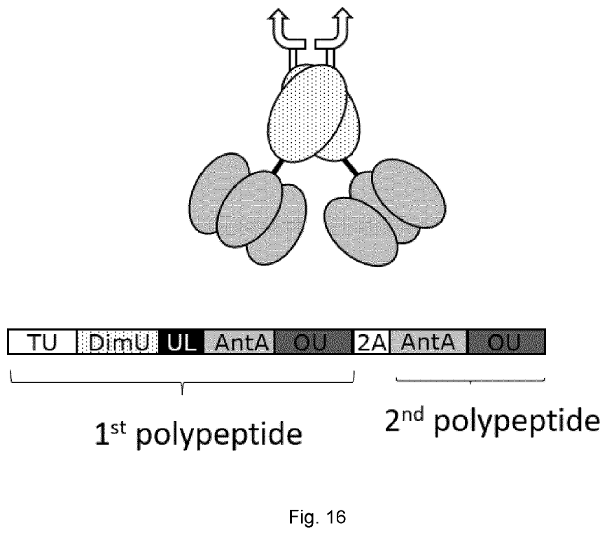

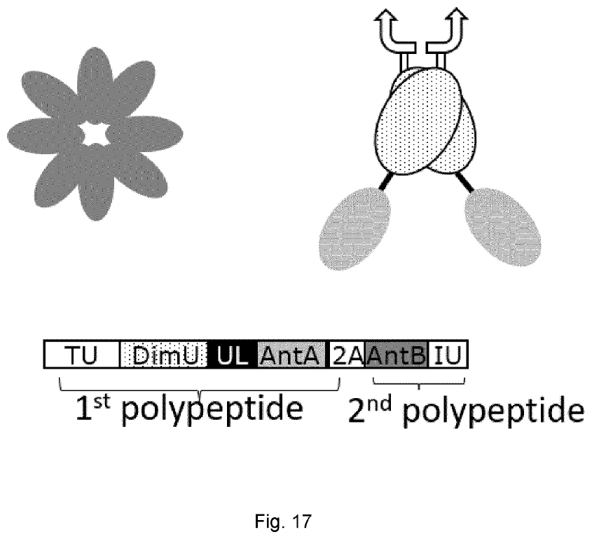

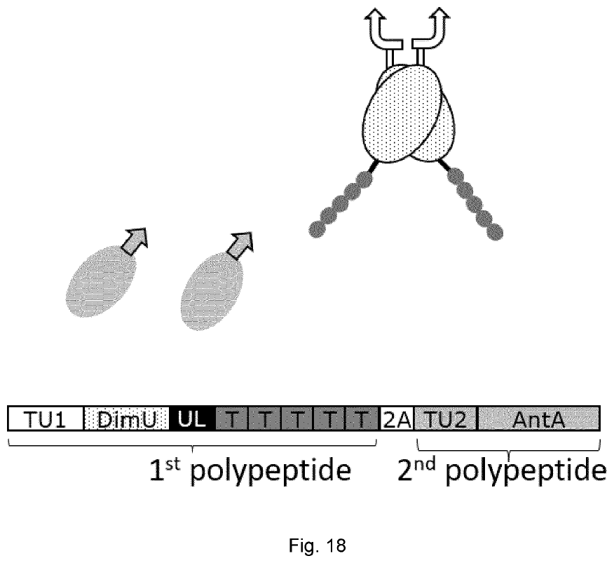

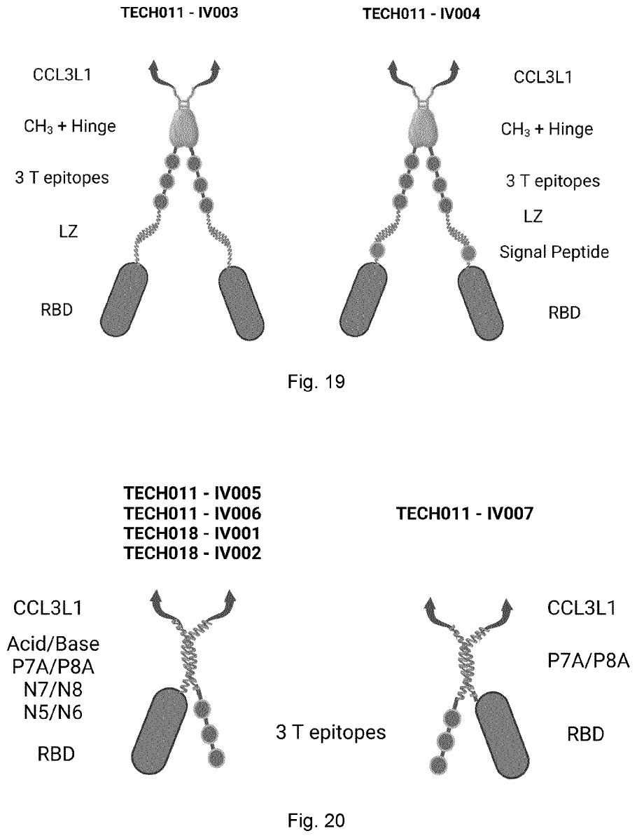

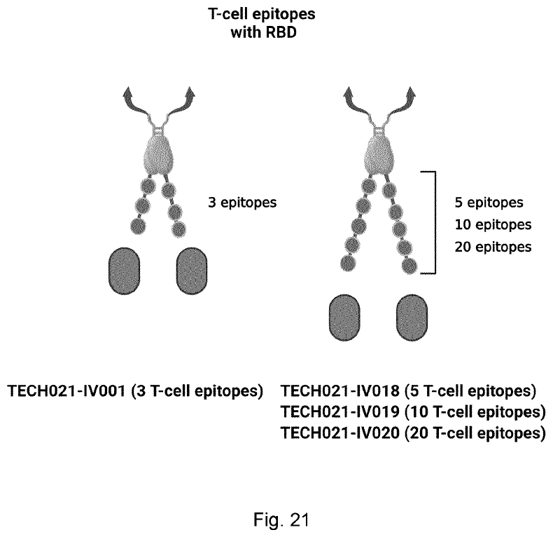

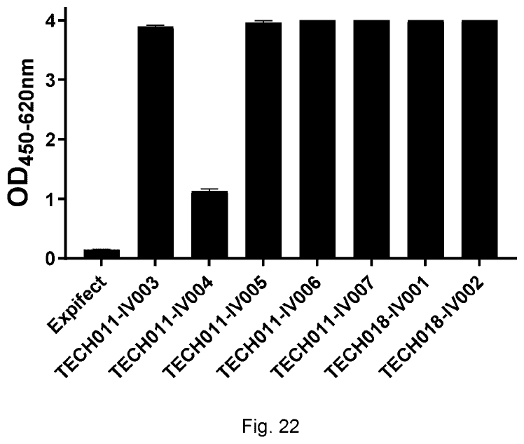

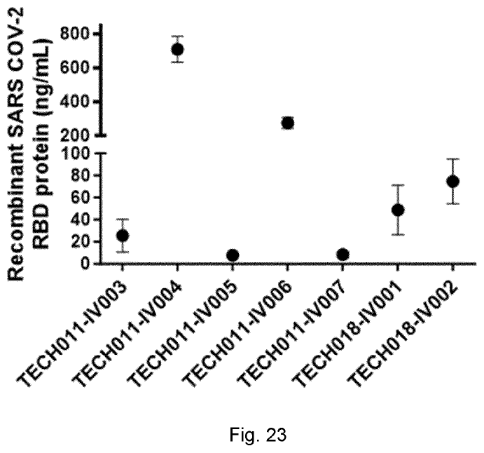

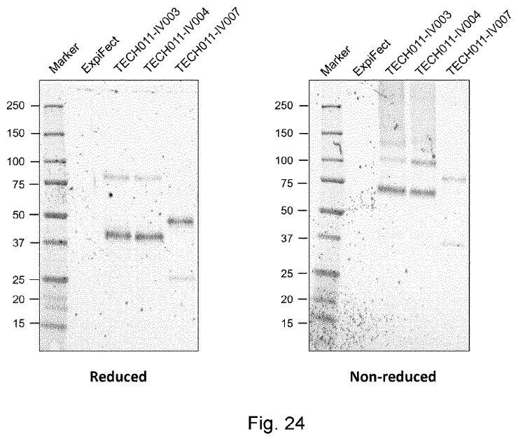

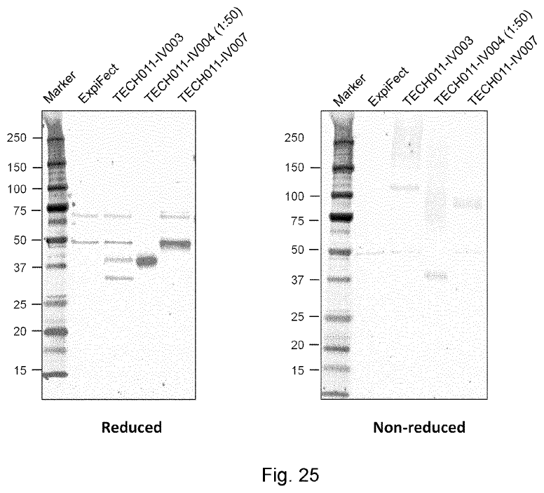

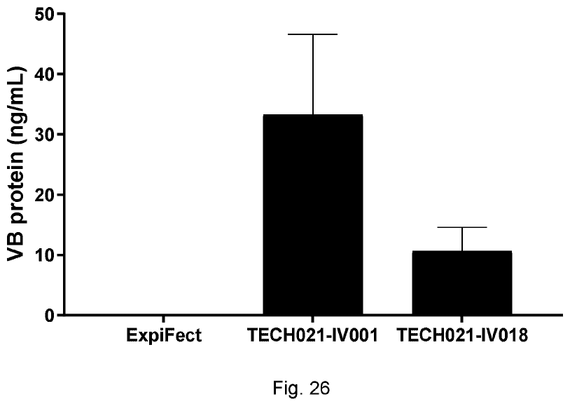

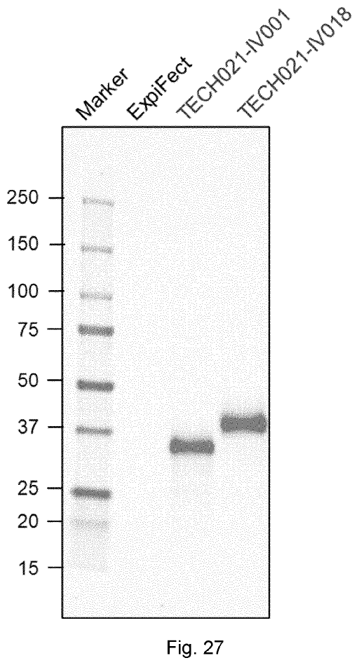

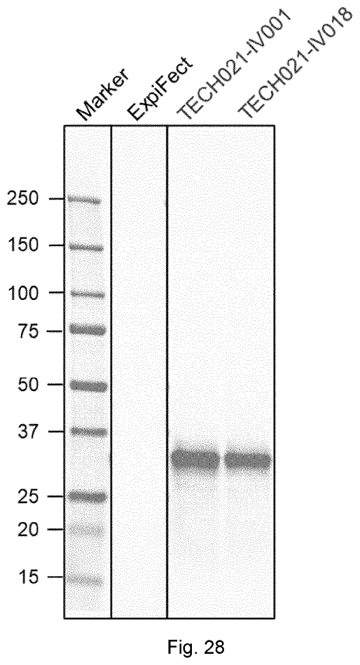

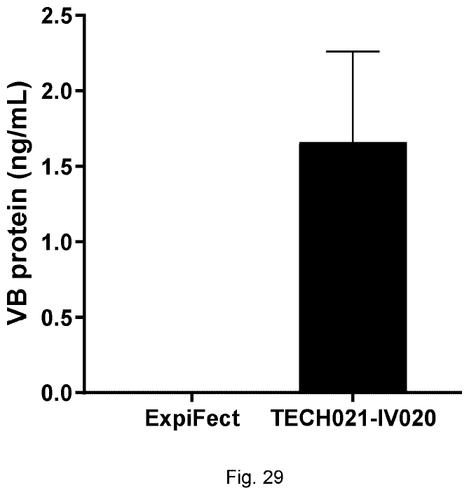

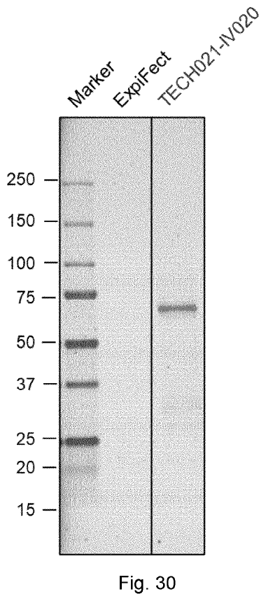

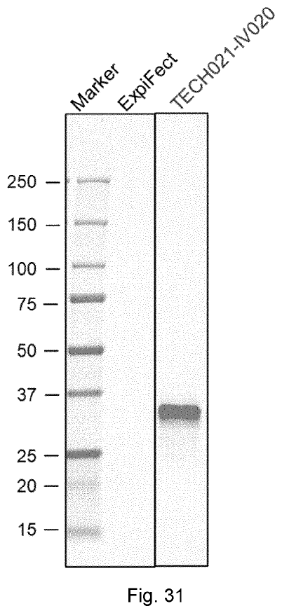

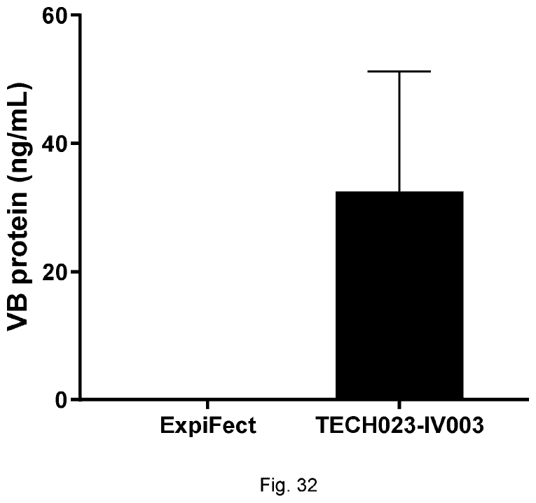

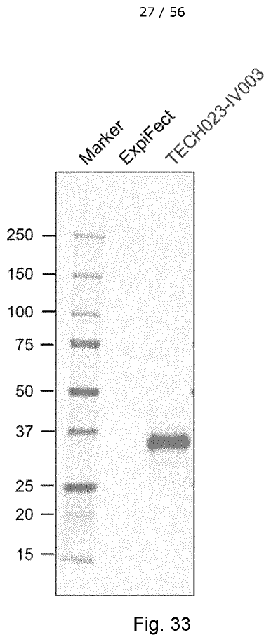

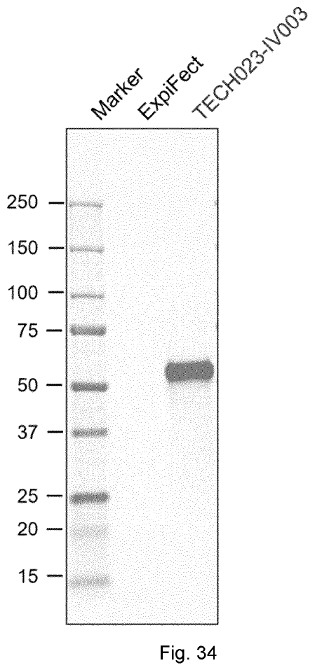

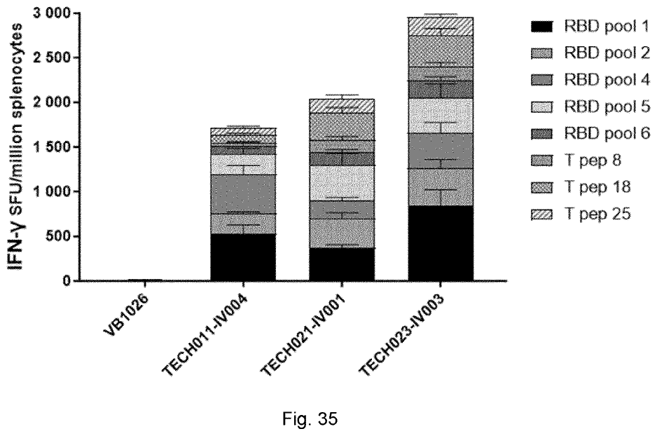

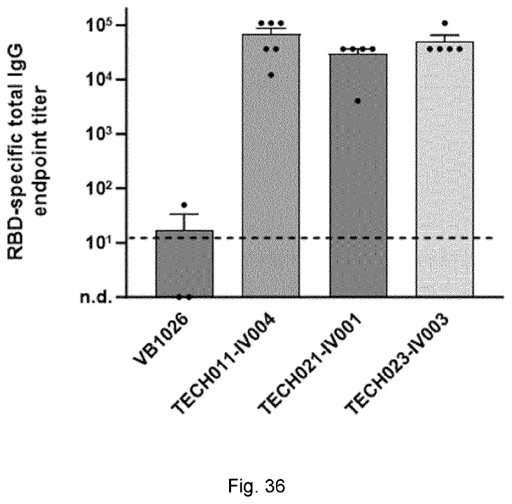

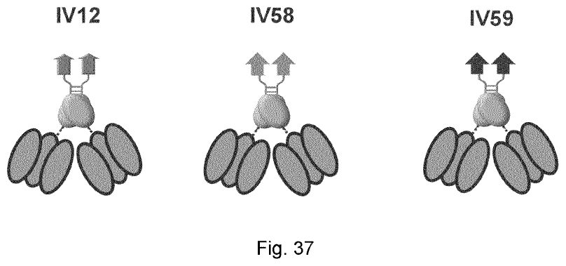

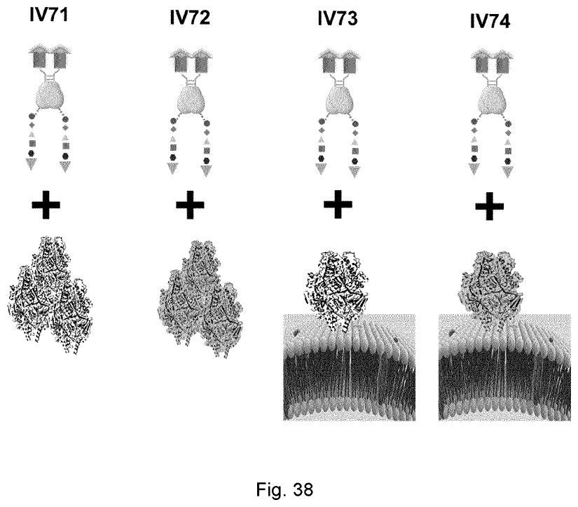

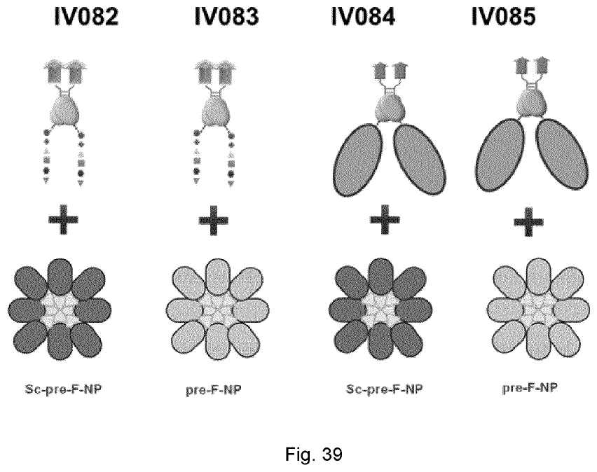

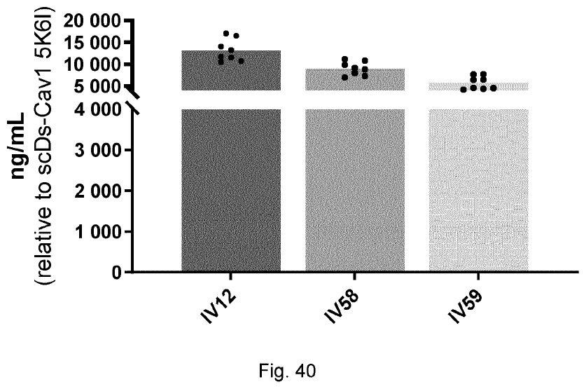

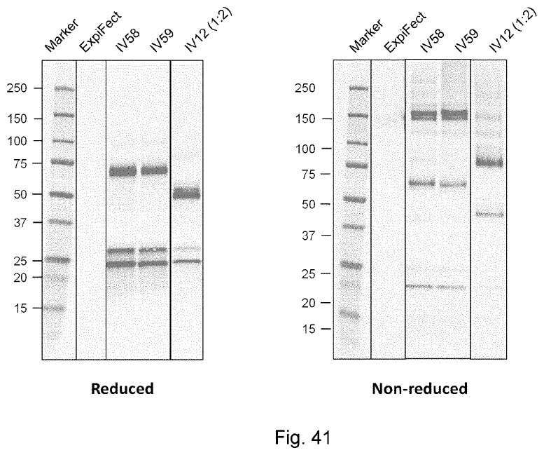

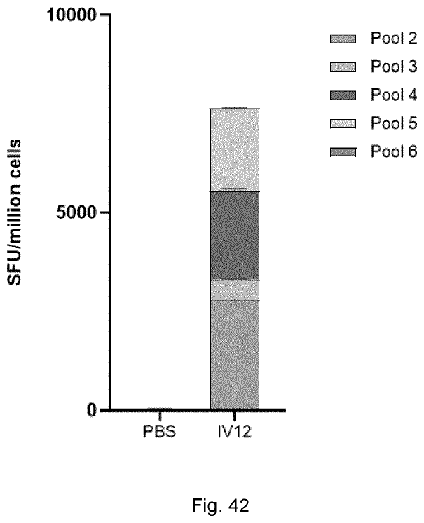

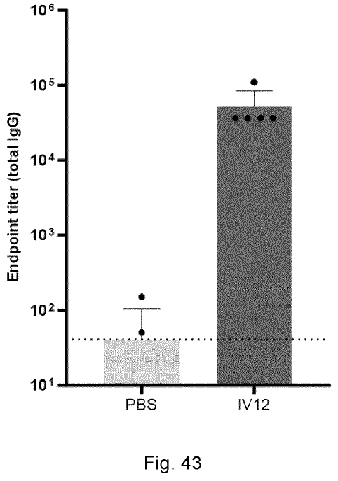

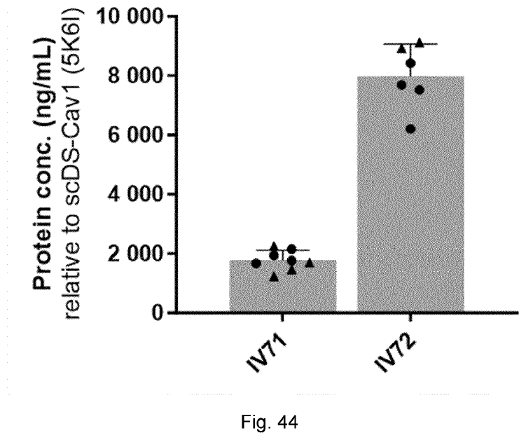

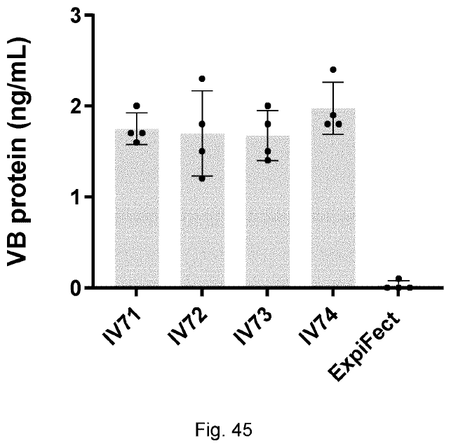

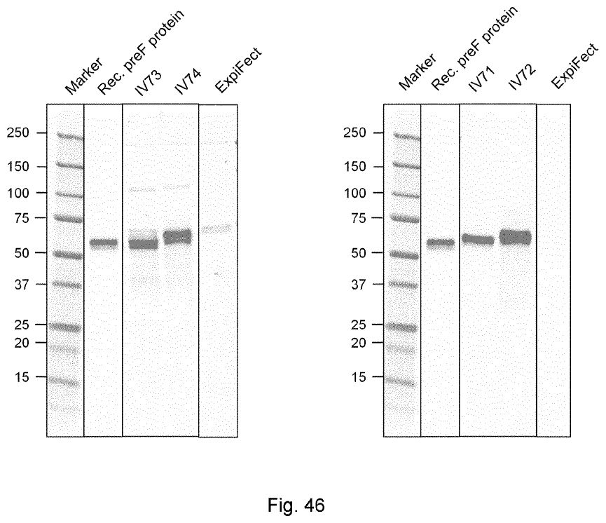

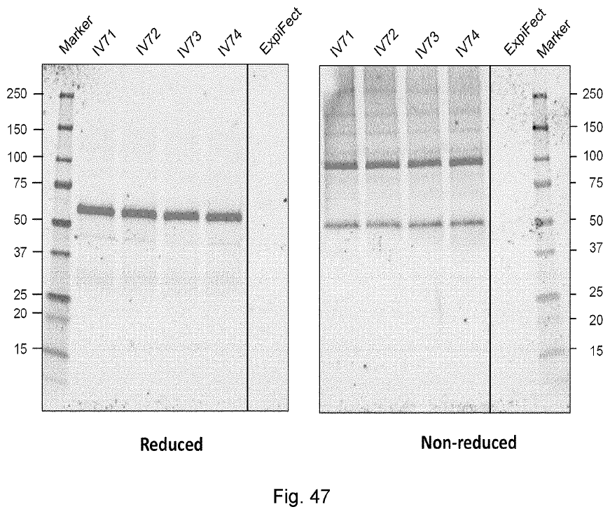

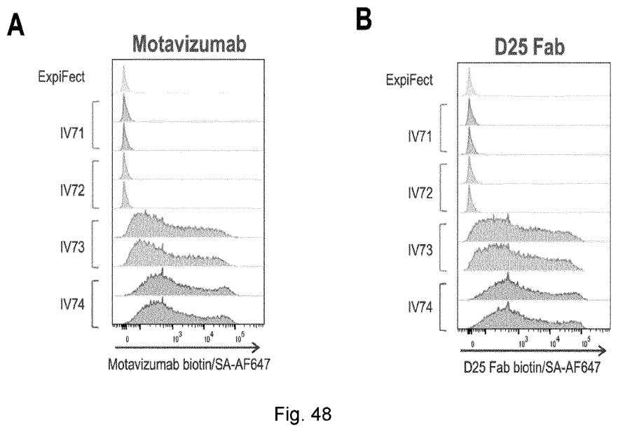

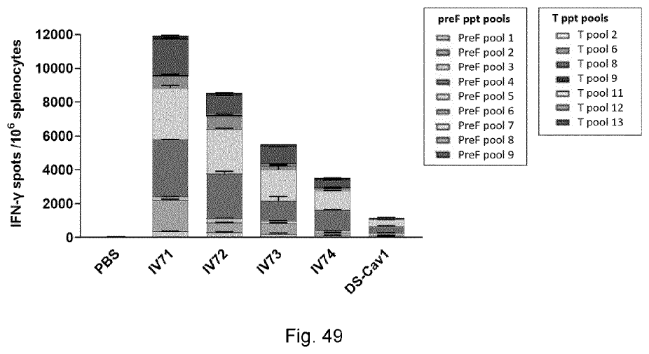

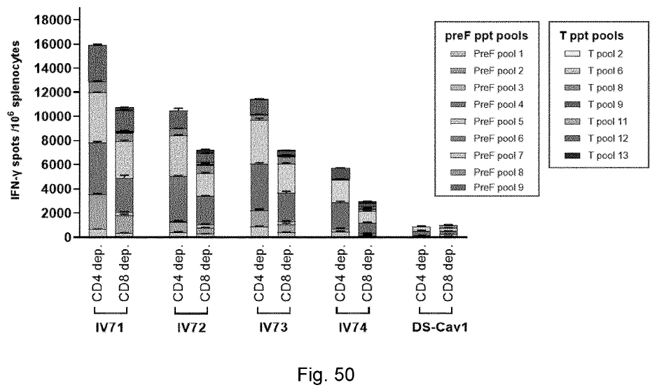

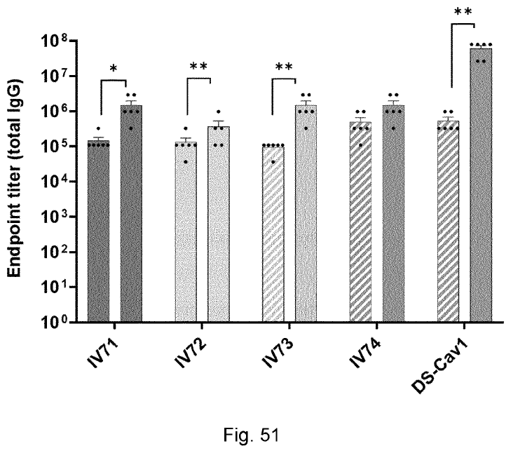

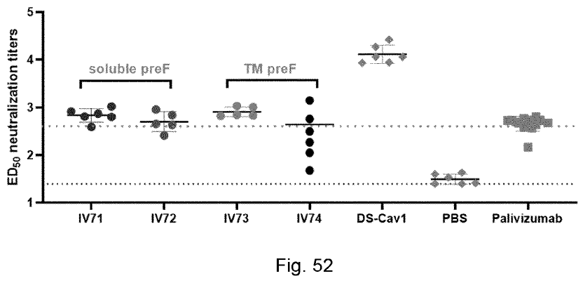

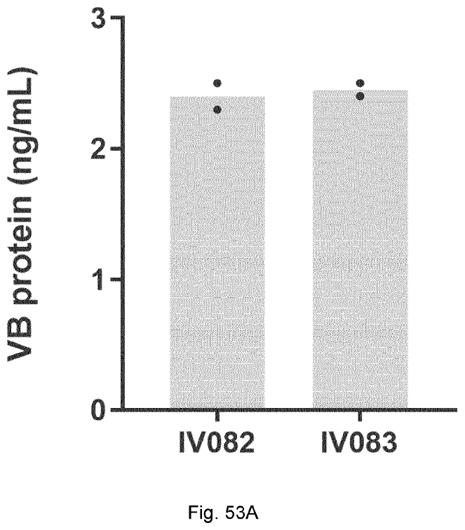

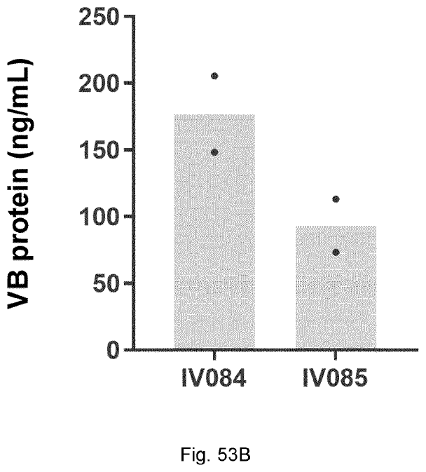

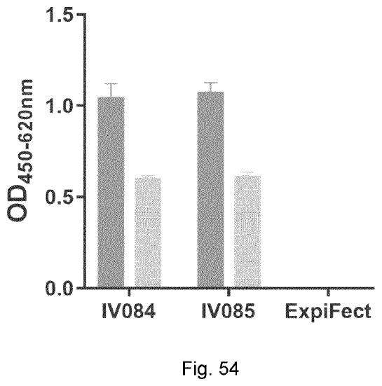

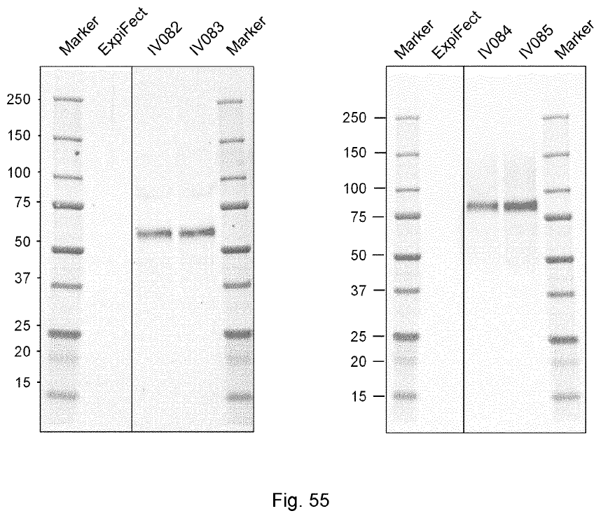

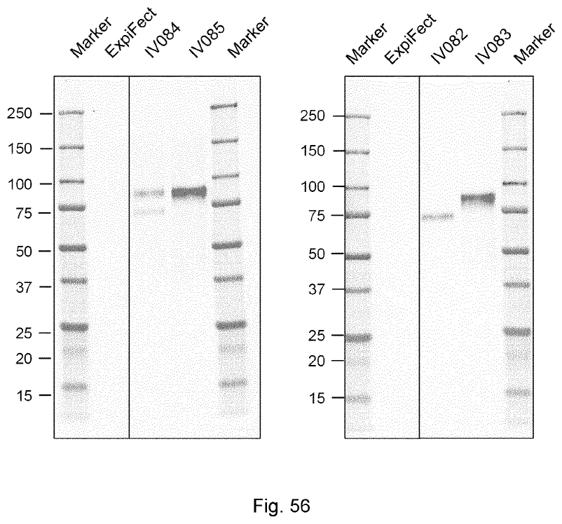

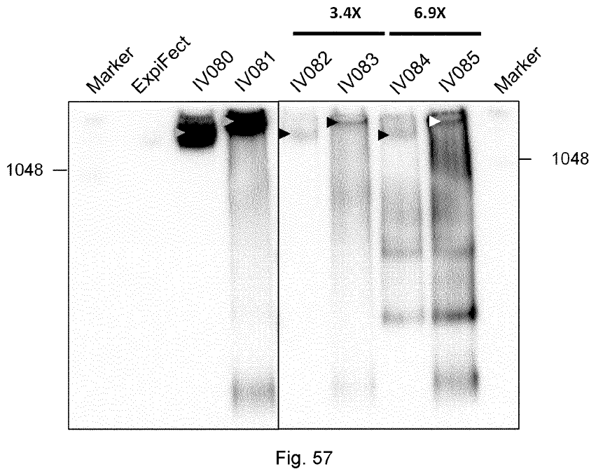

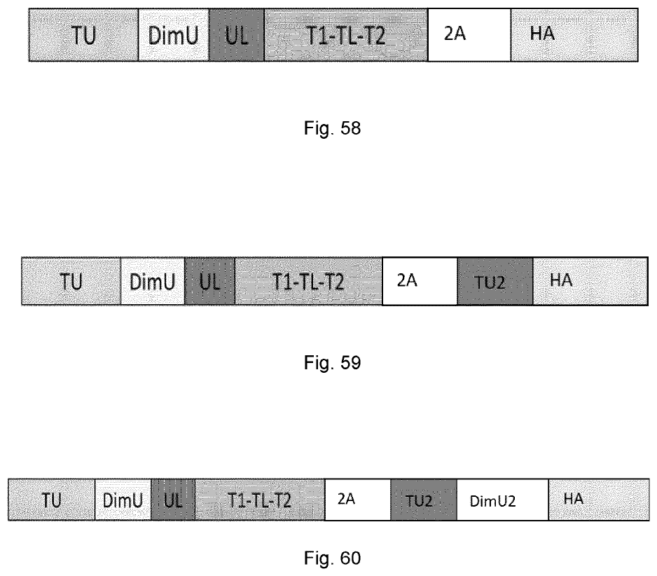

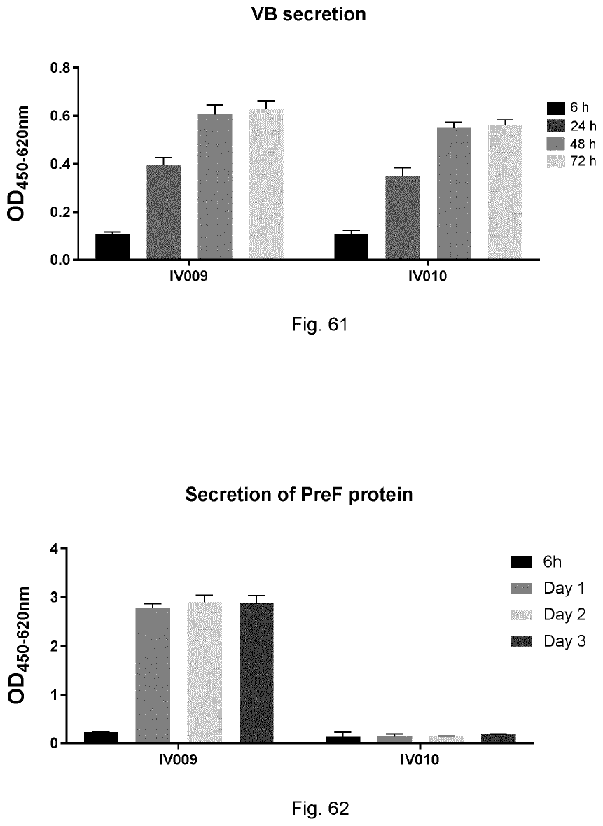

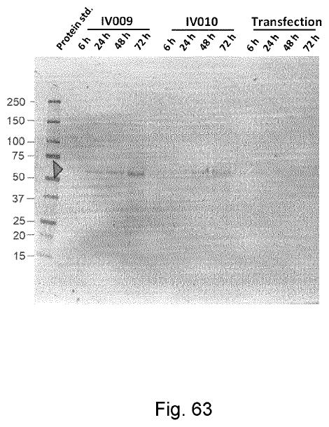

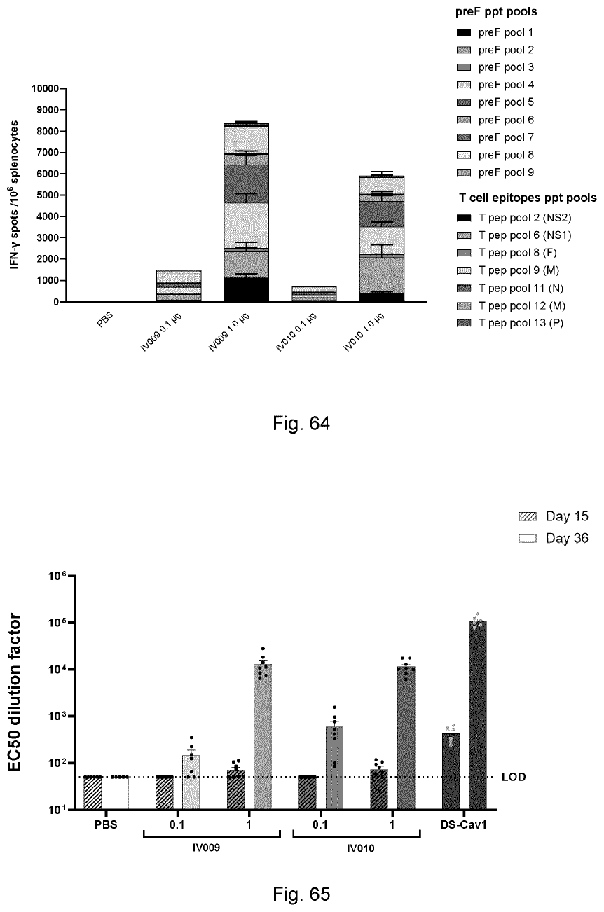

An unit linker (UL) may connect the multimerization unit, such as a dimerization unit, and the antigenic unit. FIGS. 4a and 4b illustrate an antigenic unit with 4 epitopes (epi1, epi2, epi3, epi4), which are separated by subunit linkers (SUL1, SUL2, SUL3). An alternative way to describe the arrangement of the epitopes epi1-epi4 is that these epitopes are arranged in 3 antigenic subunits, each comprising an epitope and a subunit linker (SUL1, SUL2, SUL3), and a terminal epitope (epi4), which is closest to the C-terminal end or N-terminal start of the first polypeptide. The subunits are indicated in the Figure by square brackets. FIGS. 4c and 4d illustrate an antigenic unit with 4 T cell epitopes (T1, T2, T3 and T4), which are separated by subunit linkers (SUL1, SUL2, SUL3). The order and orientation of the above-described units and elements is the same in the multimeric/dimeric protein and the polynucleotide. An alternative way to describe the arrangement of the T cell epitopes T1-T4 is that these epitopes are arranged in 3 antigenic subunits, each comprising a T cell epitope and a subunit linker (SUL1, SUL2, SUL3), and a terminal T cell epitope (T4), which is closest to the C-terminal end or N-terminal start of the first polypeptide. The subunits are indicated in the Figure by square brackets.

FIG. 5: Embodiment of the disclosure

FIG. 5 illustrates an embodiment of the disclosure, wherein the first nucleic acid encodes a polypeptide comprising an antigenic unit comprising an antigen (Ant A) and one further nucleic acid sequence that encodes a further polypeptide comprising a further antigenic unit comprising an antigen or part thereof (Ant B). A 2A self-cleaving peptide (2A) allows the co-expression of the first polypeptide and said further polypeptide. The further polypeptide may, optionally, comprise at the N terminus a signal peptide (nucleic acid sequence encoding the signal peptide not shown).

FIG. 6: Embodiment of the disclosure

FIG. 6 illustrates an embodiment of the disclosure, wherein the first nucleic acid encodes a polypeptide comprising an antigenic unit comprising at least one T cell epitope (T epi) and one further nucleic acid sequence that encodes a further polypeptide comprising an MHC II targeting unit (TU_MHC II) and a further antigenic unit comprising an antigen or part thereof (Ant B or part). A 2A self-cleaving peptide (2A) allows the co-expression of the first polypeptide and said further polypeptide. The further polypeptide may, optionally, comprise at the N terminus a signal peptide (nucleic acid sequence encoding the signal peptide not shown).

FIG. 7: Embodiment of the disclosure

FIG. 7 illustrates an embodiment of the disclosure, wherein the first nucleic acid encodes a polypeptide comprising an antigenic unit comprising a B cell antigen (B ant) and one further nucleic acid sequence that encodes a further polypeptide comprising a ubiquitination sequence (US) and an antigenic unit comprising at least one T cell epitope (T epi). A 2A self-cleaving peptide (2A) allows the co-expression of the first polypeptide and said further polypeptide.

FIG. 8: Embodiment of the disclosure

FIG. 8 illustrates an embodiment of the disclosure, wherein the first nucleic acid encodes a polypeptide comprising an antigenic unit comprising at least one neoantigen (neo ant) and a first leucine zipper motif (Zip B) and one further nucleic acid sequence that encodes a further polypeptide comprising an antigenic unit comprising a protein (prot) and a second leucine zipper motif (Zip A). A 2A self-cleaving peptide (2A) allows the co-expression of the first polypeptide and said further polypeptide The further polypeptide may, optionally, comprise at the N terminus a signal peptide sequence (nucleic acid sequence encoding the signal peptide not shown).

FIG. 9: Embodiment of the disclosure

FIG. 9 illustrates an embodiment of the disclosure, wherein the first nucleic acid encodes a polypeptide comprising a targeting unit (TU), a dimerization unit (DU), an unit linker (UL) and an antigenic unit comprising an antigen (Ant A), and one further nucleic acid sequence that encodes a further polypeptide comprising a signal peptide (SP) and a further antigenic unit comprising an antigen (Ant B). A 2A self-cleaving peptide (2A) allows the co-expression of the first polypeptide and said further polypeptide. The first polypeptide and/or the further polypeptide may, optionally, comprise an interaction unit, such as a heterotrimerization unit (not shown). When the first polypeptide and the further polypeptide both comprise an interaction unit, such as a heterotrimerization unit, the antigenic unit of the first polypeptide (Ant A) is capable of interacting with the antigenic unit of the further polypeptide (Ant B). In some embodiments, the first polypeptide and the further polypeptide both comprise a heterotrimerization unit so that the antigenic unit of the first polypeptide (Ant A) is capable of forming a trimer with two antigenic units of two further polypeptide (Ant B). The interaction unit, which is not shown in the picture, may be located between the further signal peptide and the further antigenic unit, or alternatively, after the further antigenic unit.

FIG. 10: Embodiment of the disclosure

FIG. 10A illustrates an embodiment of the disclosure, wherein the first nucleic acid encodes a polypeptide comprising a targeting unit (TU), a heterodimerization unit (HD1), an unit linker (UL), and an antigenic unit comprising an antigen (Ant A) and one further nucleic acid sequence that encodes a further polypeptide comprising a targeting unit (TU), a different heterodimerization unit (HD2), an unit linker (UL), and a further antigenic unit comprising at least one T cell epitope (T epi). A 2A self-cleaving peptide (2A) allows the co-expression of the first polypeptide and said further polypeptide. The further polypeptide may, optionally, comprise at the N terminus a signal peptide sequence (nucleic acid sequence encoding the signal peptide not shown). Due to the presence of the heterodimerization units, the first and further polypeptide form a heterodimer like illustrated in FIG. 13A.

FIG. 10B illustrates an embodiment of the disclosure, wherein the first nucleic acid encodes a polypeptide comprising a targeting unit (TU), a heterodimerization unit (HD1), an unit linker (UL), and an antigenic unit comprising an antigen A (Ant A) and one further nucleic acid sequence that encodes a polypeptide comprising a targeting unit (TU), a different heterodimerization unit (HD2), an unit linker (UL), and an antigen B (Ant B). A 2A self-cleaving peptide (2A) allows the co-expression of the first polypeptide and said further polypeptide. The further polypeptide may, optionally, comprise at the N terminus a further signal peptide sequence (nucleic acid sequence encoding the signal peptide not shown). Due to the presence of the heterodimerization units, the first and further polypeptide form a heterodimer like illustrated in FIG. 13B.

FIG. 11: Embodiment of the disclosure

FIG. 11 illustrates an embodiment of the disclosure, wherein the first nucleic acid encodes a polypeptide comprising an antigenic unit comprising an antigen (Ant A) and one further nucleic acid sequence that encodes a further polypeptide comprising a further antigenic unit comprising an antigen (Ant B) and a self-assembly unit (SU), which promotes the formation of antigen oligomers, e.g. nanoparticles, displaying Ant B. A 2A self-cleaving peptide (2A) allows the co-expression of the first polypeptide and said further polypeptide. The further polypeptide may, optionally, comprise at the N terminus a signal peptide sequence (nucleic acid sequence encoding the signal peptide not shown).

FIG. 12: Embodiment of the disclosure

FIG. 12A illustrates an embodiment of the disclosure, wherein the first nucleic acid encodes a polypeptide comprising a targeting unit (TU), a dimerization unit (DimU), an unit linker (UL) and an antigenic unit (AU); and one nucleic acid sequence that encodes a polypeptide comprising a further antigenic unit comprising an antigen (Ant A), a furin linker (FL) and at least one universal CD4+ T cell epitope (uni T epi). A 2A self-cleaving peptide (2A) allows the co-expression of the first polypeptide and said further polypeptide.

FIG. 12B illustrates an embodiment of the disclosure, wherein the first nucleic acid encodes a polypeptide comprising a targeting unit (TU), a dimerization unit (DimU), an unit linker (UL) and an antigenic unit comprising an antigen (Ant A); and one further nucleic acid sequences that encodes a further polypeptide comprising a further antigenic unit comprising a class II-associated invariant chain peptide (CLIP) and at least one universal CD4+ T cell epitope (uni T epi). A 2A self-cleaving peptide (2A) allows the co-expression of the first polypeptide and said further polypeptide.

FIG. 13: Embodiment of the disclosure

FIG. 13A illustrates an embodiment of the disclosure, here a heterodimer of a first polypeptide and of a second polypeptide, together with the corresponding nucleic acid sequence. The first nucleic acid encoding the first polypeptide comprises a targeting unit (TU, such as CCL3L1, arrows and lines in white), a first heterodimerization unit (HD1), an unit linker (UL), and an antigenic unit comprising an antigen (AntA, light grey). The second nucleic acid encoding the second polypeptide comprises a targeting unit (TU, arrows and lines in white), a second heterodimerization unit (HD2), an unit linker (UL), and a further antigenic unit comprising T cell epitopes (T, dark grey). The T cell epitopes may optionally be separated by subunit linkers (not shown). The sequences encoding the first polypeptide and the second polypeptide are separated by a 2A sequence.

FIG. 13B illustrates an alternative embodiment of a heterodimer, where the first polypeptide comprises an antigenic unit comprising an antigen A (AntA, light grey), and the second polypeptide comprises a further antigenic unit comprising an antigen B (AntB, dark grey). For instance, antigen A and antigen B are two different antigens from a same pathogen, or from different strains or serotypes of a pathogen.

FIG. 14: Embodiment of the disclosure

FIG. 14 illustrates an embodiment of the disclosure. The nucleic acid sequences encodes a first polypeptide comprising a targeting unit (TU, arrows and lines in white), a dimerization unit (DimU), an unit linker (UL), and an antigenic unit comprising an antigen A (AntA, light grey). The nucleic acid sequence further comprises a sequence encoding a 2A peptide, and a sequence encoding a second polypeptide, which here consists of a signal peptide (SP) and an antigen B (AntB, dark grey). Upon expression from the single vector, a homodimer of the first polypeptide comprising Antigen A is formed, while antigen B is expressed separately.

FIG. 15: Embodiment of the disclosure

FIG. 15 illustrates an embodiment of the disclosure where the interaction unit is a leucine zipper motif. The first polypeptide comprises a targeting unit (TU, arrows and lines in white), a dimerization unit (Dim U), an unit linker (UL), an antigenic unit comprising several T cell epitopes (T, dark grey) and a first leucine zipper motif (LZ1). The T cell epitopes may optionally be separated by subunit linkers (not shown). The second polypeptide comprises a protein, for example a large protein (Prot. X), a linker (in black) and a second leucine zipper motif (LZ2).

FIG. 16: Embodiment of the disclosure

FIG. 16 illustrates a construct of the disclosure, comprising a nucleic acid sequence encoding a first polypeptide and a second polypeptide. The first polypeptide comprises a targeting unit (TU, arrows and lines in white), a dimerization unit (DimU), an unit linker (UL), an antigenic unit comprising an antigen (AntA, light grey), and an oligomerization unit (dark grey) (OU). The second polypeptide comprises a sequence encoding a further antigenic unit comprising the same antigen (AntA, light grey) and in this embodiment an oligomerization unit (dark grey) (OU). A signal peptide (not shown) may be present at the N-terminus of the antigen. Upon expression, the antigen comprised in the antigenic unit of the first polypeptide and the antigen comprised in the further antigenic unit of the further polypeptide are capable of oligomerizing due to the presence of oligomerization units. In some instances, oligomerization units on the first polypeptide and second polypeptide may be required for the second polypeptide to efficiently oligomerize with the antigen on the first polypeptide. These oligomerization units may be heterooligomerization units such as heterotrimerization units.

FIG. 17: Embodiment of the disclosure

FIG. 17 illustrates a construct of the disclosure, comprising a nucleic acid sequence encoding a first polypeptide and a second polypeptide. The first polypeptide comprises a targeting unit (TU, arrows and lines in white), a dimerization unit (DimU), an unit linker (UL), and an antigenic unit comprising an antigen A (AntA, light grey). The second polypeptide comprises a sequence encoding an antigenic unit comprising an antigen B (AntB, dark grey) and an interaction unit (IU), here a self-assembly domain. Upon expression, antigen B self-assembles into a nanoparticle.

FIG. 18: Embodiment of the disclosure

FIG. 18 illustrates a construct of the disclosure, comprising a nucleic acid sequence encoding a first polypeptide and a second polypeptide. The first polypeptide comprises a first targeting unit (TU1, white arrows and lines, e.g. CCL3L1), a dimerization unit (DimU), an unit linker (UL), and an antigenic unit comprising T cell epitopes. The T cell epitopes may optionally be separated by subunit linkers (not shown). The second polypeptide comprises a sequence encoding a second targeting unit (TU2, light grey arrows, e.g. an scFv targeting MHCII) and a further antigenic unit comprising an antigen (AntA). Upon expression, the first polypeptide will induce strong T cell responses, while the second polypeptide will target the antigen to MHCII and induce functional antibodies. The second polypeptide may optionally comprise an unit linker (UL) between the second targeting unit and the further antigenic unit.

FIG. 19: SARS-COV-2 protein constructs

Schematic representation of post-translational attachment SARS-COV-2 protein constructs TECH011-IV003 and TECH011-IV004.

FIG. 20: SARS-COV-2 protein constructs

Schematic representation of heterodimer SARS-COV-2 protein constructs expressed from TECH011-IV005, TECH011-IV006, TECH011-IV007, TECH018-IV001 and TECH018-IV002.

FIG. 21: SARS-COV-2 protein constructs

Schematic representation of SARS-COV-2 protein constructs (first polypeptides with antigenic units comprising a different number of T cell epitopes and further polypeptides comprising RBD) expressed from TECH021-IV001, TECH021-IV018, TECH021-IV019 and TECH021-IV020.

FIG. 22: Expression and secretion level of SARS-COV-2 protein constructs

SARS-COV-2 construct protein expression and secretion levels of the polypeptides encoded by DNA plasmids TECH011-IV003, TECH011-IV004, TECH011-IV005, TECH011-IV006, TECH011-IV007, TECH018-IV001 and TECH018-IV002 detected in the supernatant of Expi293F cells transfected with said DNA plasmids by using anti-CCL3/MIP1a antibody and anti-SARS-COV-2 RBD antibody in the enzyme-linked immunosorbent assay (ELISA). Supernatant of cells transfected only with Expifectamine (Expifect) was used as negative controls.

FIG. 23: Expression of SARS-COV-2 protein constructs

Secretion levels of SARS-COV-2 RBD (Wuhan variant) encoded by DNA plasmids TECH011-IV003, TECH011-IV004, TECH011-IV005, TECH011-IV006, TECH011-IV007, TECH018-IV001 and TECH018-IV002 detected in the supernatant of Expi293F cells transfected with said DNA plasmids by the enzyme-linked immunosorbent assay (ELISA).

FIG. 24: Expression of SARS-COV-2 protein constructs

Protein expression encoded by DNA plasmids TECH011-IV003, TECH011-IV004 and TECH011-IV007 detected in the supernatant of Expi293F cells transfected with said DNA plasmids. Both reduced and non-reduced samples are shown. Supernatant of cells transfected only with Expifectamine (ExpiFect) were used as negative control.

FIG. 25: Expression of SARS-COV-2 protein constructs

RBD protein expression encoded by DNA plasmids TECH011-IV003, TECH011-IV004 (diluted 1:50) and TECH011-IV007 detected in the supernatant of Expi293F cells transfected with said DNA plasmids. Both reduced and non-reduced samples are shown. Supernatant of cells transfected only with Expifectamine (ExpiFect) were used as negative control.

FIG. 26: Expression and secretion level of SARS-COV-2 protein constructs

Protein expression and secretion levels of the polypeptides encoded by DNA plasmids TECH021-IV001 and TECH021-IV018 detected in the supernatant of Expi293F cells transfected with said DNA plasmids by using anti-CCL3/MIP1a antibody and anti-CH3 antibody in the enzyme-linked immunosorbent assay (ELISA). Supernatant of cells transfected only with Expifectamine (Expifect) was used as negative controls.

FIG. 27: Expression of SARS-COV-2 protein constructs Protein expression encoded by DNA plasmids TECH021-IV001 and TECH021-IV018, detected by anti-CCL3/MIP1a antibody under reducing conditions, in WB in the supernatant of Expi293F cells transfected with said DNA plasmids. Supernatant of cells transfected only with Expifectamine (ExpiFect) was used as negative control.

FIG. 28: Expression of SARS-COV-2 protein constructs

Protein expression encoded by DNA plasmids TECH021-IV001 and TECH021-IV018, detected by anti-SARS-COV-2 RBD antibody under reducing conditions, in WB in the supernatant of Expi293F cells transfected with said DNA plasmids. Supernatant of cells transfected only with Expifectamine (ExpiFect) were used as negative control.

FIG. 29: Expression and secretion level of SARS-COV-2 protein constructs

Protein expression and secretion levels of the polypeptides encoded by DNA plasmid TECH021-IV020 detected in the supernatant of Expi293F cells transfected with said DNA plasmids by using anti-CCL3/MIP1a antibody and anti-CH3 antibody in the enzyme-linked immunosorbent assay (ELISA). Supernatant of cells transfected only with Expifectamine (Expifect) was used as negative controls.

FIG. 30: Expression of SARS-COV-2 protein constructs

Protein expression encoded by DNA plasmid TECH021-IV020 detected by anti-CCL3/MIP1a antibody under reducing conditions, in WB in the supernatant of Expi293F cells transfected with said DNA plasmids. Supernatant of cells transfected only with Expifectamine (ExpiFect) was used as negative control.

FIG. 31: Expression of SARS-COV-2 protein constructs

Protein expression encoded by DNA plasmid TECH021-IV020 detected by anti-SARS-COV-2 RBD antibody under reducing conditions, in WB in the supernatant of Expi293F cells transfected with said DNA plasmids. Supernatant of cells transfected only with Expifectamine (ExpiFect) were used as negative control.

FIG. 32: Expression and secretion levels of SARS-COV-2 protein constructs

Protein expression and secretion levels of the polypeptides encoded by DNA plasmid TECH023-IV003 detected in the supernatant of Expi293F cells transfected with said DNA plasmids by using anti-CCL3/MIP1a antibody and anti-CH3 antibody in the enzyme-linked immunosorbent assay (ELISA). Supernatant of cells transfected only with Expifectamine (Expifect) was used as negative controls.

FIG. 33: Expression of SARS-COV-2 protein constructs

Protein expression encoded by DNA plasmid TECH023-IV003 detected by anti-CCL3/MIP1a antibody under reducing conditions, in WB in the supernatant of Expi293F cells transfected with said DNA plasmids. Supernatant of cells transfected only with Expifectamine (ExpiFect) were used as negative control.

FIG. 34: Expression of SARS-COV-2 protein constructs

Protein expression encoded by DNA plasmid TECH023-IV003 detected by anti-SARS-COV-2 RBD antibody under reducing conditions, in WB in the supernatant of Expi293F cells transfected with said DNA plasmids. Supernatant of cells transfected only with Expifectamine (ExpiFect) were used as negative control.

FIG. 35: SARS-COV-2 constructs induce T cell response

Cellular immunogenicity of DNA plasmids TECH011-IV004 (leucine zipper with signal peptide), TECH021-IV001 (free RBD as further polypeptide) and TECH023-IV003 (separately secreted, targeted antigen) against the SARS COV-2 T cell epitopes pep8, pep18 and pep25 and the RBD antigen in mice vaccinated with these plasmids by measuring the IFN-γ secretion from T cells (total T cell response), compared to the negative control VB1026. The splenocytes from immunized mice were re-stimulated with peptides corresponding to the plasmid encoding antigens either as single peptides (pep8, pep18, pep25) or as pools of 15-mer peptides with 3 aa overlapping sequence to span the full RBD protein.

FIG. 36: SARS-COV-2 constructs induce humoral immune response

Humoral immunogenicity of DNA plasmids TECH011-IV004 (Leucine Zipper with signal peptide), TECH021-IV001 (RBD as further polypeptide) and TECH023-IV003 (Separately secreted, targeted antigen) against the SARS COV-2 T cell epitopes pep8, pep18 and pep25 and RBD peptides pools in mice vaccinated with these plasmids by measuring the RBD-specific total IgG endpoint titers, compared to the negative control VB1026.

FIG. 37: RSV protein constructs

Schematic illustration of a dimeric protein formed by 2 first polypeptides of IV12 (targeting unit hCCL3L1), IV58 (targeting unit anti-mouse MHCII I-E scFv) and IV59 (targeting unit anti-mouse CD40 scFv), wherein the PreF protein of the first polypeptide and the further polypeptide (same PreF protein) form a trimer.

FIG. 38: RSV protein constructs

Schematic illustration of the dimeric proteins formed by 2 first polypeptides expressed from IV71-IV74 (T cell construct), and further polypeptides (soluble or membrane-bound PreF proteins) expressed from each of IV71-IV74.

FIG. 39: RSV protein constructs

Schematic illustration of the dimeric proteins formed by 2 first polypeptides expressed from IV082-IV085 and further polypeptides (PreF proteins) which comprise a ferritin sequence and form nanoparticles, once expressed.

FIG. 40: Expression of first polypeptide of RSV protein constructs

FIG. 40 shows the amount of first polypeptide present in the supernatant of Expi293F cells transfected with DNA plasmids IV12, IV58 and IV59 as determined by ELISA.

FIG. 41: Expression of RSV protein constructs

FIG. 41 shows in a Western Blot the proteins expressed by DNA plasmids IV12, IV58 and IV59 and detected in the supernatant of Expi293F cells transfected with said DNA plasmids. Both reduced and non-reduced samples are shown. Supernatant of Expi293F cells transfected only with Expifectamine (ExpiFect) was used as negative controls.

FIG. 42: RSV constructs induce T cell response

FIG. 42 shows the T cell responses induced in mice by administration of DNA plasmid IV12 determined by the IFN-γ secretion from T cells (total T cell response). PBS was included as negative control.

FIG. 43: RSV constructs induce humoral response

FIG. 43 shows the humoral response induced in mice by administration of DNA plasmid IV12 determined by measuring the PreF total IgG endpoint titers PBS was included as negative control.

FIG. 44: Expression of further polypeptide of RSV protein constructs

FIG. 44 shows the amount of further polypeptide (soluble and membrane-bound PreF protein) present in the supernatant of Expi293F cells transfected with DNA plasmids IV71-IV72 as determined by ELISA.

FIG. 45: Expression of first polypeptide of RSV protein constructs

FIG. 45 shows the amount of first polypeptide present in the supernatant of Expi293F cells transfected with DNA plasmids IV71-IV74 as determined by ELISA. Supernatant of Expi293F cells transfected only with Expifectamine (ExpiFect) was used as negative controls.

FIG. 46: Expression of further polypeptides of RSV protein constructs

FIG. 46 shows in a Western Blot (reduced) the further polypeptides expressed by DNA plasmids IV71-IV74 and detected in the supernatant (IV71 and IV72) or lysate (IV73 and IV74) of Expi293F cells transfected with said DNA plasmids. Recombinant PreF protein was used as positive control and and Expifectamine (ExpiFect) as negative controls.

FIG. 47: Expression of first polypeptide of RSV protein constructs

FIG. 47 shows in a Western Blot (reduced/non-reduced) the first polypeptides expressed by DNA plasmids IV71-IV74 and detected in the supernatant of Expi293F cells transfected with said DNA plasmids. Recombinant PreF protein was used as positive control and Expifectamine (ExpiFect) as negative controls.

FIG. 48: Detection of membrane-bound PreF antigen from RSV protein constructs

FIG. 48 shows the detection of membrane-bound PreF antigen on the surface of Expi293F cells transfected with either DNA plasmid IV73 or DNA plasmid IV74 using Motavizumab (A) and D25 Fab (B) antibodies. Expi293F cells transfected with DNA plasmids IV71 or IV72 and Expifectamine (ExpiFect) only were included as negative controls.

FIG. 49: RSV constructs induce T cell response

FIG. 49 shows the T cell responses induced in mice by administration of DNA plasmids IV71-IV74 determined by the IFN-γ secretion from T cells (total T cell response). PBS was included as negative control, DS-Cav1 was included as positive control.

FIG. 50: RSV constructs induce T cell response

FIG. 50 shows the T cell responses induced in mice by administration of DNA plasmids IV71-IV74 determined by measuring the IFN-γ secretion from either depleted CD4⁺ T cells (corresponding to CD8+ T cell responses) or depleted CD8+ T cells (corresponding to CD4+ T cell responses). PBS was included as negative control, DS-Cav1 was included as positive control.

FIG. 51: RSV constructs induce humoral response

FIG. 51 shows the humoral responses induced in mice by administration of DNA plasmids IV71-IV74 determined by measuring the PreF total IgG endpoint titers, PBS was included as negative control, DS-Cav1 was included as positive control. Statistical analysis was done using the Kruskal-Wallis one-way analysis of variance. * p<0.05, ** p<0.01.

FIG. 52: RSV constructs induce neutralization antibodies

FIG. 52 shows neutralization titers of the antibodies present in the sera of mice administered with DNA plasmids IV71-IV74 determined by incubation of antibodies induced by the administration and measuring their later infectivity in Vero cells. Sera of mice administered with either PreF DS-Cav1 protein or palivizumab antibody were used as positive controls and sera of mice administered with PBS were used as negative controls.

FIG. 53: Intracellular and secreted amount of first polypeptide of RSV protein constructs

FIG. 53 shows the amount of first polypeptide present in the supernatant of Expi293F cells transfected with DNA plasmids IV082-IV083 (FIG. 53A) or DNA plasmids IV084-IV085 (FIG. 53B) as determined by ELISA.

FIG. 54: Expression and secretion levels of further polypeptides of RSV protein constructs

FIG. 54 shows the expression and secretion levels of the further polypeptides encoded by DNA plasmids IV084 and IV085 detected in the supernatant of Expi293F cells transfected with said DNA plasmids by ELISA Supernatant of cells transfected with Expifectamine (Expifect) were used as negative control. Darker bars: 1:500 dilution, lighter bars: 1:1000 dilution.

FIG. 55: Expression of first polypeptide of RSV protein constructs

FIG. 55 shows in a Western Blot (reduced) the first polypeptides expressed by DNA plasmids IV082-IV085 and detected in the supernatant of Expi293F cells transfected with said DNA plasmids. Supernatant of cells transfected with Expifectamine (Expifect) were used as negative control.

FIG. 56: Expression of further polypeptides of RSV protein constructs

FIG. 56 shows in a Western Blot (reduced) the further polypeptides expressed by DNA plasmids IV082-IV085 and detected in the supernatant of Expi293F cells transfected with said DNA plasmids. Supernatant of cells transfected with Expifectamine (Expifect) were used as negative control.

FIG. 57: Expression of further polypeptides of RSV protein constructs

FIG. 57 shows in a Western Blot from Native PAGE the further polypeptides expressed by DNA plasmids IV082-IV085 and detected in the supernatant of Expi293F cells transfected with said DNA plasmids. Supernatant of cells transfected with DNA plasmids only encoding the further polypeptides and no first polypeptides (IV080 and IV081, respectively) were included as positive controls and supernatant of cells transfected only with Expifectamine (Expifect) was used as negative control.

FIG. 58: Embodiment of the disclosure

FIG. 58 illustrates an embodiment of the disclosure, particularly for tolerance-inducing constructs, wherein the first nucleic acid encodes a polypeptide comprising a targeting unit (TU), a dimerization unit (DimU) and an antigenic unit comprising 2 T cell epitopes (T1, T2), which are separated by a T cell epitope linker (TL), and one further nucleic acid sequence that encodes a further polypeptide comprising a further antigenic unit comprising a hypoallergenic allergen (HA). A 2A self-cleaving peptide (2A) allows the co-expression of the first polypeptide and said further polypeptide.

FIG. 59: Embodiment of the disclosure

FIG. 59 illustrates an embodiment of the disclosure, particularly for tolerance-inducing constructs, wherein the first nucleic acid encodes a polypeptide comprising a targeting unit (TU), a dimerization unit (DimU) and an antigenic unit comprising 2 T cell epitopes (T1, T2), which are separated by a T cell epitope linker (TL), and one further nucleic acid sequence that encodes a further polypeptide comprising a further targeting unit (TU2) and a further antigenic unit comprising a hypoallergenic allergen (HA). A 2A self-cleaving peptide (2A) allows the co-expression of the first polypeptide and said further polypeptide.

FIG. 60: Embodiment of the disclosure

FIG. 60 illustrates an embodiment of the disclosure, particularly for tolerance-inducing constructs, wherein the first nucleic acid encodes a polypeptide comprising a targeting unit (TU), a first dimerization unit (DimU) and an antigenic unit comprising 2 T cell epitopes (T1, T2), which are separated by a T cell epitope linker (TL), and one further nucleic acid sequence that encodes a further polypeptide comprising further dimerization unit (DimU2), a further targeting unit (TU2), and a further antigenic unit comprising a hypoallergenic allergen (HA). A 2A self-cleaving peptide (2A) allows the co-expression of the first polypeptide and said further polypeptide. The first dimerization unit (DimU) and further dimerization unit (DimU2) may be heterodimerization units, which promote the formation of heterodimers of first and further polypeptide. Alternatively, the first and further dimerization unit do not promote the formation of heterodimers, i.e. 2 different homodimers are formed: a homodimer consisting of 2 first polypeptides and another homodimer consisting of 2 further polypeptides.

FIG. 61: Expression of first polypeptide of RSV protein constructs

FIG. 61 shows the expression and secretion levels of the first polypeptides encoded by mRNA constructs IV009 and IV010 detected by ELISA in the supernatant of Expi293F cells transfected with said mRNA constructs at 6, 24, 48 and 72 hours post transfection. Results representative of two independent experiments are shown

FIG. 62: Expression of further polypeptides of RSV protein constructs

FIG. 62 shows the expression and secretion levels of the further polypeptides encoded by mRNA constructs IV009 and IV010 detected by ELISA in the supernatant of Expi293F cells transfected with said mRNA constructs at 6, 24, 48 and 72 hours post transfection. Results representative of two independent experiments are shown

FIG. 63: Expression of first polypeptide of RSV protein constructs

FIG. 63 shows in a Western Blot (reduced) the first polypeptides encoded by mRNA constructs IV009 and IV010 and detected by ELISA in the supernatant of Expi293F cells transfected with said mRNA constructs at 6, 24, 48 and 72 hours post transfection. Supernatant of cells transfected only with Expifectamine (Transfection) were used as negative controls. The triangle indicates the expected monomer band (53 kDa) of the first polypeptide.

FIG. 64: RSV constructs induce T cell response

FIG. 64 shows the T cell responses induced in mice by administration of mRNA constructs IV009 and IV010 determined by the IFN-γ secretion from T cells (total T cell response). T cell responses of mice administered with PBS were included as negative controls.

FIG. 65: RSV constructs induce humoral response

FIG. 65 shows the humoral responses induced in mice by administration of mRNA constructs IV009 and IV010 determined by measuring the half maximal effective concentration (EC50) of PreF total IgG. Sera of mice administered with PBS and DS-Cav1 were included as negative and positive controls, respectively.

### DETAILED DESCRIPTION

The first polypeptide and/or the multimeric protein will herein also be referred to as a “construct”. The first polypeptides/multimeric proteins described herein are generally immunogenic constructs or tolerance-inducing constructs. FIG. 4 illustrates certain embodiments of such immunogenic and tolerance-inducing constructs, which comprises a targeting unit targeting APCs, a multimerization unit, and an antigenic unit comprising one or more epitopes; the construct may in some embodiments comprise an unit linker.

An “immunogenic construct” is one that elicits an immune response, particularly when administered to a subject in a form suitable for administration and in an amount effective to elicit the immune response (i.e., an immunologically effective amount).

A “tolerance-inducing construct” is one that does not elicit an immune response, such as an inflammatory immune response, but rather does induce tolerance, when administered to a subject in a form suitable for administration and in an amount effective to induce tolerance (i.e. an effective amount). Tolerance may be induced towards the T cell epitopes comprised in the antigenic unit and/or as an allergen-specific IgG response which inhibits the binding of IgE antibodies to allergens, without inducing an IgE immune response.

The terms “at least one further polypeptide”, and “a further polypeptide” are used interchangeably herein to refer to any one of the one or more further polypeptides encoded by the one or more further nucleic acid sequences comprised in the vectors of the disclosure. Throughout the disclosure, embodiments may be exemplified where the further polypeptide is a single (second) polypeptide—in other embodiments however the further polypeptide may be several further polypeptides, as will readily be understood by the skilled person.

In the context of structural elements of the vector of the present disclosure, the term “polypeptide” refers to the first polypeptide and/or any one of the one or more further polypeptides, if not explicitly specified otherwise. Similarly, the term “nucleic acid” refers to the first nucleic acid and/or at least one further nucleic acid, if not explicitly specified otherwise.

A “subject” is an animal, e.g., a mouse, or a human, preferably a human. The terms “mouse”, “murine” and “m” are used interchangeably herein to denote a mouse or refer to a mouse. The terms human and “h” are used interchangeably herein to denote a human or refer to a human. A subject may be a patient, i.e., a human suffering from a disease and who is in need of a therapeutic treatment, or it may be a subject in need of prophylactic treatment, e.g., in need of prevention from being infected with an infectious disease, or it may be a subject suspected of suffering from a disease, or e.g., in need of prevention from developing an autoimmune disease, an allergy or graft rejection, or it may be a subject suspected of suffering from an allergic disease, autoimmune disease or graft rejection. The terms “subject” and “individual” are used interchangeably herein.

A “disease” is an abnormal medical condition that is typically associated with specific signs and symptoms in a subject being affected by the disease.

An “infectious disease” is a disease caused by one or more pathogens, including viruses, bacteria, fungi and parasites.

An “allergic disease” refers to a number of conditions caused by hypersensitivity of the immune system to normally harmless substances in the environment and is typically associated with specific signs and symptoms in a subject being affected by the allergic disease.

A “cancer” refers to a broad group of various diseases characterized by the uncontrolled growth of abnormal cells in the body. A “cancer” or “cancer tissue” includes a tumor, and as used herein, encompasses both a solid tumor as well as tumor cells found in a bodily fluid such as blood, and includes metastatic cancer. Unregulated cell division and growth results in the formation of malignant tumors that can invade neighboring tissues and can also metastasize to distant parts of the body through the lymphatic system or bloodstream. Following metastasis, the distal tumors can be said to be “derived from” a pre-metastasis tumor.

The term “tolerance” as used herein refers to a decreased level of an immune response, such as an inflammatory immune response, a delay in the onset or progression of an immune response, such as an inflammatory immune response, and/or a reduced risk of the onset or progression of an immune response, such as an inflammatory immune response.

The term “tolerance-inducing universal T helper cell epitopes” refers to epitopes that may be highly promiscuous, but not necessarily universal binders to all human HLA. Examples of tolerance-inducing universal T helper cell epitopes include T regulatory epitopes (Tregitopes), inhibitory epitopes and apitopes (antigen-processing-independent epitopes). An additional example may comprise certain dominant autoepitopes from nucleosomal histones that may be cross-reactively recognized by autoimmune Th cells, as well as B cells, and that can be promiscuously presented in the context of diverse MHC class II alleles (Hee-Kap Kang 2007). A further example may include epitopes of peptides that share a consensus motif across individuals and species that may be presented by MHC class I to activate cross-reactive CD8+ T regs to induce tolerance and suppress allogeneic responses (Elodie Picarda 2019).

A “treatment” is a prophylactic treatment or a therapeutic treatment.

A “prophylactic treatment” is a treatment administered to a subject who does not (or not yet) display signs or symptoms of, or displays only early signs or symptoms of, a disease, such that treatment is administered for the purpose of preventing or decreasing the risk of developing the disease and/or symptoms associated with the disease. A prophylactic treatment functions as a preventative treatment against a disease, or as a treatment that inhibits or reduces further development or enhancement of the disease and/or its associated symptoms. The terms prophylactic treatment, prophylaxis and prevention are used interchangeably herein.

A “therapeutic treatment” is a treatment administered to a subject who displays symptoms or signs of a disease, in which treatment is administered to the subject for the purpose of diminishing or eliminating those signs or symptoms or for the purpose of delaying or stopping disease progression.

An “epitope” as used herein refers to a site on an antigen to which B and/or T cells respond. B cell epitopes can be formed both from contiguous amino acids or non-contiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation.

A “T cell epitope” as used herein refers to a discrete, single T cell epitope. A “B cell epitope” as used herein refers to a discrete, single B cell epitope.

A “B cell conformational epitope” is an epitope comprising noncontiguous amino acids juxtaposed by tertiary folding of a protein.

A “nucleotide sequence” is a sequence consisting of nucleotides. The terms “nucleotide sequence” and “nucleic acid sequence” are used interchangeably herein.

The terms “multiple”, “a plurality” and “several” are used herein interchangeably with “more than one”.

In some embodiments, the one or more further polypeptides interacts with the first polypeptide/multimeric protein. The interaction may be a direct interaction, i.e., the further polypeptide interacts physically with the first polypeptide/multimeric protein, or an indirect interaction, i.e., the further polypeptide upon expression causes a change in some properties of the first polypeptide/multimeric protein. In some embodiments, at least one of the one or more further polypeptides forms an oligomer or a multimer with the first polypeptides/multimeric protein, such as a multimer with the antigenic unit of the first polypeptide. In other embodiments, at least one of the further polypeptides is capable of oligomerizing or multimerising itself, and is expressed as an oligomer or multimer.

The advantage of the present disclosure is that by co-expressing the first polypeptide and the at least one further polypeptide from a single vector, e.g., a DNA plasmid, only such single vector needs to be administered to a subject. Hence, for instance for interacting with the first polypeptide/multimeric protein, it is not required to produce and administer additional vectors encoding further polypeptides or to co-administer such compounds in the form of proteins or peptides, thereby reducing the production costs and streamlining drug production. Administration of a single drug product may also contribute to increased patient acceptance of therapy and make handling of the drug product, e.g., reconstitution and administration to the patient, easier for health care professionals.

The section headings used herein are for organizational purposes only and are not to be construed as limiting the subject matter described.

### Vector

Herein are disclosed vectors comprising a first nucleic acid sequence encoding a first polypeptide and a second (or several further) nucleic acid sequence(s) encoding a second (or several further) polypeptides. The first polypeptide in some embodiments comprises a targeting unit targeting APCs, a multimerization unit such as a dimerization unit, and an antigenic unit comprising one or more epitopes; said epitopes may be T cell epitopes of an allergen, self-antigen or alloantigen. The second (or further) polypeptide also comprises a further antigenic unit comprising one or more further epitopes. The first polypeptide and the further polypeptides, when expressed in a cell after introduction of the single vector encoding them, are expressed as separate molecules, and each triggers an immune response; or, in embodiments where the antigenic unit comprises T cell epitopes of an allergen, self-antigen or alloantigen, the first and the further polypeptide(s) are each capable of inducing tolerance towards said allergen, self-antigen or alloantigen. Administration of a single vector of the present disclosure thus may enable several types of immune responses, e.g., immune responses against different targets.

The vectors of the disclosure may be any molecules which are suitable to carry foreign nucleic acid sequences, such as DNA or RNA, into a cell, where they can be expressed, i.e., expression vectors.

In some embodiments, the vector is a DNA vector, such as a DNA plasmid, DNA amplicons, or a DNA viral vector, such as a DNA viral vector selected from the group consisting of adenovirus, vaccinia virus, adeno-associated virus, cytomegalovirus and Sendai virus.

In other embodiments, the vector is an RNA vector, such as an RNA plasmid or an RNA viral vector, such as a retroviral vector, e.g., a retroviral vector selected from the group consisting of alphavirus, lentivirus, Moloney murine leukemia virus and rhabdovirus.

In some preferred embodiments, the vector is a DNA vector, more preferably a DNA plasmid.

### DNA Plasmids

A plasmid is a small, extrachromosomal DNA molecule within a cell that is physically separated from chromosomal DNA and can replicate independently. Plasmids are mostly found as small circular, double-stranded DNA molecules in bacteria; however, plasmids are sometimes present in archaea and eukaryotic organisms. Artificial plasmids are widely used as vectors in molecular cloning, serving to deliver and ensure high expression of recombinant DNA sequences within host organisms. Plasmids comprise several important features, including a feature for selection of cells comprising the plasmid, such as for example a gene for antibiotic resistance, an origin of replication, a multiple cloning site (MCS) and promoters for driving the expression of the inserted gene(s) of interest.

Generally, promoters are sequences capable of attracting initiation factors and polymerases to the promoter, so that a gene is transcribed. Promoters are located near the transcription start sites of genes, upstream on the DNA. Promoters can be about 100-1000 base pairs long. The nature of the promoter is usually dependent on the gene and product of transcription and type or class of RNA polymerase recruited to the site. When the RNA polymerase reads the DNA of the plasmid, a messenger RNA (mRNA) molecule is transcribed. After processing, the mRNA will be able to be translated numerous times, and thus result in many copies of the proteins encoded by the genes of interest, when the ribosome translates the mRNA into protein. Generally, the ribosome facilitates decoding by inducing the binding of complementary transfer RNA (RNA) anticodon sequences to mRNA codons. The tRNAs carry specific amino acids that are chained together into a polypeptide as the mRNA passes through and is “read” by the ribosome. Translation proceeds in three phases, initiation, elongation and termination. Following the translation process, the polypeptide folds into an active protein and performs its functions in the cell or is exported from the cell and performs its functions elsewhere, sometimes after a considerable number of posttranslational modifications.

When a protein is destined for export out of the cell, a signal peptide directs the protein into the endoplasmic reticulum, where the signal peptide is cleaved off and the protein is transferred to the cell periphery after translation has terminated.

The DNA plasmid of the present disclosure is not limited to any specific plasmid, the skilled person will understand that any plasmid with a suitable backbone can be selected and engineered by methods known in the art to comprise the elements and units of the present disclosure.

### Tolerance-Inducing Vectors

In some embodiments, particularly for tolerance-inducing DNA constructs, the DNA constructs are designed in such a way that they do not comprise stimulatory CpG motifs or that the number of stimulatory CpG motifs is minimized. Such motifs can be identified and the sequence of the DNA construct can be optimized accordingly by a pattern matching algorithm. In particular, the DNA constructs preferably do not comprise any CpG-S motifs (where S is C or G), or it comprises a reduced amount of CpG-S motifs compared to the natural and/or unmodified sequence. For instance, the DNA constructs do not comprise one or more of the following motifs:

1.
ApACpGGTpT;
2.
ApACpGCTpT;
3.
GpACpGGTpT;
4.
GpACpGCTpT;
5.
GpTCpGGTpT;
6.
GpTCpGCTpT,

or comprise a reduced number of the above sequences compared to the non-optimized DNA.

Reducing the number of such stimulatory CpG motifs in the DNA constructs may promote tolerance.

Conversely, particularly for tolerance-inducing DNA constructs, such constructs may comprise CpG motifs that antagonize the effects of stimulatory CpG motifs. For instance, the DNA constructs may comprise one or more of the following motifs:

```
  7.
CCpG;
  8.
CpGG;
  9.
CpGCpGCpG;
10.
CpG repeats.
```

Including such CpG motifs that antagonize the effects of stimulatory CpG motifs in the DNA constructs may promote tolerance.

In some embodiments, particularly for tolerance-inducing DNA constructs, the constructs comprise no CpG-S motifs, or a reduced number of such motifs compared to the non-optimized DNA sequence, and/or comprise motifs antagonizing the effects of the stimulatory CpG motifs as described above.

Modifying the motifs as described above can be done as is known in the art. The above modifications may be included anywhere in the DNA plasmid, such as any of the further polynucleotides and/or any of the additional polynucleotides described herein and/or in the plasmid backbone.

### Co-expression

The vectors of the present disclosure co-express several polypeptides. Such vectors (and plasmids) are also referred to as multicistronic or polycistronic vectors (and multicistronic or polycistronic plasmids). The skilled person knows how to engineer a vector to comprise sequences coding for these several polypeptides and can select different means and use different techniques known in the art to ensure that these proteins are co-expressed from one vector as separate proteins, as also detailed further below.

Hence, the skilled person can construct the vectors of the disclosure, co-expressing different polypeptides, i.e., a first polypeptide and one or more further polypeptides.

In some preferred embodiments, the vectors of the disclosure comprise one or more co-expression elements, i.e., nucleic acid sequences which allow for the co-expression of the first polypeptide and the one or more further polypeptides from the same vector.

In some embodiments, different types of co-expression elements are used if more than one further polypeptide is expressed from the vector of the disclosure.

In other embodiments, the same types of co-expression elements are used if more than one further polypeptide is expressed from the vector of the disclosure.

In some embodiments, the vector comprises a co-expression element (or more than one co-expression elements), which causes transcription of the first polypeptide and the one or more further polypeptides on a single transcript but their independent translation into separate polypeptides. Hence, the presence of the co-expression element results in a final production of separate translation products.

Thus, in some embodiments at least one of the one or more co-expression element causes the transcription of the first polypeptide and of the one or more further polypeptides on a single transcript and their independent translation into a separate first polypeptide and separate one or more further polypeptides.

In some embodiments at least one of the one or more co-expression element causes the transcription of the first polypeptide and of the one or more further polypeptides on a single transcript and their translation into a separate first polypeptide and separate one or more further polypeptides.

In some embodiments, at least one of the one or more co-expression elements promotes the transcription of the first polypeptide and of the one or more further polypeptides as separate transcripts.

In some embodiments, the one or more co-expression elements are internal ribosome entry site (IRES) elements, nucleic acid sequences encoding 2A self-cleaving peptides, bidirectional promoters, or multiple promoters, for example as described further below.

In some embodiments, the one or more co-expression elements are IRES elements, or nucleic acid sequences encoding 2A self-cleaving peptides.

In some embodiments, the one or more co-expression elements are a) bidirectional promoters or b) promoters, and the vector comprises a separate bidirectional promoter or separate promoter for each of the nucleic acid sequences encoding polypeptides.

### IRES

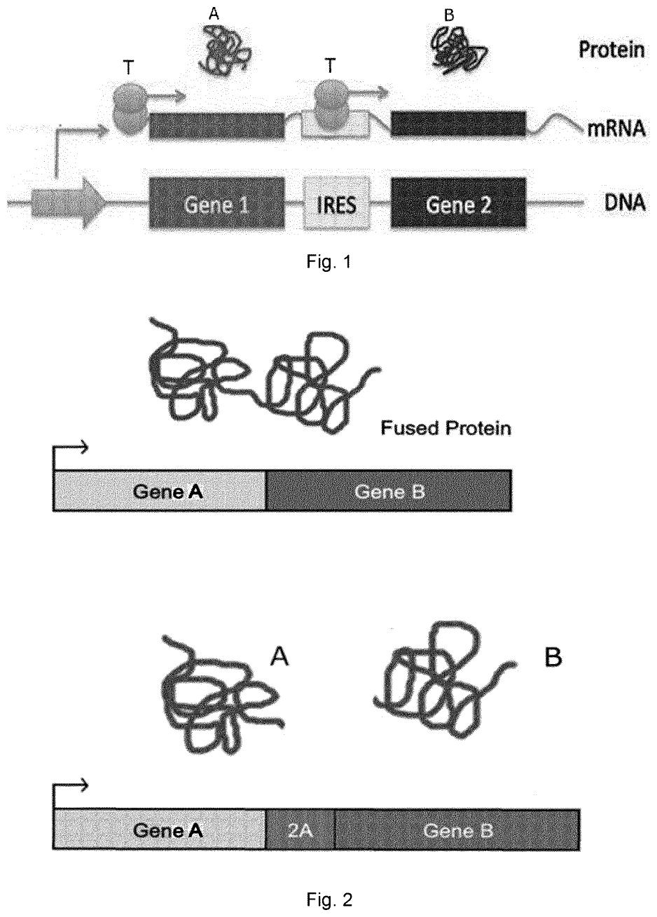

In some embodiments of the present disclosure, at least one of the co-expression elements is an internal ribosome entry site, abbreviated IRES, element, the concept of which is illustrated in FIG. 1. An IRES is an RNA element that allows for translation initiation in a cap-independent manner, as part of the greater process of protein synthesis. In eukaryotic translation, initiation typically occurs at the 5′ end of mRNA molecules, since 5′ cap recognition is required for the assembly of the initiation complex. By placing an IRES element between two coding regions, the initiation complex can be assembled at this site and allow for translation of the downstream coding region. Hence, in an embodiment of the present disclosure, the vector comprises an IRES and one single transcript is produced from the vector, which transcript subsequently is translated into separate proteins.

The IRES element allows the co-expression of the first polypeptide and the one or more further polypeptides under the control of the same promoter. The promoter directs the transcription of a single mRNA containing coding regions for the nucleic acid sequence encoding the first polypeptide and the nucleic acid sequences encoding the one or more further polypeptides. If more than one further polypeptide is expressed from the vector of the disclosure, an IRES element (or another co-expression element as known in the art or as described herein) needs to be present in the vector of the disclosure upstream of each nucleic acid sequence encoding a further polypeptide. Alternatively, another type of co-expression element may be used if more than one further polypeptide is expressed from the vector of the disclosure.

The IRES elements for use in the vectors of the disclosure may be derived from viral genomes or from cellular mRNA. Vectors comprising IRES elements, such as DNA plasmids, are commercially available.

### 2A Self-Cleaving Peptides

In some embodiments of the present disclosure, at least one co-expression element is a nucleic acid sequence encoding a 2A self-cleaving peptide (or short “2A peptide”), the concept of which is illustrated in FIG. 2.

In the context of this disclosure, the terms “2A self-cleaving peptide” and “2A peptide” are used for a peptide encoded by a nucleic acid sequence that, when positioned between two coding regions, causes the transcription of the two coding regions as a single transcript, followed by translation of the single transcript into two separate peptide chains, each corresponding to a coding region. Generally, when the ribosome translates mRNA, amino acids are covalently bonded in an N-terminal to C-terminal fashion. The presence of a nucleic acid sequence encoding a 2A self-cleaving peptide results in two separate peptide chains because the ribosome skips the synthesis of a peptide bond at the C-terminus of the 2A peptide. 2A self-cleaving peptides are typically 18-22 amino acids long and often comprise the consensus sequence DXEXNPGP (SEQ ID NO: 1), wherein X can be any amino acid. Examples of 2A peptides include P2A, E2A, F2A and T2A.

In some embodiments of the present disclosure, the ribosome skips the peptide bond between a glycine and a proline residue found on the C-terminus of the 2A self-cleaving peptide, meaning that the upstream gene product will have a few additional amino acid residues added to the end, while the downstream gene product will start with a proline.

In some embodiments, the 2A self-cleaving peptide is an 18-22 amino acid long sequence comprising the consensus sequence DXEXNPGP (SEQ ID NO: 1), wherein X can be any amino acid.

Thus, also the 2A self-cleaving peptide allows for the co-expression of the first polypeptide and the one or more further polypeptides under the control of the same promoter. As with the IRES element, if more than one further polypeptide is expressed from the vector of the disclosure, a nucleic acid sequence encoding a 2A peptide (or another co-expression element as known in the art or as described herein) needs to be present in the vector upstream of each nucleic acid sequence encoding a further polypeptide. As an example, the vector comprises a first nucleic acid sequence encoding a first polypeptide, a second nucleic acid sequence encoding a first further polypeptide and a third nucleic acid sequence encoding a second further polypeptide. The vector may comprise a nucleic acid sequence encoding a T2A peptide between the first and the second nucleic acid sequence and a nucleic acid sequence encoding a P2A peptide between the second and the third nucleic acid sequence. Alternatively, another type of co-expression element may be used if more than one further polypeptide is expressed from the vector of the disclosure.

In some embodiments, the 2A self-cleaving peptide is a 2A-peptide selected from the group consisting of T2A peptide, P2A peptide, E2A peptide and F2A peptide.

In some embodiments, the T2A peptide has an amino acid sequence identical to those T2A sequences listed in Table 1 or 2. In some embodiments, the amino acid sequence DVEENPGP (SEQ ID NO: 2) is present but the remainder of the T2A amino acid sequence has 80% to 100% sequence identity to the T2A amino acid sequence of Table 1, such as 81%, 82%, 83%, 84%, 85%, 86%, 87%, 88%, 89%, 90%, 91%, 92%, 93%, 94%, 95%, 96%, 97%, 98% or 99% sequence identity. In some embodiments, the T2A peptide has the amino acid sequence with SEQ ID NO: 3.

In some embodiments, the P2A peptide has an amino acid sequence identical to those P2A sequences listed in Table 1 or 2. In some embodiments, the sequence DVEENPGP (SEQ ID NO: 2) is present but the remainder of the P2A amino acid sequence has 80% to 100% sequence identity to the P2A amino acid sequence of Table 1, such as 81%, 82%, 83%, 84%, 85%, 86%, 87%, 88%, 89%, 90%, 91%, 92%, 93%, 94%, 95%, 96%, 97%, 98% or 99% sequence identity. In some embodiments, the P2A peptide has the amino acid sequence with SEQ ID NO: 4.

In some embodiments, the E2A peptide has an amino acid sequence identical to those E2A sequences listed in Table 1 or 2. In a further embodiment, the sequence DVESNPGP (SEQ ID NO: 5) is present but the remainder of the E2A amino acid sequence has 80% to 100% sequence identity to the E2A amino acid sequence of Table 1, such as 81%, 82%, 83%, 84%, 85%, 86%, 87%, 88%, 89%, 90%, 91%, 92%, 93%, 94%, 95%, 96%, 97%, 98% or 99% sequence identity. In other embodiments, the E2A peptide has the amino acid sequence with SEQ ID NO: 6.

In some embodiments, the F2A peptide has an amino acid sequence identical to those F2A sequences listed in Table 1 or 2. In some embodiments, the sequence DVESNPGP (SEQ ID NO: 5) is present but the remainder of the F2A amino acid sequence has 80% to 100% sequence identity to the F2A amino acid sequence of Table 1, such as 81%, 82%, 81%, 82%, 83%, 84%, 85%, 86%, 87%, 88%, 89%, 90%, 91%, 92%, 93%, 94%, 95%, 96%, 97%, 98% or 99% sequence identity. In some embodiments, the F2A peptide has the amino acid sequence with SEQ ID NO: 7.

TABLE 1

| Name | Sequence | SEQ ID NO. |
|---|---|---|
| T2A | EGRGSLLTCGDVEENPGP | SEQ ID NO: 3 |
| P2A | ATNFSLLKQAGDVEENPGP | SEQ ID NO: 4 |
| E2A | QCTNYALLKLAGDVESNPGP | SEQ ID NO: 6 |
| F2A | VKQTLNFDLLKLAGDVESNPGP | SEQ ID NO: 7 |

It is generally known that the efficiency of the 2A-peptides can be modulated to increase their efficiency in cleavage and expression, for example by inserting a GSG sequence prior to the N-terminus of the wild-type sequences, as shown in Table 2.

TABLE 2

| Name | Sequence | SEQ ID NO. |
|---|---|---|
| T2A | GSGEGRGSLLTCGDVEENPGP | SEQ ID NO: 8 |
| P2A | GSGATNFSLLKQAGDVEENPGP | SEQ ID NO: 9 |
| E2A | GSGQCTNYALLKLAGDVESNPGP | SEQ ID NO: 10 |
| F2A | GSGVKQTLNFDLLKLAGDVESNPGP | SEQ ID NO: 11 |

In some embodiments, the vector of the disclosure contains both IRES elements and nucleic acid sequences encoding 2A peptides. As an example, the vector comprises a first nucleic acid sequence encoding a first polypeptide, a second nucleic acid sequence encoding a first further polypeptide and a third nucleic acid sequence encoding a second further polypeptide. The vector may comprise an IRES element between the first and the second nucleic acid sequence and a nucleic acid sequence encoding a 2A peptide between the second and the third nucleic acid sequence. Alternatively, the vector may comprise a nucleic acid sequence encoding a 2A peptide between the first and the second nucleic acid sequence and an IRES element between the second and the third nucleic acid sequence. Further nucleic acid sequences encoding further polypeptides may be included in the vector in the same manner.

In some embodiments, the vector of the disclosure contains nucleic acid sequences encoding two 2A peptides as a continuous sequence consisting of two 2A peptides. As an example, the vector comprises a first nucleic acid sequence encoding a first polypeptide and a second nucleic acid encoding a further polypeptide. The vector may comprise a nucleic acid sequence encoding two 2A peptides as a continuous sequence between the first and the second nucleic acid sequence.

### Bidirectional Promoters

In some embodiments of the present disclosure, the vector comprises a co-expression element (or more than one co-expression element) which causes that the first polypeptide and the one or more further polypeptides are transcribed as separate transcripts, which results in separate transcription products and thus separate proteins.

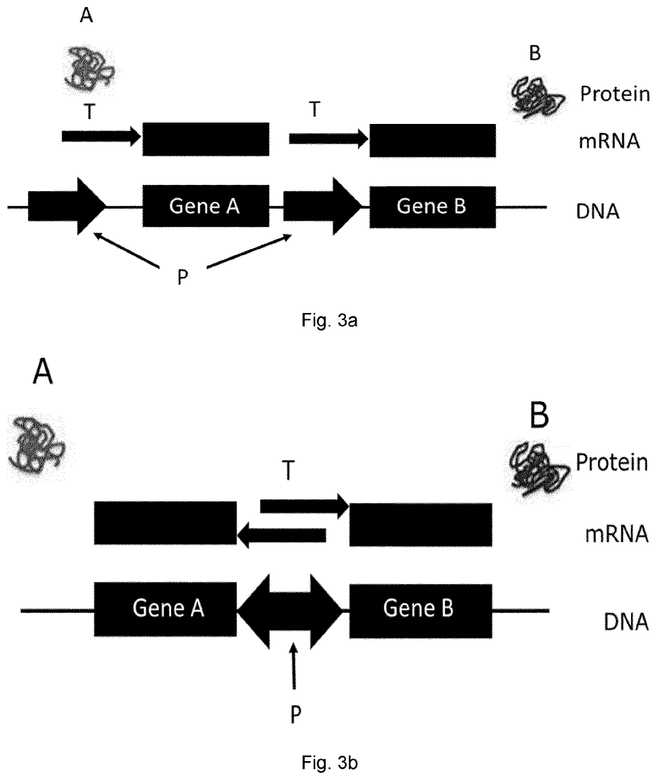

In some embodiments of the present disclosure, the co-expression element is a bidirectional promoter, the concept of which is illustrated in FIG. 3B. Bidirectional promoters are typically short (e.g., <1 kbp) intergenic regions of DNA between the 5′ ends of the genes in a bidirectional gene pair. A “bidirectional gene pair” refers to two adjacent genes coded on opposite strands, with their 5′ ends oriented toward one another.

In some embodiments of the present disclosure, the bidirectional promoter is a back-to-back arrangement of CAG promoters with four CMV enhancers (Sladitschek H L, Neveu P A et al., PLoS One 11(5), e0155177, 2016).

In some embodiments of the present disclosure, the bidirectional promoter is RPBSA (Kevin He et al., Int. J. Mol. Sci. 21(23), 9256, 2020).

In some embodiments of the present disclosure, the bidirectional promoter is a back-to-back configuration of the mouse Pgk1 and human eukaryotic translation elongation factor 1 alpha 1 promoters (Golding & Mann, Gene Therapy 18, 817-826, 2011).

In some embodiments, the vector of the disclosure is a plasmid which comprises a first nucleic acid sequence encoding a first polypeptide and a second nucleic acid sequence encoding a further polypeptide as a bidirectional gene pair comprising between their 5′ ends a bidirectional promoter.

As for the other co-expression element, another type of co-expression element may be used if more than one further polypeptide is expressed from the vector of the disclosure.

### Multiple Promoters

In some embodiments of the present disclosure, the co-expression elements are various promoters, i.e., the vector is e.g., a plasmid which comprises a separate promoter for each of the nucleic acid sequences encoding the first polypeptide and the one or more further polypeptide, i.e., for separate transcription of the first polypeptide and each of the one or more further polypeptides.

In some embodiments, each of said nucleic acid sequence will have a different promoter, the concept of which is illustrated in FIG. 3A. In some embodiments, all nucleic acid sequences have the same promoter to aim at equimolecular expression. In an alternative embodiment, one nucleic acid sequence has a stronger promoter than the other(s); that is, the nucleic acid sequence with a stronger promoter is likely to be expressed at higher levels than the other(s).

Numerous promoters are known in the art and suitable for inclusion into the plasmid of the disclosure. In some embodiments of the present disclosure, the promoter is derived from cytomegalovirus, such as the CMV promoter.

In some embodiments, the vector of the disclosure comprises one or more co-expression elements, preferably co-expression elements selected from the group consisting of IRES element, 2A peptide, bidirectional promoter and promoter.

The vector of the disclosure may comprise all potential combinations of co-expression elements.

As an example, the vector of the disclosure is a DNA plasmid which comprises a first nucleic acid sequence encoding a first polypeptide, a second nucleic acid sequence encoding a first further polypeptide and a third nucleic acid sequence encoding a second further polypeptide. In some embodiments, the DNA plasmid comprises an IRES and a 2A peptide which allows the co-expression of the first polypeptide (under control of a promoter) and of the first and second further polypeptide. In other embodiments, the DNA plasmid comprises a bidirectional promoter and another promoter.

The skilled person will know that the terms first, second and third nucleic acid sequences as in the example above do not mean that the plasmid of the disclosure comprises the nucleic acid sequences in the order of first, second and third nucleic acid sequence. The second nucleic acid sequence may be downstream or upstream of the first or third nucleic acid sequence, the third nucleic acid sequence may be downstream or upstream of the first or second nucleic acid sequence and the first nucleic acid sequence may be upstream or downstream of the second or third nucleic acid sequence. In some embodiments, the first and the second nucleic acid sequences are in opposite directions on the same DNA strand, and/or the first and third or the second and third nucleic acid sequences are in opposite directions on the same DNA strand. In further embodiments, the nucleic acid sequences encoding the first polypeptide and the further polypeptides are on opposite DNA strands.

### First Nucleic Acid or First Polypeptide

The vectors of the present disclosure comprise a first nucleic acid sequence, i.e., a DNA or RNA, including genomic DNA, cDNA, self-replicating RNA, and mRNA, either double-stranded or single-stranded, which encodes a first polypeptide. In some embodiments, the first nucleic acid sequence is a DNA. In some embodiments, the first nucleic acid sequence is optimized to the species of the subject to which it is administered. For administration to a human, in some embodiments, the first nucleic acid sequence is human codon optimized.

The first nucleic acid sequence encodes a first polypeptide, which comprises a targeting unit that targets antigen-presenting cells, a multimerization unit, such as a dimerization unit, and an antigenic unit comprising one or more epitopes, e.g., one or more disease-relevant antigens or parts thereof. Once administered to a subject, the first polypeptide is expressed and, due to the presence of the multimerization unit, forms a multimeric protein, which elicits an immune response against the antigens or parts thereof, e.g., epitopes, comprised in the antigenic unit, resulting in the activation of the subject's immune system.

In other embodiments, the antigenic unit comprises one or more T cell epitopes of an allergen, self-antigen or alloantigen. Once administered to a subject, the first polypeptide is expressed and, due to the presence of the multimerization unit, forms a multimeric protein, which allows the presentation of the epitopes comprised in the antigenic unit in a tolerance-inducing manner and is thus suitable for use as a prophylactic or therapeutic treatment of autoimmune diseases, allergic diseases and graft rejection.

Structures like the first polypeptide and dimeric proteins or multimeric proteins comprising the first polypeptide are known in the art (e.g., WO 2004/076489A1, WO 2011/161244A1, WO 2017/118695A1 and WO 2022/013277A1, the disclosures of all are included herein by reference) and the skilled person can select a targeting unit that targets antigen-presenting cells, a multimerization unit, and an antigenic unit according to the envisaged use of the vector and the desired results following its administration. The first polypeptide has an N-terminal start and a C-terminal end (illustrated in FIG. 4). The elements and units of the first polypeptide—targeting unit (TU), multimerization unit, such as, in this FIG. 4, a dimerization unit (DimU), and antigenic unit—may be arranged in the first polypeptide such that the antigenic unit is located at the C-terminal end of the first polypeptide (FIG. 4a) or at the N-terminal start of the first polypeptide (FIG. 4b). In embodiments where the antigenic unit comprises T cell epitopes of an allergen, a self-antigen or an alloantigen, the first polypeptide is a tolerance-inducing polypeptide. The resulting tolerance-inducing construct can be described as a polypeptide having an N-terminal start and a C-terminal end (illustrated in FIG. 1). The elements and units of the first polypeptide may be arranged in the first polypeptide such that the antigenic unit is located at the C-terminal end of the first polypeptide (FIG. 4c) or at the N-terminal start of the first polypeptide (FIG. 4d). Preferably, the antigenic unit is located at the C-terminal end of the first polypeptide.

An unit linker (UL) may connect the multimerization unit, such as a dimerization unit, and the antigenic unit comprising one or more epitopes, such as one or more T cell epitopes. FIG. 4 illustrates an antigenic unit with 4 epitopes (epi1, epi2, epi3, epi4), which are separated by linkers (SUL1, SUL2, SUL3). An alternative way to describe the arrangement of the epitopes epi1-epi4 is that these epitopes are arranged in 3 antigenic subunits, each comprising a epitope and a subunit linker (SUL1, SUL2, SUL3), and a terminal epitope (epi4), which is closest to the C-terminal end or N-terminal start of the first polypeptide. The subunits are indicated in the Figure by square brackets. The order and orientation of the above-described units and elements is preferably the same in the multimeric/dimeric protein and the polynucleotide.

In the following, the various units and elements of the first polypeptide will be discussed in detail. They are present in the first nucleic acid sequence as nucleic acid sequences encoding the units/elements while they are present in the first polypeptide or multimeric protein as amino acids sequences. For the ease of reading, in the following, the units/elements are mainly explained in relation to the first polypeptide/multimeric protein, i.e., on the basis of their amino acid sequences.

### Further Nucleic Acids or Further Polypeptides

The present vectors also comprise one or more further nucleic acid sequences. These encode one or more further polypeptides comprising a further antigenic unit (described in detail further below) comprising one or more further epitopes, or one or more allergens, hypoallergenic allergens, self-antigens or alloantigens. The further polypeptides may trigger a different immune response, for example a different tolerance-inducing immune response in constructs that induce such, than the first polypeptide, e.g., they may act on a different target, activate other immune cells than the first polypeptide, and/or induce tolerance via other immune cells than the first polypeptide.

The vectors of the present disclosure allow for the co-expression of the first polypeptide and the one or more further polypeptides as separate molecules.

The terms “at least one further polypeptide”, and “a further polypeptide” are used interchangeably herein to refer to at least one of the one or more further polypeptides encoded by the one or more further nucleic acid sequences comprised in the vectors of the disclosure.

By reading the present disclosure, it will be evident to the skilled person that, in some embodiments, the vector described herein comprises nucleic acid sequences encoding two or more further polypeptides, wherein the further polypeptides are different. For example, the first polypeptide may be co-expressed with 1, 2, 3, 4 or 5 further polypeptides, such as 1, 2, 3, 4 or 5 different further polypeptides. Furthermore, any embodiment of the first polypeptide may also refer to an embodiment of a further polypeptide, wherein the further polypeptide comprises a further targeting unit that targets antigen-presenting cells, an interaction unit, such as a further multimerization unit, such as a further dimerization unit, and a further antigenic unit comprising one or more further epitopes. The one or more further polypeptide may be as described herein, e.g. an allergen, a hypoallergenic allergen, a self-antigen or an alloantigen.

In some embodiments of the present disclosure, the vector comprises nucleic acid sequences encoding 2, 3, 4, 5, 6, 7 or 8 further polypeptides. In some embodiments, the vector comprises nucleic acid sequences encoding 2 to 6 further polypeptides, i.e., 2 or 3 or 4 or 5 or 6 further polypeptides. The further polypeptides may be the same or different, preferably different.

In some embodiments, each further nucleotide encodes a single further polypeptide.

In some embodiments, the different further polypeptides enhance and/or complement the effect of the first polypeptide/multimeric protein by different modes of action.

In some embodiments, the vector comprises:
i. A first nucleic acid encoding a first polypeptide comprising a targeting unit, a dimerization unit, optionally an unit linker, and an antigenic unit comprising a first antigen;
ii. A further nucleic acid sequence encoding a further polypeptide comprising a further antigenic unit comprising a further antigen or part thereof;
iii. A nucleic acid sequence encoding a 2A self-cleaving peptide;
wherein the 2A self-cleaving peptide allows co-expression of the first polypeptide and the further polypeptide.

In some embodiments, the vector comprises:
i. A first nucleic acid encoding a first polypeptide comprising a targeting unit, a dimerization unit, optionally an unit linker, and an antigenic unit comprising at least one T cell epitope;
ii. A further nucleic acid sequence encoding a further polypeptide comprising a further targeting unit, such as an MHC II targeting unit, and a further antigenic unit comprising a further antigen or part thereof;
iii. A nucleic acid sequence encoding a 2A self-cleaving peptide;
wherein the 2A self-cleaving peptide allows co-expression of the first polypeptide and the further polypeptide.

In some embodiments, the vector comprises:
i. A first nucleic acid encoding a first polypeptide comprising a targeting unit, a dimerization unit, optionally an unit linker, and an antigenic unit comprising a B cell antigen;
ii. A further nucleic acid sequence encoding a further polypeptide comprising a ubiquitination sequence and a further antigenic unit comprising at least one further T cell epitope;
iii. A nucleic acid sequence encoding a 2A self-cleaving peptide;
wherein the 2A self-cleaving peptide allows co-expression of the first polypeptide and the further polypeptide.

In some embodiments, the vector comprises:
i. A first nucleic acid encoding a first polypeptide comprising a targeting unit, a dimerization unit, optionally an unit linker, and an antigenic unit comprising at least one antigen and a first leucine zipper motif;
ii. A further nucleic acid sequence encoding a further polypeptide comprising a further antigenic unit comprising a protein and a further leucine zipper motif;
iii. A nucleic acid sequence encoding a 2A self-cleaving peptide;
wherein the 2A self-cleaving peptide allows co-expression of the first polypeptide and the further polypeptide.

In some embodiments, the vector comprises:
i. A first nucleic acid encoding a first polypeptide comprising a targeting unit, a dimerization unit, optionally an unit linker, and an antigenic unit comprising an antigen;
ii. A further nucleic acid sequence encoding a further polypeptide comprising a signal peptide and a further antigenic unit comprising a further antigen;
iii. A nucleic acid sequence encoding a 2A self-cleaving peptide;
wherein the 2A self-cleaving peptide allows co-expression of the first polypeptide and the further polypeptide.

In some embodiments, the vector comprises:
i. A first nucleic acid encoding a first polypeptide comprising a targeting unit, a dimerization unit, optionally an unit linker, an antigenic unit comprising a first antigen and a first interaction unit;
ii. A further nucleic acid sequence encoding a further polypeptide comprising a signal peptide, a further antigenic unit comprising a further antigen and a further interaction unit;
iii. A nucleic acid sequence encoding a 2A self-cleaving peptide;
wherein the 2A self-cleaving peptide allows co-expression of the first polypeptide and the further polypeptide;
wherein the first interaction unit and the further interaction unit facilitate interaction between the first antigen and the further antigen.

In some embodiments, the vector comprises:
i. A first nucleic acid encoding a first polypeptide comprising a targeting unit, a dimerization unit, optionally an unit linker, an antigenic unit comprising a first antigen and a first heterotrimerization unit;
ii. A further nucleic acid sequence encoding a further polypeptide comprising a signal peptide, a further antigenic unit comprising a further antigen and a further heterotrimerization unit;
iii. A nucleic acid sequence encoding a 2A self-cleaving peptide;
wherein the 2A self-cleaving peptide allows co-expression of the first polypeptide and the further polypeptide;
wherein the first heterotrimerization unit and the further heterotrimerization unit facilitate formation of a heterotrimer comprising the first antigen and the further antigen.

In some embodiments, the heterotrimer comprises one first antigen and two second antigens.

In some embodiments, the vector comprises:
i. A first nucleic acid encoding a polypeptide comprising a targeting unit, a first heterodimerization unit, optionally an unit linker, and an antigenic unit comprising a first antigen;
ii. A further nucleic acid sequence that encoding a further polypeptide comprising a targeting unit, a further heterodimerization unit which is different from the first heterodimerization unit, optionally an unit linker, and a further antigenic unit comprising at least one further T cell epitope;
iii. A nucleic acid sequence encoding a 2A self-cleaving peptide;
wherein the 2A self-cleaving peptide allows co-expression of the first polypeptide and the further polypeptide;
wherein the first heterodimerization unit and the further heterodimerization unit facilitate formation of a heterodimer comprising the first polypeptide and the further polypeptide.

In some embodiments, the vector comprises:
i. A first nucleic acid encoding a polypeptide comprising a targeting unit, a first heterodimerization unit, optionally an unit linker, and an antigenic unit comprising a first antigen;
ii. A further nucleic acid sequence that encoding a further polypeptide comprising a targeting unit, a further heterodimerization unit which is different from the first heterodimerization unit, optionally an unit linker, and a further antigenic unit comprising a further antigen;
iii. A nucleic acid sequence encoding a 2A self-cleaving peptide;
wherein the 2A self-cleaving peptide allows co-expression of the first polypeptide and the further polypeptide;
wherein the first heterodimerization unit and the further heterodimerization unit facilitate formation of a heterodimer comprising the first polypeptide and the further polypeptide.

In some embodiments, the present disclosure relates to a heterodimer comprising:
i. A first polypeptide comprising a targeting unit, a first heterodimerization unit, optionally an unit linker, and an antigenic unit comprising a first antigen; and
ii. A further polypeptide comprising a targeting unit, a further heterodimerization unit, an unit linker, and a further antigenic unit comprising least one further T cell epitope, optionally wherein the T cell epitopes are separated by subunit linkers.

In some embodiments, the present disclosure relates to a heterodimer comprising:
i. A first polypeptide comprising a targeting unit, a first heterodimerization unit, optionally an unit linker, and an antigenic unit comprising a first antigen; and
ii. A further polypeptide comprising a targeting unit, a further heterodimerization unit, an unit linker, and a further antigenic unit comprising a further antigen.

In some embodiments, the vector comprises:
i. A first nucleic acid encoding a first polypeptide comprising a targeting unit, a dimerization unit, optionally an unit linker, and an antigenic unit comprising a first antigen;
ii. A further nucleic acid sequence encoding a further polypeptide comprising a further antigenic unit comprising a further antigen, and a self-assembly unit;
iii. A nucleic acid sequence encoding a 2A self-cleaving peptide;
wherein the 2A self-cleaving peptide allows co-expression of the first polypeptide and the further polypeptide;
wherein the self-assembly unit facilitates formation of further antigen oligomers, such as further antigen nanoparticles.

In some embodiments, the vector comprises:
i. A first nucleic acid encoding a first polypeptide comprising a targeting unit, a dimerization unit, optionally an unit linker, and an antigenic unit;
ii. A further nucleic acid sequence encoding a further polypeptide comprising a further antigenic unit comprising a further antigen and at least one universal CD4+ T cell epitope;
iii. A nucleic acid sequence encoding a 2A self-cleaving peptide;
wherein the 2A self-cleaving peptide allows co-expression of the first polypeptide and the further polypeptide.

In some embodiments, the vector comprises:
i. A first nucleic acid encoding a first polypeptide comprising a targeting unit, a dimerization unit, optionally an unit linker, and an antigenic unit;
ii. A further nucleic acid sequence encoding a further polypeptide comprising a class II-associated invariant chain peptide (CLIP) and at least one universal CD4+ T cell epitope;
iii. A nucleic acid sequence encoding a 2A self-cleaving peptide;
wherein the 2A self-cleaving peptide allows co-expression of the first polypeptide and the further polypeptide.

In some embodiments, the present disclosure relates to a method for co-expression of:
i. A dimer comprising two first polypeptides comprising a targeting unit, a dimerization unit, optionally an unit linker, and an antigenic unit comprising a first antigen; and
ii. A further polypeptide comprising a further antigenic unit comprising a further antigen.

In some embodiments, the present disclosure relates to a composition, such as a pharmaceutical composition comprising:
i. A dimer comprising two first polypeptides comprising a targeting unit, a dimerization unit, optionally an unit linker, and an antigenic unit comprising a first antigen; and
ii. A further polypeptide comprising a further antigenic unit comprising a further antigen.

In some embodiments, the present disclosure relates to a dimer comprising two polypeptides, wherein each polypeptide comprises:
i. A first polypeptide comprising a targeting unit, a dimerization unit, optionally an unit linker, an antigenic unit comprising at least one T cell epitope, and a first leucine zipper motif; and
ii. A further polypeptide comprising a further antigenic unit comprising a protein and a further leucine zipper motif.

In some embodiments, the present disclosure relates to a dimer comprising two polypeptides, wherein each polypeptide comprises:
i. A first polypeptide comprising a targeting unit, a dimerization unit, optionally an unit linker, an antigenic unit comprising a first antigen, and an oligomerization unit; and
ii. A further polypeptide comprising a further antigenic unit comprising a further antigen which is identical to the first antigen, and a further oligomerization unit.

In some embodiment, the oligomerization unit and the further oligomerization unit are identical. In some embodiment, the oligomerization unit and the further oligomerization unit are different. In some embodiment, the oligomerization unit and the further oligomerization unit are heterooligomerization units, such as heterotrimerization units.

In some embodiments, the present disclosure relates to a method for co-expression of:
i. A dimer comprising two first polypeptides comprising a targeting unit, a dimerization unit, optionally an unit linker, and an antigenic unit comprising a first antigen; and
ii. A further polypeptide comprising a further antigenic unit comprising a further antigen, and an interaction unit, such as a self-assembly domain, optionally wherein several copies of the further antigen self-assembly into a nano-particle.

In some embodiments, the present disclosure relates to a composition, such as a pharmaceutical composition, comprising:
i. A dimer comprising two first polypeptides comprising a targeting unit, a dimerization unit, optionally an unit linker, and an antigenic unit comprising a first antigen; and
ii. A further polypeptide comprising a further antigenic unit comprising a further antigen, and an interaction unit, such as a self-assembly domain, optionally wherein several copies of the further antigen self-assembly into a nano-particle.

In some embodiments, the present disclosure relates to a method for co-expression of:
i. A dimer comprising two first polypeptides comprising a targeting unit, a dimerization unit, optionally an unit linker, and an antigenic unit comprising at least one T cell epitope; and
ii. A further polypeptide comprising a further targeting unit and a further antigenic unit comprising a further antigen.

In some embodiments, the present disclosure relates to a composition, such as a pharmaceutical composition, comprising:
i. A dimer comprising two first polypeptides comprising a targeting unit, a dimerization unit, optionally an unit linker, and an antigenic unit comprising at least one T cell epitope; and
ii. A further polypeptide comprising a further targeting unit and a further antigenic unit comprising a further antigen.

In some embodiments, the first and/or the further polypeptide may, optionally, comprise a signal peptide.

In some embodiments, the first nucleic acid sequence and/or the one or more further nucleic acid sequences is selected from DNA sequence and RNA sequence.

In some embodiments, at least one of the one or more further polypeptides is a folded antigen, which may be expressed as an oligomer or multimer as detailed further below.

For example, the first polypeptide may be co-expressed with a further polypeptide, wherein:
i. the first polypeptide comprises a targeting unit, a dimerization unit, optionally an unit linker, an antigenic unit comprising an antigen, and an oligomerization unit, such as a coiled coil peptide A,
ii. the further polypeptide comprises:
   a) a further antigenic unit comprising a further antigen,
   b) a further oligomerization unit, such as a coiled coil peptide B, wherein the oligomerization unit and the further oligomerization unit facilitate formation of oligomers comprising one copy of the antigen and two copies of the further antigens.

For example, the first polypeptide may be co-expressed with 2 further polypeptides, wherein:
i. the first further polypeptide comprises:
   a) a first further antigenic unit comprising a first further antigen,
   b) a first oligomerization unit, such as a coiled coil peptide A,
i. the second further polypeptide comprises:
   c) A second further antigenic unit comprising a second further antigen,
   d) A second oligomerization unit, such as a coiled coil peptide B,
wherein the first oligomerization unit and the second oligomerization unit facilitates formation of oligomers comprising the first further antigen the second further antigen.

For example, the first polypeptide may be co-expressed with 3 further polypeptides, wherein:
i. the first further polypeptide comprises:
   a) a first further antigenic unit comprising a first further antigen; and
   b) a first interaction unit, such as a further multimerization unit, optionally wherein the interaction unit comprises the amino acid sequence

(SEQ ID NO: 12)
AEIAAIEYEQAAIKEEIAAIKDKIAAIKEYIAAI,

ii. the second further polypeptide comprises:
   c) A second further antigenic unit comprising a second further antigen,
   d) A second interaction unit, such as a further multimerization unit, optionally wherein the interaction unit comprises the amino acid sequence EKIAAIKEEQAAIEEEIQAIKEEIAAIKYLIAQI (SEQ ID NO: 13);
iii. the third further polypeptide comprise
   e) A third further antigenic unit comprising a third further antigen,
   f) A third interaction unit, such as a further multimerization unit, optionally wherein the interaction unit comprises the amino acid sequence

(SEQ ID NO: 14)
AEIAAIKYKQAAIKNEIAAIKQEIAAIEQMIAAI,

wherein the first interaction unit, the second interaction unit and the third interaction unit facilitate interaction of the first further antigen, the second further antigen and the third further antigen.

### Structural Components Found in Both the First and the One or More Further Polypeptides

In some embodiments, at least one of the one or more further polypeptide comprises at least some structural elements which are also comprised in the first polypeptide. Thus, in some embodiments, at least one further polypeptide comprising a further antigenic unit comprising one or more epitopes or one or more T cell epitopes of an allergen, self-antigen or allo-antigen also comprises a further targeting unit that targets antigen-presenting cells and an interaction unit, such as a further multimerization unit, such as a further dimerization unit. The further polypeptide may comprise more than one further antigenic unit comprising one or more epitopes or comprising one or more T cell epitopes of an allergen, self-antigen or allo-antigen.

In some embodiments, said at least one further polypeptide forms a hetero-multimer, such as a heterodimer with the first polypeptide via interaction of the multimerization unit of the first polypeptide and an interaction unit, such as a further multimerization unit of said one further polypeptide. In some embodiments the first polypeptide and said further polypeptide are different.

In other embodiments, at least one of the one or more further polypeptides comprises at least one unit selected from the group consisting of:
i. a further targeting unit; and
ii. an interaction unit, such as a further multimerization unit, such as a further dimerization unit.

In some embodiments, the interaction unit facilitates interaction between the antigen of the antigenic unit of the first polypeptide and one or more further antigens of the further antigenic unit of the further polypeptide. In some embodiments, the interaction unit facilitates interaction between two or more of the further antigens of the further antigenic unit of the further polypeptide.

Thus, the skilled person will understand that embodiments described herein for the targeting unit, multimerization unit, dimerization unit, unit linker and signal peptide of the first polypeptide may also be embodiments of the further polypeptide, i.e., embodiments of a further targeting unit, an interaction unit, such as a further multimerization unit, such as a further dimerization unit, a further antigenic unit, a further unit linker, and/or a further signal peptide of a further polypeptide. For embodiments where the antigenic unit comprises one or more disease-relevant epitopes, the embodiments described herein for the antigenic unit may also be embodiments of the further antigenic unit.

In some embodiments, the targeting unit of the first polypeptide, and the further targeting unit of at least one further polypeptide are different.

In some embodiments, the multimerization unit, such as the dimerization unit, of the first polypeptide, and the interaction unit, such as the further multimerization unit, such as the further dimerization unit, of at least one further polypeptide are different.

In some embodiments, the antigenic unit of the first polypeptide, and the further antigenic unit of at least one further polypeptide are different.

Similarly, the skilled person would understand that specific epitopes and specific antigens can be comprised in either the first polypeptide, in a further polypeptide, or in both.

### Immunostimulatory Compounds

In some embodiments, particularly for immunogenic constructs, the vector further comprises one or more additional polynucleotides, wherein the one or more additional polynucleotides comprise one or more nucleic acid sequences encoding one or more immunostimulatory compounds and wherein the vector allows for the co-expression of the first polypeptide, the one or more further polypeptides, and the one or more immunostimulatory compounds as separate molecules.

In some embodiments, particularly for immunogenic constructs, the vector further encodes one or more immunostimulatory compounds. Thus, the vector allows for the co-expression of the first polypeptide, the one or more further polypeptides as described herein and the one or more immunostimulatory compounds as separate molecules.

In some embodiments, particularly for immunogenic constructs, the vector further encodes at least two immunostimulatory compounds, such as at least three immunostimulatory compounds, wherein said immunostimulatory compounds are identical or different, preferably identical, and wherein the vector allows for the co-expression of the first polypeptide and the one or more immunostimulatory compounds as separate molecules.

In some embodiments, particularly for immunogenic constructs, the vector encodes for one or more additional polynucleotides encoding an immunostimulatory compound, such as a granulocyte-macrophage colony-stimulating factor (GM-CSF), wherein the vector allows for the co-expression of the first polypeptide and the one or more immunostimulatory compounds as separate molecules.

The one or more immunostimulatory compounds may enhance the effect of the immunogenic constructs of the disclosure. Without wishing to be bound by the theory, the co-expression may have marked advantages on the cellular level. When a vector comprising the first polynucleotide of the disclosure is administered intramuscularly to a subject, the first polypeptide, dimeric protein and/or multimeric protein is secreted from muscle cells and taken up by neighboring antigen-presenting cells. Since the immunostimulatory compound is expressed in and secreted from the same muscle cell, it can stimulate the same antigen-presenting cell and thereby directly affect said antigen-presenting cell, e.g., if the antigen-presenting cell is a dendritic cell, promote the activation and maturation of it.

In some embodiments, particularly for immunogenic constructs, the vector encodes for an additional polynucleotide encoding an immunostimulatory compound, such as a granulocyte-macrophage colony-stimulating factor (GM-CSF).

If more than one immunostimulatory compound is present in the one or more additional polypeptide, an IRES element and/or 2A self-cleaving peptide (e.g. as described herein elsewhere) might be present, e.g. upstream of each nucleic acid sequence encoding an immunostimulatory compound. Such multiple sequences may be co-expressed using e.g. a bidirectional promoter, or each nucleotide sequence encoding an immunostimulatory compound comprises a promoter

In some embodiments, particularly for immunogenic constructs, the immunostimulatory compound is a compound that stimulates antigen-presenting cells and the stimulation results in e.g. attraction, activation, maturation and/or proliferation of APCs.

In some embodiments, particularly for immunogenic constructs, the immunostimulatory compound is one that attracts APCs, preferably one that can interact with the following surface molecules on APCs: CCR1 (C-C motif chemokine receptor 1), CCR3 (C-C motif chemokine receptor 3), CCR4 (C-C motif chemokine receptor 4), CCR5 (C-C motif chemokine receptor 5), CCR6 (C-C motif chemokine receptor 6), CCR7 (C motif chemokine receptor 7), CCR8 (C motif chemokine receptor 8) or XCR1 (X-C motif chemokine receptor 1).

In other embodiments, particularly for immunogenic constructs, the immunostimulatory compound is selected from the list consisting of CCL3L1 (also known as MIP-1α), preferably human CCL3L1 (also known as human MIP-1α), RANTES (CCL5), MIP-1β (CCL4), MIP-3a (CCL20), CCL19, CCL 21, XCL1 or XCL2.

In other embodiments, particularly for immunogenic constructs, the immunostimulatory compound is one that promotes activation and/or maturation of APCs. In some embodiments, particularly for immunogenic constructs, the immunostimulatory compound can interact with the following surface molecules on APCs: a receptor of the TNF receptor superfamily, including CD40 (cluster of differentiation 40), CD137 (4-1BB), CD27, ICOSL (CD275) or RANK.

Such immunostimulatory compounds may be selected from the list consisting of CD40L (CD40 ligand, CD154), CD137L (4-1BBL, 4-1BB ligand), CD70, ICOS (CD278) or RANKL.

In other embodiments, particularly for immunogenic constructs, the immunostimulatory compound is a cytokine selected from IL-2, IL-10, IL-12, TNFα and IFNγ. In other embodiments, the immunostimulatory compound can be an immune signaling molecule such as MyD88 and TRIF which activate through TLR receptors. In other embodiments, the immunostimulatory compound can be a viral infection sensor such as for example RIG-1 and MDA-5. In other embodiments, the immunostimulatory compound can interact with a pattern recognition receptor on APCs, e.g. a Toll-like receptor, including TLR2, TLR4 or TLR5.

Such immunostimulatory compounds may be selected from the list consisting of pathogen-associated molecular patterns (PAMPs), such as flagellin, or protein damage-associated molecular patterns (DAMPs), such as HMGB1, HSPs (heat-shock proteins) Calrecticulin and Annexin A1. PAMPs/DAMPs include those can be included as a nucleotide sequence into the DNA plasmid of the disclosure and will be expressed as functional proteins that may comprise functional groups introduced by post-translational modifications. The aforementioned molecules in turn activate the following receptors on APCs: RAGE, TLR4, TLR9 and TIM-3 (for HMGB1), FPR (for Annexin A1), SREC1, LOX1 and CD91 (for HSP).

In some embodiments, particularly for immunogenic constructs, the immunostimulatory compound is one that promotes growth and/or expansion of APCs. In some embodiments, the immunostimulatory compound can interact with the following surface molecules on APCs: GM-CSF-receptor (granulocyte-macrophage colony-stimulating factor receptor, CD116), FLT-3R (fms like tyrosine kinase 3, CD135), IL-15R or IL-4R. In other embodiments, the immunostimulatory compound is a growth factor, such as GM-CSF (granulocyte-macrophage colony-stimulating factor), FLT-3L, IL-15 or IL-4.

In some embodiments, particularly for immunogenic constructs, the vector comprises nucleotide sequences encoding 2, 3, 4, 5, 6, 7 or 8 immunostimulatory compounds. In preferred embodiments, the DNA plasmid comprises nucleotide sequences encoding 2 to 6 immunostimulatory compounds, i.e. 2 or 3 or 4 or 5 or 6 different immunostimulatory compounds. The immunostimulatory compounds may be the same or different, preferably different.

In some embodiments, particularly for immunogenic constructs, the different immunostimulatory compounds also affect APCs differently, to stimulate the immune system on many different levels and by that maximize the therapeutic or prophylactic effect of the construct of the disclosure.

As an example, the vector comprises nucleotides encoding 3 different immunostimulatory compounds, with the first one being an immunostimulatory compound that promotes the attraction of DCs (e.g. XCL1), the second one being an immunostimulatory compound that promotes the growth of DCs (e.g. FLT-3L) and the third one being an immunostimulatory compound that promotes activation of DCs (e.g. CD40L). The selection of the particular immunostimulatory compounds will also depend on the targeting unit, since it targets APCs and may also affect APCs in a similar manner as the immunostimulatory compound, i.e. attract or activate APCs.

### Immunoinhibitory Compounds

In some embodiments, particularly for tolerance-inducing constructs, the vector further comprises one or more additional polynucleotides, wherein the one or more additional polynucleotides comprise one or more nucleic acid sequences encoding one or more immunoinhibitory compounds and wherein the vector allows for the co-expression of the first polypeptide, the one or more further polypeptides, and the one or more immunoinhibitory compounds as separate molecules.

In some embodiments, particularly for tolerance-inducing constructs, the vector further encodes one or more immunoinhibitory compounds. Thus, the vector allows for the co-expression of the first polypeptide, the one or more further polypeptides as described herein and the one or more i immunoinhibitory compounds as separate molecules.

The one or more immunoinhibitory compounds help to generate or promote an environment that favours the presentation of the epitopes in the antigenic unit in a tolerance inducing manner, or by e.g. favouring the induction of tolerance maintaining cells or helping to maintain such cells.

If more than one immunoinhibitory compound is present in the one or more additional polypeptide, an IRES element and/or 2A self-cleaving peptide (e.g. as described herein elsewhere) might be present, e.g. upstream of each nucleic acid sequence encoding an immunoinhibitory compound. Such multiple sequences may be co-expressed using e.g. a bidirectional promoter, or each nucleotide sequence encoding an immunoinhibitory compound comprises a promoter.

In some embodiments of the present disclosure, the immunoinhibitory compound is a compound that is known to induce, increase or maintain immune tolerance.

In some embodiments of the present disclosure, the immunoinhibitory compound is an extracellular part of inhibitory checkpoint molecules.

In some embodiments, the inhibitory checkpoint molecule is selected from the group consisting of CTLA-4 (SEQ ID NO: 248), PD-1 (SEQ ID NO: 273), BTLA and TIM-3. In some embodiments, the inhibitory checkpoint molecule is CTLA-4 (SEQ ID NO: 248). In some embodiments, the inhibitory checkpoint molecule is PD-1 (SEQ ID NO: 273). In some embodiments, the inhibitory checkpoint molecule is BTLA. In some embodiments, the inhibitory checkpoint molecule is TIM-3. In some embodiments of the present disclosure, the immunoinhibitory compound is a cytokine selected from the group consisting of IL-10 (SEQ ID NO: 260), TGFβ1 (SEQ ID NO: 249), TGFβ2 (SEQ ID NO: 250), TGFβ3 (SEQ ID NO: 251), IL-27, IL-2, IL-37 and IL-35. In some embodiments, the cytokine is IL-10 (SEQ ID NO: 260). In some embodiments, the cytokine is TGFβ1 (SEQ ID NO: 249). In some embodiments, the cytokine is TGFβ2 (SEQ ID NO: 250). In some embodiments, the cytokine is TGFβ3 (SEQ ID NO: 251). In some embodiments, the cytokine is IL-27. In some embodiments, the cytokine is IL-2. In some embodiments, the cytokine is IL-37. In some embodiments, the cytokine is IL-35.

In some embodiments of the present disclosure, the construct comprises further nucleic acid sequences encoding 2, 3, 4, 5, 6, 7 or 8 immunoinhibitory compounds. In preferred embodiments, the construct comprises nucleic acid sequences encoding 2 to 6 immunoinhibitory compounds, e.g. 2 or 3 or 4 or 5 or 6 different immunoinhibitory compounds. The immunoinhibitory compounds may be the same or different, preferably different.

In preferred embodiments, the different immunoinhibitory compounds generate or promote a tolerance-inducing environment on many different levels. By way of example, the plasmid of the disclosure may comprise nucleic acid sequences encoding 3 different immunoinhibitory compounds, wherein the first induces tolerance, the second increases tolerance and the third maintains tolerance.

In some embodiments, particularly for tolerance-inducing constructs useful for treating or preventing autoimmune diseases or allergies, one of the one or more additional polynucleotides encodes an immunoinhibitory compound which is an inhibitor of the cGAS-STING pathway.

The detection of foreign DNA is a crucial element of immunity. In mammalian cells, the cyclic GMP-AMP synthase (cGAS)-stimulator of interferon genes (STING) pathway (cGAS-STING) has emerged as a critical mechanism for coupling the sensing of DNA to the induction of innate immune responses. The cGAS-STING pathway is triggered by DNA and lacks pathogen-specific attributes.

Thus, in order to minimize undesirable immune responses triggered by the tolerance-inducing constructs of the disclosure, in some embodiments the one or more additional polypeptides comprise or consist of one or more inhibitors of cGAS, such as Vaccinia E5. This may lead to downregulation of inflammatory cytokine production. Such constructs may be particularly useful for treating autoimmune diseases, such as rheumatoid arthritis, psoriasis, Aicardi-Goutières syndrome, systemic lupus erythematosus and primary biliary liver disease.

In some embodiments, the one or more inhibitors of cGAS is selected from a full length Vaccinia E5 or a fragment of Vaccinia E5. In some embodiments, the Vaccinia E5 is derived from an organism selected from the group consisting of virulent poxviruses, such as VACV (WR and Copenhagen strains), cowpox, and/or ectromelia virus.

In some embodiments, the full length Vaccinia E5 comprises an amino acid sequence as set forth in SEQ ID NO: 292. In some embodiments, the fragment of Vaccinia E5 comprises a part of the amino acid sequence as set forth in SEQ ID NO: 292. In some embodiments, the one or more inhibitors of cGAS is a modified version of a full length Vaccinia E5 or a fragment of Vaccinia E5, such as a full length Vaccinia E5 or a fragment of Vaccinia E5 comprising at least one amino acid modification, such as at least one amino acid deletion, amino acid insertion and/or amino acid substitution. In some embodiments, the full length Vaccinia E5 or the fragment of Vaccinia E5 comprises 1, 2, 3, 4, 5, 6, 7, 8, 9 or 10 amino acid modifications, such as 1, 2, 3, 4, 5, 6, 7, 8, 9 or 10 amino acid deletions, amino acid insertions and/or amino acid substitutions. In some embodiments, the full length Vaccinia E5 or the fragment of Vaccinia E5 comprises 11, 12, 13, 14, 15, 16, 17, 18, 19 or 20 amino acid modifications, such as 11, 12, 13, 14, 15, 16, 17, 18, 19 or 20 amino acid deletions, amino acid insertions and/or amino acid substitutions. In some embodiments, the full length Vaccinia E5 or the fragment of Vaccinia E5 comprises 1-10, 1-15, 1-20, 1-25, 1-30, 1-35, 1-40, 1-45, or 1-50 amino acid modifications, such as 1-10, 1-15, 1-20, 1-25, 1-30, 1-35, 1-40, 1-45, or 1-50 amino acid deletions, amino acid insertions and/or amino acid substitutions. In some embodiments, the full length Vaccinia E5 comprises a nucleotide sequence as set forth in SEQ ID NO: 293. In some embodiments, the fragment of Vaccinia E5 comprises a part of the nucleotide sequence as set forth in SEQ ID NO: 293.

While the cGAS-STING pathway is not pathogen-specific, other types of responses exist within the cells, which are pathogen-specific and limit early proliferation and spreading of pathogens. Such an innate immunity response relies on the detection of evolutionarily conserved molecular structures termed pathogen-associated molecular patterns (PAMPs), which are expressed by a wide variety of infectious microorganisms. PAMPs are recognized by pattern recognition receptors, including Toll-like receptors (TLRs), Nod-like receptors and RIG-I-like receptors. Activation of the innate immune response by e.g. TLR activation is typically followed by the production of proinflammatory cytokines, chemokines, type I interferons and antimicrobial pesticides.

Bacterial DNA is an example of a PAMP which can activate the innate immune system, as bacterial DNA is unmethylated and thus contains a high frequency of GC dinucleotides compared to mammalian cells, in which 70 to 80% of the CpG dinucleotides are methylated. Upon infection, the bacterial DNA released exposes cells expressing TLR-9 to unmethylated CpG motifs, which consist of an unmethylated CG dinucleotide surrounded by flanking regions. This triggers the immune response and improves the host's capability to eliminate the pathogen.

### Targeting Unit

### Targeting Unit of Immunogenic Constructs

The first polypeptide encoded by the first nucleic acid comprised in the vectors of the disclosure comprises a targeting unit that targets APCs for immunogenic constructs. APCs include dendritic cells (DCs) and subsets thereof. In some embodiments at least one of the one or more further polypeptides also comprises a further targeting unit, which can be any targeting unit described in this section.

The term “targeting unit” as used herein for immunogenic constructs refers to a unit that delivers the construct of the disclosure to an antigen-presenting cell (APC) and interacts with surface molecules on the APC. The term “targeting unit” as used herein refers to the targeting unit comprised in the first polypeptide, as well as to any targeting unit comprised by any further targeting unit comprised by any further polypeptide of the immunogenic constructs, if present.

Due to the presence of the targeting unit in immunogenic constructs, the multimeric protein attracts DCs, neutrophils and other immune cells. Thus, the multimeric protein will not only target the antigenic unit comprised therein to specific cells, but also facilitate a response-amplifying effect (adjuvant effect) by recruiting specific immune cells to the administration site of the vector. Similarly, in some embodiments, the further polypeptide can recruit specific immune cells to the administration site of the vector.

The targeting unit is designed to target the multimeric protein, or the further polypeptide, to surface molecules expressed on the APCs, such as molecules expressed on any or many types of APCs or molecules exclusively on subsets of APCs, such as on subsets of DCs.

In some embodiments, in particular for immunogenic constructs, the targeting unit delivers the polypeptide/multimeric protein, or the further polypeptide, to an antigen-presenting cell for MHC class II-restricted presentation to CD4+ T cells or for providing cross presentation to CD8+ T cells by MHC class I restriction.

In some embodiments, the targeting unit binds to surface receptors on the APC, without activating the cell. In some embodiments, the targeting unit binds to surface receptors on the APC, without inducing maturation of the cell.

In some embodiments, the APC internalizes the construct and presents the T cell epitopes comprised in the antigenic unit, or in the further polypeptide, on MHC on its surface in an anti-inflammatory, tolerogenic manner, e.g., by not upregulating co-stimulatory signals and/or by upregulating inhibitory surface molecules and/or by promoting the secretion of inhibitory cytokines.

Examples of such surface molecules on APCs are HLA, cluster of differentiation 14 (CD14), cluster of differentiation 40 (CD40), CLEC9A, chemokine receptors and Toll-like receptors (TLRs). Chemokine receptors include C-C motif chemokine receptor 1 (CCR1), C-C motif chemokine receptor 3 (CCR3), C-C motif chemokine receptor 4 (CCR4), C-C motif chemokine receptor 5 (CCR5), C-C motif chemokine receptor 6 (CCR6), C-C motif chemokine receptor 7 (CCR7), C-C motif chemokine receptor 8 (CCR8) and XCR1. Toll-like receptors include TLR-2, TLR-4 and TLR-5. In preferred embodiments, the targeting unit is or comprises a moiety that interacts with these surface molecules.

In some embodiments, particularly for immunogenic constructs, the aforementioned surface molecules are present on human APCs.

Thus, in some embodiments, particularly for immunogenic constructs, the targeting unit comprises or consists of an antibody-binding region, such as the antibody variable domains (VL and VH), with specificity for MHC/HLA, CD14, CD40, CLEC9A or Toll-like receptors, preferably with specificity for hCD14, hCD40, hCLEC9A or human Toll-like receptors. In some embodiments, the targeting unit comprises or consists of a synthetic or natural ligand. Examples include soluble CD40 ligand (CD40L), preferably hCD40L, natural ligands like chemokines, preferably such as in their human forms, e.g., chemokine ligand 5, also called C-C motif ligand 5 (CCL5 or RANTES), preferably hCCL5, such as hCCL5 with SEQ ID NO: 152, macrophage inflammatory protein alpha and its isoforms, including mouse CCL3 (or MIP-1α), and human isoforms hCCL3, hCCL3L1, hCCL3L2 and hCCL3L3, chemokine ligand 4 (CCL4) and its isoform CCL4L, preferably hCCL4 and hCCL4L, chemokine ligand 19 (CCL19), preferably hCCL19, chemokine ligand 20 (CCL20), preferably hCCL20, chemokine ligand 21 (CCL21), preferably hCCL21, chemokine motif ligand 1 or 2 (XCL1 or XCL2), preferably hXCL1 or hXCL2, and bacterial antigens like for example flagellin.

In some embodiments, particularly for immunogenic constructs, the targeting unit has affinity for an MHC class II protein. Thus, in some embodiments, the targeting unit comprises or consists of an antibody-binding region, such as the antibody variable domains (VL and VH), with specificity for MHC class II proteins selected from the group consisting of anti-HLA-DP, anti-HLA-DR and anti-pan HLA class II.

In other embodiments, particularly for immunogenic constructs, the targeting unit has affinity for a surface molecule selected from the group consisting of CD14, CD40, TLR-2, TLR-4 and TLR-5, preferably affinity for a surface molecule selected from the group consisting of hCD14, hCD40, hTLR-2, hTLR-4 and hTLR-5. Thus, in some embodiments the targeting unit comprises or consist of an antibody-binding region, such as the antibody variable domains (VL and VH), with specificity for CD14, CD40, TLR-2, TLR-4 or TLR-5, such as anti-CD14, anti-CD40, anti-TLR-2, anti-TLR-4 or anti-TLR-5, preferably with specificity for hCD14, hCD40, hTLR-2, hTLR-4 or hTLR-5, such as anti-hCD14, anti-hCD40, anti-hTLR-2, anti-hTLR-4 or anti-hTLR-5.

In yet other embodiments, particularly for immunogenic constructs, the targeting unit comprises or consists of flagellin, which has affinity for TLR-5, such as hTLR-5. In yet other embodiments, the targeting unit comprises or consists of an antibody-binding region with specificity for CLEC9A, such as anti-CLEC9A or variants thereof, such as anti-CLEC9A scFv or the targeting unit comprises or consists of a CLEC9 ligand, e.g., a CLEC9 ligand comprising or consisting of the nucleic acid sequence with SEQ ID NO: 153 or an amino acid sequence encoded by said nucleic acid sequence. In preferred embodiments, the targeting unit comprises or consists of an antibody-binding region with specificity for hCLEC9A, such as anti-hCLEC9A or variants thereof, such as anti-hCLEC9A scFv or the targeting unit comprises or consists of a human CLEC9 ligand.

In some embodiments, particularly for immunogenic constructs, the targeting unit has affinity for a chemokine receptor selected from CCR1, CCR3, CCR5 and CCR7, more preferably for a chemokine receptor selected from CCR1, CCR3 and CCR5. In further preferred embodiments, the targeting unit has affinity for a chemokine receptor selected from hCCR1, hCCR3, hCCR5 and hCCR7, more preferably for a chemokine receptor selected from hCCR1, hCCR3 and hCCR5.

In some embodiments, particularly for immunogenic constructs, the targeting unit has affinity for the chemokine receptor CCR7, preferably for the human chemokine receptor CCR7. In other embodiments, the targeting unit comprises or consists of CCL19, such as CCL19 comprising or consisting of a nucleotide sequence of SEQ ID NO: 154 or an amino acid sequence encoded by said nucleotide sequence, or CCL21, such as the human forms of CCL19 or CCL21.

In some embodiments, particularly for immunogenic constructs, the targeting unit comprises or consists of chemokine human macrophage inflammatory protein alpha (human MIP-1α (hMIP-1α)) variant, referred to as “CCL3L1” variant within this disclosure, which binds to its cognate receptors, including CCR1, CCR3 and CCR5, expressed on the cell surface of APCs. The binding of the targeting unit to its cognate receptors leads to internalization of the multimeric protein into the APC and degradation of the protein into small peptides that are loaded onto MHC molecules and presented to CD4+ and CD8+ T cells to induce specific immune responses. Once stimulated, and with help from activated CD4+ T cells, CD8+ T cells will target and kill cells expressing the same antigens, e.g., cancer cells expression such same antigens.

In some embodiments, particularly for immunogenic constructs, both a T cell response and a B cell response are induced. This also enables for an antibody response, i.e., antibodies binding to, for example, a viral surface protein when the virus is in circulation and neutralizing the virus by inhibiting it from entering, attaching to, and/or fusing to the host cell.

In some preferred embodiments, particularly for immunogenic constructs, the targeting unit comprises an amino acid sequence having at least 80% sequence identity to the amino acid sequence 24-93 of SEQ ID NO: 128, such as comprising the amino acid sequence 26-93 of SEQ ID NO: 128 or comprising the amino acid sequence 28-93 of SEQ ID NO: 128.

In some further preferred embodiments, particularly for immunogenic constructs, the targeting unit comprises an amino acid sequence having at least 85% sequence identity to the amino acid sequence 24-93 of SEQ ID NO: 128, such as at least 86% or at least 87%, 88%, 89%, 90%, 91%, 92%, 93%, 94%, 95%, 96%, 97%, 98% or at least 99% sequence identity. In yet some further preferred embodiments, the targeting unit comprises the amino acid sequence 24-93 of SEQ ID NO: 128.

In some more preferred embodiments, particularly for immunogenic constructs, the targeting unit consists of an amino acid sequence having at least 80% sequence identity to the amino acid sequence 24-93 of SEQ ID NO: 128, such as consisting of the amino acid sequence 26-93 of SEQ ID NO: 128 or consisting of the amino acid sequence 28-93 of SEQ ID NO: 128.

In some further preferred embodiments, particularly for immunogenic constructs, the targeting unit consists of an amino acid sequence having at least 85% sequence identity to the amino acid sequence 24-93 of SEQ ID NO: 128, such as at least 86% or at least 87%, 88%, 89%, 90%, 91%, 92%, 93%, 94%, 95%, 96%, 97%, 98% or at least 99% sequence identity. In yet other preferred embodiments, the targeting unit consists of the amino acid sequence 24-93 of SEQ ID NO: 128.

In some preferred embodiments, particularly for immunogenic constructs, the targeting unit comprises the amino acid sequence 24-93 of SEQ ID NO: 128, except that at the most six amino acids have been substituted, deleted or inserted, such as at the most five amino acids, such as at the most four amino acids, such as at the most three amino acids, such as at the most two amino acids or such as at the most one amino acid. An embodiment of such a targeting unit is one comprising the amino acid sequence 26-93 of SEQ ID NO: 128 or one comprising the amino acid sequence 28-93 of SEQ ID NO: 128.

In some preferred embodiments, particularly for immunogenic constructs, the targeting unit consists of the amino acid sequence 24-93 of SEQ ID NO: 128, except that at the most six amino acids have been substituted, deleted or inserted, such as at the most five amino acids, such as at the most four amino acids, such as at the most three amino acids, such as at the most two amino acids or such as at the most one amino acid. Some embodiments of such a targeting unit are one consisting of the amino acid sequence 26-93 of SEQ ID NO: 128 or one consisting of the amino acid sequence 28-93 of SEQ ID NO: 128.

In some preferred embodiments, particularly for immunogenic constructs, the targeting unit comprises a nucleic acid sequence having at least 80% sequence identity to the nucleic acid sequence with SEQ ID NO: 129.

In some further preferred embodiments, the targeting unit comprises a nucleic acid sequence having at least 85% sequence identity to the nucleic acid sequence with SEQ ID NO: 129, such as at least 86% or at least 87%, 88%, 89%, 90%, 91%, 92%, 93%, 94%, 95%, 96%, 97%, 98% or at least 99% sequence identity. In yet some further preferred embodiments, the targeting unit comprises the nucleic acid sequence of SEQ ID NO: 129.

In more preferred embodiments, the targeting unit consists of a nucleic acid sequence having at least 80% sequence identity to the nucleic acid sequence with SEQ ID NO: 129.

In some further preferred embodiments, the targeting unit consists of a nucleic acid sequence having at least 85% sequence identity to the nucleic acid sequence of SEQ ID NO: 129, such as at least 86% or at least 87%, 88%, 89%, 90%, 91%, 92%, 93%, 94%, 95%, 96%, 97%, 98% or at least 99% sequence identity. In some other preferred embodiments, the targeting unit has the nucleic acid sequence of SEQ ID NO: 129.

### Targeting Unit of Tolerance-Inducing Constructs

The first polypeptide encoded by the first nucleic acid comprised in the vectors of the disclosure comprises a targeting unit that binds to surface receptors on the APC without activating the cell and/or without inducing maturation of the cell for tolerance-inducing constructs. In some embodiments at least one of the one or more further polypeptides also comprises a further targeting unit, which can be any targeting unit described in this section.

The term “targeting unit” as used herein for tolerance-inducing constructs refers to a unit that delivers the construct of the disclosure to an APC and interacts with surface molecules on the APC, e.g. binds to surface receptors on the APC, without activating the cell and/or without inducing maturation of the cell. The APC internalizes the construct and presents the T cell epitopes comprised in the antigenic unit on MHC on its surface in an anti-inflammatory, tolerogenic manner, e.g. by not upregulating co-stimulatory signals and/or upregulating inhibitory surface receptors and/or secretion of inhibitory cytokines.

In some embodiments, in particular for tolerance-inducing constructs, the targeting unit delivers the polypeptide/multimeric protein, or the further polypeptide, to an antigen-presenting cell for MHC class II-restricted presentation to CD4+ T cells or for providing presentation regulatory to CD8+ T cells by classical or non-classical MHC class I restriction.

In some embodiments, particularly for tolerance-inducing constructs, the targeting unit comprises or consists of a moiety that binds to a receptor selected from the group consisting of TGFβ receptor, such as TGFβR1, TGFβR2, or TGFβR3, IL10R, such as IL-10RA and IL10-RB, IL2R, IL4R, IL6R, IL11R and IL13R, IL27R, IL35R, IL37R, GM-CSFR, FLT3, CCR7, CD11b, CD11c, CD103, CD14, CD36, CD205, CD109, VISTA, MARCO, MHCII, CD83, SIGLEC, MGL/Clec10A, ASGR (ASGR1/ASGR2), CD80, CD86, Clec9A, Clec12A, Clec12B, DCIR2, Langerin, MR, DC-Sign, Treml4, Dectin-1, PDL1, PDL2, HVEM, CD163, CD32b and CD141.

In some embodiments, particularly for tolerance-inducing constructs, the targeting unit comprises or consists of a moiety that binds to a human (h) receptor selected from the group consisting of hTGFβ receptor, such as hTGFβR1, hTGFβR2, or hTGFβR3, hIL10R, such as hIL-10RA and hIL10-RB, hIL2R, hIL4R, hIL6R, hIL11R and hIL13R, hIL27R, hIL35R, hIL37R, hGM-CSFR, hFLT3, hCCR7, hCD11b, hCD11c, hCD103, hCD14, hCD36, hCD205, hCD109, hVISTA, hMARCO, hMHCII, hCD83, hSIGLEC, hMGL/hClec10A, hASGR (hASGR1/hASGR2), hCD80, hCD86, hClec9A, hClec12A, hClec12B, hDCIR2, hLangerin, hMR, hDC-Sign, hTreml4, hDectin-1, hPDL1, hPDL2, hHVEM, hCD163, hCD32b and hCD141.

The moiety may be a natural ligand, an antibody or part thereof, e.g. a scFv, or a synthetic ligand.

In some embodiments, particularly for tolerance-inducing constructs, the moiety is an antibody or part thereof, e.g. a scFv, with specificity for any of the aforementioned receptors, whose binding to the receptor results in the T cell epitopes being presented in an anti-inflammatory, tolerogenic manner.

In other embodiments, particularly for tolerance-inducing constructs, the moiety is a synthetic ligand with specificity for any of the aforementioned receptors, where binding to the receptor results in the T cell epitopes being presented in an anti-inflammatory, tolerogenic manner. Protein modelling may be used to design such synthetic ligands.

In other embodiments, the moiety is a natural ligand.

In some embodiments, particularly for tolerance-inducing constructs, the natural ligand is selected from the group consisting of TGFβ, such as TGFβ1, TGFβ2 or TGFβ3, IL-10, IL2, IL4, IL6, IL11, IL13, IL27, IL35, IL37, GM-CSF, FLT3L, CCL19, CCL21, ICAM-1 (Intercellular Adhesion Molecule 1 also known as CD54), keratin, VSIG-3, SCGB3A2, CTLA-4, preferably the extracellular domain of CTLA-4, PD-1, preferably the extracellular domain of PD-1 and BTLA, preferably the extracellular domain of BTLA. In other embodiments, particularly for tolerance-inducing constructs, the targeting unit is or comprises IL2, preferably human IL2. In other embodiments, the targeting unit comprises or consists of an amino acid sequence having at least 80% sequence identity to that of human IL2, such as an amino acid sequence having at least 80% sequence identity to SEQ ID NO: 296. In other embodiments, the targeting unit comprises or consists of or a nucleotide sequence encoding human IL2, such as the nucleotide sequence of SEQ ID NO: 252.

In other embodiments, particularly for tolerance-inducing constructs, the targeting unit is or comprises IL-10 or TGFβ, preferably human IL-10 or human TGFβ, including its isoforms TGFβ-1, TGFβ-2 and TGFβ-3.

In other embodiments, particularly for tolerance-inducing constructs, the targeting unit comprises or consists of an amino acid sequence having at least 80% sequence identity to that of human TGFβ, such as an amino acid sequence having at least 80% sequence identity to any of SEQ ID NO: 249-251 or 253-255, preferably SEQ ID NO: 253-255.

In yet other embodiments, particularly for tolerance-inducing constructs, the targeting unit comprises or consists of an amino acid sequence having at least 85% sequence identity to the amino acid sequence of human TGFβ, such as an amino acid sequence having at least 85% sequence identity to any of SEQ ID NO: 249-251 or 253-255, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In another embodiment, particularly for tolerance-inducing constructs, the targeting unit comprises or consists of an amino acid sequence of human TGFβ, such as an amino acid sequence selected from SEQ ID NO: 250-251 or 253-255, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, particularly for tolerance-inducing constructs, the targeting unit comprises or consists of an amino acid sequence of human TGFβ, or a nucleotide sequence encoding human TGFβ.

In other embodiments, particularly for tolerance-inducing constructs, the targeting unit comprises or consists of or a nucleotide sequence encoding human TGFβ, such as a nucleotide sequence selected from SEQ ID NO: 256-258.

In yet other embodiments, particularly for tolerance-inducing constructs, the targeting unit comprises or consists of an amino acid sequence having at least 80% sequence identity to that of murine TGFβ, such as murine TGFβ as set forth in SEQ ID NO: 259.

In yet other embodiments, particularly for tolerance-inducing constructs, the targeting unit comprises or consists of an amino acid sequence having at least 80% sequence identity to that of human IL-10, such as an amino acid sequence having at least 80% sequence identity to SEQ ID NO: 260.

In yet other embodiments, particularly for tolerance-inducing constructs, the targeting unit comprises or consists of an amino acid sequence having at least 85% sequence identity to the amino acid sequence of human IL-10, such as an amino acid sequence having at least 85% sequence identity to SEQ ID NO: 260, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, particularly for tolerance-inducing constructs, the targeting unit comprises or consists of an amino acid sequence of human IL-10, such as the amino acid sequence of SEQ ID NO: 260, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, particularly for tolerance-inducing constructs, the targeting unit comprises or consists of an amino acid sequence of human IL-10, or a nucleotide sequence encoding human IL-10.

In other embodiments, particularly for tolerance-inducing constructs, the targeting unit comprises or consists of or a nucleotide sequence encoding human IL-10, such as the nucleotide sequence of SEQ ID NO: 261.

In yet other embodiments, particularly for tolerance-inducing constructs, the targeting unit comprises or consists of an amino acid sequence having at least 80% sequence identity to that of murine IL-10, such as murine IL-10 as set forth in SEQ ID NO: 262.

In some embodiments, particularly for tolerance-inducing constructs, the targeting unit is or comprises SCGB3A2 or VSIG-3, preferably human VSIG-3 or human SCGB3A2.

In other embodiments, particularly for tolerance-inducing constructs, the targeting unit comprises or consists of an amino acid sequence having at least 80% sequence identity to that of human SCGB3A2, such as an amino acid sequence having at least 80% sequence identity to SEQ ID NO: 263.

In yet other embodiments, particularly for tolerance-inducing constructs, the targeting unit comprises or consists of an amino acid sequence having at least 85% sequence identity to the amino acid sequence of human SCGB3A2, such as an amino acid sequence having at least 85% sequence identity to SEQ ID NO: 263, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, particularly for tolerance-inducing constructs, the targeting unit comprises or consists of an amino acid sequence of human SCGB3A2, such as the amino acid sequence of SEQ ID NO: 263, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, particularly for tolerance-inducing constructs, the targeting unit comprises or consists of an amino acid sequence of human SCGB3A2, or a nucleotide sequence encoding human SCGB3A2.

In other embodiments, particularly for tolerance-inducing constructs, the targeting unit comprises or consists of or a nucleotide sequence encoding human SCGB3A2, such as the nucleotide sequence of SEQ ID NO: 264.

In yet other embodiments, particularly for tolerance-inducing constructs, the targeting unit comprises or consists of an amino acid sequence having at least 80% sequence identity to that of murine SCGB3A2, such as murine SCGB3A2 as set forth in SEQ ID NO: 265.

In yet other embodiments, particularly for tolerance-inducing constructs, the targeting unit comprises or consists of an amino acid sequence having at least 80% sequence identity to that of human VSIG-3, such as an amino acid sequence having at least 80% sequence identity to SEQ ID NO: 266.

In yet other embodiments, particularly for tolerance-inducing constructs, the targeting unit comprises or consists of an amino acid sequence having at least 85% sequence identity to the amino acid sequence of human VSIG-3, such as an amino acid sequence having at least 85% sequence identity to SEQ ID NO: 266, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In another embodiment, particularly for tolerance-inducing constructs, the targeting unit comprises or consists of an amino acid sequence of human VSIG-3, such as the amino acid sequence of SEQ ID NO: 266, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, particularly for tolerance-inducing constructs, the targeting unit comprises or consists of an amino acid sequence of human VSIG-3, or a nucleotide sequence encoding human VSIG-3.

In other embodiments, the targeting unit comprises or consists of or a nucleotide sequence encoding human VSIG-3, such as the nucleotide sequence of SEQ ID NO: 267.

In yet other embodiments, the targeting unit comprises or consists of an amino acid sequence having at least 80% sequence identity to that of murine VSIG-3, such as murine VSIG-3 as set forth in SEQ ID NO: 268.

In yet other embodiments, particularly for tolerance-inducing constructs, the targeting unit is or comprises an antibody or part thereof, e.g. a scFv, with specificity for CD205, such as scFv with specificity for human or murine CD205 or an scFv anti-DEC205. In some embodiments, the scFv with specificity for murine CD205 comprises or consists of SEQ ID NO: 269.

In other embodiments, particularly for tolerance-inducing constructs, the targeting unit comprises or consists of an amino acid sequence having at least 80% sequence identity to that of human CTLA4, such as an amino acid sequence having at least 80% sequence identity to SEQ ID NO: 270.

In yet other embodiments, the targeting unit comprises or consists of an amino acid sequence having at least 85% sequence identity to the amino acid sequence of human CTLA4, such as an amino acid sequence having at least 85% sequence identity to SEQ ID NO: 270, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, particularly for tolerance-inducing constructs, the targeting unit comprises or consists of an amino acid sequence of human CTLA4, such as the amino acid sequence of SEQ ID NO: 270, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, particularly for tolerance-inducing constructs, the targeting unit comprises or consists of an amino acid sequence of human CTLA4, or a nucleotide sequence encoding human CTLA4.

In other embodiments, particularly for tolerance-inducing constructs, the targeting unit comprises or consists of or a nucleotide sequence encoding human CTLA4, such as the nucleotide sequence of SEQ ID NO: 271.

In yet other embodiments, the targeting unit comprises or consists of an amino acid sequence having at least 80% sequence identity to that of murine CTLA4, such as murine CTLA4 as set forth in SEQ ID NO: 272.

In other embodiments, particularly for tolerance-inducing constructs, the targeting unit comprises or consists of an amino acid sequence having at least 80% sequence identity to that of human PD-1, such as an amino acid sequence having at least 80% sequence identity to SEQ ID NO: 273.

In yet other embodiments, the targeting unit comprises or consists of an amino acid sequence having at least 85% sequence identity to the amino acid sequence of human PD-1, such as an amino acid sequence having at least 85% sequence identity to SEQ ID NO: 273, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, the targeting unit comprises or consists of an amino acid sequence of human PD-1, such as the amino acid sequence of SEQ ID NO: 273, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, particularly for tolerance-inducing constructs, the targeting unit comprises or consists of an amino acid sequence of human PD-1, or a nucleotide sequence encoding human PD-1.

In other embodiments, the targeting unit comprises or consists of or a nucleotide sequence encoding human PD-1, such as the nucleotide sequence of SEQ ID NO: 274.

In yet other embodiments, the targeting unit comprises or consists of an amino acid sequence having at least 80% sequence identity to that of murine PD-1, such as murine PD-1 as set forth in SEQ ID NO: 275.

In yet other embodiments, particularly for tolerance-inducing constructs, the targeting unit comprises or consists of an amino acid sequence having at least 80% sequence identity to that of human IL-10, such as an amino acid sequence having at least 80% sequence identity to SEQ ID NO: 260.

In yet other embodiments, particularly for tolerance-inducing constructs, the targeting unit comprises or consists of an amino acid sequence having at least 85% sequence identity to the amino acid sequence of human IL-10, such as an amino acid sequence having at least 85% sequence identity to SEQ ID NO: 260, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98%, such as at least 99% or such as 100% sequence identity thereto.

In other embodiments, particularly for tolerance-inducing constructs, the targeting unit comprises or consists of an amino acid sequence of human IL-10, such as the amino acid sequence of SEQ ID NO: 260, except that at the most 22 amino acids have been substituted, deleted or inserted, such as at the most 21, 20, 19, 18, 17, 16, 15, 14, 13, 12, 11, 10, 9, 8, 7, 6, 5, 4, 3, 2, or 1 amino acid.

In other embodiments, particularly for tolerance-inducing constructs, the targeting unit comprises or consists of an amino acid sequence of human IL-10, or a nucleotide sequence encoding human IL-10.

In other embodiments, particularly for tolerance-inducing constructs, the targeting unit comprises or consists of or a nucleotide sequence encoding human IL-10, such as the nucleotide sequence of SEQ ID NO: 261.

### Further Targeting Unit

In some embodiments, at least one of the one or more further polypeptides comprises a further targeting unit.

This results in the expression of at least two polypeptides comprising targeting units, wherein the first and the one or more further polypeptides can target the same type of cells or different types of cells. In some embodiments, the first polypeptide targets APCs such as dendritic cells and the other polypeptide targets APCs such as MHCII expressing cells.

The skilled person will understand that any specific targeting unit of the first polypeptide described herein may also be a further targeting unit of at least one of the one or more further polypeptide.

In some embodiments, the further targeting unit of at least one further polypeptide is different from the targeting unit of the first polypeptide.

In some embodiments, the further targeting unit of at least one further polypeptide is a MHC II targeting unit.

In some embodiments, the further targeting unit of at least one further polypeptide targets antigen-presenting cells.

In some embodiments, the further targeting unit of at least one further polypeptide is an antibody or part thereof.

In some embodiments, the further targeting unit is a scFv or an alpaca derived VHH, such as a VHH_{MHCII}. VHHs are derived from the variable region of heavy chain-only antibodies present in camelid serum and are the smallest antibody fragments still able to demonstrate antigen specificity.

In some embodiments, the further targeting unit the multimeric protein attracts DCs and other immune cells. Thus, the multimeric protein will not only target the antigenic unit comprised therein to specific cells, but also facilitate a response-amplifying effect (adjuvant effect) by recruiting specific immune cells to the administration site of the vector. Similarly, in some embodiments, the further polypeptide can recruit specific immune cells to the administration site of the vector.

In some embodiments, the further targeting unit is designed to target the multimeric protein, or the further polypeptide, to surface molecules expressed on the APCs, such as molecules expressed on any or many types of APCs or molecules exclusively on subsets of APCs, such as on subsets of DCs.

In some embodiments, the further targeting unit delivers the polypeptide/multimeric protein, or the further polypeptide, to an antigen-presenting cell for MHC class II-restricted presentation to CD4+ T cells or for providing presentation regulatory to CD8+ T cells by classical or non-classical MHC class I restriction.

In some embodiments, the further targeting unit comprises sequences that target APCs for increased promotion of Th1-mediated IgG skewing of the immune response. Targeting for increased promotion of Th1-mediated IgG skewing of the immune response has previously been investigated (Weinberger et al. 2013).

In some embodiments, the further targeting unit binds to surface receptors on the APC, without activating the cell. In some embodiments, the targeting unit binds to surface receptors on the APC, without inducing maturation of the cell.

In some embodiments, the APC internalizes the construct and presents the T cell epitopes comprised in the antigenic unit, or in the further polypeptide, on MHC on its surface in an anti-inflammatory, tolerogenic manner, e.g., by not upregulating co-stimulatory signals and/or by upregulating inhibitory surface molecules and/or by promoting the secretion of inhibitory cytokines.

In some embodiments, the further targeting unit binds to surface molecules on the APCs to induce Th1 polarization and an IgG-dominant antibody response. An example of such surface molecule on APCs is variants or homologs of human or mouse XCL1. In some embodiments, the targeting unit comprises or consists of XCL1. In some embodiments, the targeting unit comprises or consists of human XCL1. In some embodiments, the targeting unit comprises or consists of mouse XCL1. In some embodiments, the targeting unit comprises or consists of an XCL1 homolog or variant. In some embodiments, the targeting unit comprises or consists of a human XCL1 homolog or variant. In some embodiments, the targeting unit comprises or consists of a mouse XCL1 homolog or variant.

In some embodiments, the further targeting unit binds to surface molecules on the APCs to induce Th2 polarization. An example of such surface molecule on APCs is variants or homologs of human or mouse MHCII. In some embodiments, the targeting unit comprises or consists of MHCII. In some embodiments, the targeting unit comprises or consists of human MHCII. In some embodiments, the targeting unit comprises or consists of mouse MHCII. In some embodiments, the targeting unit comprises or consists of a MHCII homolog or variant. In some embodiments, the targeting unit comprises or consists of a human MHCII homolog or variant. In some embodiments, the targeting unit comprises or consists of a mouse MHCII homolog or variant.

In some embodiments, the further targeting unit is an immunogenic targeting unit as described further below.

In some embodiments, the further targeting unit is a tolerance-inducing targeting unit as described further below.

### Further Targeting Unit of Immunogenic Constructs

For embodiments relating to immunogenic constructs, The further targeting unit targets the at least one further polypeptide to APCs. APCs include MHCII expressing cells, dendritic cells (DCs) and subsets thereof.

The term “further targeting unit” as used herein for immunogenic constructs refers to a unit that delivers the construct of the disclosure to an antigen-presenting cell (APC) and interacts with surface molecules on the APC.

For embodiments relating to immunogenic constructs, the further targeting unit may be any targeting unit described in the section “Targeting unit of immunogenic constructs”.

In some embodiments, the first polypeptide comprises an antigenic unit comprising one or more T cell epitopes and a targeting unit that targets said T cell epitopes to APCs, and at least one of the one or more further polypeptides comprises: a further antigenic unit comprising a full-length antigen; and a further targeting unit that targets said antigen to MHCII expressing cells, which may lead to the production of functional antibodies. In some embodiments the further targeting unit is as an antibody-binding region, such as the antibody variable domains (VL and VH), with specificity for MHC class II proteins selected from the group consisting of anti-HLA-DP, anti-HLA-DR and anti-pan HLA class II.

In some embodiments particularly for immunogenic constructs:
i. the targeting unit of the first polypeptide comprises or consists of human CCL3L1;
ii. the antigenic unit of the first polypeptide comprises one or more T cell epitopes, wherein the one or more T cell epitopes are CD4+ and CD8+ T cell epitopes, wherein the one or more T cell epitopes are separated by linkers;
iii. the further antigenic unit of at least one of the one or more further polypeptides comprises one or more epitopes, preferably wherein the one or more epitopes are part of an antigen; and
iv. at least one of the one or more further polypeptides comprises a further targeting unit, wherein the further targeting unit is a scFv or a VHH targeting MHC II, such as a VHH_{MHCII}.

In some embodiments, particularly for immunogenic constructs, the further targeting unit is α-MHC-II scFv. In some embodiments, the further targeting unit is α-MHC-II scFv comprising or consisting of the sequence

(SEQ ID NO: 187)
DIVLTQSPASLAVSLGQRATISCRASKSVSTSGYSYMHWYQQKPGQPPK
LLIYLTSNLESGVPARFSGSGSGTDFTLNIHPVEEEDAATYYCQHSREL
PWTFGGGTKLEIKGGGGSGGGGSGGGGSQVQLQQSGPDLVKPGASVTIS
CKASGYAFSSSWMSWLKQRPGKGLEWIGWIFPRDGDTNYNGKFKGKATL
TADKSSSTAYMQLSSLTSEDSAVYFCARRGDYHYGMDYWGQGTSVTVSS

### Further Targeting Unit of Tolerance-Inducing Constructs

For tolerance-inducing constructs, the further targeting unit binds to surface receptors on the APC without activating the cell and/or without inducing maturation of the cell.

The term “further targeting unit” as used herein for tolerance-inducing constructs refers to a unit that delivers the construct of the disclosure to an APC and interacts with surface molecules on the APC, e.g. binds to surface receptors on the APC, without activating the cell and/or without inducing maturation of the cell. The APC internalizes the construct and presents the T cell epitopes comprised in the antigenic unit on MHC on its surface in an anti-inflammatory, tolerogenic manner, e.g. by not upregulating co-stimulatory signals and/or upregulating inhibitory surface receptors and/or secretion of inhibitory cytokines.

For embodiments relating to tolerance-inducing constructs, the further targeting unit may be any targeting unit described in the section “Targeting unit of tolerance-inducing constructs”.

In some embodiments of the tolerance-inducing constructs:
The first polypeptide comprises an antigenic unit comprising one or more T cell epitopes from an allergen, self-antigen or alloantigen and a targeting unit that targets said T cell epitopes to APCs without activating the APCs, and
At least one of the one or more further polypeptides comprises a further antigenic unit comprising one or more allergens, hypoallergenic allergens, self-antigens or alloantigens and a further targeting unit that targets said allergens or hypoallergenic allergens to APCs, which may lead to the production of functional antibodies without inducing an IgE immune response or in the case of self-antigens or alloantigens to alternative APCs.

In some embodiments the further targeting unit is as an antibody-binding region, such as the antibody variable domains (VL and VH), with specificity for MHC class II proteins selected from the group consisting of anti-HLA-DP, anti-HLA-DR and anti-pan HLA class II, or other targeting units that targets surface receptors on APCs to influence the induced Th1/Th2 polarization and antibody subtypes.

### Signal Peptide

In some embodiments of the present disclosure, the first nucleic acid sequence encodes a first polypeptide comprising a signal peptide. This can be relevant for any embodiment described herein where secretion of an antigen comprised in an antigenic unit, the first polypeptide and/or the one or more further polypeptides is desired, e.g. where the vector is used to transfect a cell and the antigen comprised in an antigenic unit, the first polypeptide and/or the one or more further polypeptides is secreted from such transfected cell.

In some embodiments, the first nucleic acid sequence and/or at least one of the one or more further nucleic acid sequences encode polypeptides comprising a signal peptide. This can be relevant for any embodiment described herein where secretion of T cell epitopes comprised in an antigenic unit and/or of an allergen, a hypoallergenic allergen, a self-antigen or an alloantigen comprised in a further antigenic unit is desired.

The signal peptide encoded by at least one of the one or more further nucleic acid sequences may be referred to as a “further signal peptide”. The further signal peptide may be any signal peptide described in this section.

The signal peptide is preferably located at the N-terminal end of the targeting unit in the first polypeptide, and/or the further polypeptide. The signal peptide is designed to allow secretion of the first polypeptide/further polypeptides from cells comprising a vector of the disclosure. In some preferred embodiments, the first nucleic acid sequence and each of the further nucleic acid sequences also encodes a signal peptide. In some preferred embodiments, the signal peptide is that which is naturally present at the N-terminus of the targeting unit(s) or further polypeptides described herein.

In some embodiments, the first nucleic acid sequence encodes a first polypeptide which comprises a signal peptide, and optionally at least one of the one or more further nucleic acid sequences encodes a further signal peptide.

In some embodiments, the signal peptide encoded by the first nucleic acid sequence and the further signal peptide encoded by at least one further nucleic acid sequences are identical. In some embodiments, the signal peptide comprised in the first polypeptide and in the at least one of the one or more further polypeptides are identical.

In other embodiments, the signal peptide encoded by the first nucleic acid sequence and the further signal peptide encoded by at least one further polypeptide are different.

Any suitable signal peptide may be used. Examples of suitable peptides are an Ig VH signal peptide, preferably a human Ig VH signal peptide, such as SEQ ID NO: 130, preferably if the targeting unit is an antibody or part thereof, such as a scFv. In some embodiments, the signal peptide is the natural leader sequence of the protein which is the targeting unit, i.e., the signal peptide which is naturally present at the N-terminus of any of the protein which is encoded in the vector of the disclosure as the targeting unit. In other embodiments, the signal peptide is the natural leader sequence of the further polypeptide, i.e., the signal peptide which is naturally present at the N-terminus of the further polypeptide.

Examples of signal peptides are a human TPA signal peptide, such as SEQ ID NO: 131, a human CCL3L1 signal peptide, such as the amino acid sequence 1-23 of SEQ ID NO: 128, a human GM-CSF signal peptide, such as the amino acid sequence of SEQ ID NO: 132, a human CCL5 signal peptide, such as the amino acid sequence of SEQ ID NO: 133, a human IL-12A signal peptide, such as the amino acid sequence of SEQ ID NO: 134, a human IL-12B signal peptide, such as the amino acid sequence of SEQ ID NO: 135 or a human IL-21 signal peptide, such as the amino acid sequence of SEQ ID NO: 136.

In some preferred embodiments, at least one signal peptide encoded by the vector comprises an amino acid sequence having at least 85% sequence identity to the amino acid sequence 1-23 of SEQ ID NO: 128, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In other preferred embodiments, at least one signal peptide encoded by the vector comprises the amino acid sequence 1-23 of SEQ ID NO: 128, except that at the most three amino acids have been substituted, deleted or inserted, such as at the most two amino acids or such as at the most one amino acid.

In other preferred embodiments, at least one signal peptide encoded by the vector comprises the amino acid sequence 1-23 of SEQ ID NO: 128.

In some more preferred embodiments, at least one signal peptide encoded by the vector consists of an amino acid sequence having at least 85% sequence identity to the amino acid sequence 1-23 of SEQ ID NO: 128, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In other preferred embodiments, at least one signal peptide encoded by the vector consists of the amino acid sequence 1-23 of SEQ ID NO: 128, except that at the most three amino acids have been substituted, deleted or inserted, such as at the most two amino acids or such as at the most one amino acid.

In other preferred embodiments, at least one signal peptide encoded by the vector consists of the amino acid sequence 1-23 of SEQ ID NO: 128.

In some preferred embodiments, the nucleotide sequence of at least one signal peptide encoded by the vector has at least 80% sequence identity to the nucleic acid sequence with SEQ ID NO: 137.

In some further preferred embodiments, the nucleotide sequence of at least one signal peptide encoded by the vector has at least 85% sequence identity to the nucleic acid sequence with SEQ ID NO: 137, such as at least 86% or at least 87%, 88%, 89%, 90%, 91%, 92%, 93%, 94%, 95%, 96%, 97%, 98% or at least 99% sequence identity.

In yet some further preferred embodiments, the nucleotide sequence of at least one signal peptide encoded by the vector is SEQ ID NO: 137.

In some embodiments, the signal peptide and/or the further signal peptide of at least one further polypeptide is selected from the group consisting of Ig VH signal peptide, human serum albumin signal peptide (SEQ ID NO: 138), human modified IgG H signal peptide (SEQ ID NO: 139), human HC H6 signal peptide (SEQ ID NO: 140), human TPA signal peptide and human CCL3L1 signal peptide.

In some embodiments, the targeting unit is human CCL3L1 and the signal peptide and/or the further signal peptide of at least one further polypeptide comprises an amino acid sequence having at least 85% sequence identity to the amino acid sequence 1-23 of SEQ ID NO: 128

In some embodiments, the signal peptide and/or the further signal peptide of at least one further polypeptide consists of an amino acid sequence having at least 85% sequence identity to the amino acid sequence 1-23 of SEQ ID NO: 128.

In some embodiments, the signal peptide and/or the further signal peptide of at least one further polypeptide consists of the amino acid sequence 1-23 of SEQ ID NO: 128.

For some embodiments relating to tolerance-inducing constructs, additional examples of suitable peptides are a human Ig VH signal peptide or the signal peptides which are naturally present at the N-terminus of any of the targeting units described herein, e.g. a human signal peptide of human IL-10 or a human signal peptide of human TGFβ.

Thus, in some embodiments, the vector comprises a nucleotide sequence encoding a human IL-10 signal peptide and preferably comprises a nucleotide sequence encoding a human IL-10 targeting unit. In other embodiments, the vector comprises a nucleotide sequence encoding a human Ig VH signal peptide and preferably comprises a nucleotide sequence encoding a scFv, e.g. human anti-DEC205.

In some embodiments, in particular for tolerance-inducing constructs, the vector comprises a nucleotide sequence encoding a signal peptide that comprises an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%, sequence identity to the amino acid sequence of SEQ ID NO: 130 or SEQ ID NO: 278.

In preferred embodiments, in particular for tolerance-inducing constructs, the vector comprises a nucleotide sequence encoding a signal peptide that comprises the amino acid sequence of SEQ ID NO: 130 OR SEQ ID NO: 278.

In other embodiments, in particular for tolerance-inducing constructs, the vector comprises a nucleotide sequence encoding a signal peptide that consists of an amino acid sequence having at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% to the amino acid sequence of SEQ ID NO: 130 OR SEQ ID NO: 278.

In other preferred embodiments, the vector comprises a nucleotide sequence encoding a signal peptide with the amino acid sequence of SEQ ID NO: 130 OR SEQ ID NO: 278.

In other embodiments, in particular for tolerance-inducing constructs, the vector comprises a nucleotide sequence encoding a signal peptide that comprises or consists of an amino acid sequence of SEQ ID NO: 130 OR SEQ ID NO: 278, except that at the most five amino acids have been substituted, deleted or inserted, such as at the most four amino acids, such as at the most three amino acids, such as at the most two amino acids or such as at the most one amino acid.

In some embodiments, in particular for tolerance-inducing constructs, the vector comprises a nucleotide sequence encoding a murine IL-10 signal peptide, such as the IL-10 signal peptide set forth in SEQ ID NO: 279, and preferably comprises a nucleotide sequence encoding a murine IL-10 targeting unit, such as the murine IL-10 targeting unit set forth in SEQ ID NO: 261.

In some embodiments, in particular for tolerance-inducing constructs, the signal peptide is selected from the group consisting of IL-10 signal peptide, SCGB3A2 signal peptide, VSIG-3 signal peptide, CTLA4 signal peptide, or PD-1 signal peptide, such as selected from the group consisting of murine IL-10 signal peptide, murine SCGB3A2 signal peptide, murine VSIG-3 signal peptide, murine CTLA4 signal peptide, or murine PD-1 signal peptide. In some embodiments, the signal peptide comprises a sequence having 80% sequence identity to a sequence selected from the group consisting of SEQ ID NO: 279, 280, 281, 282, 283 and 284.

### Further Signal Peptide

In some embodiments, the first nucleic acid sequence encodes a signal peptide and/or at least one of the one or more further nucleic acid sequences encodes a further signal peptide.

The skilled person will understand that any specific signal peptide described herein in relation to the first polypeptide may also be a further signal peptide of at least one of the one or more further polypeptide.

In some embodiments, the first nucleic acid sequence encodes a first polypeptide which further comprises a signal peptide, and preferably also at least one of the one or more further nucleic acid sequences further encodes a further signal peptide.

In some embodiments, the first polypeptide comprises a signal peptide and/or at least one of the one or more further polypeptides comprises a further signal peptide.

In some embodiments, the first polypeptide does not comprise a signal peptide and at least one of the one or more further polypeptides comprises a further signal peptide.

In some embodiments, the further signal peptide of at least one further polypeptide is different from the signal peptide of the targeting unit of the first polypeptide.

In some embodiments, the signal peptide and/or the further signal peptide of at least one further polypeptide is the natural leader sequence of the protein which is the targeting unit.

In some embodiments, at least one of the one or more further polypeptides comprises:
i. a further signal peptide; and
ii. a further antigenic unit comprising one or more further epitopes.

In some embodiments, at least one of the one or more further polypeptides comprise:
i. a further signal peptide;
ii. an interaction unit; and
iii. a further antigenic unit comprising one or more further epitopes.

In some embodiments, at least one of the one or more further polypeptides comprise:
i. a further signal peptide;
ii. an interaction unit, such as a further multimerization unit; and
iii. a further antigenic unit comprising one or more further epitopes.

In some embodiments, the antigenic unit of the first polypeptide comprises one or more B cell epitopes.

In some embodiments, at least one of the one or more further polypeptides comprises:
i. a further signal peptide; and
ii. a further antigenic unit comprising one or more allergens, hypoallergenic allergens, self-antigens or alloantigens.

In some embodiments, at least one of the one or more further polypeptides comprise:
i. a further signal peptide;
ii. an interaction unit, such as a further dimerization unit; and
iii. a further antigenic unit comprising one or more allergens, hypoallergenic allergens, self-antigens or alloantigens.

In some embodiments, at least one of the one or more further polypeptides comprise:
i. a further signal peptide;
ii. a further targeting unit;
iii. a further dimerization unit unit; and
iv. a further antigenic unit comprising one or more allergens, hypoallergenic allergens, self-antigens, or alloantigens.

### Multimerization Unit/Dimerization Unit

The first polypeptide encoded by the first nucleic acid comprised in the vector of the disclosure comprises a multimerization unit, such as a dimerization unit. The multimerization unit may allow the formation of a multimer of first polypeptides, for example a dimerization unit allows the formation of a dimer of first polypeptides, or it may allow the formation of a multimer of the first polypeptide and at least one of the further polypeptides as described below.

Multimerization or dimerization units that can be used in the context of the present invention are described in application PCT/EP2022/061819, in particular in the section entitled “Dimerization/multimerization unit”.

The term “multimerization unit” as used herein refers both to the multimerization unit of the first polypeptide, and the interaction unit of a further polypeptide, wherein the interaction unit is a further multimerization unit. In some embodiments, the multimerization unit facilitates multimerization of multiple first polypeptides. In some embodiments, the multimerization unit facilitates multimerization of multiple further polypeptides. In some embodiments, the multimerization unit facilitates multimerization of one or more first polypeptides and one or more further polypeptides.

By reading the disclosure, the skilled will understand that the interaction unit of the further polypeptide encompasses further embodiments that are not encompassed by the multimerization unit of the first polypeptide, i.e., wherein the interaction unit is not a further multimerization unit. These embodiments are described in the section “Interaction unit”.

In the context of the first polypeptide, and of a further polypeptide comprising equivalent structural elements, the term “multimerization unit” as used herein refers to a sequence of nucleotides or amino acids between the antigenic unit and the targeting unit of the first polypeptide. In addition to connecting the antigenic unit and the targeting unit, the multimerization unit facilitates multimerization of/joins multiple polypeptides, such as two, three, four or more polypeptides, into a multimeric protein, such as a dimeric protein, a trimeric protein or a tetrameric protein. Furthermore, the multimerization unit also provides flexibility in the multimeric protein to allow optimal binding of the targeting unit to the surface molecules on the APCs, even if they are located at variable distances. The multimerization unit may be any unit that fulfils one or more of these requirements.

In some embodiments, at least one of the one or more further polypeptides comprises an interaction unit, such as a further a further multimerization unit, such as a further dimerization unit, a further trimerization unit, or a further tetramerization unit. In some embodiments, the first polypeptide and at least one of the one or more further polypeptides can form a multimer, such as a dimer, optionally as heteromultimer, such as a heterodime. Multimerization between the first polypeptide and the further polypeptides occurs via interaction between the multimerization unit and the interaction unit, such as the further multimerization unit, of the first polypeptide and the further polypeptides, respectively. In some embodiments, the multimerization unit of the first polypeptide and the interaction unit of the further polypeptide do not interact, i.e. the interaction occurs between the interaction units of several further polypeptides. As an example, the vector encodes a first and a further polypeptide, wherein the first polypeptide comprises a dimerization unit and the further polypeptides comprises an interaction unit in the form of a further dimerization unit which leads to the formation of homodimers of 2 first polypeptides and homodimers of 2 further polypeptides, i.e. the first and further polypeptides do not comprise dimerization units which are able to interact with each other.

In some embodiments, the multimerization unit of the first polypeptide is selected from the group consisting of dimerization unit, trimerization unit, such as a collagen-derived trimerization unit, such as a human collagen-derived trimerization domain, such as human collagen derived XVIII trimerization domain or human collagen XV trimerization domain or the C-terminal domain of T4 fibritin and tetramerization unit, such as a domain derived from p53 and wherein said multimerization unit optionally comprises a hinge region, such as hinge exon h1 and hinge exon h4. In some embodiments, the trimerization unit comprises or consists of the C-terminal domain of T4 fibritin. In some embodiments, the trimerization unit comprises or consist of amino acid residues 460-481 of SEQ ID NO: 200.

In some embodiments, at least one further polypeptide comprises an interaction unit, such as a further multimerization unit, such as a further dimerization unit, a further trimerization unit or a further tetramerization unit, which allows the formation of a multimer, such as a dimer, a trimer or a tetramer of the further polypeptides, or which allows the formation of a multimer, such as a dimer, a trimer or a tetramer of the first polypeptide and at least one of the one or more further polypeptides. Thus, in some embodiments, the first polypeptide and at least one further polypeptide are capable of forming a multimer, such as a dimer. In some embodiments, the multimerization unit and the further multimerization unit are capable of forming a multimer, such as a dimer, such as a heterodimer. In some embodiments, the multimer is a heterodimer.

Multimerization Unit that Facilitates Multimerization of/Joins More than Two Polypeptides

In some embodiments, the multimerization unit is a trimerization unit, such as a collagen-derived trimerization unit, such as a human collagen-derived trimerization domain, such as human collagen derived XVIII trimerization domain (see for instance A. Alvarez-Cienfuegos et al., Sci Rep 6, 28643 (2016)) or human collagen XV trimerization domain. Thus, in some embodiments, the multimerization unit is a trimerization unit that comprises or consists of the nucleic acid sequence with SEQ ID NO: 141, or an amino acid sequence encoded by said nucleic acid sequence. In other embodiments, the trimerization unit is the C-terminal domain of T4 fibritin. Thus, in some embodiments, the multimerization unit is a trimerization unit that comprises or consists of the amino acid sequence with SEQ ID NO: 142. In other embodiments the trimerization unit is a C-terminal coiled-coil region of mouse and human cartilage matrix protein (CMP), as described in Kim et al. Biochemistry (2013). In some embodiment, the trimerization unit is a heterotrimerization unit, i.e. a trimerization unit that leads to the formation of a trimer comprising at least two different polypeptides. In other embodiments, the multimerization unit is a tetramerization unit, such as a domain derived from p53, optionally further comprising a hinge region as described below. Thus, in some embodiments, the multimerization unit is a tetramerization unit that comprises or consists of the nucleic acid sequence with SEQ ID NO: 143, or an amino acid sequence encoded by said nucleic acid sequence, optionally further comprising a hinge region as described below.

### Dimerization Unit

The term “dimerization unit” as used herein, refers to a sequence of nucleotides or amino acids between the antigenic unit and the targeting unit. In addition to connecting the antigenic unit and the targeting unit, the dimerization unit facilitates dimerization of/joins two monomeric polypeptides into a dimeric protein. Furthermore, the dimerization unit also provides the flexibility in the dimeric protein to allow optimal binding of the targeting unit to the surface molecules on the APCs, even if they are located at variable distances. The dimerization unit may be any unit that fulfils these requirements.

Accordingly, in some embodiments the first polypeptide and/or at least one further polypeptide comprises a dimerization unit comprising a hinge region. In other embodiments, the dimerization unit comprises a hinge region and another domain that facilitates dimerization. In yet other embodiments, the dimerization unit comprises a hinge region, a dimerization unit linker and another domain that facilitates dimerization, wherein the dimerization unit linker connects the hinge region and the other domain that facilitates dimerization. In some embodiments, the dimerization unit linker is a glycine-serine rich linker, preferably GGGSSGGGSG (SEQ ID NO: 15), i.e., the dimerization unit comprises a glycine-serine rich dimerization unit linker and preferably the dimerization unit linker GGGSSGGGSG (SEQ ID NO: 15).

The term “hinge region” refers to an amino acid sequence comprised in the dimerization unit that contributes to joining two of the polypeptides, i.e., facilitates the formation of a dimeric protein. In the context of a multimerization unit that facilitates multimerization of/joins more than two polypeptides, the term “hinge region” refers to an amino acid sequence comprised in such multimerization unit that contributes to joining more than two polypeptides, e.g., three or four polypeptides and/or functioning as a flexible spacer, allowing the two targeting units of the multimeric protein to bind simultaneously to multiple surface molecules on APCs, even if they are located at variable distances.

Moreover, the hinge region functions as a flexible spacer, allowing the two targeting units of the dimeric protein to bind simultaneously to two surface molecules on APCs, even if they are located at variable distances. In some embodiments, the hinge region is Ig derived, such as derived from IgG, e.g., IgG1 or IgG2 or IgG3. In some embodiments, the hinge region is derived from IgM, e.g., comprising or consisting of the nucleotide sequence with SEQ ID NO: 144 or an amino acid sequence encoded by said nucleic acid sequence.

The hinge region may contribute to the dimerization through the formation of covalent bond(s), e.g., disulfide bridge(s) between cysteines. Thus, in some embodiments, the hinge region has the ability to form one or more covalent bonds. Preferably, the covalent bond is a disulfide bridge.

In some embodiments, the dimerization unit comprises or consists of a hinge exon h1 and hinge exon h4 (human hinge region 1 and human hinge region 4), preferably hinge exon h1 and hinge exon h4 from IgG3, more preferably having an amino acid sequence of at least 80% sequence identity to the amino acid sequence 94-120 of SEQ ID NO: 128.

In some preferred embodiments, the dimerization unit comprises or consists of a hinge exon h1 and hinge exon h4 with an amino acid sequence of at least 85% sequence identity to the amino acid sequence 94-120 of SEQ ID NO: 128, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In some preferred embodiments, the dimerization unit comprises or consists of a hinge exon h1 and hinge exon h4 with the amino acid sequence 94-120 of SEQ ID NO: 128.

In some preferred embodiments, the dimerization unit comprises or consists of the amino acid sequence 94-120 of SEQ ID NO: 128, except that at the most four amino acids have been substituted, deleted or inserted, such as at the most three amino acids, such as at the most two amino acids or such as at the most one amino acid.

In some preferred embodiments, the dimerization unit comprises or consists of a nucleic acid sequence having at least 80% sequence identity to the nucleic acid sequence with SEQ ID NO: 145.

In some further preferred embodiments, the dimerization unit comprises or consists of a nucleic acid sequence having at least 85% sequence identity to the nucleic acid sequence with SEQ ID NO: 145, such as at least 86% or at least 87%, 88%, 89%, 90%, 91%, 92%, 93%, 94%, 95%, 96%, 97%, 98% or at least 99% sequence identity.

In yet some further preferred embodiments, the dimerization unit comprises or consists of the nucleic acid sequence of SEQ ID NO: 145.

In other embodiments, the dimerization unit comprises another domain that facilitates dimerization, said other domain is an immunoglobulin domain, such as an immunoglobulin constant domain (C domain), such as a CH1 domain, a CH2 domain or a carboxyterminal C domain (i.e., a CH3 domain), or a sequence that is substantially identical to such C domains or a variant thereof. Preferably, the other domain that facilitates dimerization is a carboxyterminal C domain derived from IgG. More preferably, the other domain that facilitates dimerization is a carboxyterminal C domain derived from IgG3.

In some embodiments, the dimerization unit comprises or consists of a carboxyterminal C domain derived from IgG3 with an amino acid sequence having at least 80% sequence identity to the amino acid sequence 131-237 of SEQ ID NO: 128.

In some preferred embodiments, the dimerization unit comprises or consists of a carboxyterminal C domain derived from IgG3 with an amino acid sequence having at least 85% sequence identity to the amino acid sequence 131-237 of SEQ ID NO: 128, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In some preferred embodiments, the dimerization unit comprises or consists of a carboxyterminal C domain derived from IgG3 with the amino acid sequence 131-237 of SEQ ID NO: 128.

In some preferred embodiments, the dimerization unit comprises or consists of the amino acid sequence 131-237 of SEQ ID NO: 128, except that at the most 16 amino acids have been substituted, deleted or inserted, such as at the most 15, 14, 13, 12, 11, 10, 9, 8, 7, 6, 5, 4, 3, 2, or 1 amino acid.

In some preferred embodiments, the dimerization unit comprises or consists of a nucleic acid sequence having at least 80% sequence identity to the nucleic acid sequence with SEQ ID NO: 146.

In some further preferred embodiments, the dimerization unit comprises or consists of a nucleic acid sequence having at least 85% sequence identity to the nucleic acid sequence with SEQ ID NO: 146, such as at least 86% or at least 87%, 88%, 89%, 90%, 91%, 92%, 93%, 94%, 95%, 96%, 97%, 98% or at least 99% sequence identity. In yet some further preferred embodiments, the dimerization unit comprises or consists of the nucleic acid sequence of SEQ ID NO: 146.

The immunoglobulin domain contributes to dimerization through non-covalent interactions, e.g., hydrophobic interactions. Thus, in some embodiments, the immunoglobulin domain has the ability to form dimers via noncovalent interactions. Preferably, the noncovalent interactions are hydrophobic interactions.

It is preferred that if the dimerization unit comprises a CH3 domain, it does not comprise a CH2 domain and vice versa.

In some preferred embodiments, the dimerization unit comprises a hinge exon h1, a hinge exon h4, a dimerization unit linker and a CH3 domain of human IgG3. In some further preferred embodiments, the dimerization unit comprises a polypeptide consisting of hinge exon h1, hinge exon h4, a dimerization unit linker and a CH3 domain of human IgG3. In other preferred embodiments, the dimerization unit consists of a polypeptide consisting of hinge exon h1, hinge exon h4, a dimerization unit linker and a CH3 domain of human IgG3.

In some embodiments, the dimerization unit comprises an amino acid sequence having at least 80% sequence identity to the amino acid sequence 94-237 SEQ ID NO: 128.

In some preferred embodiments, the dimerization unit comprises an amino acid sequence having at least 85% sequence identity to the amino acid sequence 94-237 SEQ ID NO: 128, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99% sequence identity.

In an even more preferred embodiment, the dimerization unit comprises the amino acid sequence 94-237 of SEQ ID NO: 128.

In some more preferred embodiments the dimerization unit consists of an amino acid sequence having at least 80% sequence identity to the amino acid sequence 94-237 of SEQ ID NO: 128, such as at least 85%, such as at least 86%, such as at least 87%, such as at least 88%, such as at least 89%, such as at least 90%, such as at least 91%, such as at least 92%, such as at least 93%, such as at least 94%, such as at least 95%, such as at least 96%, such as at least 97%, such as at least 98% or such as at least 99%.

In an even more preferred embodiment, the dimerization unit consists of the amino acid sequence 94-237 of SEQ ID NO: 128.

In some preferred embodiments, the dimerization unit comprises or consists of the amino acid sequence 94-237 of SEQ ID NO: 128, except that at the most 28 amino acids have been substituted, deleted or inserted, such as at the most 25, 20, 15, 14, 13, 12, 11, 10, 9, 8, 7, 6, 5, 4, 3, 2 or 1 amino acids. In some preferred embodiments, the dimerization unit comprises or consists of a nucleic acid sequence having at least 80% sequence identity to the nucleic acid sequence with SEQ ID NO: 147.

In some further preferred embodiments, the dimerization unit comprises or consists of a nucleic acid sequence having at least 85% sequence identity to the nucleic acid sequence with SEQ ID NO: 147, such as at least 86% or at least 87%, 88%, 89%, 90%, 91%, 92%, 93%, 94%, 95%, 96%, 97%, 98% or at least 99% sequence identity. In yet some further preferred embodiments, the dimerization unit comprises or consists of the nucleic acid sequence of SEQ ID NO: 147.

In the first polypeptide encoded by the first nucleic acid sequence, or in a further polypeptide comprising a multimerization unit, the multimerization unit, e.g., dimerization unit, may have any orientation with respect to antigenic unit and targeting unit. In some embodiments, the antigenic unit is connected to the C-terminal end of the multimerization/dimerization unit (e.g., via an unit linker) with the targeting unit being connected to the N-terminal end of the multimerization/dimerization unit. In other embodiments, the antigenic unit is connected to the N-terminal end of the multimerization/dimerization unit (e.g., via an unit linker) with the targeting unit being connected to the C-terminal end of the multimerization/dimerization unit. It is preferred that the antigenic unit is connected to the C-terminal end of the multimerization/dimerization unit, e.g., via a linker, preferably via the unit linker, and the targeting unit is connected to the N-terminal end of the multimerization/dimerization unit.

### Interaction Unit

In some embodiments, physical interaction between the first polypeptide and the one or more further polypeptides is desired. Thus, in some embodiments, the first polypeptide comprises an interaction unit and at least one of the one or more further polypeptides comprises an interaction unit. The interaction unit can promote interaction between the one or more further polypeptides and the first polypeptide, and/or can result in the formation of dimers, such as heterodimers, trimers, such as heterotrimers, oligomers, multimers or aggregates between the one or more further polypeptides and the first polypeptide.

In some embodiments, the first polypeptide comprises a trimerization unit, such as a heterotrimerization unit, and at least one of the one or more further polypeptides comprises a trimerization unit, such as a heterotrimerization unit. In some embodiments, the antigenic unit of the first polypeptide is capable of forming a trimer with two antigenic units of two further polypeptides. In some embodiments, the first polypeptide and the at least one of the one or more further polypeptides comprises complimentary coiled coil dimer-forming peptides, such as coiled coil peptide A (SEQ ID NO: 194) and coiled coil peptide B (SEQ ID NO: 197) pair, P7A:P8A pair, N7:N8 pair, or N5:N6 pair. In some embodiments, the first polypeptide comprises a coiled coil peptide A (SEQ ID NO: 194) and the one or more further polypeptides comprises a coiled coil peptide B (SEQ ID NO: 197). In some embodiments, the first polypeptide comprises P7A and the one or more further polypeptides comprises P8A. In some embodiments, the first polypeptide comprises N7 and the one or more further polypeptides comprises N8. In some embodiments, the first polypeptide comprises N5 and the one or more further polypeptides comprises N6. In some embodiments, the first polypeptide comprises a coiled coil peptide A (SEQ ID NO: 194) and the one or more further polypeptides comprises a coiled coil peptide B (SEQ ID NO: 197) which results in the formation of a trimer between the antigenic unit of the first polypeptide and two antigenic units of two further polypeptides. In some embodiments, the first polypeptide comprises P7A and the one or more further polypeptides comprises P8A which results in the formation of a trimer between the antigenic unit of the first polypeptide and two antigenic units of two further polypeptides. In some embodiments, the first polypeptide comprises N7 and the one or more further polypeptides comprises N8 which results in the formation of a trimer between the antigenic unit of the first polypeptide and two antigenic units of two further polypeptides. In some embodiments, the first polypeptide comprises N5 and the one or more further polypeptides comprises N6 which results in the formation of a trimer between the antigenic unit of the first polypeptide and two antigenic units of two further polypeptides. In some embodiments, the first polypeptide comprises a coiled coil peptide A (SEQ ID NO: 194) and the one or more further polypeptides comprises a coiled coil peptide B (SEQ ID NO: 197) which results in the formation of a trimer comprising the antigenic unit of the first polypeptide and two antigenic units of two further polypeptides. In some embodiments, the first polypeptide comprises P7A and the one or more further polypeptides comprises P8A which results in the formation of a trimer comprising the antigenic unit of the first polypeptide and two antigenic units of two further polypeptides. In some embodiments, the first polypeptide comprises N7 and the one or more further polypeptides comprises N8 which results in the formation of a trimer comprising the antigenic unit of the first polypeptide and two antigenic units of two further polypeptides. In some embodiments, the first polypeptide comprises N5 and the one or more further polypeptides comprises N6 which results in the formation of a trimer comprising the antigenic unit of the first polypeptide and two antigenic units of two further polypeptides.

In some embodiments, the interaction unit interacts with the multimerization unit of the first polypeptide. In some embodiments, the interaction unit interacts with the antigenic unit of the first polypeptide, whereby the first polypeptide and the further polypeptide form a complex or a multimer.

In some embodiments, physical interaction between at least two of the one or more further polypeptides is desired. Thus, in some embodiments, at least two of the one or more further polypeptides comprise an interaction unit. The interaction unit can promote interaction between at least two of the one or more further polypeptides, and/or can result in the formation of dimers, such as heterodimers, trimers, such as heterotrimers, oligomers, multimers or aggregates between at least two of the one or more further polypeptides.

In some embodiments, the interaction unit is a multimerization unit selected from the group consisting of a dimerization unit, a trimerization unit, and a tetramerization unit, for example as described previously herein.

In some embodiments, the interaction unit is selected from the group consisting of a leucine zipper motif, a sequence capable of promoting oligomerization, such as a homo-trimerization domain, a heterodimerization unit, such as an heterodimerization unit comprising or consisting of a coiled coil dimer-forming peptide, an oligomerization unit and a self-assembly unit.

In some embodiments, at least one of the one or more further polypeptides comprises a self-assembly unit, such as a sequence that promotes formation of nanoparticles, such as antigen-nanoparticles.

In some embodiments, the oligomerization unit of at least one further polypeptide is sortase A.

In some embodiments, the self-assembly unit of at least one further polypeptide is ferritin.

In some embodiments, the oligomerization unit of at least one further polypeptide is sortase A.

In some embodiments, the self-assembly unit of at least one further polypeptide is derived from the self-forming structure component of a self-assembling molecule, such as ferritin, lumazine synthase from Aquifex aeolicus (LS), E2 from Geobacillus stearothermophilus, or I3-01—modified 2-dehydro-3-deoxy-phosphogluconate aldolase (=2-Keto-3-deoxy-6-phosphogluconate (KDPG) aldolase).

In some embodiments, the oligomerization unit is selected from the group consisting of: sortase A; lumazine synthase from Aquifex aeolicus (LS), E2 from Geobacillus stearothermophilus, and I3-01—modified 2-dehydro-3-deoxy-phosphogluconate aldolase (=2-Keto-3-deoxy-6-phosphogluconate (KDPG) aldolase).

Examples of other suitable oligomerization units can be found in López-Sagaseta et al., (2015); Rappuoli et al., 2019; Houser et al., 2022; Rodrigues et al., 2021; Qu et al., 2021; Joyce et al., 2022; Wang et al., 2022; Mu et al., 2022; Wichgers et al., 2021; Zottig et al., 2020; Ma et al., 2020; Walls et al., 2020; and in Curley et al., 2022, which are incorporated herein by reference.

In some embodiments, the heterodimerization unit and the further heterodimerization unit are capable of forming a heterodimer.

In some embodiments of e.g. immunogenic constructs, the vector encodes a first polypeptide wherein:
i. the targeting unit is CCL3L1;
ii. the multimerization unit is a hinge exon h1, a hinge exon h4, a dimerization unit linker and a CH3 domain of human IgG3;
iii. the antigenic unit comprises one antigen;
   and the vector further encodes
iv. at least one of the one or more further polypeptides comprises an interaction unit which is a self-assembly unit, such as ferritin; or said further polypeptide is capable of spontaneously forming an oligomer or multimer.

In some embodiments of e.g. immunogenic constructs, the vector encodes a first polypeptide wherein:
i. the targeting unit is CCL3L1;
ii. the multimerization unit is a hinge exon h1, a hinge exon h4, a dimerization unit linker and a CH3 domain of human IgG3;
iii. the antigenic unit comprises one antigen;
iv. the vector further encodes at least one of the one or more further polypeptides comprises an interaction unit which is a self-assembly unit, such as ferritin; or said further polypeptide is capable of spontaneously forming an oligomer or multimer; and
v. the vector further encodes for an additional polynucleotide encoding one or more immunostimulatory compounds.

In some embodiments of e.g. immunogenic constructs, the vector encodes a first polypeptide wherein:
i. the targeting unit is CCL3L1;
ii. the multimerization unit is a hinge exon h1, a hinge exon h4, a dimerization unit linker and a CH3 domain of human IgG3;
iii. the antigenic unit comprises one antigen;
iv. the vector further encodes at least one of the one or more further polypeptides comprises an interaction unit which is a self-assembly unit, such as ferritin; or said further polypeptide is capable of spontaneously forming an oligomer or multimer; and
v. the further antigenic unit of the at least one further polypeptide comprises a further antigen, wherein the further antigen is a variant of the antigen having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.

This may allow for including of further antigens having slightly different sequences, such as antigens from different strains of pathogens.

In some embodiments of e.g. immunogenic constructs, the vector encodes a first polypeptide wherein:
i. the targeting unit is CCL3L1;
ii. the multimerization unit is a hinge exon h1, a hinge exon h4, a dimerization unit linker and a CH3 domain of human IgG3;
iii. the antigenic unit comprises one antigen;
iv. at least one of the one or more further polypeptides comprises an interaction unit which is a self-assembly unit, such as ferritin; or said further polypeptide is capable of spontaneously forming an oligomer or multimer;
v. the further antigenic unit of the at least one further polypeptide comprises a further antigen, wherein the further antigen is a variant of the antigen having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto; and
vi. the vector encodes for an additional polynucleotide encoding one or more immunostimulatory compounds.

In the case of constructs where the antigenic unit comprises one or more T cell epitope of an allergen, self-antigen or alloantigen, in some embodiments, the vector encodes a first polypeptide wherein:
i. the targeting unit binds to surface receptors on the APC without activating the cell and/or without inducing maturation of the cell;
ii. the multimerization unit is a hinge exon h1, a hinge exon h4, a dimerization unit linker and a CH3 domain of human IgG3;
iii. the antigenic unit comprises one or more T cell epitopes of an allergen, self-antigen or alloantigen; and
iv. the vector further encodes at least one of the one or more further polypeptides comprises an interaction unit which is a self-assembly unit, such as ferritin; or said further polypeptide is capable of spontaneously forming an oligomer or multimer.

In some embodiments, the vector encodes a first polypeptide wherein:
i. the targeting unit binds to surface receptors on the APC without activating the cell and/or without inducing maturation of the cell
ii. the multimerization unit is a hinge exon h1, a hinge exon h4, a dimerization unit linker and a CH3 domain of human IgG3;
iii. the antigenic unit comprises one or more T cell epitopes of an allergen, self-antigen or alloantigen;
iv. the vector further encodes at least one of the one or more further polypeptides comprises an interaction unit which is a self-assembly unit, such as ferritin; or said further polypeptide is capable of spontaneously forming an oligomer or multimer; and
v. the further antigenic unit of the at least one further comprises an allergen, a hypoallergenic allergen, a self-antigen or an alloantigen, optionally wherein the vector further encodes one or more immunoinhibitory compounds.
   Multimerization with the Multimerization Unit of the First Polypeptide.

In some embodiments, at least one of the one or more further nucleic acid sequences encodes a further polypeptide, wherein the further polypeptide comprises a further targeting unit that targets antigen-presenting cells, an interaction unit such as a further multimerization unit, such as a further dimerization unit, and a further antigenic unit comprising one or more further epitopes as described herein. In some embodiments, the first polypeptide and at least one of the one or more further polypeptides are different.

The skilled person will understand that any specific multimerization unit of the first polypeptide described herein may also be a further multimerization unit (or interaction unit) of at least one of the one or more further polypeptides.

In some embodiments, the multimerization unit of the first polypeptide and the interaction unit of at least one of the one or more further polypeptides are different and do not interact, thus, no multimer of the first polypeptide and the at least one further polypeptide is formed. Thus, in some embodiments, the first polypeptide comprises a multimerization unit allowing for the formation of a multimer of the first polypeptide, for example a homodimer consisting of 2 first polypeptides, while the further polypeptide comprises an interaction unit which is a further multimerization unit which differs from the multimerization unit of the first polypeptide, allowing for the formation of a multimer of the further polypeptide, for example a homodimer consisting of 2 further polypeptides. This allows for the expression of two different homodimeric proteins from the same construct. In some particular embodiments, the antigenic unit of the first polypeptide comprises for example T cell epitopes as described herein elsewhere, and the further antigenic unit of the further polypeptide comprises for example antigens, allergens, hypoallergenic allergens, self-antigens or alloantigens such as described in the section “Antigenic unit”.

In some embodiments, the first polypeptide and at least one of the one or more further polypeptides are capable of forming a multimer, such as a dimer, such as a heterodimer, via the interaction between the multimerization unit and the interaction unit.

In some embodiments, the multimerization unit and the interaction unit are capable of forming a multimer, such as a heteromultimer, such as a dimer, such as a heterodimer.

In some embodiments, the multimerization unit and the interaction unit are different and capable of forming a multimer consisting of first and further polypeptide(s), such as a heterodimer.

In some embodiments, the multimer is a heterodimer of a first and a further polypeptide, wherein the first polypeptide comprise a targeting unit and a heterodimerization unit, wherein the further polypeptide comprises a further targeting unit and an interaction unit, and wherein the antigenic unit of the first polypeptide comprises one or more antigens comprising B-cell epitopes, and the further antigenic unit of the further polypeptide comprises one or more further antigens comprising B-cell epitopes, wherein at least one antigen and at least one further antigen are derived from the same pathogen but from different strains or serotypes.

In some other embodiments, the multimer is a heterodimer of a first polypeptide and a further polypeptide, wherein the first polypeptide comprise a targeting unit and a heterodimerization unit, wherein the further polypeptide comprises a further targeting unit and a further heterodimerization unit, and wherein the antigenic unit of the first polypeptide comprises T cell epitopes of a pathogen and the further antigenic unit of the further polypeptide comprises a further antigen of the same pathogen. For example, the antigen is derived from or is a surface protein from a pathogen, such as the spike protein or RBD from SARS-COV-2, hemagglutinin of the influenza virus or gp120 of the HIV virus (human immunodeficiency virus), or the antigen is a full-length protein which is secreted by the pathogen into the cytoplasm of infected subjects.

In some other embodiments, the multimer is a heterodimer of a first polypeptide and a further polypeptide, wherein the first polypeptide comprise a targeting unit and a heterodimerization unit, wherein the further polypeptide comprises a further targeting unit and a further heterodimerization unit, and wherein the antigenic unit of the first polypeptide comprises T cell epitopes of several different pathogens and the antigenic unit of the second polypeptide comprises an antigen of a pathogen, whose T cell epitopes are included in the antigenic unit of the first polypeptide.

The aboce.described constructs may thus be used to target different seasonal viruses, for example for targeting betacoronavirus and influenza simultaneously, or to target different strains of betacoronavirus or influenza, or to target different mutations of the same strain of betacoronavirus or influenza.

Tolerance-inducing constructs comprising one or more epitopes from an allergen, self-antigen or alloantigen, may thus be used to target several epitopes such as several allergens simultaneously. For example, the antigenic unit of the first polypeptide could comprise some T cell epitopes derived from a first grass pollen allergen, and also some T cell epitopes derived from a second grass pollen allergen, and a second polypeptide could comprise a hypoallergenic allergen derived from the first grass pollen allergen.

Recently, highly stable coiled coil dimer-forming peptides (such as the P7A:P8A pair, such as the N7:N8 pair, or such as the N5:N6 pair; wherein the colon indicates the formation of coiled coil dimer consisting of the respective peptides) were designed to join two polypeptides/proteins in which they are comprised, for cellular applications. These artificial coiled coil pairs have low similarity to cellular proteins, making them unlikely to affect cellular processes. Thus, to enable the post translational joining of the first and further polypeptide, complementary coiled coil dimer-forming peptides are included as interaction unit in the first and further polypeptide. Alternatively, complementary coiled coil dimer-forming peptides are included as interaction units in a first further polypeptide and a second further polypeptide to enable the post translational joining of the first and second further polypeptide.

In some embodiments, the multimerization unit of the first polypeptide is a heterodimerization unit.

In some embodiments, the multimerization unit of the first polypeptide is a heterodimerization unit comprising or consisting of a coiled coil dimer-forming peptide.

In some embodiments, at least one of the one or more further polypeptides comprises an interaction unit, wherein the interaction unit is a further heterodimerization unit.

In some embodiments, at least one of the one or more further polypeptides comprises an interaction unit, wherein the interaction unit is a further heterodimerization unit comprising or consisting of a coiled coil dimer-forming peptide.

In some embodiments, at least one of the one or more further polypeptides comprises an interaction unit.

In some embodiments, at least one of the one or more further polypeptides comprises an interaction unit comprising or consisting of a coiled coil dimer-forming peptide.

In some embodiments, the heterodimerization unit and the further heterodimerization unit are different and form a coiled coil dimer.

In some embodiments, the coiled coil dimer is selected from the group of:
i. a P7A:P8A coiled coil dimer;
ii. a N7:N8 coiled coil dimer
iii. a N5:N6 coiled coil dimer.

Thus, in some embodiments, the coiled coil dimer-forming peptide is:
i. P7A or P8A;
ii. N7 or N8; or
iii. N5 or N6.

In some embodiments:
a) the first polypeptide comprises a heterodimerization unit comprising or consisting of a coiled coil dimer-forming peptide selected from P7A and P8A; and
b) at least one of the one or more further polypeptides comprises a further heterodimerization unit comprising or consisting of a coiled coil dimer-forming peptide selected from P7A and P8A;
wherein the heterodimerization unit and the further heterodimeric dimerization form a P7A:P8A coiled coil dimer.

In some embodiments, the coiled coil dimer-forming peptide is P7A or P8A.

In some embodiments, P7A comprises or consists of the amino acid sequence

(SEQ ID NO: 16)
YGEIAALEAKNAALKAEIAALEAKNAALKAGC.

In some embodiments, P8A comprises or consists of the amino acid sequence

(SEQ IS NO: 17)
YGKIAALKAENAALEAKIAALKAENAALEAGGC.

In some embodiments, particularly for immunogenic constructs, the first polypeptide comprises a coiled coil peptide A (SEQ ID NO: 194). In some embodiments, the one or more further polypeptides comprises a coiled coil peptide B (SEQ ID NO: 197). In some embodiments, the first polypeptide comprises a coiled coil peptide A (SEQ ID NO: 194) and the one or more further polypeptides comprises a coiled coil peptide B (SEQ ID NO: 197).

In some embodiments, the coiled coil dimer-forming peptide is N7 or N8.

In some embodiments, the coiled coil dimer-forming peptide is N5 or N6.

In some embodiments, N7 comprises or consists of the amino acid sequence

(SEQ ID NO: 18)
YEIAALEAKNAALKAEIAALEAKIAALKAGC.

In some embodiments, N8 comprises or consists of the amino acid sequence

(SEQ IS NO: 19)
YKIAALKAENAALEAKIAALKAEIAALEAGC.

In some embodiments, N5 comprises or consists of the amino acid sequence

(SEQ ID NO: 20)
YEIAALEAKIAALKAKNAALKAEIAALEAGC.

In some embodiments, N6 comprises or consists of the amino acid sequence

(SEQ IS NO: 21)
YKIAALKAEIAALEAENAALEAKIAALKAGC.

The skilled person will understand that the A: B pair, N5:N6 pair or the N7:N8 pair could be used to form a heterodimer between the first polypeptide and at least one of the one or more further polypeptides (or between further polypeptides) in a manner similar as described herein for embodiments where a P7A:P8A pair is used.

The skilled person will understand that when an element of the coiled coil dimer-forming peptide pair is present on a polypeptide, the other element of the pair is required on another polypeptide in order to form a coiled coil dimer. For example:
i. the first polypeptide comprises a heterodimeric dimerization unit, wherein the heterodimeric dimerization unit comprises or consists of a P7A coiled coil dimer-forming peptide, and a further polypeptide comprises a heterodimeric dimerization unit, wherein the heterodimeric dimerization unit comprises a P8A coiled coil dimer-forming peptide;
ii. the first polypeptide comprises a heterodimeric dimerization unit, wherein the heterodimeric dimerization unit comprises or consists of a P8A coiled coil dimer-forming peptide, and a further polypeptide comprises a heterodimeric dimerization unit, wherein the heterodimeric dimerization unit comprises a P7coiled coil dimer-forming peptide;
iii. the first polypeptide comprises a heterodimeric dimerization unit, wherein the heterodimeric dimerization unit comprises or consists of a N7 coiled coil dimer-forming peptide, and a further polypeptide comprises a heterodimeric dimerization unit, wherein the heterodimeric dimerization unit comprises a N8 coiled coil dimer-forming peptide;
iv. the first polypeptide comprises a heterodimeric dimerization unit, wherein the heterodimeric dimerization unit comprises or consists of a N8 coiled coil dimer-forming peptide, and a further polypeptide comprises a heterodimeric dimerization unit, wherein the heterodimeric dimerization unit comprises a N7 coiled coil dimer-forming peptide;
v. the first polypeptide comprises a heterodimeric dimerization unit, wherein the heterodimeric dimerization unit comprises or consists of a N5 coiled coil dimer-forming peptide, and a further polypeptide comprises a heterodimeric dimerization unit, wherein the heterodimeric dimerization unit comprises a N6 coiled coil dimer-forming peptide;
vi. the first polypeptide comprises a heterodimeric dimerization unit, wherein the heterodimeric dimerization unit comprises or consists of a N6 coiled coil dimer-forming peptide, and a further polypeptide comprises a heterodimeric dimerization unit, wherein the heterodimeric dimerization unit comprises a N5 coiled coil dimer-forming peptide; or
vii. the first polypeptide comprises a heterodimeric dimerization unit, wherein the heterodimeric dimerization unit comprises or consists of an A coiled coil dimer-forming peptide, and a further polypeptide comprises a heterodimeric dimerization unit, wherein the heterodimeric dimerization unit comprises or consists of a B coiled coil dimer-forming peptide.

In some embodiments:
i. the antigenic unit of the first polypeptide comprises one or more T cell epitopes;
ii. at least one of the one or more further polypeptides comprises a further targeting unit;
iii. the first polypeptide further comprises a heterodimerization unit, wherein the heterodimerization unit comprises or consists of a coiled coil dimer-forming peptide, such as P7A, P8A, N7, N8, N5, N6, A or B;
iv. the at least one further polypeptide comprises a further heterodimerization unit, wherein the further heterodimerization unit comprises or consists of a coiled coil dimer-forming peptide such as P7A, P8A, N7, N8, N5, N6, A or B;
v. the heterodimerization unit and the further heterodimeric dimerization form a coiled coil dimer; and
vi. the at least one further polypeptide further comprises a further antigenic unit comprising one or more B cell epitopes and/or one or more antigens, such as folded antigens.

In other embodiments:
i. the antigenic unit of the first polypeptide comprises one or more T cell epitopes of an allergen, self-antigen or alloantigen;
ii. at least one of the one or more further polypeptides comprises a further targeting unit;
iii. the first polypeptide further comprises a heterodimerization unit, wherein the heterodimerization unit comprises or consists of a coiled coil dimer-forming peptide, such as P7A, P8A, N7, N8, N5, N6, A or B;
iv. the at least one further polypeptide comprises a further heterodimerization unit, wherein the further heterodimerization unit comprises or consists of a coiled coil dimer-forming peptide such as P7A, P8A, N7, N8, N5, N6, A or B;
v. the heterodimerization unit and the further heterodimeric dimerization form a coiled coil dimer; and
vi. the at least one further polypeptide further comprises a further antigenic unit comprising one or more allergens, hypoallergenic allergens, self-antigens or alloantigens.

In some embodiments:
i. the antigenic unit of the first polypeptide comprises or consists of one or more B cell epitopes and/or one or more antigens, such as folded antigens;
ii. at least one of the one or more further polypeptides comprises a further targeting unit;
iii. the multimerization unit comprises a heterodimerization unit, wherein the heterodimerization unit comprises or consists of a coiled coil dimer-forming peptide such as P7A, P8A, N7, N8, N5, N6, A or B;
iv. the at least one further polypeptide comprises a further heterodimerization unit, wherein the heterodimerization unit comprises or consists of a coiled coil dimer-forming peptide such as P7A, P8A, N7, N8, N5, N6, A or B;
v. the heterodimerization unit and the further heterodimeric dimerization form a coiled coil dimer; and
vi. the at least one further polypeptide comprises a further antigenic unit comprising one or more B cell epitopes and/or one or more T cell epitopes.

In other embodiments:
i. the antigenic unit of the first polypeptide comprises or consists of one or more T cell epitopes of an allergen, self-antigen or alloantigen;
ii. at least one of the one or more further polypeptides comprises a further targeting unit;
iii. the multimerization unit comprises a heterodimerization unit, wherein the heterodimerization unit comprises or consists of a coiled coil dimer-forming peptide such as P7A, P8A, N7, N8, N5, N6, A or B;
iv. the at least one further polypeptide comprises a further heterodimerization unit, wherein the heterodimerization unit comprises or consists of a coiled coil dimer-forming peptide such as P7A, P8A, N7, N8, N5, N6, A or B;
v. the heterodimerization unit and the further heterodimeric dimerization form a coiled coil dimer; and
vi. the at least one further polypeptide comprises a further antigenic unit comprising one or more allergens, hypoallergenic allergens, self-antigens or alloantigens comprising one or more B cell epitopes and/or one or more T cell epitopes.

In some embodiments:
i. the antigenic unit of the first polypeptide comprises or consists of one or more antigens, such as folded antigens;
ii. at least one of the one or more further polypeptides comprises a further targeting unit;
iii. the multimerization unit comprises a heterodimerization unit, wherein the heterodimerization unit comprises or consists of a coiled coil dimer-forming peptide such as P7A, P8A, N7, N8, N5, N6, A or B;
iv. the at least one further polypeptides comprises a further heterodimerization unit, wherein the heterodimerization unit comprises or consists of a coiled coil dimer-forming peptide such as P7A, P8A, N7, N8, N5, N6, A or B;
v. the heterodimerization unit and the further heterodimeric dimerization form a coiled coil dimer; and
vi. the at least one further polypeptides further comprises a further antigenic unit comprising one or more T cell epitopes.

The skilled person will understand that any heterodimerization unit known in the art could be used to form a heterodimer between the first polypeptide and at least one of the one or more further polypeptides (or between further polypeptides). Useful heterodimerization units which can be included in the present constructs are described in detail e.g. in WO2019/048928 in the section “Heterodimerization units”.

Thus, in some embodiments the first polypeptide comprises one or more T cell epitopes, and a further polypeptide comprises a further targeting unit that targets antigen-presenting cells, a further heteromultimerization unit, such as a further heterodimerization unit, and a further antigenic unit comprising one or more further epitopes, comprises one or more B-cell epitopes, wherein the first polypeptide and the further polypeptide form a heterodimer. In some embodiments, the first polypeptide comprises one or more T cell epitopes, and a further polypeptide comprises a further targeting unit that targets antigen-presenting cells, a further heteromultimerization unit, such as a further heterodimerization unit, and a further antigenic unit comprising one or more B or T cell epitopes from one or more allergens, hypoallergenic allergens, self-antigens or alloantigens, wherein the first polypeptide and the further polypeptide form a heterodimer. In some embodiments the first polypeptide comprises one or more T cell epitopes, and a further polypeptide comprises a further targeting unit that targets antigen-presenting cells, a further heteromultimerization unit, such as a further heterodimerization unit, and a further antigenic unit comprising one or more B or T cell epitopes from one or more allergens, hypoallergenic allergens, self-antigens or alloantigens, wherein the first polypeptide and the further polypeptide form a heterodimer.

In some embodiments:
I. The antigenic unit of the first polypeptide comprises one or more T cell epitopes;
II. The further antigenic unit of the at least one of the one or more further polypeptides comprises one or more antigens, such as antigens comprising B-cell epitopes; and
III. the first polypeptide and at least one further polypeptide form a heterodimer.

In some embodiments:
I. The antigenic unit of the first polypeptide comprises one or more T cell epitopes of an allergen, self-antigen or alloantigen;
II. The further antigenic unit of the at least one of the one or more further polypeptides comprises one or more allergens, hypoallergenic allergens, self-antigens or alloantigens; and
III. the first polypeptide and at least one further polypeptide form a heterodimer.

In some embodiments:

I. The antigenic unit of the first polypeptide comprises one or more antigens, such as antigens comprising B-cell epitopes;

II. The further antigenic unit of at least one of the one or more further polypeptides comprises one or more T cell epitopes; and

III. the first polypeptide and at least one further polypeptide form a heterodimer.

In some embodiments:
I. The antigenic unit of the first polypeptide comprises one or more antigens, such as antigens comprising B-cell epitopes;
II. The further antigenic unit of at least one of the one or more further polypeptides comprises one or more antigens, such as antigens comprising B-cell epitopes;
III. the first polypeptide and at least one further polypeptide form a heterodimer, wherein the antigens comprising B-cell epitopes of the first polypeptide, and the antigens comprising B-cell epitopes of the at least one of the one or more further polypeptides are different.

In some embodiments, the interaction unit comprises a heterodimeric sequence B and a heterodimeric sequence A. In some embodiments, the heterodimeric sequence B heterodimerizes with the heterodimeric sequence A.

In some embodiments, heterodimeric sequence A is GGSSGGKFGGSTTAPSAQLEKELQALEKENAQLEWELQALEKELAQGGGSGGLTKF GGSTTAPSAQLEKELQALEKENAQLEWELQALEKELAQGGGSGGLTGLSGL (SEQ ID NO: 179) and heterodimeric sequence B is

(SEQ ID NO: 180)
GGSSGGKFGGSTTAPSAQLKKKLQALKKKNAQLKWKLQALKKKL
AQGGGSGGLTKFGGSTTAPSAQLKKKLQALKKKNAQLKWKLQAL
KKKLAQGGGSSGGLTGLSGL.

In some embodiments, heterodimeric sequence A is GEIAALEAKNAALKAEIAALEAKNAALKAGC (SEQ ID NO: 181) and heterodimeric sequence B is GKIAALKAENAALEAKIAALKAENAALEAGGC SEQ ID NO: 182).

In some embodiments, heterodimeric sequence A is GKIAALKAENAALEAKIAALKAENAALEAGGC (SEQ ID NO: 182) and heterodimeric sequence B is GEIAALEAKNAALKAEIAALEAKNAALKAGC (SEQ ID NO: 181).

In some embodiments, heterodimeric sequence A is GEIAALEAKNAALKAEIAALEAKIAALKAGY (SEQ ID NO: 183) and heterodimeric sequence B is YGKIAALKAENAALEAKIAALKAEIAALEAGY (SEQ ID NO: 184).

In some embodiments, heterodimeric sequence A is GEIAALEAKIAALKAKNAALKAEIAALEAG (SEQ ID NO: 185) and heterodimeric sequence B is GKIAALKAEIAALEAENAALEAKIAALKAG (SEQ ID NO: 186).

Multimerization with an Antigenic Unit

If the antigenic unit of the first polypeptide comprises an antigen, said antigen is expressed as a “monomeric” antigen. Yet, many pathogenic proteins, in particular surface proteins and receptors, are oligomers, e.g., hemagglutinin is a homotrimeric glycoprotein found on the surface of influenza viruses, which is crucial for efficient viral replication and a well-known target for anti-influenza vaccines.

While a first polypeptide comprising in its antigenic unit an antigen of a pathogen, that in nature exists as an oligomer (oligomerized native antigen), still may elicit an immune response in a subject to which it has been administered, oligomerization typically stabilizes the protein conformation by inter-molecular interactions and occlusion of hydrophobic patches. Thus, the display of a functional, biologically relevant oligomer hides epitopes that are normally not exposed by the oligomerized native antigen, avoiding unwanted immune responses. Furthermore, the oligomerization may also protect the antigen against denaturation.

Thus, in some embodiments, the further antigenic unit of at least one of the one or more further polypeptides comprises an antigen which is capable of oligomerizing with at least one antigen comprised in the antigenic unit of the first polypeptide.

In some embodiments, the further antigenic unit of at least one of the one or more further polypeptides comprises an antigen which is capable of oligomerizing with at least one antigen comprised in the antigenic unit of the first polypeptide, and/or with at least one antigen comprised in the further antigenic unit of another further polypeptide. In some embodiments, the further antigenic unit of at least one of the one or more further polypeptides comprises a hypoallergenic allergen which is capable of oligomerizing with at least one hypoallergenic allergen comprised in the further antigenic unit of another further polypeptide. In some embodiments, the further antigenic unit of at least one of the one or more further polypeptides comprises an alloantigen which is capable of oligomerizing with at least one alloantigen comprised in the further antigenic unit of another further polypeptide. In some embodiments, the further antigenic unit of at least one of the one or more further polypeptides comprises a self-antigen which is capable of oligomerizing with at least one self-antigen comprised in the further antigenic unit of another further polypeptide. In some embodiments, the further antigenic unit of at least one of the one or more further polypeptides comprises an allergen and is capable of oligomerizing with at least one allergen comprised in the further antigenic unit of another further polypeptide.

In some embodiments, the antigenic unit of at least one of the one or more further polypeptides comprises an antigen and the interaction unit of said further polypeptide is capable of oligomerizing with the antigenic unit of the first polypeptide, and/or with the interaction unit of another further polypeptides. In some embodiments, the antigenic unit of at least one of the one or more further polypeptides comprises a hypoallergenic allergen and the interaction unit of said further polypeptide is capable of oligomerizing with the hypoallergenic allergen of the first polypeptide, and/or with the interaction unit of another further polypeptide.

In some embodiments, the antigenic unit of at least one of the one or more further polypeptides comprises an antigen and the interaction unit of said further polypeptide is capable of oligomerizing with the antigenic unit of the first polypeptide, and/or with at least one antigen comprised in the further antigenic unit of another further polypeptides.

In some embodiments, the antigenic unit of at least one of the further polypeptides forms an oligomer, such as a dimer, such as a trimer, such as a tetramer, with at least one antigen comprised in the antigenic unit of the first polypeptide. In some embodiments, the interaction unit of at least one of the further polypeptides forms an oligomer, such as a dimer, such as a trimer, such as a tetramer, with at least one hypoallergenic allergen comprised in the interaction unit of another further polypeptide.

In some embodiments, the antigenic unit of at least one of the further polypeptides forms an oligomer, such as a dimer, such as a trimer, such as a tetramer, with at least one antigen comprised in the antigenic unit of the first polypeptide and/or with at least one antigen comprised in the further antigenic unit of another further polypeptide.

In some embodiments, the interaction unit of at least one of the further polypeptides forms an oligomer, such as a dimer, such as a trimer, such as a tetramer, with at least one antigen comprised in the antigenic unit of the first polypeptide.

In some embodiments, the interaction unit of at least one of the further polypeptides forms an oligomer, such as a dimer, such as a trimer, such as a tetramer, with at least one antigen comprised in the antigenic unit of the first polypeptide and/or with at least one antigen comprised in the interaction unit of another further polypeptide.

In some embodiments, the antigenic unit of at least one of the further polypeptides forms a hetero-oligomer, such as a hetero-dimer, such as a hetero-trimer, such as a hetero-tetramer, with at least one antigen comprised in the antigenic unit of the first polypeptide and/or with at least one antigen comprised in the further antigenic unit of at least one further polypeptide. In some embodiments, the antigenic unit of at least one of the further polypeptides forms a hetero-oligomer, such as a hetero-dimer, such as a hetero-trimer, such as a hetero-tetramer, with at least one hypoallergenic allergen comprised in the further antigenic unit of at least one further polypeptide.

In some embodiments, the afore-described oligomerization occurs naturally or spontaneously, i.e., the antigens oligomerize/multimerize after they have been expressed from the vector.

In some other embodiments, the first polypeptide and/or the interaction unit of at least one of the one or more further polypeptides comprise sequences that encode an amino acid sequence that facilitates the oligomerization of the antigens and/or further antigens, or that facilitates the oligomerization of the allergens, hypoallergenic allergens, self-antigens or alloantigens, such as a homo-oligomerization sequence or a hetero-oligomerization sequence. The use of a hetero-oligomerization sequence is preferred, since it will result in defined protein products e.g., the further antigen of the further polypeptide will oligomerize with the antigen of the first polypeptide and not with itself, or e.g. the hypoallergenic allergen of a first further polypeptide will oligomerize with the hypoallergenic allergen of a second further polypeptide and not with itself.

Thus, in some embodiments, the interaction unit of at least one of the one or more further polypeptides comprises a sequence that encodes an amino acid sequence that facilitates the dimerization of the antigens, such as a hetero-dimerization sequence, e.g., a heterodimeric coiled coil pair.

Thus, in some embodiments, the antigenic unit of the first polypeptide comprises a heterodimeric coiled coil pair. This may be particularly relevant for immunogenic constructs. In some embodiments, the antigenic unit of at least one of the one or more further polypeptides comprises a heterodimeric coiled coil pair. This may be particularly relevant for tolerance-inducing constructs.

Thus, in some embodiments, the heterodimeric coiled coil pair of the antigenic unit of the first polypeptide and the heterodimeric coiled coil pair of the interaction unit of at least one further polypeptide facilitates the oligomerization of an antigen comprised in the antigenic unit of the first polypeptide and a further antigen in the further antigenic unit of the further polypeptide.

Thus, in some embodiments, the interaction unit of at least one of the one or more further polypeptides comprises a sequence that encodes an amino acid sequence that facilitates the trimerization of the antigens, such as a hetero-trimerization sequence, e.g., a heterotrimeric coiled coil pair.

Thus, in some embodiments, the antigenic unit of the first polypeptide comprises a heterotrimeric coiled coil pair. This may be particularly relevant for immunogenic constructs.

Thus, in some embodiments, the heterotrimeric coiled coil pair of the antigenic unit of the first polypeptide and the heterotrimeric coiled coil pair of the interaction unit of at least one further polypeptide facilitates the oligomerization of an antigen comprised in the antigenic unit of the first polypeptide and a further antigen in the further antigenic unit of the further polypeptide.

Examples of heterodimeric and heterotrimeric coiled coil pair are described in Litowski et al. J Biol Chem (2002); Kiyokawa et al. Chemistry (2004); and Nautiyal et al. Protein Sci. 1999.

In some embodiments, the antigenic unit of the first polypeptide, and/or the interaction unit of at least one of the one or more further polypeptides comprises the amino acid sequence YGGIEAKIEAIEAKAEAIEAKIEAIEAKIEA (SEQ ID NO: 22). In some embodiments, particularly for tolerance-inducing constructs, the interaction unit of at least one of the one or more further polypeptides comprises SEQ ID NO: 22.

In some embodiments, the antigenic unit of the first polypeptide, and/or the interaction unit of at least one of the one or more further polypeptides comprises the amino acid sequence GGIEQKIEAIEWKWEAIEQKIEAIEQKIEA (SEQ ID NO: 23). In some embodiments, particularly for tolerance-inducing constructs, the interaction unit of at least one of the one or more further polypeptides comprises SEQ ID NO: 23.

Thus, in some embodiments:
i. the targeting unit comprises or consists of human CCL3L1;
ii. the antigenic unit comprises one or more B cell epitopes;
iii. the interaction unit comprises the amino acid sequence

(SEQ ID NO: 22)
YGGIEAKIEAIEAKAEAIEAKIEAIEAKIEA;

iv. the interaction unit of at least one of the one or further polypeptides comprise a further signal peptide, wherein the further signal peptide comprises or consists of the signal peptide of human CCL3L1; and
v. the interaction unit of the at least one further polypeptide comprises the amino acid sequence GGIEQKIEAIEWKWEAIEQKIEAIEQKIEA (SEQ ID NO: 23).

Thus, in some embodiments:
i. the targeting unit comprises or consists of human CCL3L1;
ii. the antigenic unit comprises one or more B cell epitopes;
iii. the interaction unit comprises the amino acid sequence

(SEQ ID NO: 22)
YGGIEAKIEAIEAKAEAIEAKIEAIEAKIEA;

iv. at least one of the one or further polypeptides comprise a further signal peptide, wherein the further signal peptide comprises or consists of the signal peptide of human CCL3L1;
v. the further antigenic unit of the at least one further polypeptide comprises a further antigen comprising one or more epitopes; and
vi. the interaction unit of at least one further polypeptide comprises the amino acid sequence GGIEQKIEAIEWKWEAIEQKIEAIEQKIEA (SEQ ID NO: 23).

In some embodiments:
i. the targeting unit comprises or consists of human CCL3L1;
ii. the antigenic unit comprises an antigen comprising one or more B cell epitopes;
iii. the interaction unit comprises the amino acid sequence

(SEQ ID NO: 22)
YGGIEAKIEAIEAKAEAIEAKIEAIEAKIEA;

iv. at least one of the one or further polypeptides comprise a further signal peptide, wherein the further signal peptide comprises or consists of the signal peptide of human CCL3L1;
v. the further antigenic unit of the at least one further polypeptide comprises a further antigen, wherein said antigen and further antigen are identical;
and
vi. the interaction unit of at least one further polypeptide comprises the amino acid sequence GGIEQKIEAIEWKWEAIEQKIEAIEQKIEA (SEQ ID NO: 23).

In some embodiments:
i. the targeting unit comprises or consists of human CCL3L1;
ii. the antigenic unit comprises an antigen comprising one or more B cell epitopes;
iii. the interaction unit comprises the amino acid sequence

(SEQ ID NO: 22)
YGGIEAKIEAIEAKAEAIEAKIEAIEAKIEA;

iv. at least one of the one or further polypeptides comprise a further signal peptide, wherein the further signal peptide comprises or consists of the signal peptide of human CCL3L1;
v. the further antigenic unit of the at least one further polypeptide comprises a further antigen, wherein said antigen and further antigen are naturally found in the same protein or protein complex or are identical; and
vi. the interaction unit of at least one further polypeptide comprises the amino acid sequence GGIEQKIEAIEWKWEAIEQKIEAIEQKIEA (SEQ ID NO: 23).

In some embodiments, the interaction unit of at least two of the one or more further polypeptides comprise a heterotrimeric coiled coil pair.

In some embodiments, the antigenic unit of the first polypeptide, and/or the interaction unit of at least one of the one or more further polypeptides comprise the amino acid sequence AEIAAIEYEQAAIKEEIAAIKDKIAAIKEYIAAI (SEQ ID NO: 12). In some embodiments, particularly for tolerance-inducing constructs, the interaction unit of at least one of the one or more further polypeptides comprises SEQ ID NO: 12.

In some embodiments, the antigenic unit of the first polypeptide, and/or the interaction unit of at least one of the one or more further polypeptides comprise the amino acid sequence EKIAAIKEEQAAIEEEIQAIKEEIAAIKYLIAQI (SEQ ID NO: 13). In some embodiments, particularly for tolerance-inducing constructs, the interaction unit of at least one of the one or more further polypeptides comprises SEQ ID NO: 13.

In some embodiments, the antigenic unit of the first polypeptide, and/or the interaction unit of at least one of the one or more further polypeptides comprise the amino acid sequence AEIAAIKYKQAAIKNEIAAIKQEIAAIEQMIAAI (SEQ ID NO: 14). In some embodiments, particularly for tolerance-inducing constructs, the interaction unit of at least one of the one or more further polypeptides comprises SEQ ID NO: 14.

In some embodiments, e.g. of immunogenic constructs:
i. the targeting unit comprises or consists of human CCL3L1;
ii. the antigenic unit comprises one or more B cell epitopes;
iii. the antigenic unit comprises the amino acid sequence

(SEQ ID NO: 12)
AEIAAIEYEQAAIKEEIAAIKDKIAAIKEYIAAI;

iv. at least one of the one or further polypeptides comprise a further signal peptide, wherein the further signal peptide comprises or consists of the signal peptide of human CCL3L1;
v. the interaction unit of at least one other further polypeptide comprises the amino acid sequence EKIAAIKEEQAAIEEEIQAIKEEIAAIKYLIAQI (SEQ ID NO: 13); and
vi. the interaction unit of at least one other further polypeptide comprises the amino acid sequence AEIAAIKYKQAAIKNEIAAIKQEIAAIEQMIAAI (SEQ ID NO: 14).

### Leucine Zipper Motifs

Leucine zipper motifs are highly conserved protein dimerization motifs found in eukaryotic cells. The use of leucine zipper motifs to post-translationally join two proteins has been extensively described in the art. As with coiled-coil peptides, one part of the leucine zipper (a first leucine zipper motif) has to present in a first protein and the other part of the leucine zipper (a second leucine zipper motif) has to be present in a second protein to post-translationally join the first and second protein via the leucine zipper motifs.

In some embodiments, leucine zipper motifs can be used to connect the further antigenic unit of a further polypeptide to the antigenic unit of a first polypeptide. For example leucine zipper motifs can be used to connect an antigen comprised in the further antigenic unit, such as a hypoallergenic allergen, a self-antigen or an alloantigen to the antigenic unit of a first polypeptide comprising T cell epitopes, such as T cell epitopes of an allergen, self-antigen or alloantigen. Thus, in some embodiments the first polypeptides comprises antigenic unit comprising an antigen and an interaction unit which is a first leucine zipper motif and at least one of the further polypeptides comprises an interaction unit which is a second leucine zipper motif, wherein the first leucine zipper motif and second leucine zipper motif connect an antigen comprised in the first to polypeptide an antigen comprised in one of the further polypeptides.

Leucine zipper motifs may be used to connect the further antigenic unit of at least one of the further polypeptides to the antigenic unit of the first polypeptide, and/or to the antigenic unit of another further polypeptide.

Examples of suitable leucine zipper motifs can be found in in Moll et al. Prot Science (2001); Glasgow et al., Plos One (2009); Walseng et al., Plos One (2015); and Craig et al., Biomacromolecules (2012) which are incorporated herein by reference.

In some embodiments, the antigenic unit of the first polypeptide comprises T cell epitopes from a pathogen, as described herein, and the further antigenic unit of at least one of the further polypeptides comprises an antigen from the same pathogen or from a different pathogen. For example, the T cell epitopes are derived from betacoronavirus, such as SARS-COV-2, for instance from the spike protein, while the antigen are RBD from SARS-COV-2. Alternatively, the T cell epitopes of the antigenic unit of the first polypeptide comprises T cell epitopes derived from different pathogens, and the antigen could be an antigen from one of these pathogens. For instance, the antigenic unit of the first polypeptide comprises some T cell epitopes derived from influenza virus, and also some T cell epitopes derived from SARS-COV-2, and the further antigenic unit of at least one of the further polypeptides comprises an antigen from influenza virus.

In some embodiments, the antigenic unit of the first polypeptide comprises T cell epitopes of an allergen, as described herein, and the further antigenic unit of at least one of the further polypeptides comprises an allergen or hypoallergenic allergen from the same allergen or from a different allergen. For example, the T cell epitopes are derived from a food allergen. Alternatively, the T cell epitopes of the antigenic unit of the first polypeptide comprises T cell epitopes derived from allergens. For instance, the antigenic unit of the first polypeptide comprises some T cell epitopes derived from a first grass pollen allergen, and also some T cell epitopes derived from a second grass pollen allergen, and the further antigenic unit of at least one of the further polypeptides comprises an hypoallergenic allergen derived from the first grass pollen allergen.

In some embodiments, the antigenic unit of the first polypeptide comprises T cell epitopes of a self-antigen as described herein, and the further antigenic unit of at least one of the further polypeptides comprises a self-antigen from the same self-antigen or from a different self-antigen. In some embodiments, the antigenic unit of the first polypeptide comprises T cell epitopes of an alloantigen as described herein, and the further antigenic unit of at least one of the further polypeptides comprises an alloantigen from the same alloantigen or from a different alloantigen.

In some embodiments, the first polypeptide comprises a first leucine zipper motif.

In some embodiments, the interaction unit of at least one of the one or more further polypeptides comprise or consists of a second leucine zipper motif.

In some embodiments, the first leucine zipper motif is at the C terminal end of the first polypeptide and the second leucine zipper motif is at the C terminal end or at the N terminal end of the one or more further polypeptides.

In some embodiments, an interaction unit comprising or consisting of a first leucine zipper motif is at the C terminal end or at the N terminal end of one further polypeptide, and an interaction unit comprising or consisting of a second leucine zipper motif is at the C terminal end or at the N terminal end of one other further polypeptide.

The first leucine zipper motif and the second leucine zipper motif are capable of forming a dimer, thereby allowing e.g., the interaction unit of a further polypeptide to bind to the C terminal end of the first polypeptide or to bind to the C-terminal end or N-terminal end of another further polypeptide.

In some embodiments, the interaction unit of at least one of the one or more further polypeptides comprise or consists of a second leucine zipper motif, and the further antigenic unit of said further polypeptide comprises one or more antigens.

In some embodiments, the interaction unit of at least one of the one or more further polypeptides comprises or consists of a second leucine zipper motif, and the further antigenic unit of said further polypeptide comprises one or more allergens, hypoallergenic allergens, self-antigens or alloantigens.

In some embodiments, at least two of the one or more further polypeptides comprise an interaction unit comprising or consisting of a leucine zipper motif, wherein the leucine zipper motifs are capable of forming a dimer between the at least two further polypeptides.

In some embodiments:
i. the first polypeptide comprises a first leucine zipper motif at the C terminal end of the antigenic unit of the first polypeptide;
ii. at least one of the one or more further polypeptides comprises an interaction unit comprising or consisting of a second leucine zipper motif at the C terminal end or at the N terminal end of the further antigenic unit;
iii. the further antigenic unit comprises an antigen; and
iv. the first leucine zipper motif forms a dimer with the second leucine zipper motif.

In some embodiments:
i. the antigenic unit of the first polypeptide comprises one or more T cell epitopes, e.g. T cell epitopes of an allergen, self-antigen or alloantigen
ii. the first polypeptide comprises a first leucine zipper motif at the C terminal end of antigenic unit;
iii. at least one of the one or more further polypeptides comprises an interaction unit comprising or consisting of a second leucine zipper motif at the C terminal end or at the N terminal end of the further antigenic unit;
iv. the further antigenic unit comprises one or more antigens, such as allergens, hypoallergenic allergens, self-antigens or alloantigens; and
v. the first leucine zipper motif forms a dimer with the second leucine zipper motif.

In some embodiments, at least one leucine zipper motif comprises the amino acid sequence LEIRAAFLRQRNTALRTEVAELEQEVQRLENEVSQYETRYGPLGGGK (SEQ ID NO: 24).

In some embodiments, at least one leucine zipper motif comprises the amino acid sequence LEIEAAFLERENTALETRVAELRQRVQRLRNRVSQYRTRYGPLGGGK (SEQ ID NO: 25).

In some embodiments, at least one leucine zipper motif comprises the amino acid sequence LEIRAAFLRQRNTALRTEVAELEQEVQRLENEVSQYETRYGPL (SEQ ID NO: 155).

### Unit Linker

In the context of the first polypeptide, the antigenic unit is connected to the multimerization unit, preferably by an unit linker. Thus, in some embodiments, the first nucleic acid sequence comprised in the vectors of the disclosure encodes a first polypeptide that further comprises an unit linker that connects the antigenic unit to the multimerization unit.

Similarly, in some embodiments, at least one of the one or more further nucleic acids comprised in the vectors of the disclosure encodes one or more further polypeptides that comprises a further unit linker that connects the further antigenic unit to the interaction unit. In some embodiments, the unit linker is a non-immunogenic linker and/or flexible or rigid linker.

The unit linker may comprise a restriction site in order to facilitate the construction of the nucleic acid sequence. In some embodiments, the unit linker is GLGGL (SEQ ID NO: 26) or GLSGL (SEQ ID NO: 27). In other embodiments, the unit linker comprises or consists of GGGGS (SEQ ID NO: 28), GGGGSGGGGS (SEQ ID NO: 29), (GGGGS) m (SEQ ID NO: 30), EAAAK (SEQ ID NO: 31), (EAAAK) m (SEQ ID NO: 32), (EAAAK) mGS (SEQ ID NO: 33), (EAAK) mGS (SEQ ID NO: 34), GPSRLEEELRRRLTEPG (SEQ ID NO: 35), AAY or HEYGAEALERAG (SEQ ID NO: 36).

### Antigenic Unit

Generally, and particularly for immunogenic constructs, the antigenic unit comprised in the first polypeptide/multimeric protein, or the further antigenic unit comprised in a further polypeptide, can comprise any type of epitope(s) and/or antigen(s) or parts thereof, e.g., antigens or parts thereof which are disease-relevant. Examples include one or more cancer antigens or parts thereof, for example as described in application PCT/EP2022/057955, in particular in the sections entitled “Antigenic unit”, “Antigenic unit of individualized anticancer vaccines”, “Antigenic unit of individualized anticancer vaccines comprising one or more neoantigens or parts thereof”, “Antigenic unit of non-individualized anticancer vaccines” and “Further embodiments of the antigenic unit”. Other examples include one or more antigens or parts thereof relevant for an infectious disease, i.e., a disease caused by a pathogen, including viruses, bacteria, fungi and parasites, for example as described in application PCT/EP2022/061819, in particular in the section entitled “Antigens or parts or fragments thereof”.

Further, and particularly for tolerance-inducing constructs, the antigenic unit comprised in the first polypeptide/multimeric protein comprises T cell epitopes of an allergen, self-antigen or alloantigen, and the further antigenic unit comprised in a further polypeptide comprises allergens, hypoallergenic allergens, self-antigens or alloantigens.

Thus, in some embodiments, the antigenic unit comprises one or more antigens or parts thereof comprising the one or more epitopes, and/or a further antigenic unit of at least one of the one or more further polypeptides comprises one or more further antigens or parts thereof comprising the one or more further epitopes. Thus, in some embodiments, the antigenic unit comprises one antigen or part thereof comprising the one or more epitopes, and/or a further antigenic unit of at least one of the one or more further polypeptides comprises one further antigen or part thereof comprising the one or more further epitopes.

In some embodiments, the antigenic unit comprises one or more T cell epitopes of an allergen, self-antigen or alloantigen, and the further antigenic unit of at least one of the one or more further polypeptides comprises one or more allergens, hypoallergenic allergens, self-antigens or alloantigens comprising the one or more further epitopes, such as one or more B or T cell epitopes.

In some embodiments, the antigenic unit comprises one or more antigens or parts thereof comprising the one or more epitopes. Thus, in some embodiments, the antigenic unit comprises one antigen or part thereof comprising the one or more epitopes.

In some embodiments, a further antigenic unit of at least one of the further polypeptides comprises one or more further antigens or parts thereof comprising the one or more further epitopes. Thus, in some embodiments, a further antigenic unit of at least one of the one or more further polypeptides comprises one further antigen or part thereof comprising the one or more further epitopes.

The skilled person will understand that any antigenic unit of the first polypeptide described herein may also be a further antigenic unit of at least one of the one or more further polypeptides.

“Disease-relevant antigen(s)” or “antigen(s) which is/are relevant for a disease” is used herein to describe that the antigen(s) or parts thereof included in the antigenic unit play a role and have a relevance for a certain disease for which the vector of the disclosure comprising such antigenic unit is designed to be used. As an example, the antigenic unit comprises one or more cancer antigens or parts thereof and a vector comprising such antigenic unit is designed for use in the treatment of cancer. In another example, the antigenic unit comprises one or more infectious antigens or parts thereof, e.g., antigens derived from a pathogen and a vector comprising such antigenic unit is designed for use in the treatment of an infectious disease caused by such pathogen or wherein such pathogen is involved.

Similarly, “disease-relevant epitope(s)” or “epitope(s) which is/are relevant for a disease” is used herein to describe that the epitopes(s) or parts thereof included in the antigenic unit play a role and have a relevance for a certain disease for which the vector of the disclosure comprising such antigenic unit is designed to be used.

A “part” refers to a part/fragment of an antigen, i.e., part/fragment of the amino acid sequence of an antigen, or the nucleotide sequence encoding same, e.g., an epitope. For tolerance constructs, the term “part” refers to a part or fragment of an allergen, hypoallergenic allergen, self-antigen, alloantigen, i.e. part or fragment of the amino acid sequence of an allergen, hypoallergenic allergen, self-antigen, alloantigen, or the nucleotide sequence encoding same.

In some embodiments of immunogenic constructs, the antigenic unit comprises one or more epitopes which are relevant for infectious diseases, e.g., antigens derived from pathogens.

Such antigens or T cell epitopes of an allergen, self-antigen or alloantigen, as well as allergens, hypoallergenic allergens, self-antigens or alloantigens may be known or have been predicted in the art, i.e., have been studied, proposed and/or verified to be involved and of relevance for a certain disease or allergic disease and published, e.g., in the scientific literature.

Thus, in some embodiments of immunogenic constructs, the antigenic unit comprises one or more epitopes which are relevant for cancer, e.g., cancer antigens such as neoantigens or shared cancer antigens.

In other embodiments, the antigenic unit comprises one or more epitopes which are relevant for infectious diseases. For example, in some embodiments, the antigenic unit comprises one or more antigens derived from surface proteins of pathogens, e.g. viral surface proteins such as the spike protein from SARS-COV-2, hemagglutinin of the influenza virus or gp120 of the HIV virus (human immunodeficiency virus). In some embodiments, the antigenic unit comprises or consist of or more antigens or parts or fragments thereof comprising a Hemagglutinin H1N1 sequence, such as the Hemagglutinin H1N1 sequence set forth in SEQ ID NO: 148. In some embodiments, the antigenic unit comprises or consist of one or more antigens or parts or fragments thereof derived from RSV virus, such as one or more antigens or parts or fragments thereof derived from RSV F protein or RSV PreF protein. In some embodiments, the antigenic unit comprises or consists of soluble RSV F protein or soluble RSV PreF protein. In some embodiments, the antigenic unit comprises or consists of RSV preF protein comprising a transmembrane domain.

In some embodiments, the antigen is a full-length protein of a pathogen, preferably a full-length surface protein, e.g. a full-length viral surface protein or bacterial surface protein or a full-length surface protein of any other pathogen. In other embodiments, the antigen is a full-length bacterial protein which is secreted by the bacterium, e.g. secreted into the cytoplasm of infected subjects. In other embodiments, the antigenic unit comprises more than one antigen, i.e. several antigens, each of which being a full-length protein.

In some embodiments, the epitope is a T cell epitope from a conserved region of the pathogen, i.e. conserved between several subgenus, species or strains of respective pathogens. In other words, the epitope may be encoded by a nucleotide sequence which is found in a conserved region of the genome of the pathogen, i.e. conserved between several subgenus, species or strains of respective pathogens. The epitope may thus be conserved between several subgenus, species or strains of respective pathogens, i.e. the amino acid sequence of the epitope is conserved between these.

As an example, the epitope may be a T cell epitope from a conserved region of a betacoronavirus, e.g. a region which is conserved between viruses from the same subgenus, such as the subgenus Sarbecovirus, e.g. conserved between SARS-COV-2, which causes coronavirus disease 2019 (COVID-19) and SARS-COV, which causes severe acute respiratory syndrome (SARS). By including such T cell epitope in the construct of the disclosure, a vaccine comprising the construct will, or is at least expected to, also provide protection against multiple variants of a betacoronavirus, e.g. variants of SARS-COV or variants of SARS-COV-2, which is important for protection against future variants. Viruses are known to mutate, e.g. undergo viral antigen drift or antigen shift. Finding conserved regions across the genome of betacoronavirus genus indicates that these conserved regions are needed to maintain essential structures or functions, thus it can be assumed that future mutations will take place in the less-conserved regions. By raising an immune response against the conserved regions, protection also against future variants can be achieved, or at least is expected to have a higher likelihood of being achieved.

As another example, the epitope may be a T cell epitope from a region of a human papilloma virus (HPV), e.g. from HPV16 or HPV18. HPV antigens may be any antigens selected from the list consisting of E1, E2, E6, E7, L1 and L2, e.g. E6 and/or E7 of HPV16 and/or HPV18. By including such T cell epitopes in the construct of the disclosure, a vaccine comprising the construct will provide protection against HPV. HPV infections are involved in certain cancers, such as squamous cell carcinoma of the head and neck, cervical cancer and vulvar squamous cell carcinoma. Indeed, HPV16 viral antigens are expressed in about 50% of all patients with said cancers.

As another example, the epitope may be a T cell epitope from a region of a human Influenza virus, such as human Influenza virus A, human Influenza virus B, human Influenza virus C and human Influenza virus D. As an example, the human Influenza virus may be a specific hemagglutinin (HA) subtype, such as H1, H2, and H3, and/or a specific neuraminidase (NA) subtype, such as N1 or N5. As an example, the human Influenza virus may be a H1N1 subtype. By including such T cell epitope in the construct of the disclosure, protection against Influenza infection can be achieved upon administration of the construct to a subject.

In some embodiments, the antigenic unit comprises one part of one antigen. The RBD domain of the spike protein of SARS-COV-2 or the head or stem domain of hemagglutinin of the influenza virus are examples of parts of an antigen. Sequences of the spike protein and of the RBD domain are available in databases. As an example, in the spike protein of the “Wuhan” strain (NCBI accession number YP_009724390), the RBD sequence is positioned at residues 319 to 542. As another example, hemagglutinin from influenza H1N1, e.g. having SEQ ID NO: 148, can be used. In some embodiments, the antigenic unit comprises or consists of RBD (aa319-542) of the spike protein of SARS-COV-2 (Wuhan variant).

In some embodiments, an antigen or a part or region of an antigen comprises multiple epitopes, such as T cell epitopes, e.g., multiple minimal T cell epitopes, such as a hotspot.

In some embodiments, the antigenic unit includes one T cell epitope. In other embodiments, the antigenic unit includes more than one T cell epitope, i.e., multiple T cell epitopes.

T cell epitopes suitable for inclusion into the antigenic unit may be known in the art, i.e., have been studied, proposed and/or verified to be involved and of relevance for a certain disease, e.g. an allergic disease, and published, e.g., in the scientific literature.

In some embodiments, the antigenic unit comprises T cell epitopes with a length of from 7 to 150 amino acids, preferably of from 7 to 100 amino acids, e.g., from 9 or 10 to 100 amino acids or from 15 to 100 amino acids or from 9 to 60 amino acids or from 9 to 30 amino acids or from 15 to 60 of from 15 to 30 or from 20 to 75 amino acids or from 25 to 50 amino acids.

In some embodiments, the antigenic unit comprises one or more T cell epitopes of an allergen, self-antigen or alloantigen, i.e., one T cell epitope of an allergen or more than one T cell epitope of an allergen, i.e., multiple T cell epitopes of an allergen, self-antigen or alloantigen. In some embodiments, the multiple T cell epitopes are of the same allergen, i.e., comprised in the same allergen. In another embodiment, the multiple T cell epitopes are of multiple different allergens, i.e., comprised in different allergens.

By way of example, Fel d 1, Fel d 4 and Fel d 7 are three of the most prominent cat allergens, accounting for most human cat allergies and the antigenic unit may comprise e.g., one or more T cell epitopes of Fel d 1, i.e., one T cell epitope of Fel d 1 or multiple T cell epitopes of Fel d 1. Further, the antigenic unit may comprise multiple T cell epitopes of e.g., Fel d 4 and Fel d 7, e.g., one or multiple T cell epitopes of Fel d 4 and one or multiple T cell epitopes of Fel d 7.

In some embodiments, the vectors of the disclosure/the constructs encoded by such vectors are for use in an individualized treatment, i.e., designed specifically for a particular subject/one patient. In other embodiments, the vectors of the disclosure/construct encoded by such vectors are for general use in a patient population or patients, i.e., an off-the-shelf treatment.

The one or more T cell epitopes may be derived from any of the allergens described in the section “Allergens”.

### Antigenic Unit of Individualized Polypeptides

In some embodiments, the first polypeptide encoded by the first nucleic acid comprised in the vectors of the disclosure comprises an antigenic unit, which is designed specifically and only for the patient who is to be treated with such vector. In some embodiments, at least one further polypeptide encoded by the one or more further nucleic acids comprised in the vectors of the disclosure comprises an antigenic unit, which is designed specifically and only for the patient who is to be treated with such vector. In some embodiments, the first polypeptide and at least one further polypeptide comprises an antigenic unit, which is designed specifically and only for the patient who is to be treated with such vector. This will increase the therapeutic effect compared to an off-the-shelf treatment comprising the construct. This may be particularly relevant for immunogenic constructs.

For example, in some embodiments, the antigenic unit comprises one or more patient-specific cancer antigens or parts thereof, such antigens including neoantigens or patient-present shared cancer antigens.

“Patient-present shared cancer antigen” is used herein to describe a shared cancer antigen or shared tumor antigen that has been identified to be present in the patient's tumor cells.

“Neoantigen” is used herein to describe a cancer antigen or tumor antigen found in a patient's tumor cells that comprises one or more mutations compared to the same patient's normal (i.e., healthy, non-cancerous) cells.

“Patient-present shared cancer epitope” is used herein to describe an amino acid sequence, or a nucleic acid sequence encoding same, comprised in a patient-present shared cancer antigen, which is known to be immunogenic or which has been predicted to be immunogenic.

“Neoepitope or patient-specific cancer epitope” is used herein to describe an amino acid sequence, or a nucleic acid sequence encoding same, comprised in a neoantigen or in a patient-specific cancer antigen, which comprises one or more mutations, which are predicted to be immunogenic.

In some embodiments, the antigenic unit comprises one or more patient-present shared cancer antigens or parts thereof, e.g., one patient-present shared cancer antigen or one or more parts of such patient-present shared cancer antigen, e.g., one or more epitopes, or several patient-present shared cancer antigens or one or more parts of such several patient-present shared cancer antigens, e.g., one or more epitopes.

The term “several” herein is used interchangeably with the term “multiple”, “a plurality” and “more than one”.

In other embodiments, the antigenic unit comprises one or more neoantigens or parts thereof, e.g., one neoantigen or one or more parts of such neoantigen, e.g., one or more neoepitopes or several neoantigens or one or more parts of such several neoantigens, e.g., one or more neoepitopes.

In yet other embodiments, the antigenic unit comprises any combinations of the aforementioned embodiments, i.e., any combination of one or more patient-present shared cancer antigens or parts thereof and of one or more neoantigens or parts thereof mentioned above.

### Antigenic Unit of Individualized Polypeptides Comprising One or More Neoantigens or Parts Thereof

Cancers develop from the patient's normal tissue by one or a few cells starting an abnormal, uncontrolled proliferation of the cells due to mutations. Although the cancer cells are mutated, most of the genome is intact and identical to the remaining cells in the patient. One approach of attacking a tumor is based on the knowledge that any tumor in any patient is unique: patient-specific mutations lead to expression of patient-specific mutated proteins, i.e., neoantigens that are unique for the particular patient. These neoantigens are not identical to any proteins in the normal cells of the patient.

Therefore, such neoantigens are suitable targets for a therapeutic pharmaceutical composition comprising vector of the disclosure which is manufactured specifically and only for the patient in question, i.e., an individualized anticancer vaccine. This may be particularly relevant for immunogenic constructs.

The mutation may be any mutation leading to a change in at least one amino acid. Accordingly, the mutation may be one of the following:
a non-synonymous mutation leading to a change in the amino acid.
a mutation leading to a frame shift and thereby a completely different open reading frame in the direction after the mutation.
a read-through mutation in which a stop codon is modified or deleted leading to a longer protein with a tumor-specific epitope
splice mutations that lead to a unique tumor-specific protein sequence
chromosomal rearrangements that give rise to a chimeric protein with a tumor-specific epitope at the junction of the two proteins. When the mutation is due to a chromosomal rearrangement, the tumor-specific epitope can arise from a change in at least one amino acid or from a combination of two in-frame coding sequences.

In some embodiments, the antigenic unit comprises one or more neoantigens or parts thereof, such as one or more parts of one neoantigen or one or more parts of several neoantigens, preferably one or more neoepitopes and more preferably several neoepitopes. Such neoepitopes may be selected for inclusion into antigenic unit according to their predicted therapeutic efficacy, see WO 2017/118695A1, the disclosure of which is incorporated herein by reference.

In some embodiments, the antigenic unit comprises one or more parts of one neoantigen or one or more parts of several neoantigens, preferably one or more neoepitopes. In some preferred embodiments, in the antigenic unit, the neoepitopes are separated by linkers. An alternative way to describe the separation of all neoepitopes by linkers is that all but the terminal neoepitope, i.e., the neoepitope at the N-terminal start of the first or further polypeptide or the C-terminal end of the first or further polypeptide, are arranged in antigenic subunits, wherein each subunit comprises a neoepitope and a subunit linker. Due to the separation of the neoepitopes by a linker, each neoepitope is presented in an optimal way to the immune system.

Hence, an antigenic unit that comprises n neoepitopes comprises n−1 antigenic subunits, wherein each subunit comprises a neoepitope and a subunit linker, and further comprises a terminal neoepitope. In some embodiments, n is an integer of from 1 to 50, e.g., 3 to 50 or 15 to 40 or 10 to 30 or 10 to 25 or 10 to 20 or 15 to 30 or 15 to 25 or 15 to 20. In some preferred embodiments, the antigenic subunit consists of a neoepitope and a subunit linker.

The neoepitope preferably has a length suitable for presentation by HLA molecules. Thus, in some preferred embodiments, the neoepitope has a length of from 7 to 30 amino acids. More preferred are neoepitopes having a length of from 7 to 10 amino acids or of from 13 to 30 amino acids, e.g., from 20 to 30 amino acids, e.g., 27 amino acids.

Preferably, the antigenic unit comprises a plurality of neoepitopes. In some embodiments, the antigenic unit comprises a plurality of different neoepitopes. In other embodiments, the antigenic unit comprises multiple copies of the same neoepitope. In yet other embodiments, the antigenic unit comprises several different neoepitopes and multiple copies of the same neoepitope.

Accordingly, a preferred approach is to include as many neoepitopes as possible in the antigenic unit (i.e., different and/or multiple copies of the same neoepitope) to thereby attack the cancer efficiently whilst not compromising the ability to activate T cells against the neoepitopes due to dilution of the desired T cell effect. Further, to secure that all neoepitopes are loaded efficiently to the same antigen-presenting cell, all neoepitope-encoding nucleotide sequences are comprised in a continuous polynucleotide chain resulting in the expression of a protein comprising all the neoepitopes instead of expressing each neoepitope as a discrete peptide.

To design the antigenic unit, the patient's tumor exome is analyzed to identify neoantigens. Preferably, the sequences of the most immunogenic neoepitopes from one or more neoantigens are selected for inclusion into the antigenic unit.

In some embodiments, the antigenic unit comprises at least 1 neoepitope. Preferably, the antigenic unit comprises at least 3 neoepitopes, more preferably at least 5 neoepitopes, such as 7 neoepitopes. In another more preferred embodiment, the antigenic unit comprises at least 10 neoepitope. In another more preferred embodiment, the antigenic unit comprises at least 15 neoepitopes, such as at least 20 or at least 25 or at least 30 or at least 35 or at least 40 or at least 45 neoepitopes.

Antigenic units comprising one or more neoepitopes are described in detail in WO 2017/118695A1. Any of such antigenic units can be used as antigenic unit in a first polypeptide and/or as a further antigenic unit in a further polypeptide encoded by a vector of the disclosure for use in individualized anticancer therapy.

### Antigenic Unit of Individualized Polypeptides Comprising One or More Patient-Present Shared Cancer Antigens or Parts Thereof

Shared tumor antigens are expressed by many tumors, either across patients with the same cancer type, or across patients and cancer types. An example is the HPV16 antigen, a viral antigen that is expressed in about 50% of all patients with squamous cell carcinoma of the head and neck, but also in patients with other cancers such as cervical cancer and vulvar squamous cell carcinoma. Many of these shared antigens have previously been characterized as immunogenic and/or are known, i.e., their immunogenicity has been confirmed by appropriate methods and the results have been published, e.g., in a scientific publication. Others have already been predicted to be presented on specific HLA class I or class II alleles, e.g., by algorithms known in the art and their predicted immunogenicity has been published, e.g., in a scientific publication, without having confirmed their immunogenicity by appropriate methods.

In some embodiments, the antigenic unit comprises one or more patient-present shared cancer antigens or parts thereof, e.g., patient-present shared cancer epitopes, which are known to be immunogenic, have known expression patterns and/or are known or have already been predicted to bind to specific HLA class I and class II molecules.

T cells specific to patient-present shared cancer antigens can travel to the tumor and affect the tumor microenvironment, thus increasing the likelihood that additional tumor-specific T cells are able to attack the cancer.

Some patient-present shared cancer antigens are proteins comprising an amino acid sequence that comprise one or more mutations, i.e., patient-present shared cancer epitopes which are known to be immunogenic or which have been predicted to be immunogenic. Other patient-present shared cancer antigens are proteins which do not comprise mutations, e.g., overexpressed cellular proteins.

In some embodiments, the patient-present shared cancer antigen is selected from the group consisting of overexpressed cellular proteins, aberrantly expressed cellular proteins, cancer testis antigens, viral antigens, differentiation antigens, mutated oncogenes and mutated tumor suppressor genes, oncofetal antigens, shared fusion antigens, shared intron retention antigens, dark matter antigens and shared antigens caused by spliceosome mutations or frameshift mutations.

In some embodiments, the patient-present shared cancer antigen is an overexpressed or aberrantly expressed human cellular protein, i.e., a cellular protein found at increased levels in tumors compared with normal healthy cells and tissues. Examples of such overexpressed or aberrantly expressed cellular proteins include tumor protein D52, Her-2/neu, hTERT (telomerase) and survivin.

In other embodiments, the patient-present shared cancer antigen is a cancer testis antigen which is normally expressed in male germ cells in the testis but not in adult somatic tissues. In some cases, such antigens are also expressed in ovary and trophoblast. In malignancy, this gene regulation is disrupted, resulting in antigen expression in a proportion of tumors of various types. Examples of cancer testis antigens include MAGE-A, MAGE-B, GAGE, PAGE-1, SSX, HOM-MEL-40 (SSX2), NY-ESO-1, LAGE-1 and SCP-1.

In yet other embodiments, the patient-present shared cancer antigen is a differentiation antigen, for example tyrosinase.

In yet other embodiments, the patient-present shared antigen is a viral antigen. Examples of viral antigens include those comprised in human papilloma virus (HPV), Epstein-Barr virus (EBV), Kaposi's sarcoma-associated herpesvirus (KSHV), Merkel cell polyomavirus (MCV or MCPyV), human cytomegalovirus (HCMV) and human T-lymphotropic virus (HTLV).

In yet other embodiments, the patient-present shared cancer antigen is a mutated oncogene. Examples of mutated oncogenes include KRAS, CALR and TRP-2.

In yet other embodiments, the patient-present shared cancer antigen is a mutated tumor suppressor gene. Examples include mutated p53, mutated pRB, mutated BCL2 and mutated SWI/SNF.

In yet other embodiments, the patient-present shared cancer antigen is an oncofetal antigen, for example alpha-fetoprotein or carcinoembryonic antigen.

In yet other embodiments, the patient-present shared antigen is a shared intron retention antigen or shared antigen caused by frameshift mutation, for example CDX2 or CALR.

In yet other embodiments, the patient-present shared antigen is a shared antigen caused by spliceosome mutations. An example is an antigen caused by mutations like SF3B1 mut.

Generally, for any cancer antigen, immune tolerance has likely occurred when a patient presents with cancer. An anticancer vaccine should specifically trigger immune response to the antigens incorporated in the vaccine. In some embodiments, the first polypeptide encoded by the plasmid functions as an anticancer vaccine. In some embodiments, the first polypeptide and/or at least one further polypeptide encoded by the plasmid functions as an anticancer vaccine. The peripheral immune tolerance to the selected antigens may be weak or strong. By incorporating such patient-present shared cancer antigens or one or more parts thereof in the antigenic unit—either alone or together with other patient-present shared cancer antigens or parts thereof and/or neoantigens or neoepitopes—a polypeptide comprising such antigenic unit elicits an immune response which is strong and broad enough to affect the tumor microenvironment and change the patient's immune response against the tumor from a suppressive/tolerated type to a pro-inflammatory type. This may help to break tolerance to several other antigens, thus representing a considerable clinical benefit for the patient. The afore-described concept may be referred to as tipping the cancer immunity set point.

In some embodiments, the antigenic unit comprises one or more patient-present shared cancer antigens or parts thereof that is a human cellular protein, preferably an overexpressed or aberrantly expressed human cellular protein or a differentiation antigen.

The patient-present shared cancer antigen can be detected in the tissue or body fluid of the patient by methods known in the art, including:
sequencing the patient's genome or exome and optionally searching, e.g., by tailor made software in whole genome/exome-seq data to, e.g., identify mutated oncogenes or mutated tumor suppressor genes;
immunohistochemistry of the patient's tumor tissue, e.g., to detect the presence of mutated proteins;
RT-PCR, e.g., to detect the presence of viral antigens or known mutations in oncogenes;
ELISA using antibodies against, e.g., mutated tumor proteins in serum samples;
RNA-seq of tumor tissue and comparison to healthy tissue to, e.g., detect expression/over-expression of shared cancer antigens;
Searching, e.g., by tailor-made software in raw RNA sequence data to identify intron retention antigens;
searching, e.g., by tailor-made software, in whole genome-seq data to identify transposable elements which are elements of dark matter antigens;
detection of short repeats in raw whole exome/RNA sequence data to, e.g., identify dark matter antigens;
RNA-seq data to, e.g., identify shared viral antigens; and
comparing RNA-seq of the patient's tumor samples with either patient's own healthy tissue or a cohort/database (e.g., TCGA) versus consensus transcript expression, such as GTEX/HPA gene expression data.

In some preferred embodiments, the antigenic unit comprises one or more patient-present shared cancer antigens or part(s) of such antigen(s) that is known to be immunogenic, e.g., has previously been described to elicit an immune response in other patients, or has been predicted to bind to the patient's HLA class I and/or class II alleles.

In some embodiments, the antigenic unit comprises one or more patient-present shared cancer epitopes. In some preferred embodiments, such epitopes have a length suitable for presentation by the patient's HLA alleles.

In some embodiments, the antigenic unit comprises one or more patient-present shared cancer epitopes having a length suitable for specific presentation on HLA class I or HLA class II. In some embodiments, the epitope has a length of from 7 to 11 amino acids for HLA class I presentation. In other embodiments, the epitope has a length of from 13 to 30 amino acids for HLA class II presentation.

In some embodiments, the antigenic unit comprises one or more patient-present shared cancer epitopes having a length of from 7 to 30 amino acids, e.g., from 7 to 10 amino acids (such as 7, 8, 9, or 10 amino acids) or from 13 to 30 amino acids (such as 13, 14, 15, 16, 17, 18, 19, 20, 21, 22, 23, 24, 25, 26, 27, 28, 29 or 30 amino acids), such as 7, 8, 9, 10, 11, 12, 13, 14 or 15 amino acids.

The antigenic unit may comprise one or more patient-present shared cancer antigens either in full-length or one or more parts thereof.

In some embodiments, the antigenic unit comprises one patient-present shared cancer antigen in full-length. In other embodiments, the antigenic unit comprises several patient-present shared cancer antigens, each of them in full-length.

In yet other embodiments, the antigenic unit comprises one or more parts of a patient-present shared cancer antigen, e.g., one or more patient-present shared cancer epitopes. In yet other embodiments, the antigenic unit comprises one or more parts of several patient-present shared cancer antigens, e.g., one or more epitopes of several patient-present shared cancer antigens.

In yet other embodiments, the antigenic unit comprises one or more patient-present shared antigens in full-length and one or more parts of one or more patient-present shared cancer antigens. Examples include:
antigenic units comprising one patient-present shared antigen in full-length and one or more epitopes of one patient-present shared cancer antigen; and
antigenic units comprising several patient-present shared cancer antigens, each of them in full-length and one or more epitopes of one patient-present shared cancer antigen; and
antigenic units comprising one patient-present shared antigen in full-length and one or more epitopes of several patient-present shared cancer antigens; and
antigenic units comprising several patient-present shared cancer antigens, each of them in full-length and one or more epitopes of several patient-present shared cancer antigens.

In some preferred embodiments, the aforementioned epitopes are already known to be immunogenic, e.g., have been described to be immunogenic in the literature, or have already been predicted to bind to the patient's HLA class I and class II alleles, e.g., as described in the literature, preferably have already been predicted to bind to the patient's HLA class I alleles. In other preferred embodiments, the immunogenicity of the aforementioned epitopes is predicted, e.g., the binding of the epitopes to one or more of the patient's HLA class I and/or HLA class II molecules is predicted by methods known in the art, such as those disclosed in WO 2021/205027 A1, the disclosure of which is incorporated herein by reference, or those described herein, including those described in the section “Methods for designing an antigenic unit of an individualized polypeptide”.

In some embodiments, the antigenic unit comprises 1 to 10 patient-present shared antigens in full-length.

In other embodiments, the antigenic unit comprises 1 to 30 parts of one or more patient-present shared antigens, wherein these parts include multiple epitopes that are predicted to bind to a patient's HLA class I or class II alleles. In yet other embodiments, the antigenic unit comprises 1 to 50 patient-present shared cancer epitopes, preferably epitopes that are predicted to bind to the patient's HLA class I or class II alleles.

### Antigenic Units of Individualized Polypeptides Comprising One or More Patient-Present Shared Cancer Antigens or Parts Thereof and One or More Neoantigens or Parts Thereof

In further embodiments, the antigenic units are a combination of all of the afore-described embodiments relating to antigenic units, which comprise one or more patient-present shared cancer antigens or parts thereof and all of the afore-described embodiments relating to antigenic units, which comprise one or more neoantigens or parts thereof.

Antigenic units comprising one or more patient-present shared cancer antigens or parts thereof and optionally one or more neoantigens and parts thereof are described in detail in WO 2021/205027A1, the content of which is included herein by reference. Any of such antigenic units can be used as antigenic unit in the first polypeptide and/or at least in one further polypeptide encoded for in the vector of the disclosure for use in individualized anticancer therapy.

### Methods for Designing an Antigenic Unit of an Individualized Polypeptide

The patient-present shared cancer antigens and neoantigens identified in a particular patient are preferably further processed to find those antigens that will render the first polypeptide and/or a further polypeptide most effective, when those antigens are included into the antigenic unit. The way and order in which such processing is done depends on how said antigens were identified, i.e., the data that form the basis for such processing.

In some embodiments, the processing and selecting of the antigen(s) to be included in the antigenic unit is carried out as follows:
1) A search in the literature and/or in one or more databases is carried out to retrieve information about and sequences of shared cancer antigens and preferably information about their expression pattern, immunogenicity or predicted immunogenicity, epitopes and HLA presentation. Such search is also carried out to determine whether the identified antigen is a patient-present shared cancer antigen or a neoantigen.
2) If it was determined that the identified antigen is a patient-present shared cancer antigen, the sequence thereof is studied to identify epitopes, preferably all epitopes, that are predicted to bind to the patient's HLA class I/II alleles. The prediction may be carried out by using prediction tools known in the art, e.g., prediction software known in the art, such as NetMHCpan and similar software.
3) The most promising sequences of the patient-present shared cancer antigen which are most immunogenic or predicted to be most immunogenic, i.e., those that show predicted binding to one or more of the patient's HLA class I/II alleles, are selected for inclusion into the antigenic unit. In some embodiments, minimal epitopes are selected, e.g., if only a few promising epitopes were identified in step 2 or if longer stretches of non-immunogenic sequences are present between the epitopes. In other embodiments, a longer sequence is selected which comprises several epitopes that bind to the patient's specific HLA alleles. In yet other embodiments, the full-length sequence of the antigen is selected for inclusion into the antigenic unit.
4) The most promising parts of neoantigen sequences, e.g., neoepitopes, are selected for inclusion into the antigenic unit based on predicted immunogenicity and binding to the patient's HLA class I/II alleles of such sequences.

Tumor mutations are discovered by sequencing of tumor and normal tissue and comparing the obtained sequences from the tumor tissue to those of the normal tissue. A variety of methods is available for detecting the presence of a particular mutation or allele in a patient's DNA or RNA. Such methods include dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. Alternatively, mutations may be identified by direct protein sequencing.

Out of the maybe hundreds or thousands of mutations in the tumor exome, the most promising sequences are selected in silico based on predictive HLA-binding algorithms. The intention is to identify all relevant epitopes and after a ranking or scoring, determine the sequences to be included in the antigenic unit. Methods known in the art may be suitable for scoring, ranking and selecting neoepitopes include those disclosed in WO 2020/065023A1 and WO 2020/221783A1.

Further, any suitable algorithm for such scoring and ranking may be used, including the following:

Available free software analysis of peptide-MHC binding (IEDB and NetMHCpan) that can be downloaded from the following websites:
www.iedb.org/
www.cbs.dtu.dk/services/NetMHC/

Commercially available advanced software to predict optimal sequences for vaccine design are found here:
www.oncoimmunity.com/
omictools.com/T cell-epitopes-category
github.com/griffithlab/pVAC-Seq
crdd.osdd.net/raghava/cancertope/help.php
www.epivax.com/tag/neoantigen/

Each mutation is scored with respect to its antigenicity, and the most antigenic neoepitopes are selected and optimally arranged in the antigenic unit.

### Antigenic Unit of Non-Individualized Polypeptides

### Antigenic Units of Polypeptides Comprising One or More Shared Cancer Antigens or Parts Thereof

In some embodiments, a non-individualized or “off-the-self” comprises a polynucleotide sequence encoding an antigenic unit, which comprises one or more shared cancer antigens or parts thereof.

“Shared cancer antigen” or “shared tumor antigen” is used herein to describe an antigen that has been described to be expressed by many tumors, either across patients with the same cancer type, or across patients and cancer types.

“Shared cancer epitope” is used herein to describe an amino acid sequence comprised in a shared cancer antigen, which is known or predicted to be immunogenic.

In some embodiments, the antigenic unit non-individualized polypeptides for use in the treatment of cancer comprises one or more shared cancer antigens or parts thereof, e.g., shared cancer epitopes, which are known to be immunogenic, have known expression patterns and/or are known or have already been predicted to bind to specific HLA class I and class II molecules.

Some shared cancer antigens are proteins comprising an amino acid sequence that comprise one or more mutations, i.e., shared cancer epitopes which are known to be immunogenic or which have been predicted to be immunogenic. Other shared cancer antigens are proteins which do not comprise mutations, e.g., overexpressed cellular proteins.

In some embodiments, the shared cancer antigen is selected from the group consisting of overexpressed cellular proteins, aberrantly expressed cellular proteins, cancer testis antigens, viral antigens, differentiation antigens, mutated oncogenes and mutated tumor suppressor genes, oncofetal antigens, shared fusion antigens, shared intron retention antigens, dark matter antigens and shared antigens caused by spliceosome mutations or frameshift mutations.

In some embodiments, the shared cancer antigen is an overexpressed or aberrantly expressed human cellular protein, i.e., a cellular protein found at increased levels in tumors compared with normal healthy cells and tissues. Examples of such overexpressed or aberrantly expressed cellular proteins include tumor protein D52, Her-2/neu, hTERT (telomerase) and survivin.

In other embodiments, the shared cancer antigen is a cancer testis antigen which is normally expressed in male germ cells in the testis but not in adult somatic tissues. In some cases, such antigens are also expressed in ovary and trophoblast. In malignancy, this gene regulation is disrupted, resulting in antigen expression in a proportion of tumors of various types. Examples of cancer testis antigens include MAGE-A, MAGE-B, GAGE, PAGE-1, SSX, HOM-MEL-40 (SSX2), NY-ESO-1, LAGE-1 and SCP-1.

In yet other embodiments, the shared cancer antigen is a differentiation antigen, for example tyrosinase.

In yet other embodiments, the shared antigen is a viral antigen. Examples of viral antigens include those comprised in human papilloma virus (HPV), Epstein-Barr virus (EBV), Kaposi's sarcoma-associated herpesvirus (KSHV), Merkel cell polyomavirus (MCV or MCPyV), human cytomegalovirus (HCMV) and human T-lymphotropic virus (HTLV).

In yet other embodiments, the shared cancer antigen is a mutated oncogene. Examples of mutated oncogenes include KRAS, CALR and TRP-2.

In yet other embodiments, the shared cancer antigen is a mutated tumor suppressor gene. Examples include mutated p53, mutated pRB, mutated BCL2 and mutated SWI/SNF.

In yet other embodiments, the shared cancer antigen is an oncofetal antigen, for example alpha-fetoprotein or carcinoembryonic antigen.

In yet other embodiments, the shared antigen is a shared intron retention antigen or shared antigen caused by frameshift mutation, for example CDX2 or CALR.

In yet other embodiments, the shared antigen is a shared antigen caused by spliceosome mutations. An example is an antigen caused by mutations like SF3B1 mut.

Further examples of shared cancer antigens include scFvs derived from a monoclonal Ig produced by myeloma or lymphoma, also called the myeloma/lymphoma M component in patients with B cell lymphoma or multiple myeloma, HIV derived sequences like e.g., gpl20 or Gag derived sequences, tyrosinase related protein (TRP)-1, melanoma antigen, prostate specific antigen and idiotypes, HPV antigens selected from the list consisting of E1, E2, E6, E7, L1 and L2, e.g., E6 and/or E7 of HPV16 and/or HPV18.

Any shared cancer antigen sequence of sufficient length that includes a specific epitope may be used as the antigenic unit. Accordingly, in some embodiments, the antigenic unit comprises an amino acid sequence of at least 7 amino acids, such as at least 8 amino acids, corresponding to at least about 21 nucleotides, such as at least 24 nucleotides, in a nucleic acid sequence encoding such antigenic unit.

In yet other embodiments, the antigenic unit comprises one or more parts of a shared cancer antigen, e.g., one or more shared cancer epitopes. In yet other embodiments, the antigenic unit comprises one or more parts of several shared cancer antigens, e.g., one or more epitopes of several shared cancer antigens. In yet other embodiments, the antigenic unit comprises one or more shared antigens in full-length and one or more parts of one or more shared cancer antigens. Examples include:
antigenic units comprising one shared antigen in full-length and one or more epitopes of one shared cancer antigen; and
antigenic units comprising several shared cancer antigens, each of them in full-length and one or more epitopes of one shared cancer antigen; and
antigenic units comprising one shared antigen in full-length and one or more epitopes of several shared cancer antigens; and
antigenic units comprising several shared cancer antigens, each of them in full-length and one or more epitopes of several shared cancer antigens.

Examples of polypeptides comprising shared antigens against HPV are disclosed in WO 2013/092875A1, the content of which is incorporated herein by reference.

### Methods for Designing an Antigenic Unit of a Polypeptide Comprising Shared Cancer Antigen(s)

Also, for vectors encoding polypeptides comprising shared cancer antigen(s) in an antigenic unit, the antigenic unit is designed to include those sequences that are likely to render the polypeptide effective in a variety of patients, e.g., patients having a certain type of cancer.

In some embodiments, the selection of the antigen to be included in the antigenic unit is carried out by performing a search in the literature and/or in one or more databases to retrieve information about and sequences of shared cancer antigens and preferably information about their expression pattern, immunogenicity or predicted immunogenicity, epitopes and/or HLA presentation. Epitopes are then identified that are known or predicted to bind to a variety of HLA class I/II alleles of many patients or that bind a certain subset of HLA class I/II alleles which is dominant in a certain cancer indication and/or a certain patient population across different cancer indications. Preferably, the most promising, i.e., the sequences of the shared cancer antigen which are most immunogenic or predicted to be most immunogenic, are selected for inclusion into the antigenic unit.

### Antigenic Units of Polypeptides Comprising One or More Infectious Antigens or Parts Thereof

In another embodiment of the disclosure, the antigenic unit of the first polypeptide and/or of a further polypeptide is designed for the treatment of an infectious disease and the vector/first polypeptide is for use in the treatment of an infectious disease.

In some embodiments, the antigenic unit comprised in the first polypeptide and/or in a further polypeptide comprises a further antigenic unit comprising one or more further epitopes which are relevant for infectious diseases, e.g., one or more infectious antigens, i.e., antigens or parts thereof derived from pathogens.

“Infectious disease” is used herein to describe a condition caused by a pathogen or a condition wherein a pathogen is involved in causing it. An example of the latter are eggs of a parasite, which do not cause the disease itself but develop into larvae which cause it.

“A pathogen” includes viruses, bacteria, fungi and parasites.

The antigens described in this section are “infectious antigens”, i.e., antigens derived from pathogens, i.e., they are comprised (or naturally found) in proteins of a pathogen which causes the disease or is involved in causing it. The terms “infectious antigen” and “antigen derived from a pathogen” may be used herein interchangeably.

In some embodiments, the antigenic unit comprises one or more epitopes derived from a pathogen, e.g., the antigenic unit comprises one antigen derived from a pathogen or more than one antigen derived from a pathogen, i.e., multiple antigens derived from a pathogen, e.g., comprised in the same or different proteins of such pathogen.

In some embodiments, the antigenic unit comprises one or more epitopes derived from multiple pathogens. In some embodiments, the multiple pathogens are multiple different pathogens. In that context, a “different pathogen” may, for example, be a different virus or bacterium or a different strain of the same virus or bacterium or it may be the same strain, but comprising one or more mutations.

A vector comprising one or more epitopes derived from multiple pathogens may be for use in a pan-vaccine, e.g., a vaccine targeting different (seasonal) viruses. For example, the pan-vaccine could target betacoronavirus and influenza or target different strains of e.g., betacoronaviruses or different mutations of the same strain.

Examples of infectious antigens/antigens that are derived from pathogens are such of bacterial origin, e.g., tuberculosis antigens and OMP31 from brucellosis, or viral origin, e.g., HIV derived sequences like e.g. gp120 derived sequences, glycoprotein D from HSV-2, and influenza virus antigens like hemagglutinin, nucleoprotein and M2, and HPV derived antigens such as E1, E2, E6, E7, L1 or L2, such as E6 and E7 of HPV16 or HPV18.

In some embodiments, the antigenic unit comprises one or more betacoronavirus antigens or parts thereof.

Betacoronaviruses denotes a genus in the subfamily Orthocoronaviridae. Betacoronaviruses are enveloped, positive-sense single-stranded RNA viruses. Within the genus, four lineages are commonly recognized: lineage A (subgenus Embecovirus), lineage B (subgenus Sarbecovirus), lineage C (Merbecovirus) and lineage D (Nobecovirus). Betacoronaviruses include the following viruses which caused/cause epidemics/pandemics in humans or can infect humans: SARS-COV, which causes severe acute respiratory syndrome (SARS), MERS-COV, which causes Middle East respiratory syndrome (MERS), SARS-COV-2, which causes coronavirus disease 2019 (Covid-19), HCoV-OC43 and HCoV-HKU1. SARS-COV and SARS-COV-2 belong to the lineage B (subgenus Sarbecovirus), MERS-COV belongs to the lineage C (Merbecovirus) and HCoV-OC43 and HCoV-HKU1 belong to the lineage A (subgenus Embecovirus).

In some embodiments, the antigenic unit and/or the further antigenic unit comprises or consist of one or more antigens or parts or fragments thereof derived from RSV virus, such as one or more antigens or parts or fragments thereof derived from RSV F protein or RSV PreF protein. In some embodiments, the antigenic unit and/or the further antigenic unit comprises or consists of soluble RSV F protein or soluble RSV PreF protein. In some embodiments, the antigenic unit and/or the further antigenic unit comprises or consists of RSV preF protein comprising a transmembrane domain.

In some embodiments, the antigen is the spike protein of SARS-COV or SARS-COV-2, or a part thereof.

In some embodiments of the present disclosure, the antigen may be a T cell epitope which is a part of the sequence of the spike protein or the membrane protein or the envelope protein or the nucleocapsid protein or the ORF1a/b or ORF3a protein. In other embodiments, the T cell epitope is part of the following genes/proteins: NCAP, AP3A, spike, ORF1a/b, ORF3a, VME1 and VEMP.

In some embodiments, the antigenic unit of the vector of the disclosure comprises one or more epitopes derived from one or more pathogens selected from the list consisting of influenza virus, Herpes simplex virus, CMV, HPV, brucella bacteria, HIV, HSV-2 and Mycobacterium tuberculosis bacteria.

The vector of the disclosure for use in the treatment of infectious diseases is ideal for fighting pandemics and epidemics as it can induce rapid, strong immune response. Such a vector is designed to induce an antigenic effect through inclusion into the antigenic unit of the full-length or a part of one or more infectious antigens, such parts may for example be selected T cell epitopes, or through combinations thereof.

In some embodiments, the targeting unit of such a first polypeptide, and/or of a further polypeptide, is anti-pan-HLA class II, human CCL3, human CCL3L1, or human CCL3L1, and an immune response will be raised through B cells and/or T cells. In some embodiments, the vector can be used in a prophylactic setting or a therapeutic setting or both a prophylactic and a therapeutic setting.

### Antigenic Units of Polypeptides Comprising One or More T Cell Epitopes

In some embodiments, the antigenic unit of a first polypeptide, and/or of a further polypeptide, includes one or more discrete T cell epitopes, hotspots of minimal T cell epitopes or both. Such antigenic unit preferably includes hotspots of minimal T cell epitopes, i.e., one or more regions of an antigen or allergen that contain multiple minimal T cell epitopes (e.g., having a length of from 7-15 amino acids) that are predicted to be presented by different HLA alleles to cover a broad range of subjects, e.g., an ethnic population or even a world population. By including such hotspots, chances are maximized that the construct will induce tolerance in a broad range of subjects.

The number of T cell epitopes in the antigenic unit may vary, and depends on the length and number of other elements included in the antigenic unit, e.g., linkers.

In some embodiments, the antigenic unit comprises 1 to 10 T cell epitopes such as 1, 2, 3, 4, 5, 6, 7, 8 or 9 or 10 T cell epitopes or 11 to 20 T cell epitopes, such as 11, 12, 13, 14, 15, 16, 17, 18, 19 or 20 T cell epitopes or 21 to 30 T cell epitopes, such as 21, 22, 23, 24, 25, 26, 27, 28, 29 or 30 T cell epitopes or 31 to 40 T cell epitopes, such as 31, 32, 33, 34, 35, 36, 37, 38, 39 or 40 T cell epitopes or 41 to 50 T cell epitopes, such as 41, 42, 43, 44, 45, 46, 47, 48, 49 or 50 T cell epitopes.

In some embodiments, the at least one T cell epitope comprised in the antigenic unit is comprised in an antigen or a part thereof, wherein the antigen or a part thereof has a length of from 7 to about 200 amino acids, possibly including hotspots of minimal T cell epitopes. A “hotspot of minimal epitopes is a region that contains several minimal T cell epitopes (e.g., having a length of from 7-15 amino acids) that are predicted to be presented by different HLA alleles to cover a broad range of world population. In some embodiments, the at least one T cell epitope comprised in the further antigenic unit is comprised in an allergen, a hypoallergenic allergen, a self-antigen or an alloantigen, wherein the allergen, hypoallergenic allergen, self-antigen or alloantigen has a length of from 7 to about 200 amino acids, possibly including hotspots of minimal T cell epitopes. A “hotspot of minimal epitopes is a region that contains several minimal T cell epitopes (e.g., having a length of from 7-15 amino acids) that are predicted to be presented by different HLA alleles to cover a broad range of world population.

In some embodiments, the antigenic unit comprises at least one T cell epitope comprised in an antigen or part thereof, wherein the antigen or part thereof has a length of from 7 to 150 amino acids, preferably of from 7 to 100 amino acids, e.g., from about 10 to about 100 amino acids or from about 15 to about 100 amino acids or from about 20 to about 75 amino acids or from about 25 to about 50 amino acids. In some embodiments, the further antigenic unit comprises at least one T cell epitope comprised in an allergen, a hypoallergenic allergen, a self-antigen, or an alloantigen, which has a length of from 7 to 150 amino acids, preferably of from 7 to 100 amino acids, e.g., from about 10 to about 100 amino acids or from about 15 to about 100 amino acids or from about 20 to about 75 amino acids or from about 25 to about 50 amino acids.

A T cell epitope comprised in an antigen or part thereof, wherein the antigen or part thereof has a length of about 60 to 200 amino acids may be split into shorter sequences and included into the antigenic unit separated by linkers, e.g., linkers as described herein. By way of example, a T cell epitope comprised in an antigen or part thereof, wherein the antigen or part thereof has a length of 150 amino acids may be split into 3 sequences of 50 amino acids each, and included into the antigenic unit, with linkers separating the 3 sequences from each other. A T cell epitope comprised in an allergen, a hypoallergenic allergen, a self-antigen or an alloantigen having a length of about 60 to 200 amino acids may be split into shorter sequences and included into the antigenic unit separated by linkers, e.g., linkers as described herein. By way of example, a T cell epitope comprised in a hypoallergenic allergen, wherein the hypoallergenic allergen has a length of 150 amino acids may be split into 3 sequences of 50 amino acids each, and included into the antigenic unit, with linkers separating the 3 sequences from each other.

In some embodiments, the antigenic unit and/or the further antigenic unit comprises multiple T cell epitopes which are separated from each other by linkers, e.g., linkers as discussed herein, e.g., linkers as discussed in the “linkers comprised in the antigenic unit” section herein.

In some embodiments, the at least one T cell epitope has a length suitable for presentation by MHC. Thus, in some embodiments, the antigenic unit or the further antigenic unit comprises at least one T cell epitopes having a length suitable for specific presentation on MHC class I or MHC class II. In some embodiments, the at least one T cell epitope has a length of from 7 to 11 amino acids for MHC class I presentation. In other embodiments, the at least one T cell epitope has a length of about 15 amino acids for MHC class II presentation.

In some embodiments, the T cell epitope is known in the art, e.g., one that has been studied and described in the literature, e.g., known to be immunogenic, e.g., its immunogenicity has been confirmed by appropriate methods and the results have been published, e.g., in a scientific publication. In some embodiments, the antigenic unit and/or the further antigenic unit includes multiple T cell epitopes that are known to be immunogenic.

In other embodiments, the T cell epitope is predicted to be immunogenic, e.g., is selected based on the predicted ability to bind to HLA class I/II alleles. In some embodiments, the antigenic unit includes multiple T cell epitopes, e.g., multiple T cell epitopes that are separated from each other by linkers, e.g., linkers as discussed herein, e.g., as discussed in the “linkers in the antigenic unit” section herein, that are predicted to bind to HLA class I/II alleles. The T cell epitopes are selected in silico based on predictive HLA-binding algorithms. After having identified all relevant epitopes, the epitopes are ranked according to their ability to bind to HLA class I/II alleles and the epitopes that are predicted to bind best are selected to be included in the antigenic unit and/or in the further antigenic unit.

Suitable HLA binding algorithms are known in the art.

In yet other embodiments, the antigenic unit comprises multiple T cell epitopes some of which are known to be immunogenic and others that are predicted to be immunogenic. In some embodiments, the T cell epitopes are separated from each other by linkers, e.g., linkers as discussed herein, e.g., as discussed in the “linkers comprised in the antigenic unit” section herein.

Antigenic Units of Polypeptides Comprising One or More T Cell Epitopes from One or More Pathogens

In some embodiments, the antigenic unit of a first polypeptide, and/or of a further polypeptide, for use in the treatment of an infectious disease comprises at least one T cell epitope from one or more pathogens. Such T cell epitopes are comprised (or naturally found) in proteins of pathogens. Conserved parts of the genome among many pathogens comprise T cell epitopes capable of initiating immune responses.

In some embodiments, the antigenic unit comprises at least one T cell epitope of a pathogen, i.e., one T cell epitope of a pathogen or more than one T cell epitope of a pathogen, i.e., multiple T cell epitopes of a pathogen. In some embodiments, the multiple T cell epitopes are of the same pathogen, i.e., (naturally) comprised in the same or different proteins of the pathogen. In other embodiments, the multiple T cell epitopes are of multiple different pathogens, i.e., (naturally) comprised in protein of different pathogens.

In some preferred embodiments, the at least one T cell epitope is from a conserved region of the pathogen, i.e., is conserved between several subgenera, species or strains of a respective pathogen.

The T cell epitopes may be comprised in any of the pathogen's proteins, i.e., in surface proteins but also in the internal proteins such as viral nucleocapsid proteins or viral replicase polyproteins or in other structural and non-structural proteins.

A vector comprising an antigenic unit comprising T cell epitopes from conserved regions of pathogens will provide protection against several species/strains of the pathogen. Such a vector will also provide protection against multiple variants of a pathogen, which is important for the efficacy of such a first polypeptide, and/or of a further polypeptide, against future mutated pathogens. Viruses are known to mutate, e.g., undergo viral antigen drift or antigen shift. The finding of conserved regions across a viral genus makes it likely that these conserved regions are needed to maintain essential structures or functions, thus it is anticipated that future mutations will take place in the less-conserved regions. By raising an immune response against the conserved regions, the individual treated with plasmid will be protected also against mutated (and thus novel) strains of the future.

In some embodiments of the present disclosure, the antigenic unit is therefore designed to evoke a cell-mediated immune response through activation of T cells against the T cell epitopes of the infectious antigen/from a pathogen included in such antigenic unit. T cells recognize epitopes when they have been processed and presented complexed to an MHC molecule.

For example, useful T cell epitopes known in the art are those against infection by SARS-COV2 in humans can be found in Grifoni et al., Cell Host Microbe. 2021 Jul. 14; 29 (7): 1076-1092. Such T cell epitopes may thus be included in the antigenic unit of vectors for use in treating SARS-COV2 in humans. Another example of such T cell epitopes is the T cell epitope with the sequence CTELKLSDY (SEQ ID NO: 149) of the nucleoprotein from influenza A virus, the T cell epitope with the sequence NLVPMVATV (SEQ ID NO: 150) of the 65 kDa phosphoprotein from human herpesvirus 5 (human cytomegalovirus) and the T cell epitope with the sequence KLVANNTRL (SEQ ID NO: 151) of diacylglycerol acyltransferase/mycolyltransferase Ag85B from Mycobacterium tuberculosis. In some embodiments, the antigenic unit comprises at least one T cell epitope selected from the group consisting of T cell epitopes set forth in SEQ ID NOs: 159-178.

As an example, the at least one T cell epitope may be from a region of a human papilloma virus (HPV), e.g., from HPV16 or HPV18, e.g., at least one T cell epitope comprised in HPV antigens from the group consisting of E1, E2, E6, E7, L1 and L2, e.g., E6 and/or E7 of HPV16 and/or HPV18. By including such T cell epitopes in the vectors of the disclosure, a pharmaceutical composition comprising such vector may will provide protection against HPV. HPV infections are involved in certain cancers, such as squamous cell carcinoma of the head and neck, cervical cancer and vulvar squamous cell carcinoma. Indeed, HPV16 viral antigens are expressed in about 50% of all patients with said cancers.

As another example, the at least one T cell epitope may be from a region of a human influenza virus, such as human influenza virus A, human influenza virus B, human influenza virus C and human influenza virus D. As an example, the human influenza virus may be a specific hemagglutinin (HA) subtype, such as H1, H2, and H3, and/or a specific neuraminidase (NA) subtype, such as N1 or N5. As an example, the human influenza virus may be a H1N1 subtype. Such T cell epitopes may thus be included in the antigenic unit of a vector of the disclosure for use in the treatment of influenza infections.

Antigenic units comprising T cell epitopes for use in a vector for the prophylactic and therapeutic treatment of betacoronavirus infections and generally applicable methods for selecting T cell epitopes for vectors of the disclosure used in the prophylactic and therapeutic treatment of infectious diseases are disclosed in detail in WO2021/219897A1, the disclosure of which is incorporated herein by reference.

Antigenic Units of Polypeptides Comprising One or More Full-Length Infectious Antigens or Parts Thereof or One or More B Cell Epitopes from One or More Pathogens

In another aspect of the disclosure, a subject, e.g., a human individual, is a healthy individual and the vector of the disclosure is used prophylactically, e.g., to prevent a disease. Typically, the vector will be used to induce immunity in individuals where it is desired to raise neutralizing antibodies against a pathogen in a prophylactic setting, e.g., to prevent an infection.

In some embodiments, the vector of the disclosure encodes a first polypeptide, and/or of a further polypeptide, that comprises an antigenic unit comprising at least one infectious antigen which is a full-length protein of a pathogen or a part of such a protein. As such, in some embodiments, the at least one infectious antigen is a full-length surface protein or a part thereof, e.g., a full-length viral surface protein or bacterial surface protein or a full-length surface protein of any other pathogen.

In other embodiments, the infectious antigen is a full-length bacterial protein which is secreted by the bacterium, e.g., secreted into the cytoplasm of the cells of infected subjects.

In other embodiments, the antigenic unit comprises more than one infectious antigen or parts of more than one infectious antigen, e.g., multiple full-length infectious antigens.

In yet other embodiments, the antigenic unit comprises one or more epitopes derived from multiple pathogens or parts of such antigens, e.g., the antigenic unit comprises multiple full-lengths infectious antigens from multiple pathogens. In some embodiments, the multiple pathogens are multiple different pathogens.

In some embodiments such a protein of a pathogen is selected from a betacoronavirus protein, e.g., selected from the group consisting of envelope protein, spike protein, membrane protein and, if the betacoronvirus is an Embecovirus, spike-like protein hemagglutinin esterase.

In other embodiments, the antigenic unit comprises one part of one infectious antigen. The RBD domain of the spike protein of SARS-COV-2 or the head or stem domain of hemagglutinin of the influenza virus are examples of parts of an infectious antigen.

In other embodiments, the antigenic unit comprises several parts of one infectious antigen. In other embodiments, the antigenic unit comprises one part of several infectious antigens, e.g., one part of infectious antigen 1 and one part of infectious antigen 2 and 1 part of infectious antigen 3. In other embodiments, the antigenic unit comprises several parts of several infectious antigens, e.g., 2 parts of infectious antigen 1 and 3 parts of infectious antigen 2. The infectious antigens 1, 2 and 3 may be derived from one pathogen or from multiple, different pathogens.

If more than one infectious antigen is comprised in the antigenic unit, or more than 1 part of one or more infectious antigens, the antigens or parts thereof may be separated by linkers, e.g., by linkers as discussed herein, e.g., as discussed in the “linkers in the antigenic unit” section herein.

The one or more infectious antigens or parts thereof comprise conformational B cell epitopes, but may also comprise linear B cell epitopes and/or T cell epitopes. In contrary to the T cell epitopes discussed in the previous section herein, these T cell epitopes are not isolated, but are presented to the immune system in their natural environment, i.e., flanked by the amino acid residues which are present in the antigen.

In some embodiments, the antigenic unit comprises at least a B cell epitope derived from a pathogen, e.g., from a full-length protein of a pathogen, such as a full-length surface protein, e.g., comprised in any of the aforementioned proteins and preferably comprises several B cell epitopes derived from a pathogen, e.g., comprised in a full-length protein of a pathogen, such as a full-length surface protein, e.g., comprised in any of the aforementioned proteins. The at least one B cell epitope may be a linear or a conformational B cell epitope.

Once administered, the first polypeptide encoded by the first nucleic acid and/or at least one further polypeptide encoded by the one or more further nucleic acids comprised in the vectors of the disclosure as described above, i.e., a polypeptide comprising an antigenic unit, wherein the antigenic unit comprises one or more infectious full-length antigens or parts of such antigens, elicits a B cell response and T cell response and can be used as prophylactic or therapeutic treatment.

Such antigens may be selected for inclusion into the antigenic unit according to their predicted therapeutic efficacy, see WO2021/219897A1, the disclosures of which is incorporated herein by reference.

Antigenic Units of Polypeptides Comprising B Cell Epitopes and T Cell Epitopes from One or More Pathogens

In some embodiments, the first polypeptide encoded by the first nucleic acid, and/or of a further polypeptide encoded by at least one further polypeptide encoded by the one or more further nucleic acids comprised in the vectors of the disclosure will, once administered to a subject, elicit a T cell response and a B cell response. In a pandemic or an epidemic situation, it is not time efficient to first diagnose an individual to determine if he or she needs primarily a B or T cell response, neither whether prophylactic or therapeutic treatment is the highest medical need. Less so, as the determination of whether or not an individual is infected can be difficult due to lack of (sufficient) applicable tests. Thus, being able to protect and cure at the same time is important. By combining both full-length infectious antigens or parts of infectious antigens or several B cell epitopes present in an infectious antigen and T cell epitopes, such as conserved T cell epitopes, both a strong humoral and cellular response is elicited once the vector is administered. The response can be more humoral or more cellular, depending on the selected targeting unit.

Such a combination of T cell epitopes and infectious antigens or parts thereof may be selected for inclusion into the antigenic unit according to the T cell epitopes' predicted immunogenicity or by selecting T cell epitopes known in the art, see WO2021/219897A1, the disclosure of which is incorporated herein by reference.

In some embodiments, the full-lengths infectious antigens/parts thereof and the at least one T cell epitope are arranged in the antigenic unit as follows: the at least one T cell epitope is arranged in a subunit which is connected to the multimerization unit by a first linker, such as an unit linker. If multiple T cell epitopes are present in the subunit, the T cell epitopes are preferably separated by subunit linkers. Further, the subunit is separated from the one or more full-length infectious antigens or parts thereof by a second linker. Thus, the subunit with the T cell epitope(s) is closest to the multimerization unit, while the infectious antigen(s) or parts thereof constitute the terminal end of the polypeptide.

The subunit linkers, first linker/unit linker and second linker may be linkers as discussed herein, e.g., as discussed in the “linkers in the antigenic unit” and “unit linker” sections herein.

### Antigenic Unit of the First Polypeptide, Comprising Epitopes of an Allergen, Self-Antigen or Alloantigen

In some embodiments, the T cell epitope is a T cell epitope from a conserved region of the allergen, i.e. conserved between several allergens. In other words, the epitope may be encoded by a nucleotide sequence which is found in a conserved region of the genome of the allergen, i.e. conserved between several allergens. The epitope may thus be conserved between several allergens, i.e. the amino acid sequence of the epitope is conserved between these.

### Antigenic Unit of the One or More Further Polypeptides, of Constructs Comprising Epitopes of an Allergen, Self-Antigen or Alloantigen

The one or more further polypeptide in such constructs comprises an antigenic unit comprising one or more allergens, hypoallergenic allergens, self-antigens or alloantigens.

The term “hypoallergenic allergen” refers to an allergen that has been engineered to reduce allergenic activity, such a IgE reactivity.

The term “alloantigen” or “allograft antigen” as used herein refers to an antigen derived from (shed from and/or present in) a cell or tissue which, when transferred from a donor to a recipient, can be recognized and bound by an antibody of B or T cell receptor of the recipient. Alloantigens are typically products of polymorphic genes. An alloantigen is a protein or peptide which, when compared between donor and recipient (belonging to the same species), displays slight structural differences. The presence of such a donor antigen in the body of a recipient can elicit an immune response in the recipient. Such alloreactive immune response is specific for the alloantigen.

The term “self-antigen” as used herein refers to an antigen derived from (shed from and/or present in) a cell or tissue present in a subject's own body.

In some embodiments, the antigenic unit comprises multiple allergens, hypoallergenic allergens, self-antigens or alloantigens.

In some embodiments, the antigenic unit includes one allergen, hypoallergenic allergen, self-antigen or alloantigen. In other embodiments, the antigenic unit includes more than one allergens, hypoallergenic allergens, self-antigens or alloantigens, i.e., multiple allergens, hypoallergenic allergens, self-antigens or alloantigens.

Allergens, hypoallergenic allergens, self-antigens or alloantigens suitable for inclusion into the antigenic unit may be known in the art, i.e., have been studied, proposed and/or verified to be involved and of relevance for an allergic disease and published, e.g., in the scientific literature.

In some embodiments, the antigenic unit comprises allergens, hypoallergenic allergens, self-antigens or alloantigens with a length of from 7 to 1500 amino acids, preferably of from 7 to 1000 amino acids, e.g., from 9 or 10 to 1000 amino acids or from 15 to 1000 amino acids or from 9 to 600 amino acids or from 9 to 300 amino acids or from 15 to 600 of from 15 to 300 or from 20 to 250 amino acids or from 25 to 200 amino acids.

In some embodiments, the antigenic unit comprises one or more allergens, hypoallergenic allergens, self-antigens or alloantigens, i.e., one allergens, hypoallergenic allergens, self-antigens or alloantigens or more than one allergens, hypoallergenic allergens, self-antigens or alloantigens, i.e., multiple allergens, hypoallergenic allergens, self-antigens or alloantigens. In some embodiments, the multiple hypoallergenic allergens are of the same allergen, i.e., comprised in the same allergen. In another embodiment, the multiple hypoallergenic allergens are of multiple different allergens, i.e., comprised in different allergens. In some embodiments, the multiple allergens are of the same allergen, i.e. comprised in the same allergen. In other embodiments, the multiple allergens are of multiple different allergens, i.e. comprised in different allergens. In some embodiments, the multiple self-antigens are of the same self-antigen, i.e., comprised in the same self-antigen. In another embodiment, the multiple self-antigens are of multiple different self-antigens, i.e., comprised in different self-antigens. In some embodiments, the multiple alloantigens are of the same alloantigen, i.e., comprised in the alloantigen. In another embodiment, the multiple alloantigens are of multiple different alloantigens, i.e., comprised in different alloantigens.

In some embodiments, the further antigenic unit comprises only one copy of each hypoallergenic allergen. In other embodiments, the further antigenic unit comprises multiple copies of one or more allergens, hypoallergenic allergens, self-antigens or alloantigens.

In some embodiments, the further antigenic unit comprises only one copy of each hypoallergenic allergen, so that when e.g., 10 different allergens, hypoallergenic allergens, self-antigens or alloantigens are comprised in the further antigenic unit, a vector comprising said further antigenic unit may elicit a tolerance-inducing immune response against all 10 different hypoallergenic allergen.

In some embodiments, the one or more allergens, hypoallergenic allergens, self-antigens or alloantigens comprise B cell epitopes and T cell epitopes.

### Hypoallergenic Allergens

In some embodiments, the one or more hypoallergenic allergens are recombinant hypoallergenic allergens.

In some embodiments, the one or more hypoallergenic allergens are derived from one or more wild-type allergens and might be referred to as “hypoallergenic allergen derivatives” or “hypoallergenic derivatives”.

In some embodiments, the one or more hypoallergenic allergens have reduced allergenic activity. In some embodiments, the one or more hypoallergenic allergens have reduced IgE reactivity.

In some embodiments, the hypoallergenic allergens induce, upon immunization, allergen-specific IgG responses in the patient, which compete with IgE binding and thus, depending on the titers and specificities of the blocking IgG response, reduce IgE-mediated mast cell and basophil degranulation (Curin et al., 2018). This may result in reduced allergic symptoms as well as IgE-facilitated allergen presentation and thus T cell activation and late-phase allergic responses.

In some embodiments, the one or more hypoallergenic allergens exhibit reduced IgE-reactivity, such as reduced ability to bind allergen-specific IgE antibodies. In some embodiments, the one or more hypoallergenic allergens exhibit reduced allergenic activity, such as reduced ability to induce IgE-mediated mast cells or basophil degranulation. In some embodiments, the one or more hypoallergenic allergens lack IgE-reactivity, such as lack the ability to bind allergen-specific IgE antibodies. In some embodiments, the one or more hypoallergenic allergens lack allergenic activity, such as lack the ability to induce IgE-mediated mast cells or basophil degranulation. Such hypoallergenic allergens may reduce IgE-mediated side-effects during immunotherapy.

An injected vector encoding a relevant hypoallergenic allergen may induce immune responses with a T helper 1 (Th1) bias and promote the formation of IFN-γ producing CD4+ T cells that can stimulate B cells to synthesize IgG antibodies to block IgE binding to native allergen. In parallel, the Th1 biased response may skew the response away from Th2-mediated IgE production, and thereby inhibit the production of IgE.

In some embodiments, the one or more hypoallergenic allergens have lost their native conformation. In some embodiments, the one or more hypoallergenic allergens have lost their native conformation compared to the corresponding wildtype allergen which the one or more hypoallergenic allergens are derived from. In some embodiments, the one or more hypoallergenic allergens induce allergen-specific IgG antibody responses upon administration, which interfere with the IgE recognition of wildtype allergens

In some embodiments, the one or more hypoallergenic allergens comprise one or more allergen-specific T cell epitopes and/or one or more allergen-specific B cell epitopes. Preserving allergen-specific T cell epitopes may induce less allergen-specific IgE antibodies than the corresponding wildtype allergens upon immunization.

In some embodiments, the one or more hypoallergenic allergens are selected from the group consisting of mutated allergens, allergen fragments, disrupted allergens, denatured allergens, mosaic molecules comprising multiple allergens or parts thereof, hybrids comprising multiple allergens or parts thereof and allergen oligomers.

In some embodiments, the one or more hypoallergenic allergens are mutated allergens. In some embodiments, the one or more hypoallergenic allergens comprise an amino acid sequence which has been changed compared to the corresponding wildtype allergen which the one or more hypoallergenic allergens are derived from. In some embodiments, the one or more hypoallergenic allergens comprise at least one substitution, deletion or insertion compared to the wildtype allergen. In some embodiments, the one or more hypoallergenic allergens comprise 1, 2, 3, 4, 5, 6, 7, 8, 9, 10, 11, 12, 13, 14, 15, 16 17, 18, 19, 20, 21 or 22 substitutions, deletions or insertions compared to the wildtype allergen. In some embodiments, the one or more hypoallergenic allergens comprise 5, 10, 15, 20, 25, 30, 35, 40, 45, 50, 55, 60, 65, 70, 75, 80, 85, 90, 95 or 100 substitutions, deletions or insertions compared to the wildtype allergen. In some embodiments the at least one substitution, deletion or insertion results in reduced IgE reactivity.

In some embodiments, the one or more hypoallergenic allergens are allergen fragments. In some embodiments, the one or more hypoallergenic allergens comprise a fragment of the amino acid sequence of the corresponding wildtype allergen which the one or more hypoallergenic allergens are derived from. In some embodiments, the allergen fragments have an amino acid sequence comprising or consisting of 1, 2, 3, 4, 5, 6, 7, 8, 9, 10, 11, 12, 13, 14, 15, 16 17, 18, 19, 20, 21 or 22 amino acids. In some embodiments, the allergen fragments have an amino acid sequence comprising or consisting of 5, 10, 15, 20, 25, 30, 35, 40, 45, 50, 55, 60, 65, 70, 75, 80, 85, 90, 95 or 100 amino acids. In some embodiments, the allergen fragments have an amino acid sequence comprising or consisting of 50, 100, 150, 200, 250, 300, 350, 400, 450, 500, 550, 600, 650, 700, 750, 800, 850, 900, 950 or 1000 amino acids.

In some embodiments, the one or more hypoallergenic allergens are disrupted allergens. In some embodiments, the three-dimensional structure of the allergen has be disrupted. In some embodiments, the one or more hypoallergenic allergens are denatured allergens. In some embodiments, the three-dimensional structure of the allergen has been denatured. In some embodiments, the conformation of part of the allergen has been disrupted. In some embodiments, the conformation of part of the allergen has been denatured. In some embodiments, one or more conformational epitopes in the allergen have been disrupted. In some embodiments, one or more conformational epitopes in the allergen have been denatured. In some embodiments, the conformation of the whole allergen has been disrupted. In some embodiments, the conformation of the whole allergen has been denatured. Intact conformation of allergens might be crucial for IgE binding and that the disruption of the three-dimensional fold may lead to a reduction or loss of the IgE binding capacity.

Various strategies have been adopted to construct hypoallergenic allergen with reduced IgE reactivity including disruption of the three-dimensional structure of the allergen or by mutation of the amino acid residues involved in IgE-binding (Linhart and Valenta, 2011).

To map and define IgE-binding epitopes on a relevant allergen, online models as prediction tools may be used. These in silico mutation experiments enable estimation of structural changes of protein structures under sequence variation. One such example from literature is the shrimp tropomyosin Met e 1 where IgE-binding epitopes were defined by combining three online prediction tools in addition to ELISA and dot-immunoblotting using sera from shrimp allergy patients (Wai et al., 2014). With this strategy nine major IgE-binding Met e 1 epitopes were identified as the major IgE-binding epitopes. Based on the epitope data, two hypoallergenic allergens were constructed by site-directed mutagenesis and epitope deletion. The effect of such hypoallergenic allergens may be evaluated by their in vitro reactivity towards IgE from allergic patients and/or allergen-sensitized mice. Further, the effect may be measured by evaluating the effect on mast cell or basophil degranulation in a passive cutaneous anaphylaxis assays. The hypoallergens may be further assessed for their ability of inducing allergen-specific IgG antibodies. For example in mice, particularly IgG2a antibodies may strongly inhibit IgE from allergen-sensitized mice from binding the relevant allergen.

In some embodiments, the one or more hypoallergenic allergens are mosaic molecules comprising multiple hypoallergenic allergens or parts thereof. In some embodiments, the one or more hypoallergenic allergens are hybrids comprising multiple hypoallergenic allergens or parts thereof. In some embodiments, the mosaic molecules or hybrids comprise or consist of re-assembled allergen-derived fragments. In some embodiments, the mosaic molecules or hybrids comprise or consist of re-assembled allergen-derived fragments comprised in one molecule. Such mosaic molecules and hybrids may be able to retain the reduced IgE-binding activity of their components due to the loss of their three-dimensional structure and might be able to induce robust allergen-specific IgG antibody responses.

In some embodiments, the mosaic molecules or hybrids comprise multiple hypoallergenic allergens or parts thereof derived from several allergens relevant to a specific allergy. By way of example, the mosaic molecules or hybrids may comprise multiple hypoallergenic allergens or parts thereof derived from different major grass pollen allergens or house dust mite allergens. Including multiple allergens or parts thereof derived from several allergens may increase the immunogenicity of the included molecules.

Including multiple hypoallergenic allergens or parts thereof derived from several hypoallergenic allergens may increase the immunogenicity of the included molecules. Including multiple hypoallergenic allergens or parts thereof derived from several hypoallergenic allergens may increase the immunogenicity of the included molecules and may also reduce the number of molecules which need to be included.

In some embodiments, the one or more hypoallergenic allergens are allergen oligomers. In some embodiments, the one or more hypoallergenic allergens are allergen oligomers comprising 2 allergens. In some embodiments, the one or more hypoallergenic allergens are allergen oligomers comprising 3 allergens. In some embodiments, the one or more hypoallergenic allergens are allergen oligomers comprising 4 allergens. In some embodiments, the one or more hypoallergenic allergens are allergen oligomers comprising 5 allergens. Oligomerization of allergens may yield hypoallergenic oligomers for certain allergens by a mechanism of altered IgE epitope presentation. Allergen oligomers may form high molecular weight aggregates leading to altered presentation of IgE epitopes to effector cell-bound IgE. The allergens may be hypoallergenic.

By way of example, Fel d 1, Fel d 4 and Fel d 7 are three of the most prominent cat allergens, accounting for the majority of human cat allergies and the antigenic unit may comprise e.g., one or more hypoallergenic allergens of Fel d 1, i.e., one hypoallergenic allergens of Fel d 1 or multiple hypoallergenic allergens of Fel d 1. Further, the antigenic unit may comprise multiple hypoallergenic allergens of e.g., Fel d 4 and Fel d 7, e.g., one or multiple hypoallergenic allergens of Fel d 4 and one or multiple hypoallergenic allergens of Fel d 7.

The one or more hypoallergenic allergens may be derived from any of the allergens described in the below section “Allergens”.

### Allergens

In some embodiments, the vectors of the present disclosure as described herein are useful for inducing tolerance to a range of different protein allergens. T cell epitopes of allergens can be encoded by a nucleic acid sequence comprised in the first nucleic acid sequences and hypoallergenic allergens can be encoded by a nucleic acid sequence comprised in the one or more further nucleic acid sequences of the vector of the disclosure, including protein allergens that undergo post-translational modifications.

The one or more T cell epitopes of an allergen comprised in the antigenic unit of the first polypeptide and the one or more allergens or hypoallergenic allergens comprised in the further antigenic unit of the one or more further may be derived from the following allergens:

In some embodiments, the allergen is a food allergen. In some embodiments, the allergen is a shellfish allergen. In some embodiments, the allergen is tropomyosin, in other embodiments the allergen is arginine kinase, myosin light chain, sarcoplasmic calcium binding protein, troponin C or Triose-phosphate isomerase or actin. In some embodiments, the allergen is Pan b 1. In some embodiments the antigenic unit comprises Pan b 1 T cell epitope (251-270). In some embodiments the further antigenic unit comprises one or more Pan b 1 hypoallergenic allergen. In some embodiment, the antigenic unit comprises Met e 1. In some embodiment, the antigenic unit comprises one or more of the Met e 1 T cell epitopes (241-260), (210-230), (136-155), (76-95), (46-65) and (16-35). In some embodiments, the antigenic unit comprises all of the Met e 1 T cell epitopes (241-260), (210-230), (136-155), (76-95), (46-65) and (16-35). In some embodiments the further antigenic unit comprises one or more Met e 1 hypoallergenic allergen or fragments thereof.

In some embodiments, the allergen is a cow's milk allergen. In some embodiments, the cow's milk allergen is Bos d 4, Bos d 5, Bos d 6, Bos d 7, Bos d 8, Bos d 9, Bos d 10, Bos d 11 or Bos d 12.

In some embodiments, the allergen is an egg allergen. In some embodiments, the egg allergen is ovomucoid, in other embodiments the egg allergen is ovalbumin, ovotransferin, conalbumin, Gal d 3, egg lysozyme or ovomucin.

One T cell epitope that is known in the art and has been studied in the context of egg allergy is OVA (257-264), with amino acid sequence SIINFEKL (SEQ ID NO: 291).

In one embodiment, the antigenic unit of the construct encoded in the vector of the disclosure comprises the T cell epitope OVA (257-264). In some embodiments the further antigenic unit comprises one or more OVA hypoallergenic allergen. Such vector or a pharmaceutical composition comprising such vector may be used in the treatment of egg allergy.

In some embodiments, the allergen is a fish allergen. In some embodiments, the fish allergen is a parvalbumin. In other embodiments the fish allergen is enolase, aldolase or vitellogenin.

In some embodiments, the allergen is a fruit allergen. In some embodiments, the fruit allergen is pathogenesis-related protein 10, profilin, nsLTP, thaumatin-like protein, gibberellin regulated protein, isoflavone reductase related protein, class 1 chitinase, β 1,3 glucanase, germin like protein, alkaline serine protease, pathogenesis-related protein 1, actinidin, phytocystatin, kiwellin, major latex protein, cupin or 2S albumin. In some embodiments, the allergen is a vegetable allergen. In some embodiments, the vegetable allergen is pathogenesis related protein 10, profilin, nsLTP type 1, nsLTP type protein 2, osmotin-like protein, isoflavone reductase-like protein, β-fructofuranosidase, PR protein TSI-1, cyclophilin or FAD containing oxidase.

In some embodiments, the allergen is a wheat allergen. In some embodiments, the wheat allergen is Tri a 12, Tri a 14, Tri a 15, Tri a 18, Tri a 19, Tri a 20, Tri a 21, Tri a 25, Tri a 26, Tri a 27, Tri a 28, Tri a 29, Tri a 30, Tri a 31, Tri a 32, Tri a 33, Tri a 34, Tri a 35, Tri a 36, Tri a 37 or Tri a 38. In some embodiments, the allergen is a soy allergen.

In some embodiments, the soy allergen is Gly m 1, Gly m 2, Gly m 3, Gly m 4, Gly m 5, Gly m 6, Gly m 7 or Gly m 8. In other embodiments, the soy allergen is Gly m agglutinin, Gly m Bd28K, Gly m 30 kD, Gly m CPI or Gly m TI.

In some embodiments, the allergen is a peanut allergen. In some embodiments, the peanut allergen is Ara h 1, Ara h 2, Ara h 3, Ara h 4, Ara h 5, Ara h 6, Ara h 7, Ara h 8, Ara h 9, Ara h 10, Ara h 11, Ara h 12, Ara h 13, Ara h 14, Ara h 15, Ara h 16, or Ara h 17. In some embodiments, the allergen is a tree nut or seed allergen. In some embodiments, the allergen is 11S globulin, 7S globulin, 2S globulin, PR10, PR-14 nsLTP, oleosin or profilin.

In other embodiments, the food allergen is an allergen selected from the group consisting of buckwheat, celery, a color additive, egg, fish, fruit, garlic, gluten, oats, legumes, maize, milk, mustard, nuts, peanuts, poultry, meat, rice, sesame, shellfish, soy, tree nut and wheat.

In some embodiments, the allergen is a bee venom allergen. In some embodiments, the bee venom allergen is Phospholipase A2, Hyaluronidase, acid phosphatase, melittin, allergen C/DPP, CRP/icarapin or vitellogenin. In some embodiments, the allergen is a vespid allergen. In some embodiments, the vespid allergen is Phospholipase A1, hyaluronidase, protease, antigen 5, DPP IV or vitellogenin.

In some embodiments, the allergen is a latex allergen. In some embodiments, the latex allergen is Hev b 1, Hev b 2, Hev b 3, Hev b 4, Hev b 5, Hev b 6, Hev b 7, Hev b 8, Hev b 9, Hev b 10, Hev b 11, Hev b 12, Hev b 13, Hev b 14 or Hev b 15.

In some embodiments, the allergen is a dust mite allergen. In some embodiments the allergen is a house dust mite allergen. In some embodiments, the allergen is a storage dust allergen. In some embodiments, the house dust mite allergen is Der p 1, Der p 2, Der p 3, Der p 4, Der p 5, Der p 7, Der p 8, Der p 10, Der p 11, Der p 21, or Der p 23. In some embodiments, the allergen comprises the Der p 1 T cell epitope (111-139). In some embodiments the further antigenic unit comprises a Der p 1 hypoallergenic allergen. In some embodiments, the house dust mite allergen is Der f 1, Der f 2, Der f 3, Der f 7, Der f 8 or Der f 10. In some embodiments, the house dust mite allergen is Blot t 1, Blot t 2, Blot t 3, Blot t 4, Blot t 5, Blot t 8, Blot t 10, Blot t 12 or Blot t 21.

In some embodiments, the allergen is a cockroach allergen. In some embodiments, the cockroach allergen is Bla g 1, Bla g 2, Bla g 3, Bla g 4, Bla g 5, Bla g 6, Bla g 7, Bla g 8 or Bla g 11. In some embodiments, the cockroach allergen is Per a 1, Per a 2, Per a 3,

### Per a 6, Per a 7, Per a 9 or Per a 10.

In some embodiments, the allergen is a mold allergen. In some embodiments, the mold allergen is an Aspergillus fumigatus allergen. In some embodiments, the Aspergillus fumigatus allergen is Asp f 1, Asp f 2, Asp f 3, Asp f 4, Asp f 5, Asp f 6, Asp f 7, Asp f 8, Asp f 9, Asp f 10, Asp f 11, Asp f 12, Asp f 13, Asp f 14, Asp f 15, Asp f 16, Asp f 17, Asp f 18, Asp f 22, Asp f 23, Asp f 27, Asp f 28, Asp f 29 or Asp f 34.

In some embodiments, the allergen is a fungal allergen. In some embodiments, the fungal allergen is a Malassezia allergen. In some embodiments, the Malassezia allergen is Mala f 1, Mala f 2, Mala f 3, Mala f 4, Mala f 5, Mala f 6, Mala f 7, Mala f 8, Mala f 9, Mala f 10, Mala f 11, Mala f 12 or Mala f 13 or MGL_1204.

In some embodiments, the allergen is a furry animal allergen. In some embodiments, the allergen is a dog allergen. In some embodiments, the dog allergen is Can f 1, 2, 3, 4, 5, or 6. In some embodiments, the allergen is a horse allergen. In some embodiments, the horse allergen is Ecu c 1, 2, 3 or 4. In some embodiments, the allergen is a cat allergen. In some embodiments, the cat allergen is Fel d 1, Fel d 2, Fel d 3, Fel d 4, Fel d 5, Fel d 6, Fel d 7, or Fel d 8. In some embodiments, the allergen is a laboratory animal allergen. In some embodiments, the allergen is lipocalin, urinary prealbumin, secretoglobulin or serum albumin.

In some embodiments, the allergen is a pollen allergen. In some embodiments, the allergen is a grass pollen allergen. In some embodiments, the grass pollen allergen is a timothy grass, orchard grass, Kentucky bluegrass, perennial rye, sweet vernal grass, bahia grass, johnson grass or Bermuda grass allergen. In some embodiments the grass pollen allergen is Phl p 1, Phl p 2, Phi p 3, Phi p 4, Phi p 5, Phi p 6, Phi p 7, Phl p 11, Phi p 12 or Phl p 13.

In some embodiments, the allergen is a tree pollen allergen. In some embodiments, the tree pollen allergen is a alder, birch, hornbeam, hazel, European hophornbeam, chestnut, European beech, white oak, ash, privet, olive, lilac, cypress or cedar pollen allergen. In some embodiments, the tree pollen allergen is Aln g 1 or Aln g 4, Bet v 1, Bet v 2, Bet v 3, Bet v 4, Bet v 6 or Bet v 7, Car b 1, Cor a 1, Cor a 2, Cor a 6, Cor a 8, Cor a 9, Cor a 10, Cor a 11, Cor a 12, 1 Cor a 3, Cor a 14, Ost c 1, Cas 1, Cas 5, Cas 8 or Cas 9, Fag s 1, Que a 1, Fra e 1, Lig v 1, Ole e 1, Ole e 2, Ole e 3, Ole e 4, Ole e 5, Ole e 6, Ole e 7, Ole e 8, Ole e 9, Ole e 10, Ole e 11 or Ole e 12, Syr v 1, Cha o 1, Cha o 2, Cry j 1, Cry j 2, Cup s 1, Cup s 3, Jun a 1, Jun a 2, Jun a 3, Jun o 4, Jun v 1, Jun v 3, Pla a 1, Pla a 2 or Pla a 3. In some embodiments, the allergen comprises the Bet v 1 T cell epitope (139-152). In some embodiments the further antigenic unit comprises a Bet v 1 hypoallergenic allergen.

In some embodiments, the allergen is a weed pollen allergen. In some embodiments the weed allergen is a ragweed, mugwort, sunflower, feverfew, pellitory, English plantain, annual mercury, goosefoot, Russian thistle or amaranth pollen allergen. In some embodiments the ragweed pollen allergen is Amb a 1, Amb a 4, Amb a 6, Amb a 8, Amb a 9, Amb a 10, or Amb a 11. In some embodiments the mugwort pollen allergen is Art v 1, Art v 3, Art v 4, Art v 5, or Art v 6. In some embodiments, the sunflower pollen allergen is Hel a 1 or Hel a 2. In some embodiments, the pellitory pollen allergen is Par j 1, Par j 2, Par j 3 or Par j 4. In some embodiments, the English plantain pollen allergen is Pla l 1. In some embodiments, the annual mercury pollen allergen is Mer a 1. In some embodiments, the goosefoot pollen allergen is Che a 1, Che a 2 or Che a 3. In some embodiments, the Russian thistle pollen allergen is Sal k 1, Sal k 4 or Sal k 5. In some embodiments, the Amaranth pollen allergen is Ama r 2.

In yet other embodiments, the allergen is selected form environmental allergens such insects, cockroaches, house dust mites or mold.

In some embodiments, the vectors of the disclosure may be used in the treatment of allergic diseases selected from the group consisting of allergic rhinitis, asthma, atopic dermatitis, allergic gastroenteropathy, contact dermatitis and drug allergy or combinations thereof.

Allergy to drugs affect more than 7% of the general population. The constructs encoded by the vectors of the disclosure may be used to induce tolerance towards immunogenic T cell epitopes present in such a drug and thus will allow affected patients to continue treatment with the drug and receive the benefits from the drug treatment.

Thus, in some embodiments, the allergen is comprised in a drug with unwanted immunogenicity. In some embodiments, the allergen is Factor VIII. In some embodiments, the allergen is insulin. In some embodiments, the allergen is a monoclonal antibody used for therapy.

### Hypoallergenic Allergen and Epitopes

In some embodiments of constructs comprising epitopes of an allergen, self-antigen or alloantigen, the further antigenic unit of at least one of the one or more further polypeptides comprises one or more further epitopes, wherein the one or more further epitopes are comprised in one or more hypoallergenic allergen. Thus, in some embodiments, the further antigenic unit of at least one of the one or more further polypeptides comprises one or more further epitopes, wherein the one or more further epitopes are comprised in one hypoallergenic allergen.

In some embodiments, the further antigenic unit of at least one of the one or more further polypeptides comprises one or more further epitopes, wherein the one or more further epitopes are comprised in one or more hypoallergenic allergens. Thus, in some embodiments, the further antigenic unit of at least one of the one or more further polypeptides comprises one or more further epitopes, wherein the one or more further epitopes are comprised in one hypoallergenic allergen.

In some embodiments, the further antigenic unit of at least one of the one or more further polypeptides comprises one or more full length hypoallergenic allergens, wherein said one or more full length hypoallergenic allergens comprises the one or more further epitopes.

In some embodiments, the one or more epitopes and/or one or more further epitopes are derived from one or more allergens, wherein the allergens are selected from the group consisting of shellfish allergen, cow's milk allergen, egg allergen, fish allergen, fruit allergen, wheat allergen, peanut allergen, tree nut allergen, soy allergen, seed allergen, buckwheat allergen, celery allergen, garlic allergen, gluten allergen, oat allergen, legumes allergen, maize allergen, milk allergen, mustard allergen, nuts allergen, poultry allergen, meat allergen, rice allergen, sesame allergen, bee venom allergen, vespid allergen, latex allergen, dust mite allergen, insect allergen, mold allergen, fungal allergen, furry animal allergen, pollen allergen and drug allergen.

In some embodiments, the drug allergen is selected from the group consisting of Factor VIII, insulin and therapeutic monoclonal antibody.

In some embodiments, the further antigenic unit of at least one of the one or more further polypeptides comprises a hypoallergenic allergen, wherein said hypoallergenic allergen comprises the one or more further epitopes.

In some embodiments, the further antigenic unit of at least one of the one or more further polypeptides comprises a hypoallergenic allergen comprising one or more T cell epitopes and one or more B cell epitopes.

In some embodiments, the further antigenic unit of at least one of the one or more further polypeptides comprises a hypoallergenic allergen comprising one or more T cell epitopes.

In some embodiments, the further antigenic unit of at least one of the one or more further polypeptides comprises a hypoallergenic allergen comprising one or more B cell epitope.

In some embodiments, the further antigenic unit of at least one of the one or more further polypeptides comprises one or more full length hypoallergenic allergens comprising the one or more further epitopes.

In some embodiments, the further antigenic unit of at least one of the one or more further polypeptides comprises one or more full length hypoallergenic allergens.

In some embodiments, the further antigenic unit of at least one of the one or more further polypeptides comprises a hypoallergenic allergen comprising more than one epitope, such as 2, 3, 4, 5, 6, 7 or 8 epitopes, such as 2, 3, 4, 5, 6, 7 or 8 different epitopes.

In some embodiments, the further antigenic unit of at least one of the one or more further polypeptides comprises a hypoallergenic allergen comprising more than one antigen, such as 2, 3, 4, 5, 6, 7 or 8 antigens, such as 2, 3, 4, 5, 6, 7 or 8 different antigens.

In some embodiments:
i. the antigenic unit comprises or consists of T cell epitopes; and
ii. the further antigenic unit of at least one the one or more further polypeptides comprises or consists of one or more full length hypoallergenic allergens.

### Self-Antigen

In other embodiments, at least one of the one or more further polypeptide comprise T cell epitopes comprised in a self-allergen that is involved in an autoimmune disease. This allows for the antigen-specific down-regulation of the part of the immune system responsible for the autoimmune disease without inhibiting the immune system in general.

In some embodiments, the autoimmune disease is multiple sclerosis (MS). In some embodiments, the self-antigen is myelin oligodendrocyte glycoprotein (MOG). In other embodiments the self-antigen is MAG, MOBP, CNPase, S100beta or transaldolase. In some embodiments, the self-antigen is myelin basic protein (MBP). In some embodiments, the self-antigen is myelin proteolipid protein (PLP).

In the examples we provide constructs for multiple sclerosis including either a short (35-55 amino acids) or a longer (27-63 amino acids) T cell epitope from myelin oligodendrocyte glycoprotein (MOG). MOG is a member of the immunoglobulin superfamily and is expressed exclusively in the central nervous system. MOG (35-55) can induce autoantibody production and relapsing-remitting neurological disease, causing extensive plaque-like demyelination. Autoantibody response to MOG (35-55) has been observed in MS patients and MOG (35-55)-induced experimental autoimmune encephalomyelitis (EAE) in C57/BL6 mice and Lewis rats.

Other MS-relevant T cell epitopes that are known in the art and have been studied include the following:

| T cell epitope | Sequence |
|---|---|
| PLP (139-151)* | HCLGKWLGHPDKF |
| | (SEQ ID NO: 285) |
| PLP (131-159) | AHSLERVCHCLGKWLGHPDKFVGITYALT |
| | (SEQ ID NO: 286) |
| PLP (178-191)* | NTWTTCQSIAFPSK |
| | (SEQ ID NO: 287) |
| PLP (170-199) | AVPVYIYFNTWTTCQSIAFPSKTSASIGSL |
| | (SEQ ID NO: 288) |
| MBP (84-104)* | VHFFKNIVTPRTPPPSQGKGR |
| | (SEQ ID NO: 289) |
| MBP (76-112) | RTQDENPVVHFFKNIVTPRTPPPSQGKGRG |
| | LSLSRF |
| | (SEQ ID NO: 290) |

*T cell epitope-induced EAE observed

In preferred embodiments, the further antigenic unit includes one or more T cell epitopes selected from the group consisting of MOG (35-55), MOG (27-63), PLP (139-151), PLP (131-159), PLP (178-191), PLP (170-199), MBP (84-104) and MBP (76-112). A pharmaceutical composition comprising such a construct may be used in the treatment of MS.

In some embodiments, the autoimmune disease is type 1 diabetes mellitus. In some embodiments, the self-antigen is glutamic acid decarboxylase 65-kilodalton isoform (GAD65), which is a self-antigen involved in type 1 diabetes mellitus. In some other embodiments, the self-antigen is insulin, IA-2 or ZnT8. In yet some other embodiments, the self-antigen is IGRP, ChgA, IAPP, peripherin, tetraspanin-7, GRP78, Urocortin-3 or Insulin gene enhancer protein isl-1.

In some embodiments, the autoimmune disease is celiac disease. In some embodiments, the self-antigen is α-gliadin, γ-gliadin, ω-gliadin, low molecular weight glutenin, high molecular weight glutenin, hordein, secalin or avenin b. In some embodiments, the further antigenic unit comprises the T cell epitope α-gliadin (76-95).

In some embodiments, the autoimmune disease is rheumatoid arthritis. In some embodiments, the self-antigen is collagen. In some embodiments, the self-antigen is heat shock protein 60 (HSP60). In some embodiments, the self-antigen is Band 3. In some embodiments, the self-antigen is small nuclear ribonucleoprotein D1 (SmD1). In some embodiments, the self-antigen is the acetylcholine receptor (AChR). In some embodiments, the self-antigen is myelin protein zero (P0).

In some embodiments, the autoimmune disease is chronic inflammatory demyelinating polyradiculoneuropathy (CIDP) and the self-antigen is neurofascin 155. In other embodiments, the autoimmune disease is Hashimoto's thyroiditis (HT) and the self-antigen is thyroid peroxidase and/or thyroglobulin. In other embodiments, the autoimmune disease is pemphigus foliaceus and the self-antigen is desmosome-associated glycoprotein. In other embodiments, the autoimmune disease is pemphigus vulgaris and the self-antigen is desmoglein 3. In other embodiments, the autoimmune disease is thyroid eye disease (TED) and the self-antigen is calcium binding protein (calsequestrin). In other embodiments, the autoimmune disease is Grave's disease and the self-antigen is thyroid stimulating hormone receptor. In other embodiments, the autoimmune disease is primary biliary cirrhosis (PBC) and the self-antigen is antimitochondrial antibodies (AMAs), antinuclear antibodies (ANA), Rim-like/membrane (RL/M) and/or multiple nuclear dot (MND). In other embodiments, the autoimmune disease is myasthenia gravis and the self-antigen is acetylcholine receptor. In other embodiments, the autoimmune disease is insulin-resistant diabetes and the self-antigen is insulin receptor. In other embodiments, the autoimmune disease is autoimmune hemolytic anemia and the self-antigen is erythrocytes. In other embodiments, the autoimmune disease is rheumatoid arthritis and the self-antigens are citrullinated, homocitrullinated proteins and/or the Fc portion of IgG.

In other embodiments, the autoimmune disease is psoriasis and the self-antigens are cathelicidin (LL-37), disintegrin-like and metalloprotease domain containing thrombospondin type 1 motif-like 5 (ADAMTSL5), phospholipase A2 group IVD (PLA2G4D), heterogeneous nuclear ribonucleoprotein A1 (hnRNP-A1) and keratin 17.

### Alloantigen

In other embodiments, the antigenic unit comprises one or more T cell epitopes of an alloantigen i.e. one T cell epitope of an alloantigen or more than one T cell epitope of an alloantigen, i.e. multiple T cell epitopes of an alloantigen. In some embodiments, the multiple T cell epitopes are of the same alloantigen, i.e. comprised in the same alloantigen. In other embodiments, the multiple T cell epitopes are of multiple different alloantigens, i.e. comprised in different alloantigens.

In other embodiments, the further antigenic unit comprises one or more alloantigens i.e. one alloantigen or more than one alloantigen, i.e. multiple alloantigens. In some embodiments, the multiple alloantigens are of the same alloantigen, i.e. comprised in the same alloantigen. In other embodiments, the multiple alloantigens are of multiple different alloantigen, i.e. comprised in different alloantigens.

In some embodiments, the alloantigen is an antigen derived from (shed from and/or present in) a cell or tissue which, when transferred from a donor to a recipient, can be recognized and bound by an antibody of B or T cell receptor of the recipient.

In some embodiments, the one or more alloantigens are products of polymorphic genes.

In some embodiments, the one or more alloantigens are proteins or peptides which, when compared between donor and recipient (belonging to the same species), displays slight structural differences. In some embodiments, the presence of such a donor antigen in the body of a recipient can elicit an immune response in the recipient, such as an alloreactive immune response, such as an alloantigen specific immune response.

### Further Embodiments of the Antigenic Unit

The following applies to the antigenic unit in the first polypeptide encoded by the first nucleic acid, and/or to the further antigenic unit in at least one further polypeptide encoded by the one or more nucleic acids comprised in the vectors of the disclosure in general.

The term antigen is used in this section of the application for a neoantigen, a neoepitope, a patient-present shared cancer antigen, a part of a patient-present shared cancer antigen, such as a patient-present shared cancer epitope, a shared cancer antigen, a part of a shared cancer antigen, such as a shared cancer epitope, an infectious antigen or a part thereof or a T cell epitope of an infectious antigen.

In some embodiments, the antigenic unit comprises only one copy of each antigen. In other embodiments, the antigenic unit comprises multiple copies of one or more antigens.

In some embodiments, the antigenic unit comprises only one copy of each antigen, so that when e.g., 10 different antigens are comprised in the antigenic unit, a vector comprising said antigenic unit may elicit an immune response against all 10 different antigens and thus attack the cancer efficiently.

In other embodiments, if, e.g., only a few neoepitopes could be identified in a specific patient that are predicted to be sufficiently immunogenic/predicted to bind to the patient's HLA alleles, then the antigenic unit may comprise at least two copies of a particular antigen, e.g., particular neoepitope, in order to strengthen the immune response to the antigen. If in such patient one or more patient-present shared cancer antigens are identified in addition to such few neoepitopes, it is however preferred to then include such one or more patient-present shared cancer antigens or parts thereof into the antigenic unit rather than including multiple copies of the same neoepitope.

The length of the antigenic unit is determined by the length of the epitope(s) and/or antigen(s), allergen(s), hypoallergenic allergen(s), self-antigen(s) or alloantigen(s) comprised therein as well as their number.

In some embodiments, the antigenic unit comprises up to 3500 amino acids, such as from 60 to 3500 amino acids, e.g., from about 80 or about 100 or about 150 amino acids to about 3000 amino acids, such as from about 200 to about 2500 amino acids, such as from about 300 to about 2000 amino acids or from about 400 to about 1500 amino acids or from about 500 to about 1000 amino acids.

In order to enhance the immune response, particularly for a first polypeptide and/or a further polypeptide comprising neoantigens, the antigens may be arranged in the antigenic subunit as described in the following paragraphs.

The antigenic unit can be described as a polypeptide having an N-terminal start and a C-terminal end. The antigenic unit is connected to the multimerization unit, such as dimerization unit, e.g., via a linker, preferably via an unit linker. The antigenic unit is either located at the COOH-terminal end or the NH2-terminal end of the first polypeptide and/or a further polypeptide. It is preferred that the antigenic unit is in the COOH-terminal end of the first polypeptide and/or further polypeptide.

In some embodiments, the antigens, preferably epitopes, are arranged in the order of more antigenic to less antigenic in the direction from the N-terminal start of the antigenic unit to the C-terminal end of the antigenic unit. Alternatively, particularly if the hydrophilicity/hydrophobicity varies greatly among the antigens, it is preferred that the most hydrophobic antigens is/are substantially positioned in the middle of the antigenic unit and the most hydrophilic antigens is/are positioned at the N-terminal start and/or the C-terminal end of the antigenic unit.

Since a true positioning in the middle of the antigenic unit is only possible if the antigenic unit comprises an odd number of antigens, the term “substantially” in this context refers to antigenic units comprising an even number of antigens, wherein the most hydrophobic antigens are positioned as closed to the middle as possible.

By way of example, an antigenic unit comprises 5 antigenic subunits, each comprising a different epitope, e.g., a different neoepitope, which are arranged as follows: 1-2-3*-4-5; with 1, 2, 3*,4 and 5 each being a different neoepitope and - being a subunit linker and * indicating the most hydrophobic neoepitope, which is positioned in the middle of the antigenic unit. In another example, particularly for tolerance-inducing constructs, an antigenic unit comprises 5 antigenic subunits, each comprising a different T cell epitope of an allergen, self-antigen or alloantigen, e.g., a different T cell epitope, which are arranged as follows: 1-2-3*-4-5; with 1, 2, 3*,4 and 5 each being a different T cell epitope and - being a subunit linker and * indicating the most hydrophobic T cell epitope, which is positioned in the middle of the antigenic unit.

In another example, an antigenic unit comprises 6 antigenic subunits, each comprising a different epitope, e.g., a different neoepitope, which are arranged as follows: 1-2-3*-4-5-6 or, alternatively, as follows: 1-2-4-3*-5-6; with 1, 2, 3*, 4, 5 and 6 each being a different neoepitope and - being a subunit linker and * indicating the most hydrophobic neoepitope, which is positioned substantially in the middle of the antigenic unit. In yet another example, particularly for tolerance-inducing constructs, an antigenic unit comprises 6 antigenic subunits, each comprising a different T cell epitope of an allergen, self-antigen or alloantigen, e.g., a different T cell epitope, which are arranged as follows: 1-2-3*-4-5-6 or, alternatively, as follows: 1-2-4-3*-5-6; with 1, 2, 3*, 4, 5 and 6 each being a different T cell epitope and - being a subunit linker and * indicating the most hydrophobic T cell epitope, which is positioned substantially in the middle of the antigenic unit.

Alternatively, the antigenic subunits may be arranged such that they alternate between a hydrophilic and a hydrophobic antigen.

Optionally, GC rich sequences encoding antigens or T cell epitopes from an allergen, self-antigen or alloantigen or allergens, hypoallergenic allergens, self-antigens (e.g., GC rich sequences encoding neoepitopes or epitopes) are arranged in such a way, that GC clusters are avoided. In some embodiments, GC rich sequences encoding antigens or T cell epitopes from an allergen, self-antigen or alloantigen or allergens, hypoallergenic allergens, self-antigens or alloantigens are arranged such that there is at least one non-GC rich sequence between them.

In some embodiments, the antigenic unit comprises one or more linkers. In other embodiments, the antigenic unit comprises multiple antigens, e.g., multiple epitopes, e.g., neoepitopes, wherein the antigens are separated by linkers. In some embodiments, the further antigenic unit comprises multiple allergens, hypoallergenic allergens, self-antigens or alloantigens, wherein the allergens, hypoallergenic allergens, self-antigens or alloantigens are separated by linkers. In yet other embodiments, the antigenic unit comprises multiple antigens wherein each antigen is separated from other antigens by linkers. An alternative way to describe the separation of each antigen from other antigens by linkers is that all but the terminal antigen, i.e., the antigen at the N-terminal start of the polypeptide or the C-terminal end of the polypeptide (i.e., located at the end of the antigenic unit that is not connected to the multimerization unit), are arranged in antigenic subunits, wherein each subunit comprises or consists of an antigen, e.g., a neoepitope, and a subunit linker.

Hence, an antigenic unit comprising n antigens comprises n−1 antigenic subunits, wherein each subunit comprises an antigen and a subunit linker, and further comprises a terminal antigen. Hence, an antigenic unit comprising n antigens comprises n−1 antigenic subunits, wherein each subunit comprises an allergen, a hypoallergenic allergen, a self-antigen or an alloantigen, and a subunit linker, and further comprises a terminal allergen, hypoallergenic allergen, self-antigen or alloantigen. In some embodiments, wherein n is an integer of from 1 to 50, e.g., 3 to 50 or 15 to 40 or 10 to 30 or 10 to 25 or 10 to 20 or 15 to 30 or 15 to 25 or 15 to 20.

Due to the separation of the antigens by the linkers, each antigen, allergen, hypoallergenic allergen, self-antigen or alloantigen is presented in an optimal way to the immune system.

In some embodiments, the antigenic unit comprises B cell epitopes and T cell epitopes, e.g., a full-length infectious antigen or part thereof and one or more T cell epitopes comprised in a protein of a pathogen and the antigenic unit is designed such that the T cell epitopes are arranged closest to the multimerization unit and the infectious antigen is at the terminal end of the antigenic unit. The T cell epitopes are preferably separated by linkers and the infectious antigen is preferably separated from the “subunit” comprising the T cell epitopes by a linker. Such afore-mentioned antigenic unit designs are disclosed in PCT/EP2022/061819, the disclosures of which is incorporated herein by reference.

### Antigens and Epitopes

In some embodiments, particularly for immunogenic constructs of the present disclosure, the antigenic unit of the first polypeptide comprises one or more epitopes, wherein the one or more epitopes are comprised in one or more antigens and/or the further antigenic unit of at least one of the one or more further polypeptides comprises one or more further epitopes, wherein the one or more further epitopes are comprised in one or more further antigens. Thus, in some embodiments, the antigenic unit of the first polypeptide comprises one or more epitopes, wherein the one or more epitopes are comprised in one antigen and/or the further antigenic unit of at least one of the one or more further polypeptides comprises one or more further epitopes, wherein the one or more further epitopes are comprised in one further antigens.

In some embodiments, the antigenic unit of the first polypeptide comprises one or more epitopes, wherein the one or more epitopes are comprised in one or more antigens. Thus, in some embodiments, the antigenic unit of the first polypeptide comprises one or more epitopes, wherein the one or more epitopes are comprised in one antigen.

In some embodiments, the further antigenic unit of at least one of the one or more further polypeptides comprises one or more further epitopes, wherein the one or more further epitopes are comprised in one or more further antigens. Thus, in some embodiments, the further antigenic unit of at least one of the one or more further polypeptides comprises one or more further epitopes, wherein the one or more further epitopes are comprised in one further antigens.

In some embodiments, the further antigenic unit of at least one of the one or more further polypeptides comprises one or more full length proteins, wherein said one or more full length proteins comprises the one or more further epitopes.

In some embodiments, the one or more epitopes and/or one or more further epitopes or parts thereof are selected from the group consisting of non-self epitopes, non-self antigens or parts thereof, disease relevant epitopes and disease relevant antigens or parts thereof.

In some embodiments, the one or more epitopes and/or the one or more further epitopes are cancer associated, such as selected from the group consisting of tumor associated, tumor specific, patient-present shared, patient-present specific, neoantigens and neoepitopes.

In some embodiments, the one or more epitopes and/or the one or more further epitopes are derived from one or more cancer antigens, wherein said cancer antigens are selected from the group consisting of cancer antigens associated with multiple myeloma or lymphoma, malignant melanoma, HPV induced cancers, prostate cancer, breast cancer, lung cancer, ovarian cancer, and/or liver cancer. In some embodiments said cancer antigens are selected from the group consisting of cancer antigens associated with breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, bladder cancer, esophageal cancer, Hodgkin's disease and adrenocortical cancer.

In some embodiments, the one or more epitopes and/or the one or more further epitopes are derived from one or more pathogen, wherein the one or more pathogens are selected from the group consisting of viruses, bacteria, fungi and parasites. In some embodiments, the one or more epitopes and/or one or more further epitopes are derived from one pathogen, wherein the pathogen is selected from the group consisting of viruses, bacteria, fungi and parasites.

In some embodiments, the further antigenic unit of at least one of the one or more further polypeptides comprises an antigen or a part thereof, wherein said antigen or a part thereof comprises the one or more further epitopes.

In some embodiments, the further antigenic unit of at least one of the one or more further polypeptides comprises one or more T cell epitopes and one or more B cell epitopes.

In some embodiments, the further antigenic unit of at least one of the one or more further polypeptides comprises one or more T cell epitopes.

In some embodiments, the further antigenic unit of at least one of the one or more further polypeptides comprises one or more B cell epitope.

In some embodiments, the further antigenic unit of at least one of the one or more further polypeptides comprises one or more full length proteins comprising the one or more further epitopes.

In some embodiments, the further antigenic unit of at least one of the one or more further polypeptides comprises one or more full length antigens.

In some embodiments, the further antigenic unit of at least one of the one or more further polypeptides comprises one full length antigen.

In some embodiments, the further antigenic unit of at least one of the one or more further polypeptides comprises more than one epitope, such as 2, 3, 4, 5, 6, 7 or 8 epitopes, such as 2, 3, 4, 5, 6, 7 or 8 different epitopes.

In some embodiments, the further antigenic unit of at least one of the one or more further polypeptides comprises more than one antigen, such as 2, 3, 4, 5, 6, 7 or 8 antigens, such as 2, 3, 4, 5, 6, 7 or 8 different antigens.

In some embodiments, the antigenic unit of the first polypeptide, and/or the further antigenic unit of at least one of the one or more further polypeptides comprise an antigen, or a part or region of an antigen, comprising multiple epitopes, such as T cell epitopes, e.g., multiple minimal T cell epitopes, such as a hotspot.

In some embodiments, the antigenic unit of the first polypeptide, and/or the further antigenic unit of at least one of the one or more further polypeptide include one T cell epitope. In other embodiments, the antigenic unit and/or the further antigenic unit includes more than one T cell epitope, i.e., multiple T cell epitopes.

T cell epitopes suitable for inclusion into the antigenic unit of the first polypeptide, and/or in the further antigenic unit of at least one of the one or more further polypeptides may be known in the art, i.e., have been studied, proposed and/or verified to be involved and of relevance for a certain disease and published, e.g., in the scientific literature.

In some embodiments, the antigenic unit, and/or the further antigenic unit of at least one of the one or more further polypeptides comprises an antigen or part thereof with a length of from 7 to 150 amino acids, preferably of from 7 to 100 amino acids, e.g., from 9 or 10 to 100 amino acids or from 15 to 100 amino acids or from 9 to 60 amino acids or from 9 to 30 amino acids or from 15 to 60 of from 15 to 30 or from 20 to 75 amino acids or from 25 to 50 amino acids, wherein the antigen comprises at least one T cell epitope.

In some embodiments:
i. the antigenic unit comprises or consists of T cell epitopes; and
ii. the further antigenic unit of at least one the one or more further polypeptides comprises one or more full length antigens.

In some embodiments:
i. the targeting unit comprises or consists of human CCL3L1;
ii. the antigenic unit comprises or consists of T cell epitopes; and
iii. the further antigenic unit of at least one of the one or more further polypeptides comprises one or more full length antigens.

In some embodiments:
i. the antigenic unit comprises or consists of T cell epitopes; and
ii. the further antigenic unit of at least one the one or more further polypeptides comprises one or more full length antigens.

In some embodiments:
i. the targeting unit comprises or consists of human CCL3L1;
ii. the antigenic unit comprises or consists of T cell epitopes; and
iii. the further antigenic unit of at least one of the one or more further polypeptides comprises one or more full length antigens.

In some embodiments:
i. the targeting unit comprises or consists of human CCL3L1;
ii. the antigenic unit comprises or consists of T cell epitopes;
iii. the further antigenic unit of at least one of the one or more further polypeptides comprises one full length antigen.

### Further Antigens

In some cases, if the antigenic unit of the first polypeptide, particularly of an immunogenic construct, comprises more than one antigen, or if the antigenic unit is particularly large, this might result in the incorrect folding of one or more of said antigens.

Thus, to address said challenge, in some embodiments, the further antigenic unit of at least one further polypeptide comprises a further antigen and the co-expression of the first polypeptide and said further polypeptides as separate molecules allows the further antigen to fold correctly, i.e., the folding is substantially identical to the naturally occurring folding of the antigen.

Thus, in some embodiments, the antigen comprised in at least one of the one or more further polypeptides can form native oligomers and/or biologically relevant assemblies.

Thus, in some embodiments, at least one of the one or more further polypeptides comprises one or more further antigen. In some embodiments, at least one of the one or more further polypeptides comprises one further antigen.

Additionally, certain antigens might not be compatible with the structure of the first polypeptide. Examples of such antigens include large antigens, oligomeric antigens, protein complexes, membrane proteins, proteins that need a native N-terminus or which don't tolerate N-terminal fusion.

In some embodiments, the further antigen is an antigen with an amino acid sequence of at least 50, 60, 80, 100, 120, 140, 160, 180, 200, 250, 300, 350, 400, 450, 500, 550, 600, 650, 750, 800, 900, 1000, 1250, 1500, 1750, 2000, 2500, 3000 amino acids.

In some embodiments, the further antigen is an antigen of a membrane protein.

In some embodiments, the further antigen is an oligomeric antigen. Thus, in some embodiments, the further antigen forms an oligomer.

In some embodiments, the further antigen can form a multimer, such as a dimer, such as a heterodimer. Thus, in some embodiments, the further antigen forms a multimer, such as a dimer, such as a heterodimer.

In some embodiments, the further antigen forms a nanoparticle, such as an antigen nanoparticle.

In some embodiments, the further antigen comprises a sequence that promotes formation of a nanoparticles, such as antigen-presenting nanoparticles.

In some embodiments, the further antigen is fused to an oligomerization unit. In some embodiments, the self-assembly unit of at least one further polypeptide is ferritin.

In some embodiments:
i. the antigenic unit of the first polypeptide comprises one or more T cell epitopes; and
ii. the further antigenic unit of at least one of the one or more further polypeptides comprises one further antigen.

In some embodiments:
i. the one or more further nucleic acids encoding one or more further polypeptides are at least two further nucleic acids encoding at least two further polypeptides;
ii. the antigenic unit comprises one or more T cell epitopes; and
iii. the further antigenic unit of at least one other of the one or more further polypeptides comprises one further antigen.

In some embodiments:
i. the targeting unit comprises or consists of human CCL3L1;
ii. the antigenic unit comprises or consists of T cell epitopes; and
iii. the further antigenic unit of at least one of the one or more further polypeptides comprises one or more further antigens.

In some embodiments:
i. the targeting unit comprises or consists of human CCL3L1;
ii. the antigenic unit comprises or consists of T cell epitopes; and
iii. the further antigenic unit of at least one of the one or more further polypeptides comprises one further antigen.

### Universal T Helper Cell Epitopes

A productive CD4+ T cell helper response evoked by a T cell epitope is required for the priming, memory formation, and maintenance of highly cytolytic CD8+ T cells in chronic pathologies, such as cancer.

Furthermore, CD4+ T cell helper also stimulate the proliferation, and then the differentiation of antigen-binding B cells. The activation of B cells is a requirement for immunological class switching, which ensures the production of persistent and functional antibodies.

Thus, activation of CD4+ T cell helper cells is crucial in many diseases, such as certain of the diseases described herein. However, it is likely that a naturally occurring protein antigen does not comprise or only comprises few suitable T cell epitopes, or has only suboptimal T cell epitopes. Thus, alternative means may be necessary to ensure a productive CD4+ T cell helper response.

T cell helper function can be effectively provided to an antigen-specific CD8+ T cell response independently of the specificity of the CD4+ T cell. Furthermore, activated CD4+ T cells secrete chemokines and cytokines that can attract other immune-cells, thereby promoting a productive response. Thus, it can be useful to evoke a productive CD4+ T cell helper response by administering some sequence regions of protein antigens which sensitize CD4+ cells of most or all subjects.

The molecular basis for the existence of such “universal” CD4+ T cell epitopes is not clear, however, it is speculated that certain sequences may be especially effective at sensitizing CD4+ T cells because they might be easily processed and released from the antigen. This, along with the promiscuous binding of human class II molecules to peptides, may result in their universal recognition.

Thus, said universal CD4+ T cell epitopes can be used to promote a CD4+ T cells, even when a naturally occurring protein antigen does not comprise or only comprises few suitable T cell epitopes, or has only suboptimal T cell epitopes. Furthermore, T cell epitopes are not always easy to predict, and administration of universal CD4+ T cell epitopes can overcome the moderate fidelity of MHC class II helper epitopes prediction algorithms.

The main advantage of such an approach relies on the possibility to mobilize pools of CD4⁺ T memory cells to further boost the licensing of DC and therefore the CD8⁺ T cell immunity.

Furthermore, the administration of universal CD4+ T cell epitopes dispense of the assessment of host's epitope-specific CD4+ T cell precursors, as it is assumed that the universal CD4+ T cell epitopes will be bound by the CD4+ T cells of the host.

Thus, in some embodiments, particularly of immunogenic constructs of the present disclosure, a CD4+ T cell response induced by the vector as described herein is HLA independent and/or antigen independent, such as tumor-antigen independent, such as pathogen-antigen independent.

In some embodiments, a CD4+ T cell response induced by the vector as described herein is HLA independent.

In some embodiments, a CD4+ T cell response induced by the vector as described herein is antigen independent, such as tumor-antigen independent, such as pathogen-antigen independent.

In some embodiments, the CD4+ T cell response increases the licensing of dendritic cells.

In some embodiments, the antigenic unit of the first polypeptide and/or the further antigenic unit of at least one of the one or more further polypeptides comprise at least one universal CD4+ T cell epitope.

In some embodiments, the antigenic unit of the first polypeptide comprises at least one cell epitope CD8+ T cell epitope, and the further antigenic unit of at least one of the one or more further polypeptides comprises at least one universal CD4+ T cell epitope.

In some embodiments, the further antigenic unit of at least one of the one of further polypeptides comprises a least one CD8+ T cell epitope, and the further antigenic unit of at least one other of the one or more further polypeptides comprises at least one universal CD4+ T cell epitope.

In some embodiments, the further antigenic unit of at least one of the one of further polypeptides comprises a least one CD8+ cell epitope, and at least one universal CD4+ T cell epitope.

In some embodiments, the further antigenic unit of at least one of the one of further polypeptides comprises at least one further epitope, optionally at least one of the further epitopes is a universal CD4+ T cell epitope.

In some embodiments, at least one of the further epitopes is a universal CD4+ T cell epitope.

In some embodiments, the antigenic unit of the first polypeptide and/or the further antigenic unit of at least one of the one or more further polypeptides comprises at least two universal CD4+ T cell epitopes.

In some embodiments, the at least two universal CD4+ cell epitopes, are different universal CD4+ T cell epitopes.

In some embodiments, the CD4+ T cell epitope promotes activation of CD4+ T cells independently of the antigenic unit of the first polynucleotide.

In some embodiments, CD4+ T cells activated by the universal CD4+ T cell epitope promote the priming, memory formation, and maintenance of highly cytolytic CD8 T cells.

In some embodiments, the highly cytolytic CD8 T cells recognize epitopes associated with chronic pathologies, such as cancer.

In some embodiments, the universal CD4+ T cell epitope does not induce CD4+ tumor-specific cytotoxic T cells.

Universal CD4+ T cell epitopes have been shown to be present in antigens used in paediatric vaccines. Hence, it is possible to take advantage of pre-existing immunity to evoke a productive CD4+ T cell helper response.

Thus, in some embodiments, particularly of immunogenic constructs of the present disclosure, at least one universal CD4+ T cell epitope is derived from antigens used in a vaccine, such as in a paediatric vaccine.

Several studies have identified specific tetanus and diphtheria toxoids epitopes which are frequently recognized by human CD4+ T cells, irrespective of their HLA class II haplotype. Since tetanus and diphtheria toxoids are the key components of tetanus and diphtheria paediatric vaccines that are extensively used, thus universal CD4+ T cell epitopes derived from these proteins are particularly attractive.

Thus, in some embodiments, at least one CD4+ T cell epitope is derived from antigens of a pathogen, such as antigens from tetanus, and/or diphtheria, such as derived from tetanus and/or diphtheria toxoids.

In some embodiments, at least one universal CD4+ T cell epitope is derived from an antigen from tetanus.

In some embodiments, at least one universal CD4+ T cell epitope is derived from a tetanus toxoid.

In some embodiments, at least one universal CD4+ T cell epitope is derived from an antigen of diphtheria.

In some embodiments, at least one universal CD4+ T cell epitope is derived from a diphtheria toxoid.

Thus, in some embodiments, the at least one universal CD4+ T cell epitope is derived from tetanus, such as from a tetanus toxoid. In some embodiments, at least one universal CD4+ T cell epitope comprises the amino acid sequence selected from the group consisting of: FNNFTVSFWLRVPKVSASHLE (p30-SEQ ID NO: 119), ILMQYIKANSKFIGITE (p2-SEQ ID NO: 120), and variants thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto. In some embodiments, the at least one universal CD4+ cell epitope is derived from antigens of tetanus, such as from a tetanus toxoid. In some embodiments, at least one universal CD4+ T cell epitope comprises the amino acid sequence selected from the group consisting of:

(p30-SEQ ID NO: 119)
FNNFTVSFWLRVPKVSASHLE,
and
(p2-SEQ ID NO: 120)
ILMQYIKANSKFIGITE.

Thus, in some embodiments, at least one universal CD4+ T cell epitopes is derived from antigens of diphteria, such as from a diphteria toxoid. In some embodiments, at least one universal CD4+ cell epitopes comprises the amino acid sequence selected from the group consisting of: QSIQLSSLMVAQAIP (SEQ ID NO: 121), and variants thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto. In some embodiments, the at least one universal CD4+ cell epitopes is derived from antigens of diphteria, such as from diphteria toxoid. In some embodiments, at least one universal CD4+ T cell epitopes comprises the amino acid sequence QSIQLSSLMVAQAIP (SEQ ID NO: 121).

Alternatively, non-natural synthetic CD4+ T cell epitopes can be used. For example, Pan HLA DR-binding epitope (PADRE) is a universal peptide that activates antigen specific-CD4+ T cells which can be used as an agonist adjuvant in immunotherapeutic vaccine development.

Thus, in some embodiments, at least one universal CD4+ T cell epitope is non-naturally occurring. In some embodiments, at least one universal CD4+ T cell epitope is a pan HLA-DR binding epitope. In some embodiments, at least one universal CD4+ T cell epitope comprises an amino acid sequence selected from the group consisting of: AKFVAAWTLKAAA (SEQ ID NO: 122), and variants thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto. In some embodiments, at least one universal CD4+ T cell epitope is a pan HLA-DR binding epitope, such as an universal CD4+ T cell epitope comprising the amino acid sequence AKFVAAWTLKAAA (SEQ ID NO: 122).

An essential component of MHC class II-mediated antigen presentation is the invariant chain molecule (Ii). In the endoplasmic reticulum, MHC class II molecules assemble and then bind with the Ii chain. The class II-associated invariant chain peptide (CLIP) region of the Ii chain occupies the MHC class II peptide-binding grove. The Ii chain is then degraded until only the CLIP region remains; this region prevents premature binding of the antigenic peptide into the MHC class II peptide-binding groove. In the lysosomes, CLIP is later replaced by one of the antigenic peptides. Therefore, this approach relies in the use of the Ii molecule in which the CLIP is replaced with a CD4+ T-helper epitope of an antigen of interest which have been shown to lead to the presentation of the helper CD4+ T cell epitope through the MHC class II pathway.

Thus, in some embodiments, the further antigenic unit of at least one of the one of further polypeptides comprises an invariant chain-derived peptide amino acid sequence.

In some embodiments, the further antigenic unit of at least one of the one of further polypeptides comprises a class II-associated invariant chain peptide (CLIP) amino acid sequence.

In some embodiments, at least one of the further polypeptides comprises an antigen, a linker or 2A self-cleaving peptide, and the further antigenic unit comprises a class II-associated invariant chain peptide (CLIP) amino acid sequence and at least one universal CD4+ T cell epitope, such as AKFVAAWTLKAAA (SEQ ID NO: 122)

In some embodiments, the further antigenic unit of at least one of the one of further polypeptides comprises an antigen.

In some embodiments, the further antigenic unit of at least one of the one of further polypeptides comprises a furin linker amino acid sequence.

In some embodiments, the further antigenic unit of at least one of the one of further polypeptides comprises an antigen, a furin linker amino acid sequence and at least one universal CD4+ T cell epitope, such as FNNFTVSFWLRVPKVSASHLE (p30-SEQ ID NO: 119).

In some embodiments, the vector is as defined herein, and:
i. the antigenic unit comprises or consists of an antigen or parts thereof; and
ii. the further antigenic unit of one or more further polypeptides comprises the amino acid sequence FNNFTVSFWLRVPKVSASHLE (p30-SEQ ID NO: 119).

In some embodiments, the antigenic unit, comprises or consists of an antigen, wherein the antigen comprises a CD8+ T cell epitope.

In some embodiments, the vector is as defined herein, and wherein:
i. the antigenic unit comprises or consists of an antigen or parts thereof; and
ii. the further antigenic unit of at least one of the one or more further polypeptides comprises or consists of an universal CD4+ T cell epitope comprising or consisting of the amino acid sequence AKFVAAWTLKAAA (SEQ ID NO: 122) and an invariant chain-derived peptide, such as class II-associated invariant chain peptide (CLIP).

### Tolerance-Inducing Universal T Helper Cell Epitopes

In some embodiments, particularly for tolerance-inducing constructs, the antigenic unit of the first polypeptide further comprises one or more tolerance-inducing universal CD4+ or CD8+ T cell epitopes. In some embodiments, the further antigenic unit of at least one of the one or more further polypeptides further comprises one or more tolerance-inducing universal CD4+ or CD8+ T cell epitopes.

Tolerance-inducing universal T cell epitopes may be epitopes that are highly promiscuous, but not necessarily universal binders to all human HLAs.

In some embodiments, particularly for tolerance-inducing constructs, the tolerance-inducing universal T cell epitope comprises or consists of one or more T reg epitopes (Tregitopes). In some embodiments, the tolerance-inducing universal T cell epitope comprises or consists of one or more HLA class II-restricted T reg epitopes (Tregitopes). For example, HLA class II-restricted regulatory T cell (Treg) epitopes (Tregitopes) have been reported to suppress immune responses to co-administered antigens by stimulating the expansion of natural Tregs (nTregs) (Leslie Cousens, 2014).

In some embodiments, particularly for tolerance-inducing constructs, the tolerance-inducing universal T cell epitope comprises or consists of one or more inhibitory epitopes, such as one or more inhibigens. In some embodiments, the tolerance-inducing universal T cell epitope comprises or consists of one or more epitopes.

In some embodiments, particularly for tolerance-inducing constructs, the tolerance-inducing universal T cell epitope comprises or consists of one or more dominant autoepitopes from nucleosomal histones. In some embodiments, the tolerance-inducing universal T cell epitope comprises or consists of one or more dominant autoepitopes from nucleosomal histones which may be cross-reactively recognized by autoimmune Th cells, as well as B cells, and may be promiscuously presented in the context of diverse MHC class II alleles.

In some embodiments, particularly for tolerance-inducing constructs, the tolerance-inducing universal T cell epitope comprises or consists of one or more of epitopes of peptides that share a consensus motif across individuals and species. In some embodiments, the tolerance-inducing universal T cell epitope comprises or consists of one or more of epitopes of peptides that share a consensus motif across individuals and species that may be presented by MHC class I to activate cross-reactive CD8⁺ T regs to induce tolerance and suppress allogeneic responses.

In some embodiments, particularly for tolerance-inducing constructs, the further antigenic unit of at least one of the one of further polypeptides comprises at least one further epitope, optionally at least one of the further epitopes is a tolerance-inducing universal CD4+ or CD8+ T cell epitope.

In some embodiments, particularly for tolerance-inducing constructs, at least one of the further epitopes is a tolerance-inducing universal CD4+ or CD8+ T cell epitope.

In some embodiments, particularly for tolerance-inducing constructs, the antigenic unit of the first polypeptide and/or the further antigenic unit of at least one of the one or more further polypeptides comprises at least two tolerance-inducing universal CD4+ or CD8+ T cell epitopes.

In some embodiments, particularly for tolerance-inducing constructs, the at least two tolerance-inducing universal CD4+ or CD8+ T cell epitopes are different tolerance-inducing universal CD4+ or CD8+ T cell epitopes.

### Linkers Comprised in the Antigenic Unit

The antigenic unit and optionally the further antigenic unit may comprise linkers, e.g., linkers that separate the antigens and/or the epitopes comprised therein. In some embodiments the antigenic unit and the further antigenic unit may comprise linkers separating the T cell epitopes of an allergen, self-antigen, alloantigen or hypoallergenic antigen. As described above, all antigens, epitopes or T cell epitopes of an allergen, self-antigen, alloantigen or hypoallergenic antigen may be separated from each other by linkers and arranged in subunits. This ensures that each antigen and/or epitope and/or T cell epitope of of an allergen, self-antigen, alloantigen or hypoallergenic antigen is presented in an optimal way to the immune system. In the following, the term subunit linker and linker are used interchangeably, and both denote a linker in the antigenic unit and/or in the further antigenic unit.

In some embodiments, the linkers are designed to be non-immunogenic. A linker may be a rigid linker, meaning that that it does not allow the two amino acid sequences that it connects to substantially move freely relative to each other. Alternatively, it may be a flexible linker, i.e., a linker that allows the two amino acid sequences that it connects to substantially move freely relative to each other. Both types of linkers are useful. In some embodiments, the linker is a flexible linker, which allows presenting the antigen, e.g. the T cell epitope of an allergen, self-antigen, alloantigen or hypoallergenic antigen, in an optimal manner to the T cells, even if the antigenic unit comprises a large number of antigens such as T cell epitopes of an allergen, self-antigen, alloantigen or hypoallergenic antigen.

In some embodiments, the subunit linker is a peptide consisting of from 4 to 40 amino acids, e.g., 35, 30, 25 or 20 amino acids, e.g., from 5 to 20 amino acids or 5 to 15 amino acids or 8 to 20 amino acids or 8 to 15 amino acids 10 to 15 amino acids or 8 to 12 amino acids. In other embodiments, the subunit linker consists of 10 amino acids.

In some embodiments, all the subunit linkers are identical. If, however, one or more of the antigens or T cell epitopes of of an allergen, self-antigen, alloantigen or hypoallergenic antigen comprise a sequence similar to that of the linker, it may be an advantage to substitute the neighboring subunit linkers with a linker of a different sequence. Also, if a junction between an antigen and a subunit linker is predicted to constitute an immunogenic epitope in itself, then a linker of a different sequence may be used.

In some embodiments, the subunit linker is a flexible linker, preferably a flexible linker which comprises small, non-polar (e.g., glycine, alanine or leucine) or polar (e.g., serine or threonine) amino acids. The small size of these amino acids provides flexibility and allows for mobility of the connected amino acid sequences. The incorporation of serine or threonine can maintain the stability of the linker in aqueous solutions by forming hydrogen bonds with the water molecules, and therefore reduces the unfavorable interaction between the linker and antigens or allergens, hypoallergenic allergens, self-antigens or alloantigens. In some embodiments, the flexible linker is a serine(S) and/or glycine (G) rich linker, i.e., a linker comprising several serine and/or several glycine residues. Preferred examples are GGGGS (SEQ ID NO: 28), GGGSS (SEQ ID NO: 37), GGGSG (SEQ ID NO: 38), GGSGG (SEQ ID NO: 39), SGSSGS (SEQ ID NO: 40) or multiple variants thereof such as GGGGSGGGGS (SEQ ID NO: 29), (GGGGS) m (SEQ ID NO: 30), (GGGSS) m (SEQ ID NO: 41), (GGSGG) m (SEQ ID NO: 42), (GGGSG) m (SEQ ID NO: 43) or (SGSSGS) m (SEQ ID NO: 44), where m is an integer from 1 to 5, e.g., 1, 2, 3, 4, or 5. In some preferred embodiments, m is 2. In other preferred embodiments, the serine and/or glycine rich linker further comprises at least one leucine (L) residue, such as at least 1 or at least 2 or at least 3 leucine residues, e.g., 1, 2, 3 or 4 leucine residues.

In some embodiments, the subunit linker comprises or consists of LGGGS (SEQ ID NO: 45), GLGGS (SEQ ID NO: 46), GGLGS (SEQ ID NO: 47), GGGLS (SEQ ID NO: 48) or GGGGL (SEQ ID NO: 49). In other embodiments, the subunit linker comprises or consists of LGGSG (SEQ ID NO: 50), GLGSG (SEQ ID NO: 51), GGLSG (SEQ ID NO: 52), GGGLG (SEQ ID NO: 53) or GGGSL (SEQ ID NO: 54). In yet other embodiments, the subunit linker comprises or consists of LGGSS (SEQ ID NO: 55), GLGSS (SEQ ID NO: 56) or GGLSS (SEQ ID NO: 57).

In yet other embodiments, the subunit linker comprises or consists of LGLGS (SEQ ID NO: 58), GLGLS (SEQ ID NO: 59), GLLGS (SEQ ID NO: 60), LGGLS (SEQ ID NO: 61) or GLGGL (SEQ ID NO: 26). In yet other embodiments, the subunit linker comprises or consists of LGLSG (SEQ ID NO: 62), GLLSG (SEQ ID NO: 63), GGLSL (SEQ ID NO: 64), GGLLG (SEQ ID NO: 65) or GLGSL (SEQ ID NO: 66). In yet other embodiments, the subunit linker comprises or consists of LGLSS (SEQ ID NO: 67), or GGLLS (SEQ ID NO: 68).

In other embodiments, the subunit linker is serine-glycine linker that has a length of 10 amino acids and comprises 1 or 2 leucine residues.

In some embodiments, the subunit linker comprises or consists of LGGGSGGGGS (SEQ ID NO: 69), GLGGSGGGGS (SEQ ID NO: 70), GGLGSGGGGS (SEQ ID NO: 71), GGGLSGGGGS (SEQ ID NO: 72) or GGGGLGGGGS (SEQ ID NO: 73). In other embodiments, the subunit linker comprises or consists of LGGSGGGGSG (SEQ ID NO: 74), GLGSGGGGSG (SEQ ID NO: 75), GGLSGGGGSG (SEQ ID NO: 76), GGGLGGGGSG (SEQ ID NO: 77) or GGGSLGGGSG (SEQ ID NO: 78). In yet other embodiments, the subunit linker comprises or consists of LGGSSGGGSS (SEQ ID NO: 79), GLGSSGGGSS (SEQ ID NO: 80), GGLSSGGGSS (SEQ ID NO: 81), GGGLSGGGSS (SEQ ID NO: 82) or GGGSLGGGSS (SEQ ID NO: 83).

In a further embodiment, the subunit linker comprises or consists of LGGGSLGGGS (SEQ ID NO: 84), GLGGSGLGGS (SEQ ID NO: 85), GGLGSGGLGS (SEQ ID NO: 86), GGGLSGGGLS (SEQ ID NO: 87) or GGGGLGGGGL (SEQ ID NO: 88). In other embodiments, the subunit linker comprises or consists of LGGSGLGGSG (SEQ ID NO: 89), GLGSGGLGSG (SEQ ID NO: 90), GGLSGGGLSG (SEQ ID NO: 91), GGGLGGGGLG (SEQ ID NO: 92) or GGGSLGGGSL (SEQ ID NO: 93). In yet other embodiments, the subunit linker comprises or consists of LGGSSLGGSS (SEQ ID NO: 94), GLGSSGLGSS (SEQ ID NO: 95) or GGLSSGGLSS (SEQ ID NO: 96).

In yet other embodiments, the subunit linker comprises or consists of GSGGGA (SEQ ID NO: 97), GSGGGAGSGGGA (SEQ ID NO: 98), GSGGGAGSGGGAGSGGGA (SEQ ID NO: 99), GSGGGAGSGGGAGSGGGAGSGGGA (SEQ ID NO: 100) or GENLYFQSGG (SEQ ID NO: 101). In yet other embodiments, the subunit linker comprises or consists of SGGGSSGGGS (SEQ ID NO: 102), SSGGGSSGGG (SEQ ID NO: 103), GGSGGGGSGG (SEQ ID NO: 104), GSGSGSGSGS (SEQ ID NO: 105), GGGSSGGGSG (SEQ ID NO: 15), GGGSSS (SEQ ID NO: 106), GGGSSGGGSSGGGSS (SEQ ID NO: 107) or GLGGLAAA (SEQ ID NO: 108).

In other embodiments, the subunit linker is a rigid linker. Such rigid linkers may be useful to efficiently separate (larger) antigens, allergens, hypoallergenic allergens, self-antigens or alloantigens and prevent their interferences with each other. In some embodiments, the subunit linker comprises or consist of KPEPKPAPAPKP (SEQ ID NO: 109), AEAAAKEAAAKA (SEQ ID NO: 110), (EAAAK) m (SEQ ID NO: 32), PSRLEEELRRRLTEP (SEQ ID NO: 111) or SACYCELS (SEQ ID NO: 112).

In yet other embodiments, the subunit linker comprises or consists of TQKSLSLSPGKGLGGL (SEQ ID NO: 113). In yet other embodiments, the subunit linker comprises or consists of SLSLSPGKGLGGL (SEQ ID NO: 114).

In yet other embodiments, the subunit linker comprises or consists of

(SEQ ID NO: 115)
GGSAGGSGSGSSGGSSGASGTGTAGGTGSGSGTGSG;
or
(SEQ ID NO: 116)
GGSGGGSEGGGSEGGGSEGGGSEGGGSEGGGSGGGS
or
(SEQ ID NO: 117)
ELKTPLGDTTHT
or
(SEQ ID NO: 118)
EPKSCDTPPPCPRCP.

In yet other embodiments, the subunit linker is a cleavable linker, e.g., a linker which includes one or more recognition sites for endopeptidases, e.g., endopeptidases such as furin, caspases, cathepsins and the like. Cleavable linkers may be introduced to release free functional protein domains (e.g., encoded by larger antigens, allergens, hypoallergenic allergens, self-antigens or alloantigens), which may overcome steric hindrance between such domains or other drawbacks due to interference of such domains, like decreased bioactivity, altered biodistribution.

Examples of suitable linkers are disclosed in paragraphs [0098]-[0099] and in the recited sequences of WO 2020/176797A1, in paragraphs to of US 2019/0022202A1, in WO 2017/118695 A1 and in WO 2021/219897A1, all of which are incorporated herein by reference.

### Targeting to Intracellular Compartments

### Ubiquitin Unit

Targeting of a protein vaccine expressed from a vector in a transfected cell to certain intracellular compartments such as by ubiquitination has been shown to improve cytotoxic T cell responses (CTL). Such vaccines have been shown to break tolerance and induce protective immunity in mouse melanoma models. Furthermore, ubiquitination of isolated CTL, B-cell, and T-helper epitopes (as short as 8-mer peptides) in anti-viral vaccines has been shown to induce protective immunity and a six-fold-higher frequency of CTL precursors by improving epitope peptide entry into class-1 MHC pathway.

Thus, in some embodiments, particularly for immunogenic constructs of the present disclosure, at least one of the one or more further polypeptides comprises a ubiquitin unit. In some embodiments the ubiquitin unit is fused to the further antigenic unit. In some embodiments the ubiquitin unit is fused to the N-terminus of the further antigenic unit. In some embodiments the ubiquitin unit is fused to the C-terminus of the further antigenic unit.

Thus, in some embodiments, at least one of the one or more further polypeptides comprises a ubiquitin unit and the further antigenic unit comprises one or more T cell epitopes.

In some embodiments, the further antigenic unit of at least one of the one or more further polypeptides is fused to a ubiquitin unit. In some embodiments the further antigenic unit comprises one or more T cell epitopes.

In some embodiments, the ubiquitin unit is upstream of the further antigenic unit comprising one or more further epitopes.

In some embodiments, the proteasomal degradation, and/or MHC antigen presentation, of the at least one of the one or more further polypeptides is increased compared to one or more polypeptides without an ubiquitin unit but otherwise identical sequence to the at least one of the one or more further polypeptides comprising the ubiquitin unit.

In some embodiments, the ubiquitin unit consists of a motif that promotes proteasomal degradation, and/or MHC antigen presentation of the one or more further polypeptides.

In some embodiments the ubiquitin unit comprises the amino acid sequence of a ubiquitin protein. Thus, in some embodiments, the ubiquitin unit comprises or consists of the amino acid sequence

(SEQ ID NO: 123)
MQIFVKTLTGKTITLEVEPSDTIENVKAKIQDKEGIPPDQQ
RLIFAGKQLEDGRTLSDYNIQKESTLHLVLRLRGG

It has been shown in the literature that some mutations of the ubiquitin protein abolish cleavage by ubiquitin hydrolases, thereby allowing the ubiquitin protein to remain fused to the protein after translation. Examples of such mutations can be found in Andersson et al., Mol Ther (2004); Békés et al., Cell Rep. (2013); Asimaki et al., Semin Cell Dev Biol (2021); and Blount et al. 2018.

In some embodiments, the ubiquitin unit comprises or consists of an amino acid sequence selected from the group consisting of:

i.
(SEQ ID NO: 123)
MQIFVKTLTGKTITLEVEPSDTIENVKAKIQDKEGIPPDQQRLIFA
GKQLEDGRTLSDYNIQKESTLHLVLRLRGG;
ii.
(SEQ ID NO: 124)
MQIFVKTLTGKTITLEVEPSDTIENVKAKIQDKEGIPPDQQRLIFA
GKQLEDGRTLSDYNIQKESTLHLVLRLRGA;
iii.
(SEQ ID NO: 125)
MQIFVKTLTGKTITLEVEPSDTIENVKAKIQDKEGIPPDQQRLIFA
GKQLEDGRTLSDYNIQKESTLHLVLRLRGV;
iv.
(SEQ ID NO: 126)
MQIFVKTLTGKTITLEVEPSDTIENVKAKIQDKEGIPPDQQRLIFA
GKQLEDGRTLSDYNIQKESTLHLVLRPRGG;
and
v.
(SEQ ID NO: 127)
MQIFVKTLTGKTITLEVEPSDTIENVKAKIQDKEGIPPDQQRLIFA
GKQLEDGRTLSDYNIQKESTLHLVLRLR.

Thus, in some embodiments, the ubiquitin sequence has a mutation in amino acid position 73 or 76 to abolish cleavage of ubiquitin hydrolases, such as a G76A mutation, a G76V mutation, or an L73P mutation. In another embodiment, the two C-terminal glycine residues in positions 75 and 76 of the ubiquitin sequence are deleted.

In some embodiments:
i. the antigenic unit comprises or consists of B cell epitopes and/or antigens;
ii. at least one of the one or more further polypeptides comprises a ubiquitin unit;
iii. the ubiquitin unit is upstream from the further antigenic unit comprising one or more further epitopes, wherein the one or more further epitopes are T cell epitopes;
iv. the ubiquitin unit consists of a nucleic acid motif that promotes proteasomal degradation, and/or MHC antigen presentation of the at least one further polypeptide.

Targeting of an antigen for rapid proteasomal degradation by the N-end rule pathway may represent another strategy for induction of protective antigen-specific cytotoxic T cell responses in vivo. Arginine (R) has been demonstrated to represent the most destabilizing residue of the N-end rule in both yeast and mammalian reticulocytes.

Thus, in some embodiments:
i. at least one of the one or more further polypeptides comprises a ubiquitin unit; and
ii. the further antigenic unit of the at least one further polypeptide comprises a further antigen, wherein the N-terminal amino acid of the further antigen has been substituted to R compared to the natural sequence of said further antigen.

### KDEL Sequence

Addition of a KDEL sequence to the C-terminus of a protein, e.g. an antigen destined for secretion (e.g. after having been expressed in a cell transfected by a vector encoding the protein) prevents secretion by taking advantage of the KDEL receptor-mediated endoplasmatic reticulum (ER) retention pathway. Forced protein overexpression and accumulation in the ER is known to induce ER stress responses and proteasomal degradation of the antigen via the ER-associated degradation pathway which promotes antigen epitopes binding to MHC class I molecules for optimal class I antigen presentation and consequently induces a CD8+ T cell response.

Thus, in some embodiments, at least one of the one or more further polypeptides comprises at its C-terminal end the amino acid sequence KDEL (a KDEL sequence).

In some embodiments, such further polypeptide comprises a further antigenic unit comprising T cell epitopes. In some embodiments, said T cell epitopes are CD8+ T cell epitopes. In some other embodiments, such further polypeptide comprises a further antigenic unit comprising an antigen (e.g. a full-length antigen or parts thereof) known to have or predicted to have several CD8+ T cell epitopes in its sequence.

In some embodiments, the vector encoding the above-described further polypeptide encodes a first polypeptide which comprises an antigenic unit comprising
a) T cell epitopes, e.g. as discrete T cell epitopes, optionally separated by linkers) or comprised in hotspots, wherein said T cell epitopes are:
   (i.) CD4+ and CD8+ T cell epitopes
   (ii.) CD4+ T cell epitopes only
   (iii.) CD8+ T cell epitopes only; or
b) An antigen, e.g. a full-length antigen or parts thereof.

In some embodiments, the CD8+ T cell epitopes comprised in the antigenic unit and further antigenic unit are different. In some other embodiments, the antigenic unit and further antigenic unit comprise the same CD8+ T cell epitopes.

### Sequence Identity

Sequence identity may be determined as follows: A high level of sequence identity indicates likelihood that a second sequence is derived from a first sequence. Amino acid sequence identity requires identical amino acid sequences between two aligned sequences. Thus, a candidate sequence sharing 70% amino acid identity with a reference sequence requires that, following alignment, 70% of the amino acids in the candidate sequence are identical to the corresponding amino acids in the reference sequence. Identity may be determined by aid of computer analysis, such as, without limitations, the ClustalW computer alignment program (Higgins D., Thompson J., Gibson T., Thompson J. D., Higgins D. G., Gibson T. J., 1994. CLUSTAL W: improving the sensitivity of progressive multiple sequence alignment through sequence weighting, position-specific gap penalties and weight matrix choice. Nucleic Acids Res. 22:4673-4680), and the default parameters suggested therein. Using this program with its default settings, the mature (bioactive) part of a query and a reference polypeptide are aligned. The number of fully conserved residues is counted and divided by the length of the reference polypeptide. In doing so, any tags or fusion protein sequences, which form part of the query sequence, are disregarded in the alignment and subsequent determination of sequence identity.

The ClustalW algorithm may similarly be used to align nucleotide sequences. Sequence identities may be calculated in a similar way as indicated for amino acid sequences.

Another preferred mathematical algorithm utilized for the comparison of sequences is the algorithm of Myers and Miller, CABIOS (1989). Such an algorithm is incorporated into the ALIGN program (version 2.0) which is part of the FASTA sequence alignment software package (Pearson W R, Methods Mol Biol, 2000, 132:185-219). Align calculates sequence identities based on a global alignment. Align0 does not penalize to gaps in the end of the sequences. When utilizing the ALIGN and Align0 program for comparing amino acid sequences, a BLOSUM50 substitution matrix with gap opening/extension penalties of −12/−2 is preferably used.

Amino acid sequence variants may be prepared by introducing appropriate changes into the nucleotide sequence encoding the first polypeptide and/or the one or more further polypeptides, or by peptide synthesis. Such modifications include, for example, deletions from, and/or insertions into and/or substitutions of, residues within the amino acid sequences. The terms substituted/substitution, deleted/deletions and inserted/insertions as used herein in reference to amino acid sequences and sequence identities are well known and clear to the skilled person. Any combination of deletion, insertion, and substitution can be made to arrive at the final first polypeptide and/or final one or more further polypeptides, provided that the final proteins have the desired characteristics. For example, deletions, insertions or substitutions of amino acid residues may produce a silent change and result in a functionally equivalent first or further polypeptide.

Deliberate amino acid substitutions may be made based on similarity in polarity, charge, solubility, hydrophobicity, hydrophilicity, and/or the amphipathic nature of the residues as long as the desired properties of the protein in question are retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Herein encompassed are conservative substitutions, i.e., like-for-like substitution such as basic for basic, acidic for acidic, polar for polar etc. and non-conservative substitutions, i.e., from one class of residue to another or alternatively involving the inclusion of unnatural amino acids such as ornithine, diaminobutyric acid ornithine, norleucine, ornithine, pyriylalanine, thienylalanine, naphthylalanine and phenylglycine. Conservative substitutions that may be made are, for example within the groups of basic amino acids (arginine, lysine and histidine), acidic amino acids (glutamic acid and aspartic acid), aliphatic amino acids (alanine, valine, leucine, isoleucine), polar amino acids (glutamine, asparagine, serine, threonine), aromatic amino acids (phenylalanine, tryptophan, tyrosine), hydroxyl amino acids (serine, threonine), large amino acids (phenylalanine, tryptophan) and small amino acids (glycine, alanine).

Substitutions may also be made by unnatural amino acids and substituting residues include alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-a-amino butyric acid*, L-y-amino butyric acid*, L-a-amino isobutyric acid*, L-e-amino caproic acid*, 7-amino heptanoic acid*, L-methionine sulfone*, L-norleucine*, L-norvaline*, p-nitro-L-phenylalanine*, L-hydroxyproline*, L-thioproline*, methyl derivatives of phenylalanine (Phe) such as 4-methyl-Phe*, pentamethyl-Phe*, L-Phe (4-amino) #, L-Tyr (methyl)*, L-Phe (4-isopropyl)*, L-Tic (1,2,3,4-tetrahydroisoquinoline-3-carboxyl acid)*, L-diaminopropionic acid * and L-Phe (4-benzyl)*.

In the paragraph above, * indicates the hydrophobic nature of the substituting residue, whereas # indicates the hydrophilic nature of substituting residue and #* indicates amphipathic characteristics of the substituting residue. Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation involves the presence of one or more amino acid residues in peptoid form.

### Polypeptides and Multimeric/Dimeric Proteins

The vectors of the disclosure encode a first polypeptide and one or more further polypeptides as described above. The polypeptide and the one or more further polypeptides are expressed in vivo as a result of the administration of the vector to a subject.

Due to the presence of the multimerization unit, such as dimerization unit, multimeric proteins are formed when the polypeptide is expressed.

The multimeric proteins may be homomultimers or hetereomultimers, e.g., if the protein is a dimeric protein, the dimeric protein may be a homodimer, i.e., a dimeric protein wherein the two polypeptide chains are identical and consequently comprise identical units and thus antigen sequences (identical antigenic units), or the dimeric protein may be a heterodimer comprising two polypeptide chains, wherein polypeptide chain 1 comprises different antigen sequences in its antigenic unit than polypeptide 2. The latter may be relevant if the number of antigens or T cell epitopes of an allergen, hypoallergenic allergen, self-antigen or alloantigen for inclusion into the antigenic unit would exceed an upper size limit for the antigenic unit. It is preferred that the multimeric protein is a homomultimeric protein.

### Production of the Vector and Host Cells

The vectors of the disclosure are generally vectors suitable for transfecting a host cell and a) expression of the first polypeptide and formation of a multimeric protein and b) expression of the one or more further polypeptides encoded by the further nucleic acid sequences, respectively. In some embodiments, the multimeric protein is comprised of multiple of such first polypeptides encoded by the first nucleic acid sequence. In other embodiments, the multimeric protein is comprised of at least one of such first polypeptides encoded by the first nucleic acid sequence and at least one of such further polypeptides, as described in detail herein above.

In some embodiments, the host cell comprising the vector of the disclosure is a cell of a cell culture, e.g., a bacteria cell, and the proteins encoded by the vector are expressed in vitro. In other embodiments, the host cell comprising the vector of the disclosure is a cell of a subject and the proteins encoded by the vector are expressed in said subject, i.e., in vivo, as a result of the administration of the vector to a subject.

Suitable host cells for in vitro transfection include prokaryote cells, yeast cells, insect cells or higher eukaryotic cells. Suitable host cells for in vivo transfection are e.g., muscle cells.

In some embodiments, the host cell is selected from the group consisting of prokaryote cells, yeast cells, insect cells, higher eukaryotic cells such as cells from animals or humans.

In some embodiments, the vector allows for easy exchange of the various units described above, particularly the antigenic unit in case of individualized antigenic units.

In some embodiments, the vector is a pUMVC4a vector or a vector comprising NTC9385R vector backbones. The antigenic unit may be exchanged with an antigenic unit cassette restricted by the SfiI restriction enzyme cassette where the 5′ site is incorporated in the nucleotide sequence encoding the GLGGL (SEQ ID NO: 26)/GLSGL (SEQ ID NO: 27) unit linker and the 3′ site is included after the stop codon in the vector.

In other embodiments, the vector is CpG optimized vector, e.g. as described herein in the section “Tolerance-inducing vectors”. In other embodiments, the vector is a pALD-CV77 vector.

Engineering and production methods of the vectors of the disclosure, e.g., expression vectors such as DNA and RNA plasmids or viral vectors are well known, and the skilled person will be able to engineer/produce the vectors of the disclosure using such known methods. Moreover, various commercial manufacturers offer services for vector design and production.

In one aspect, the disclosure relates to a method of producing a vector comprising:
a) a first nucleic acid sequence encoding a first polypeptide, wherein the first polypeptide comprises a targeting unit that targets antigen-presenting cells, a multimerization unit, such as a dimerization unit, and an antigenic unit comprising one or more epitopes or one or more T cell epitopes of an allergen, hypoallergenic allergen, self-antigen or alloantigen; and
b) one or more further nucleic acid sequences encoding one or more further polypeptides, wherein the one or more further polypeptides comprise a further antigenic unit comprising one or more further epitopes or one or more allergens, hypoallergenic allergens, self-antigens or alloantigens,
wherein the vector allows for the co-expression of the first polypeptide and the one or more further polypeptides as separate molecules, the method comprising:
a) transfecting cells in vitro with the vector as defined herein;
b) culturing said cells;
c) optionally, lysing the cells to release the vector from the cells; and
d) collecting and optionally purifying the vector.

In some embodiments, the one or more epitopes, and/or the one or more further epitopes are disease-relevant epitopes.

### Pharmaceutical Compositions

In some embodiments of the present disclosure, the vector, e.g., the DNA plasmid is for use as a medicament.

Thus, in some embodiments of the present disclosure, the vector is provided in a pharmaceutical composition comprising the vector and a pharmaceutically acceptable carrier or diluent.

Thus, in one aspect, the disclosure relates to a pharmaceutical composition comprising
i. a pharmaceutically acceptable carrier or diluent; and
ii. a vector comprising:
   a) a first nucleic acid sequence encoding a first polypeptide, wherein the first polypeptide comprises a targeting unit that targets antigen-presenting cells, a multimerization unit, such as a dimerization unit, and an antigenic unit comprising one or more epitopes; and
   b) one or more further nucleic acid sequences encoding one or more further polypeptides, wherein the one or more further polypeptides comprise a further antigenic unit comprising one or more further epitopes,
wherein the vector allows for the co-expression of the first polypeptide and the one or more further polypeptides as separate molecules.

In some embodiments, the one or more epitopes, and/or the one or more further epitopes are disease-relevant epitopes.

In another aspect, the disclosure relates to a pharmaceutical composition comprising
i. a pharmaceutically acceptable carrier or diluent; and
ii. a vector comprising:
   a) a first nucleic acid sequence encoding a first polypeptide, wherein the first polypeptide comprises a targeting unit that targets antigen-presenting cells, a multimerization unit, such as a dimerization unit, and an antigenic unit comprising one or more T cell epitopes of an allergen, self-antigen or alloantigen; and
   b) one or more further nucleic acid sequences encoding one or more further polypeptides, wherein the one or more further polypeptides comprise a further antigenic unit comprising one or more further allergens, hypoallergenic allergens, self-antigens or alloantigens,
wherein the vector allows for the co-expression of the first polypeptide and the one or more further polypeptides as separate molecules.

Suitable pharmaceutically acceptable carriers or diluents include, but are not limited to, saline, buffered saline, such as PBS, dextrose, water, glycerol, ethanol, isotonic aqueous buffers, and combinations thereof.

In some embodiments, the pharmaceutically acceptable carrier or diluent is an aqueous buffer. In other embodiments, the aqueous buffer is Tyrode's buffer, e.g., Tyrode's buffer comprising 140 mM NaCl, 6 mM KCl, 3 mM CaCl2, 2 mM MgCl2, 10 mM 4-(2-hydroxyethyl)-1-piperazineethanesulfonic acid (Hepes) pH 7.4, and 10 mM glucose.

In some embodiments, the pharmaceutical composition comprises molecules that ease the transfection of host cells, i.e., a transfection agent.

In some specific embodiments pharmaceutical composition comprises a pharmaceutically acceptable amphiphilic block co-polymer comprising blocks of poly(ethylene oxide) and polypropylene oxide).

An “amphiphilic block co-polymer” as used herein is a linear or branched co-polymer comprising or consisting of blocks of poly(ethylene oxide) (“PEO”) and blocks of poly(propylene oxide) (“PPO”). Typical examples of useful PEO-PPO amphiphilic block co-polymers have the general structures PEO-PPO-PEO (poloxamers), PPO PEO PPO, (PEO PPO-)4ED (a poloxamine), and (PPO PEO-)4ED (a reverse poloxamine), where “ED” is a ethylenediaminyl group.

A “poloxamer” is a linear amphiphilic block co-polymer constituted by one block of poly(ethylene oxide) coupled to one block of poly(propylene oxide) coupled to one block of PEO, i.e., a structure of the formula EOa-POb-EOa, where EO is ethylene oxide, PO is propylene oxide, a is an integer from 2 to 130, and b is an integer from 15 to 67. Poloxamers are conventionally named by using a 3-digit identifier, where the first 2 digits multiplied by 100 provides the approximate molecular mass of the PPO content, and where the last digit multiplied by 10 indicates the approximate percentage of PEO content. For instance, “Poloxamer 188” refers to a polymer comprising a PPO block of a molecular weight of about 1800 (corresponding to b being about 31 PPO) and approximately 80% (w/w) of PEO (corresponding to a being about 82). However, the values are known to vary to some degree, and commercial products such as the research grade Lutrol® F68 and the clinical grade Kolliphor® P188, which according to the producer's data sheets both are Poloxamer 188, exhibit a large variation in molecular weight (between 7,680 and 9,510) and the values for a and b provided for these particular products are indicated to be approximately 79 and 28, respectively. This reflects the heterogeneous nature of the block co-polymers, meaning that the values of a and b are averages found in a final formulation.

A “poloxamine” or “sequential poloxamine” (commercially available under the trade name of Tetronic®) is an X-shaped block co-polymers that bears four PEO-PPO arms connected to a central ethylenediamine moiety via bonds between the free OH groups comprised in the PEO-PPO-arms and the primary amine groups in ethylenediamine moiety. Reverse poloxamines are likewise X-shaped block co-polymers that bear four PPO-PEO arms connected to a central ethylenediamine moiety via bonds between the free OH groups comprised in the PPO-PEO arms and the primary amine groups in ethylenediamine.

Preferred amphiphilic block co-polymers are poloxamers or poloxamines. Preferred are poloxamer 407 and 188, in particular poloxamer 188. Preferred poloxamines are sequential poloxamines of formula (PEO-PPO) 4-ED. Particularly preferred poloxamines are those marketed under the registered trademarks Tetronic® 904, 704, and 304, respectively. The characteristics of these poloxamines are as follows: Tetronic® 904 has a total average molecular weight of 6700, a total average weight of PPO units of 4020, and a PEO percentage of about 40%. Tetronic® 704 has a total average molecular weight of 5500, a total average weight of PPO units of 3300, and a PEO percentage of about 40%; and Tetronic® 304 has a total average molecular weight of 1650, a total average weight of PPO units of 990, and a PEO percentage of about 40%.

In some embodiments, the pharmaceutical composition comprises the amphiphilic block co-polymer in an amount of from 0.2% w/v to 20% w/v, such as of from 0.2% w/V to 18% w/v, 0.2% w/v to 16% w/v, 0.2% w/v to 14% w/v, 0.2% w/v to 12% w/v, 0.2% w/v to 10% w/v, 0.2% w/v to 8% w/v, 0.2% w/v to 6% w/v, 0.2% w/v to 4% w/v, 0.4% w/v to 18% w/v, 0.6% w/v to 18% w/v, 0.8% w/v to 18% w/v, 1% w/v to 18% w/v, 2% w/v to 18% w/v, 1% w/v to 5% w/v, or 2% w/v to 4% w/v. Particularly preferred are amounts in the range of from 0.5% w/v to 5% w/v. In other embodiments, the pharmaceutical composition comprises the amphiphilic block co-polymer in an amount of from 2% w/v to 5% w/v, such as about 3% w/V.

The pharmaceutical composition may be formulated in any way suitable for administration to a subject, e.g., such as a liquid formulation for injection, e.g., for intradermal or intramuscular injection.

The pharmaceutical composition may be administered in any way suitable for administration to a subject, such as administered by intradermal, intramuscular, or subcutaneous injection, or by mucosal or epithelial application, such as intranasal or oral.

In some preferred embodiments, the pharmaceutical composition is administered by intramuscular or intradermal injection.

The amount of vector, e.g., DNA plasmid, in the pharmaceutical composition may vary depending on whether the pharmaceutical composition is administered for prophylactic or therapeutic treatment.

The pharmaceutical composition of the disclosure typically comprises the vector, e.g., DNA plasmid, in a range of from 0.1 to 10 mg, e.g., about 0.1, 0.2, 0.3, 0.4, 0.5, 0.6, 0.7, 0.8, 0.9 or 1 mg or e.g., 2, 3, 4, 5, 6, 7, 8, 9 or 10 mg.

In some preferred embodiments, the pharmaceutical composition is a sterile pharmaceutical composition.

### Treatment

In some aspects of the present disclosure, the vector, e.g., the DNA plasmid, is for use in the therapeutic or prophylactic treatment of a disorder, such as a disorder in humans. In some aspects of the present disclosure, the vector, e.g., the DNA plasmid, is for use in the prophylactic or therapeutic treatment of autoimmune diseases, allergic diseases and/or graft rejection.

Thus, in one aspect, the disclosure relates to a method of treating a subject having a disease or being in need of prevention of said disease, the method comprising administering to the subject a vector comprising:
a) a first nucleic acid sequence encoding a first polypeptide, wherein the first polypeptide comprises a targeting unit that targets antigen-presenting cells, a multimerization unit, such as a dimerization unit, and an antigenic unit comprising one or more epitopes, which are relevant for said disease; and
b) one or more further nucleic acid sequences encoding one or more further polypeptides, wherein the one or more further polypeptides comprise a further antigenic unit comprising one or more further epitopes,
wherein the vector allows for the co-expression of the first polypeptide and the one or more further polypeptides as separate molecules.

In another aspect, the disclosure relates to a method of treating a subject having an disease selected from autoimmune diseases, allergic diseases and graft rejection or being in need of prevention of said disease, the method comprising administering to the subject a vector comprising:
a) a first nucleic acid sequence encoding a first polypeptide, wherein the first polypeptide comprises a targeting unit that targets antigen-presenting cells, a multimerization unit, such as a dimerization unit, and an antigenic unit comprising one or more T cell epitopes of an allergen, self-antigen or alloantigen; and
b) one or more further nucleic acid sequences encoding one or more further polypeptides, wherein the one or more further polypeptides comprise a further antigenic unit comprising one or more allergens, hypoallergenic allergens, self-antigens or alloantigens,
wherein the vector allows for the co-expression of the first polypeptide and the one or more further polypeptides as separate molecules.

In some aspects of the present disclosure, the vector, e.g., the DNA plasmid, is for use in the prophylactic or therapeutic treatment an allergic disease, such as an allergic disease in humans.

Thus, in one aspect, the disclosure relates to a method of treating a subject having an allergic disease or being in need of prevention of said allergic disease, the method comprising administering to the subject a vector comprising:
a) a first nucleic acid sequence encoding a first polypeptide, wherein the first polypeptide comprises a targeting unit that targets antigen-presenting cells, a multimerization unit, such as a dimerization unit, and an antigenic unit comprising one or more T cell epitopes of an allergen, a self-antigen or an alloantigen; and
b) one or more further nucleic acid sequences encoding one or more further polypeptides, wherein the one or more further polypeptides comprise a further antigenic unit comprising one or more allergens, hypoallergenic allergens, self-antigens or alloantigens,
wherein the vector allows for the co-expression of the first polypeptide and the one or more further polypeptides as separate molecules.

In some embodiments, the vector or the pharmaceutical composition is administered in a therapeutically or prophylactically effective amount, such as administered by intradermal, intramuscular, or subcutaneous injection, or by mucosal or epithelial application, such as intranasal or oral.

In the method of treatment, the vector is preferably administered in a therapeutically effective or prophylactically effective amount. Such an amount of vector may be administered in one administration, i.e., one dose, or in several administrations, i.e., repetitive doses, i.e., in a series of doses, e.g., over the course of several days, weeks or months.

The actual dose to be administered may vary and depend on whether the treatment is a prophylactic or therapeutic treatment, the age, weight, gender, medical history, pre-existing conditions and general condition of the subject, the severity of the disease being treated and the judgment of the health care professionals.

In the method of treatment, the vector may be administered in the form of the pharmaceutical composition and in the mode of administration as described herein.

The method of treating according to the disclosure can continue for as long as the clinician overseeing the patient's care deems the method to be effective and the treatment to be needed.

In some embodiments of the present disclosure, the vector, e.g., the DNA plasmid, is for use in the treatment of a cancer. Such vectors and antigenic units of such vectors, including antigenic units of individualized and non-individualized polypeptides and various embodiments thereof, have been described in detail herein.

Thus, in some embodiments, the disclosure relates to a method of treating a subject having cancer, the method comprising administering to the subject a vector comprising:
a) a first nucleic acid sequence encoding a first polypeptide, wherein the first polypeptide comprises a targeting unit that targets antigen-presenting cells, a multimerization unit, such as a dimerization unit, and an antigenic unit, comprising one or more cancer antigens or parts thereof; and
b) one or more further nucleic acid sequences encoding one or more further polypeptides, wherein the one or more further polypeptides comprise a further antigenic unit comprising one or more further epitopes,
wherein the vector allows for the co-expression of the first polypeptide and the one or more further polypeptides as separate molecules.

The cancer may be a solid or a liquid cancer. Examples of solid cancers are cancers forming a solid mass, e.g., a tumor. Examples of liquid cancers are cancers present in body fluid, such as lymphomas or blood cancers.

In some embodiments of the present disclosure, the vector, e.g., the DNA plasmid is for use in the treatment of a cancer selected from the group consisting of breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, bladder cancer, esophageal cancer, Hodgkin's disease and adrenocortical cancer.

In other embodiments of the present disclosure, the vector, e.g., the DNA plasmid, is for use in the treatment of an infectious disease. Such vectors and antigenic units of such vectors have been described in detail herein.

Thus, in some embodiments, the disclosure relates to a method of treating a subject having an infectious disease or being in need of prevention of an infectious disease, the method comprising administering to the subject a vector comprising:
a) a first nucleic acid sequence encoding a first polypeptide, wherein the first polypeptide comprises a targeting unit that targets antigen-presenting cells, a multimerization unit, such as a dimerization unit, and an antigenic unit comprising one or more epitopes, which are relevant for said infectious disease; and
b) one or more further nucleic acid sequences encoding one or more further polypeptides, wherein the one or more further polypeptides comprise a further antigenic unit comprising one or more further epitopes,
wherein the vector allows for the co-expression of the first polypeptide and the one or more further polypeptides as separate molecules.

Epitopes and antigens which are relevant for infectious diseases, e.g., which are derived from pathogens, have been described in detail herein.

In some embodiments, the allergic disease is selected from the group consisting of food allergies, animal allergies, pollen allergies, mold allergies, dust mite allergies, latex allergies and drug allergies.

Indicators of treatment success are known in the art. In some embodiments, indicators of treatment success include increased levels of antigen-specific regulatory T cells, reduced levels of antigen-specific effector T cells, (and increased levels of regulatory T cells), reduced levels of effector T cells, reduced level of T cell activation in ELISPOT when stimulated with the antigenic unit/T cell epitopes in the antigenic unit, reduced level of basophil activation in a basophil activation test (BAT).

In some embodiments, a radioallergosorbent test (RAST) may likewise be used to compare the allergen-specific IgE antibody level in a blood sample from a subject before and after administration of the immunotherapy construct, wherein a lower allergen-specific IgE antibody level indicates successful tolerance induction.

In other embodiments of the present disclosure, the vector, e.g., the DNA plasmid, is for use in the treatment of an allergy. Such vectors and antigenic units of such vectors have been described in detail herein.

Thus, in some embodiments, the vector, e.g., the DNA plasmid, is for use in treating a subject having an allergic disease or being in need of prevention an allergic disease, the method comprising administering to the subject a vector comprising:
a) a first nucleic acid sequence encoding a first polypeptide, wherein the first polypeptide comprises a targeting unit that targets antigen-presenting cells, a multimerization unit, such as a dimerization unit, and an antigenic unit comprising one or more T cell epitopes of an allergen, self-antigen or alloantigen, which are relevant for said allergic disease; and
b) one or more further nucleic acid sequences encoding one or more further polypeptides, wherein the one or more further polypeptides comprise a further antigenic unit comprising one or more allergens, hypoallergenic allergens, self-antigens or alloantigens,
wherein the vector allows for the co-expression of the first polypeptide and the one or more further polypeptides as separate molecules.

### EXAMPLES

### Example 1: Design and Production of DNA Constructs of the Disclosure for Use as a Vaccine Against Infection with SARS-COV-2

All gene sequences of tested constructs were ordered from Genscript (Genscript Biotech B.V., Netherlands) and cloned into the expression vector pUMVC4a.

DNA plasmids TECH011-IV003 and TECH011-IV004 (FIG. 15 and FIG. 19) comprise a nucleotide sequence encoding a first polypeptide comprising: the signal peptide, targeting unit, multimerization unit which here consists of a dimerization unit, and unit linker described in Table 3, followed by an antigenic unit containing T cell epitopes pep08, pep18 and pep25 (Table 4), linked among them with the linker GGGGSGGGGS (SEQ ID NO: 29), followed in frame by the leucine zipper motif

(SEQ ID NO: 155)
LEIRAAFLRQRNTALRTEVAELEQEVQRLENEVSQYETRYGPL

(LZ1 on FIG. 15), followed by the linker GSG. The nucleotide sequence encoding the first polypeptide is then followed by a sequence, the 2A self-cleaving peptide from Thosea asigna virus capsid protein EGRGSLLTCGDVEENPGP (SEQ ID NO: 3), which in turn is followed by a nucleotide sequence encoding the second polypeptide. The second polypeptide comprises the RBD domain (corresponding to amino acids 319-542) of the spike protein of SARS-COV-2 (Wuhan variant), followed by the GSAT linker GGSAGGSGSGSSGGSSGASGTGTAGGTGSGSGTGSG (SEQ ID NO: 115) and a second leucine zipper motif LEIEAAFLERENTALETRVAELRQRVQRLRNRVSQYRTRYGPL (SEQ ID NO: 25) (LZ2 on FIG. 15, which heterodimerizes with the leucine zipper motif in the first polypeptide. Furthermore, in TECH01-IV004, the second polypeptide also comprises a signal peptide sequence MNFGLRLIFLVLTLKGVQC (SEQ ID NO: 130) immediately upstream of the RBD antigen.

TABLE 3

| Elements/ | |
|---|---|
| Units | Description and sequence |
| Signal | Native leader sequence |
| peptide | for human CCL3L1 |
| | MQVSTAALAVLLCTMALCNQVLS, |
| | SEQ ID NO: 156 |
| Targeting | Full length human CCL3L1 sequence |
| unit | APLAADTPTACCFSYTSRQIPQNFIADYFETSS |
| | QCSKPSVIFLTKRGRQVCADPSEEWVQKYVSDL |
| | ELSA, SEQ ID NO: 157 |
| Dimer- | Human hinge-region 1 from lgG3 |
| ization | ELKTPLGDTTHT, SEQ ID NO: 117 |
| unit | Human hinge-region 4 from IgG3 |
| | EPKSCDTPPPCPRCP, SEQ ID NO: 118 |
| | Glycine-serine-linker |
| | GGGSSGGGSG, SEQ ID NO: 15 |
| | Human CH3 domain from lgG3 |
| | GQPREPQVYTLPPSREEMTKNQVSLTCLVKGFYP |
| | SDIAVEWESSGQPENNYNTTPPMLDSDGSFFLYS |
| | KLTVDKSRWQQGNIFSCSVMHEALHNRFTQKSLS |
| | LSPGK, SEQ ID NO: 158 |
| Unit | Glycine-leucine linker |
| linker | GLGGL, SEQ ID NO: 26 |

TABLE 4

| T cell | | |
|---|---|---|
| epitope | Sequence | SEQ ID NO: |
| Pep 01 | RSFIEDLLFNKVTLA | 159 |
| pep 02 | MTYRRLISMMGFKMNYQVNGYPNMF | 160 |
| pep 03 | LMIERFVSLAIDAYP | 161 |
| pep 04 | RAMPNMLRIMASLVL | 162 |
| pep 05 | MVYMPASWVMRIMTW | 163 |
| pep 06 | FLNRFTTTLNDFNLVAM | 164 |
| pep 07 | SSVELKHFFFAQDGNAAI | 165 |
| pep 08 | HFAIGLALYYPSARIVYTACSHAAV | 166 |
| pep 09 | YFIKGLNNLNRGMVL | 167 |
| pep 10 | YLNTLTLAVPYNMRV | 168 |
| pep 11 | AQFAPSASAFFGMSRI | 169 |
| pep 12 | EIVDTVSALVYDNKL | 170 |
| pep 13 | SSGDATTAYANSVFNICQAVTANVNALL | 171 |
| pep 14 | HVISTSHKLVLSVNPYV | 172 |
| pep 15 | MLSDTLKNLSDRVVFVLWAHGFEL | 173 |
| pep 16 | TANPKTPKYKFVRIQPGQTF | 174 |
| pep 17 | ASIKNFKSVLYYQNNVFM | 175 |
| pep 18 | FVNEFYAYLRKHFSMM | 176 |
| pep 19 | RVWTLMNVLTLVYKV | 177 |
| pep 20 | FAYANRNRFLYIIKL | 178 |

DNA plasmids TECH011-IV005, TECH011-IV006, TECH011-IV007, TECH018-IV001 and TECH018-IV002 (FIG. 13B and FIG. 20) are heterodimeric constructs that comprise a nucleic acid sequence encoding a first polypeptide comprising a targeting unit (Table 3) followed by a heterodimeric sequence A (HD1 on FIG. 13B), an antigenic unit A (AntA on FIG. 13B), and the linker GSG. Downstream of the first polynucleotide, the plasmids further comprise a nucleotide sequence encoding the self-cleaving 2A peptide from Thosea asigna virus capsid protein with sequence EGRGSLLTCGDVEENPGP (SEQ ID NO: 3). The vectors further comprise a second polynucleotide downstream of this, which second polynucleotide encodes a signal peptide, a targeting unit and unit linker (Table 3), a heterodimeric sequence B (HD2 on FIG. 13B) which heterodimerizes with heterodimeric sequence A, and an antigenic unit B (AntB on FIG. 13B).

Heterodimeric sequences A and B are respectively:

(SEQ ID NO: 179)
GGSSGGKFGGSTTAPSAQLEKELQALEKENAQLEWELQALEKELAQGGGSG
GLTKFGGSTTAPSAQLEKELQALEKENAQLEWELQALEKELAQGGGSGGLT
GLSGL
and
(SEQ ID NO: 180)
GGSSGGKFGGSTTAPSAQLKKKLQALKKKNAQLKWKLQALKKKLAQGGGSG
GLTKFGGSTTAPSAQLKKKLQALKKKNAQLKWKLQALKKKLAQGGGSSGGL
TGLSGL for TECH011-IV005 (Acid/Base),
(SEQ ID NO: 181)
GEIAALEAKNAALKAEIAALEAKNAALKAGC
and
SEQ ID NO: 182)
GKIAALKAENAALEAKIAALKAENAALEAGGC for TECH011-IV006
(P7A/P8A),
(SEQ ID NO: 182)
GKIAALKAENAALEAKIAALKAENAALEAGGC
and
(SEQ ID NO: 181)
GEIAALEAKNAALKAEIAALEAKNAALKAGC for TECH011-IV007
(P8A/P7A),
(SEQ ID NO: 183)
GEIAALEAKNAALKAEIAALEAKIAALKAGY
and
(SEQ ID NO: 184)
YGKIAALKAENAALEAKIAALKAEIAALEAGY for TECH018-
IV001(N7/N8),
and
(SEQ ID NO: 185)
GEIAALEAKIAALKAKNAALKAEIAALEAG
and
(SEQ ID NO: 186)
GKIAALKAEIAALEAENAALEAKIAALKAG for TECH018-IV002
(N5/N6).

Antigenic units A and B were, respectively, the RBD (corresponding to amino acids 319-542) of the spike protein of SARS-COV-2 (Wuhan variant) and epitopes pep08, pep18 and pep25 (Table 4), linked among them with the linker GGGGGGGGS (SEQ ID NO: 29), for constructs TECH011-IV005, TECH011-IV006, TECH018-IV001 and TECH018-IV002.

Antigenic units A and B were, respectively, epitopes pep08, pep18 and pep25 (Table 4), linked among them with the linker GGGGSGGGGS (SEQ ID NO: 29), and the RBD (aa319-542) of the spike protein of SARS-COV-2 (Wuhan variant) for constructs TECH011-IV007.

DNA plasmids TECH021-IV001 (FIG. 14 and FIG. 21) and TECH023-IV003 (Separately secreted, targeted antigen) (FIG. 18) comprise a nucleotide sequence encoding a first polypeptide comprising the signal peptide, targeting unit, dimerization unit and unit linker described in Table 3, followed by an antigenic unit containing epitopes pep08, pep18 and pep25 (Table 4), followed by the linker GSG. The vectors comprise downstream of this a polynucleotide encoding the self-cleaving 2A peptide from Thosea asigna virus capsid protein EGRGSLLTCGDVEENPGP (SEQ ID NO: 3). The vectors further comprise a polynucleotide encoding a second polypeptide downstream of the self-cleaving 2A peptide, which second polypeptide comprises the signal peptide sequence MNFGLRLIFLVLTLKGVQC (SEQ ID NO: 130) and the RBD (corresponding to amino acids 319-542) of the spike protein of SARS-COV-2 (Wuhan variant). Furthermore, in TECH023-IV003, the targeting unit α-MHC-II scFv DIVLTQSPASLAVSLGQRATISCRASKSVSTSGYSYMHWYQQKPGQPPKLLIYLTSNL ESGVPARFSGSGSGTDFTLNIHPVEEEDAATYYCQHSRELPWTFGGGTKLEIKGGGG SGGGGSGGGGSQVQLQQSGPDLVKPGASVTISCKASGYAFSSSWMSWLKQRPGK GLEWIGWIFPRDGDTNYNGKFKGKATLTADKSSSTAYMQLSSLTSEDSAVYFCARRG DYHYGMDYWGQGTSVTVSS (SEQ ID NO: 187) was added between the signal peptide and the RBD antigen.

DNA constructs TECH021-IV018, TECH021-IV019 and TECH021-IV020 (FIG. 21) comprise a nucleotide sequence encoding a first polypeptide comprising the signal peptide, targeting unit, dimerization unit and unit linker described in Table 3, followed by an antigenic unit containing SARS-COV-2 epitopes (Table 4), linked among them with the linker GGGGSGGGGS (SEQ ID NO: 29), followed by the linker GSG. The vectors further comprise a nucleotide sequence encoding the self-cleaving 2A peptide from Thosea asigna virus capsid protein EGRGSLLTCGDVEENPGP (SEQ ID NO: 3), followed by a nucleotide sequence encoding the second polypeptide comprising the signal peptide MNFGLRLIFLVLTLKGVQC (SEQ ID NO: 130) and the RBD (corresponding to amino acids 319-542) of the spike protein of SARS-COV-2 (Wuhan variant) DNA construct. The epitopes of the antigenic unit of TECH021-IV018 were pep08-pep18-pep09-pep13-pep25, connected among them with the linker GGGGSGGGGS (SEQ ID NO: 29). The epitopes of the antigenic unit of TECH021-IV020 peptides were pep02-pep14/pep17-pep9/pep10-pep15/pep08-pep01/pep03-pep04/pep05-pep19/pep20/pep11/pep12-pep06/pep07-pep13/pep18-pep25, where “-” means represents the linker GGGGSGGGGS (SEQ ID NO: 29) and “/” represents no linker between epitopes.

### Example 2: In Vitro Assessment of Expression and Secretion of Proteins Encoded by TECH011-IV003, TECH011-IV004, TECH011-IV005, TECH011-IV006, TECH011-IV007, TECH018-IV001 and TECH018-IV002

The purpose of this study was to characterize the protein expression level post transient transfection of mammalian cells with the TECH011-IV003, TECH011-IV004, TECH011-IV005, TECH011-IV006, TECH011-IV007, TECH018-IV001 and TECH018-IV002 DNA plasmids by measuring the presence of secreted proteins in the cell supernatant by an ELISA assay using antibodies detecting the targeting and antigenic units. In addition, Western blot (WB) analysis was performed on supernatant samples from transfected Expi293F cells to further characterize the proteins encoded by TECH011-IV003, TECH011-IV004 and TECH011-IV007.

Briefly, Expi293F cells (2×10⁶ cells/mL, 1 mL) were seeded in a 96-well culture plate. The cells were transfected with 0.64 μg/mL plasmid DNA using ExpiFectamine 293 Reagent (100014994 Thermo Fisher Sci.), and the plates were incubated on an orbital shaker (3 mm diameter, 850-900 rpm) in a humidified CO₂ cell incubator (8% CO₂, 37° C.). The plates were incubated for 72 h before the supernatant was harvested.

The secreted proteins were characterized in a sandwich ELISA of the supernatant using SARS-COV-2/2019-nCOV Spike/RBD Antibody (capture antibody, 1:1000, 100 μL/well, 40592-T62, Sino Biological), anti-hCCL3/MIP-1α biotinylated antibody (detection antibody, 0.2 μg/mL, 100 μL/well, BAF270, R&D Systems), and Pierce Streptavidin Poly-HRP (1:8000, 100 μL/well, 21140, Thermo Fisher Scientific).

The secreted proteins were characterized in a sandwich ELISA of the supernatant using SARS-COV-2/2019-nCOV Spike/RBD Antibody (capture antibody, 1:500, 100 μL/well, 40592-T62, Sino Biological, Beijing China), mouse anti-SARS-COV-2 spike antibody (detection antibody, 100 μL/well, 40591-MM41, Sino Biological), and goat anti-mouse IgG-HRP antibody (secondary HRP-conjugated antibody, 1:1000, 100 μL/well, 1079-05, Southern Biotech, Birmingham, AL, USA).

Further, the samples were prepared for SDS-PAGE by mixing 35 μL supernatant from transfected Expi293F cells with 12.5 μl 4× Laemmli sample buffer (1610747, Bio-Rad) with 2.5 μL DTT (700416, Cayman Chemical), for reducing conditions. The samples were heated at 70° C. for 10 minutes (min) before adding 25 μL per lane to 4%-20% Criterion TGX Stain-Free precast gels (5678094, Bio-Rad). SDS-PAGE was performed in 1× Tris/Glycine/SDS running buffer (1610732, Bio-Rad) with Precision Plus Protein All Blue Prestained (1610373, Bio-rad) and Unstained protein standards (1610363, Bio-Rad). Proteins were transferred from the gel onto EtOH activated low fluorescence (LF) 0.45 μm PVDF membranes (1704275, Bio-Rad) by using the Trans-Blot Turbo semi-dry transfer system (Bio-Rad). PVDF membranes were blocked in EveryBlot buffer (12010020, Bio-Rad) for 5 min and probed with the primary antibody (goat anti-human MIP-1α, AF270, R&D Systems or rabbit anti-human RBD, 40925-T62, Sino Biological Inc.) diluted 1:1000 in EveryBlot at 4° C. overnight The membranes were washed, incubated with the corresponding fluorochrome-conjugated secondary antibody (donkey anti-goat antibody Dylight 800-conjugated, SA5-10092, Invitrogen or donkey anti-rabbit Daylight 800-conjugated, SA5-10044, Invitrogen) diluted 1:5000 in EveryBlot with 0.01% SDS (444062F, VWR) for 1 h at room temperature. The membranes were then washed, rinsed in 96% ethanol, and dried in the dark. Images were acquired by using a ChemiDoc™ MP Imaging System (setting Dylight 650 and 800, Auto Optimal).

FIG. 22 shows that all constructs were expressed and secreted from transfected Expi293F cells. Moreover, the ELISA results for TECH011-IV003 and TECH011-IV004 using the anti-RBD antibody and anti-CCL3/MIP1a antibody sandwich suggest that the VB-LZ1 and RBD-LZ2 protein domains occur dimerized in the supernatant. FIG. 23 shows the detection of RBD protein in the supernatant of transfected Expi293F cells using two different antibodies binding to the RBD in an ELISA. For constructs TECH011-IV003 and TECH011-IV004, the RBD-LZ2 protein was detected at higher levels in supernatant from cells transfected with TECH011-IV004 compared to TECH011-IV003. For the heterodimeric constructs, TECH011-IV006 was detected at a higher level compared to TECH018-IV001, and TECH018-IV002, whereas TECH011-IV005 and TECH011-IV007 were detected at lower levels compared among these heterodimeric constructs.

The WB analysis for the detection of the targeting unit CCL3L1 of the constructs (FIG. 24) showed presence of the VB-LZ1 molecule of TECH011-IV003 and TECH011-IV004 in the supernatant of transfected cells as full-length monomer (reduced condition) and as dimers (non-reducing condition) at the expected MW (39 kDa and 77 kDa, respectively). For the heterodimeric construct TECH011-IV007, two monomers were successfully detected at the expected MW (28 and 50 kDa), and the non-reducing conditions indicated that the monomers were able to form a dimer through the heterodimerization domain, although the dimer was larger than expected (87 vs 78 kDa), suggesting post-translational modifications, such as glycosylation.

The WB analysis for the detection of the antigen part SARS-COV-2 RBD of these new formats (FIG. 25) showed the presence of free RBD-LZ2 for TECH011-IV003 and TECH011-IV004, in higher amounts in the latter construct. For these two constructs, no unskipped monomers were detected, suggesting successful skipping of the 2A sequence between the two proteins. Dimer was just detected for TECH011-IV003, but at a lower MW (110 vs 145 kDa) which suggested a dimer with only one RBD molecule attached. For TECH011-IV007, its monomer containing RBD was successfully detected at the expected MW (50 kDa), although its dimer was larger than expected (87 vs 78 kDa), suggesting glycosylation of this VB protein, as also observed in FIG. 24.

Taken together, the ELISA and WB data demonstrate expression from the above constructs. The WBs using either anti-RBD antibody or anti-CCL3/MIP1a antibody under reducing conditions showed that the self-cleaving peptide sequence encoded in constructs TECH011-IV003 and TECH011-IV004 resulted in production of two separate proteins, VB-LZ1 and RBD-LZ2, while the ELISA, using SARS-COV-2/2019-nCOV Spike/RBD antibody and anti-hCCL3/MIP-1α biotinylated antibody, demonstrated that the RBD-LZ2 protein was bound to the VB-LZ1 protein. The WB of supernatants from TECH011-IV007 transfected cells demonstrated the detection of two separate monomers (reducing conditions) that were able to form a heterodimer (non-reducing conditions).

### Example 3: In Vitro Assessment of Expression and Secretion of Proteins Encoded by TECH021-IV001, TECH021-IV018 and TECH021-IV020 (Varying Length of T Cell Epitopes)

The purpose of this study was to characterize the protein expression and secretion post transient transfection of mammalian cells with the TECH021-IV001, TECH021-IV018 and TECH021-IV020 DNA plasmids by measuring the presence of secreted proteins in the cell supernatant by an ELISA assay using antibodies detecting the targeting and dimerization units. In addition, WB analysis was performed on supernatant samples from transfected Expi293F cells to further characterize the proteins encoded by TECH021-IV001, TECH021-IV018 and TECH021-IV020.

Briefly, Expi293F cells (2×10⁶ cells/mL, 1 mL) were seeded in a 96-well culture plate. The cells were transfected with 0.64 μg/mL plasmid DNA using ExpiFectamine 293 Reagent (100014994 Thermo Fisher Sci.), and the plates were incubated on an orbital shaker (3 mm diameter, 850-900 rpm) in a humidified CO₂ cell incubator (8% CO₂, 37° C.). The plates were incubated for 72 h before the supernatant was harvested.

The secreted TECH021-IV001, TECH021-IV018, and TECH021-IV020 proteins were characterized in sandwich ELISA of the supernatant using mouse anti human IgG CH3 domain antibody (capture antibody, 100 μL/well, 2 μg/mL, MCA878G, Bio-Rad), and goat anti-human CCL3/MIP-1α biotinylated antibody (detection antibody, 100 μL/well, 0.2 μg/ml, BAF270, R&D Systems) followed by incubation with HRP-conjugated Streptavidin (secondary HRP-conjugated reagent, 100 L/well, S2438-250UG, Sigma-Aldrich). A standard curve of an in-house purified Nykode control construct (VB4219, containing the sequences described for the first polypeptide (Table 3) but without any antigenic unit) was included in each experiment. The amount of protein present in the various cell supernatants was estimated using this standard curve.

Further, the samples were prepared for SDS-PAGE by mixing 45.5 μL supernatant from transfected Expi293F cells with 17.5 μl 4× Laemmli sample buffer (1610747, Bio-Rad) with 7 μL DTT (700416, Cayman Chemical) for reducing condition. The samples were heated at 80° C. for 10 minutes (min) before adding 25 μL per lane to 4%-20% Criterion TGX Stain-Free precast gels (5678094, Bio-Rad). SDS-PAGE was performed in 1× Tris/Glycine/SDS running buffer (diluted by 1610732, Bio-Rad) with Precision Plus Protein All Blue Prestained (1610373, Bio-rad) and Unstained protein standards (1610363, Bio-Rad). Proteins were transferred from the gel onto EtOH activated low fluorescence (LF) 0.45 μm PVDF membranes (1704275, Bio-Rad) by using the Trans-Blot Turbo semi-dry transfer system (Bio-Rad). PVDF membranes were blocked in EveryBlot buffer (12010020, Bio-Rad) for 5 min and probed with the primary antibody (goat anti-human MIP-1α, AF270, R&D Systems or rabbit anti-human RBD, 40925-T62, Sino Biological Inc.) diluted 1:1000 in EveryBlot at 4° C. overnight The membranes were washed, incubated with the corresponding fluorochrome-conjugated secondary antibody (donkey anti-goat antibody Alexa Fluor 800-conjugated, A32930, Invitrogen or donkey anti-rabbit Daylight 800-conjugated, SA5-10044, Invitrogen) diluted 1:10000 or 1:5000 in EveryBlot with 0.01% SDS (444062F, VWR) for 1 h at room temperature. The membranes were then washed, rinsed in 96% ethanol, and dried in the dark. Images were acquired by using a ChemiDoc™ MP Imaging System (setting Dylight 650 and 800, Auto Optimal).

The results shown in FIG. 26 demonstrate expression and secretion of the VB protein comprising an antigenic unit with either 3 or 5 T cell epitopes from Expi293F cells transfected with the pDNA TECH021-IV001 and TECH021-IV018, respectively. Moreover, FIG. 27 shows that the CCL3L1 targeted VB proteins with 3 or 5 T cell epitopes were secreted from TECH021-IV001 and TECH021-IV018 transfected cells, respectively, as intact monomers of the expected sizes (34 kDa and 40 kDa, respectively), while FIG. 28 shows that the RBD protein was also secreted by the transfected Expi293F cells. Combined, the WB analysis in FIGS. 27 and 28 demonstrate that, in both TECH021-IV001 and TECH021-IV018, the 2A self-cleaving peptide encoded between the VB and RBD proteins successfully resulted in the two encoded polypeptides being secreted as separate proteins.

The results shown in FIGS. 29-31 demonstrate that the number of T cell epitopes in the antigenic unit of the constructs can be increased to at least 20 T cell epitopes. FIG. 29 shows the expression and secretion of polypeptides comprising a targeting unit, a dimerization unit, and an antigenic unit with 20 T cell epitopes from Expi293F cells transfected with the pDNA TECH021-IV020. Moreover, FIG. 30 shows that the CCL3L1 targeted VB proteins with 20 T cell epitopes was secreted from TECH021-IV020 (74 kDa) transfected cells as intact monomers and FIG. 31 shows that the RBD protein was also secreted by the transfected Expi293F cells. Combined, the WB analysis in FIGS. 30 and 31 demonstrate that in TECH021-IV020, the 2A self-cleaving peptide sequence encoded between the VB and RBD proteins successfully resulted in the two encoded polypeptides being secreted as separate proteins.

Taken together, these results demonstrate that from a single construct, a first polypeptide comprising a targeting unit, a dimerization unit, and an antigenic unit with several T cell epitopes, can be co-expressed with a second, freely secreted second polypeptide consisting of a B cell antigen.

### Example 4: In Vitro Assessment of Expression and Secretion of Proteins Encoded by TECH023-IV003, Separately Secreted, Targeted Antigen

The purpose of this study was to characterize the protein expression and secretion post transient transfection of mammalian cells with the TECH023-IV003 DNA plasmid by measuring the presence of secreted proteins in the cell supernatant by an ELISA assay using antibodies detecting the targeting and dimerization units. In addition, WB analysis was performed on supernatant samples from transfected Expi293F cells to further characterize the proteins encoded by TECH023-IV003.

Briefly, Expi293F cells (2×10⁶ cells/mL, 1 mL) were seeded in a 96-well culture plate. The cells were transfected with 0.64 μg/mL plasmid DNA using ExpiFectamine 293 Reagent (100014994 Thermo Fisher Sci.), and the plates were incubated on an orbital shaker (3 mm diameter, 850-900 rpm) in a humidified CO₂ cell incubator (8% CO₂, 37° C.). The plates were incubated for 72 h before the supernatant was harvested. The secreted TECH023-IV003 proteins were characterized in a sandwich ELISA of the supernatant using mouse anti human IgG CH3 domain antibody (capture antibody, 100 μL/well, 2 μg/mL, MCA878G, Bio-Rad), and goat anti-human CCL3/MIP-1α biotinylated antibody (detection antibody, 100 L/well, 0.2 μg/ml, BAF270, R&D Systems) followed by incubation with HRP-conjugated Streptavidin (secondary HRP-conjugated reagent, 100 μL/well, S2438-250UG, Sigma-Aldrich). A standard curve of an in-house purified Nykode control construct (VB4219, with no antigenic unit) was included in each experiment. The amount of protein present in the various cell supernatants was estimated using this standard curve.

Further, the samples were prepared for SDS-PAGE by mixing 45.5 μL supernatant from transfected Expi293F cells with 17.5 μl 4× Laemmli sample buffer (1610747, Bio-Rad) with 7 μL DTT (700416, Cayman Chemical) for reducing condition. The samples were heated at 80° C. for 10 minutes (min) before adding 25 μL per lane to 4%-20% Criterion TGX Stain-Free precast gels (5678094, Bio-Rad). SDS-PAGE was performed in 1× Tris/Glycine/SDS running buffer (diluted by 1610732, Bio-Rad) with Precision Plus Protein All Blue Prestained (1610373, Bio-rad) and Unstained protein standards (1610363, Bio-Rad). Proteins were transferred from the gel onto EtOH activated low fluorescence (LF) 0.45 μm PVDF membranes (1704275, Bio-Rad) by using the Trans-Blot Turbo semi-dry transfer system (Bio-Rad). Afterwards, PVDF membranes were blocked in EveryBlot buffer (12010020, Bio-Rad) for 5 min and probed with the primary antibody (goat anti-human MIP-1α, AF270, R&D Systems or rabbit anti-human RBD, 40925-T62, Sino Biological Inc.) diluted 1:1000 in EveryBlot at 4° C. overnight The membranes were washed, incubated with the corresponding fluorochrome-conjugated secondary antibody (donkey anti-goat antibody Alexa Fluor 800-conjugated, A32930, Invitrogen or donkey anti-rabbit Daylight 800-conjugated, SA5-10044, Invitrogen) diluted 1:10000 or 1:5000 in EveryBlot with 0.01% SDS (444062F, VWR) for 1 h at room temperature. The membranes were then washed, rinsed in 96% ethanol, and dried in the dark. Images were acquired by using a ChemiDoc™ MP Imaging System (setting Dylight 650 and 800, Auto Optimal).

The results shown in FIG. 32 demonstrate expression and secretion of a protein comprising an antigenic unit of 3 T cell epitopes from Expi293F cells transfected with TECH023-IV003. Moreover, FIG. 33 shows that the CCL3L1-targeted polypeptide was secreted as an intact monomer of the expected size (34 kDa) while FIG. 34 shows that the anti-MHC class II scFv-targeted RBD polypeptide was also secreted as a separate protein (51 kDa) by the transfected Expi293F cells.

Taken together, these results demonstrate that from a single construct, a protein comprising a targeting unit, a dimerization unit, and an antigenic unit of T cell epitopes can be co-expressed with a second, separate protein comprising a second targeting unit and a B cell antigen using a 2A self-cleaving peptide.

### Example 5: Assessment of T Cell Response Induced Against T Cell Epitopes and RBD (Amino Acids 319-542) from SARS-COV-2 Induced by TECH011-IV004, TECH021-IV001 and TECH023-IV003

The immunogenicity elicited by the novel formats encoded in the constructs TECH011-IV004, TECH021-IV001 and TECH023-IV003, all comprising the 3 SARS-COV-2 T cell antigens pep08, pep18, and pep25, and the SARS-COV-2 RBD domain (Wuhan variant), was determined by immunizing mice and measuring the number of antigen specific, IFN-γ secreting T cells in splenocytes by a fluorospot assay.

Female, 6-week-old BALB/c mice were obtained from Janvier Labs (France). All animals were housed in the animal facility at the Radium Hospital, Oslo University Hospital (Oslo, Norway). All animal protocols were approved by the Norwegian Food Safety Authority (Oslo, Norway). Six mice/group were used for the testing of the constructs comprising an antigenic unit, whereas 3 mice/group were used for the negative control VB1026 (empty antigenic unit).

A final dose of 5 μg DNA plasmid dissolved in sterile 1×PBS was administered by intramuscular needle injection to each tibialis anterior, followed by electroporation with AgilePulse in vivo electroporation system (BTX, USA). Mice were immunized twice, at day 0 and day 21.

The spleens were collected 35 days after first vaccination and mashed in a cell strainer to obtain a single cell suspension. The red blood cells were lysed using ammonium-chloride-potassium (ACK) lysing buffer. The splenocytes from each of the individual mouse were counted using the NucleoCounter NC-202 (ChemoMetec, Copenhagen, Denmark) and resuspended to a final concentration of 6×10⁶ cells/mL. The splenocytes were then seeded 6×10⁵ cells/well and re-stimulating with 4 μg/mL of single peptides corresponding to the 3 T cell epitopes endoded in the pDNA construct (Table 4) and 2 μg/mL RBD peptide pools (Table 5) for 24 h. No-peptide-stimulation was used as negative control. The stimulated splenocytes were analyzed for IFN-γ responses using the IFN-γ FluoroSpot kit (Mabtech AB, Stockholm, Sweden). Spot-forming cells were measured in an IRIS Fluorospot and ELISpot plate reader (Mabtech AB) and analyzed using the Apex software (Mabtech AB). Results are shown as the mean number of IFN-γ⁺ spots/10⁶ splenocytes.

TABLE 5

| | Composition | |
|---|---|---|
| Pool ID | (AA position) | Sequence |
| Pool 1 | RBD (1-43)-15-mers | NITNLCPFGEVFNATRFASVYAWNRK |
| | with 3 aa overlap | RISNCVADYSVLYNSAS (SEQ ID NO: |
| | | 188) |
| Pool 2 | RBD (31-73)-15-mers | CVADYSVLYNSASFSTFKCYGVSPTKL |
| | with 3 aa overlap | NDLCFTNVYADSFVIR (SEQ ID NO: |
| | | 189) |
| Pool 3 | RBD (61-103-15-mers | CFTNVYADSFVIRGDEVRQIAPGQTG |
| | with 3 aa overlap | KIADYNYKLPDDFTGCV (SEQ ID NO: |
| | | 190) |
| Pool 4 | RBD (91-133)-15-mers | YNYKLPDDFTGCVIAWNSNNLDSKV |
| | with 3 aa overlap | GGNYNYLYRLFRKSNLKP (SEQ ID |
| | | NO: 191) |
| Pool 5 | RBD (121-166)-15-mers | YLYRLFRKSNLKPFERDISTEIYQAGST |
| | with 3 aa overlap | PCNGVEGENCYFPLQSYG (SEQ ID |
| | | NO: 192) |
| Pool 6 | RBD (154-194)-15-mers | GFNCYFPLQSYGFQPTNGVGYQPYR |
| | with 3 aa overlap | VVVLSFELLHAPAT (SEQ ID NO: |
| | | 193) |

FIG. 35 shows the combined IFN-γ T cell responses against the predicted T cell epitopes from multiple SARS-COV-2 strains and the RBD-specific T cell response detected in spleens at day 35 from mice vaccinated twice with 5 μg of DNA encoding the constructs TECH011-IV004, TECH021-IV001, and TECH023-IV003. All three constructs encoded a polypeptide comprising a targeting unit, a dimerization unit, and an antigenic unit with the 3 predicted T cell epitopes pep08, pep18 and pep25, and all three constructs elicited T cell responses against these 3 T cell epitopes. In addition, all constructs were also able to elicit an RBD-specific T cell response (FIG. 35). The combined T cell responses elicited against the RBD (amino acids 319-542) and the 3 T cell epitopes encoded in the antigenic unit indicates that combining T cell epitopes with a B cell antigen in either design, targeted antigen can broaden the T cell response induced by the vaccine.

### Example 6: Assessment of Humoral Immune Response Induced in Mice Against SARS-COV-2 RBD (Aa319-542, Wuhan Variant) Induced by TECH011-IV004, TECH021-IV001 and TECH023-IV003

Sera from the mice vaccinated with TECH011-IV004, TECH021-IV001 and TECH023-IV003, all 3 constructs encoding the 3 SARS-COV-2 T cell epitopes pep08, pep18, and pep25, and the RBD (Wuhan variant), or the VB1026 control plasmid were collected 35 days after first vaccination and tested for anti-RBD IgG antibodies binding the RBD (Wuhan variant) protein.

Briefly, blood was collected by cardiac puncture of the vaccinated mice. Coagulated blood was centrifuged twice (1000 g, 15 min) and the serum was collected and transferred to a clean tube. The humoral immune response was evaluated in an ELISA assay detecting total IgG in the sera binding to RBD (amino acids 319-542) from SARS-COV2 (Wuhan variant). ELISA plates (MaxiSorp Nunc-Immuno plates) were coated with 1 μg/mL recombinant RBD-His protein antigen in PBS overnight at 4° C. Plates were blocked with 4% BSA in 1×PBS for 1 hour at RT. Plates were then incubated with serial dilutions of mouse sera and incubated for 2 h at 37° C. Plates were washed 3× and incubated with 1:50 000 dilution of anti-mouse total IgG-HRP antibody (Southern Biotech) and incubated for 1 h at 37° C. After final washing, plates were developed using TMB substrate (Merck, cat. CL07-1000). Plates were read at 450 nm wavelength within 30 min using a SPARK® Multimode Microplate Reader (Tecan). Binding antibody endpoint titers were calculated as the reciprocal of the highest dilution resulting in a signal above the cutoff. Binding antigens tested included SARS-COV-2 antigens: RBD (Sino Biological 40592-V08H).

The results shown in FIG. 36 demonstrate that TECH011-IV004, TECH021-IV001 and TECH023-IV003, encoding RBD (amino acids 319-542) from SARS-COV-2 (Wuhan variant) induced total IgG responses against said antigenic unit. This indicates that all three constructs were able to induce RBD-antigen specific antibody response.

Taken together, the results from examples 5 and 6 demonstrate that a construct encoding a first polypeptide comprising a targeting unit, a dimerization unit, and an antigenic unit with T cell epitopes, and a second polypeptide comprising a second targeting unit and a B cell antigen (TECH023-IV003), a free B cell antigen (TECH021-IV001), or a B cell antigen-LZ fusion protein (TECH011-IV003) can elicit broad T and B cell immune responses in vivo.

### Example 7: Design and Production of RSV DNA Plasmids and mRNA Constructs

All gene sequences were ordered from Genscript (Genscript Biotech B.V., Netherlands) and cloned into the expression vector (DNA plasmid) pUMVC4α.

The following RSV DNA plasmids were designed and produced:

### IV12, IV58 and IV59

TABLE 6

| | | | |
|---|---|---|---|
| Elements/Units | IV12 | IV58 | IV59 |

First polypeptide

| | | | |
|---|---|---|---|
| Signal peptide | Native leader sequence | Human Ig VH | Human Ig VH |
| | for human CCL3L1 | SEQ ID NO: 130 | SEQ ID NO: 130 |
| | Amino acids 1-23 of | | |
| | SEQ ID NO: 198 | | |
| Targeting unit | Full length human | Anti-mouse MHCII I-E | Anti-mouse CD40 |
| | CCL3L1 sequence | scFv | scFv |
| | Amino acids 24-93 of | SEQ ID NO: 187 | SEQ ID NO: 297 |
| | SEQ ID NO: 198 | | |

| | |
|---|---|
| Dimerization | Human hinge-region 1 from IgG |
| unit | Amino acids 94-105 of SEQ ID NO: 198 |
| | Human hinge-region 4 from IgG3 |
| | Amino acids 106-120 of SEQ ID NO: 198 |
| | Glycine-serine-linker |
| | Amino acids 121-130 of SEQ ID NO: 198 |
| | Human CH3 domain from IgG3 |
| | Amino acids 131-237 of SEQ ID NO: 198 |
| Unit linker | Glycine-leucine linker |
| | Amino acids 238-242 of SEQ ID NO: 198 |
| Antigenic unit | As described below |
| Linker | GSG |
| Co-expression | T2A |
| element | SEQ ID NO: 8 |

Further polypeptide

| | |
|---|---|
| Signal peptide | Native leader sequence for human CCL3L1 |
| | Amino acids 1-23 of SEQ ID NO: 198 |
| Antigenic unit | As described below |

IV12 comprises a nucleotide sequence encoding:
a first polypeptide comprising the elements/units listed in Table 6, and an antigenic unit comprising a coiled coil peptide A (SEQ ID NO: 194), a GGGGS linker (SEQ ID NO: 195) and the membrane distal head of the PreF protein i447 with a sequence of SEQ ID NO: 196;
the GSG linker and a co-expression element (T2A peptide from Thosea asigna virus capsid protein, (SEQ ID NO: 3)) as listed in Table 6; and
a further polypeptide comprising the signal peptide listed in Table 6, a coiled coil peptide B (SEQ ID NO: 197) and an antigenic unit comprising the same membrane distal head of the PreF protein with a sequence of SEQ ID NO: 196 as comprised in the antigenic unit of the first polypeptide.

IV58 was designed as IV12, but with different signal peptide and targeting unit in the first polypeptide, as listed in Table 6.

IV59 was designed as IV12, but with different signal peptide and targeting unit in the first polypeptide, as listed in Table 6.

The coiled coil peptides A and B promote the heterotrimerization (ABB) of the PreF protein comprised in the first polypeptide and two molecules of PreF protein (further polypeptide) as shown in FIG. 37.

### IV71-IV74

TABLE 7

| | | | | |
|---|---|---|---|---|
| Elements/Units | IV71 | IV72 | IV73 | IV74 |

First polypeptide

| | |
|---|---|
| Signal peptide | Native leader sequence for human CCL3L1 |
| | Amino acids 1-23 of SEQ ID NO: 198 |
| Targeting unit | Full length human CCL3L1 sequence |
| | Amino acids 24-93 of SEQ ID NO: 198 |
| Dimerization | Human hinge-region 1 from IgG |
| unit | Amino acids 94-105 of SEQ ID NO: 198 |
| | Human hinge-region 4 from IgG3 |
| | Amino acids 106-120 of SEQ ID NO: 198 |
| | Glycine-serine-linker |
| | Amino acids 121-130 of SEQ ID NO: 198 |
| | Human CH3 domain from IgG3 |
| | Amino acids 131-237 of SEQ ID NO: 198 |
| Unit linker | Glycine-leucine linker |
| | Amino acids 238-242 of SEQ ID NO: 198 |
| Antigenic unit | RSV T cell epitopes as described below |
| Linker | GSG |
| Co-expression | T2A |
| element | SEQ ID NO: 3 |

Further polypeptide

| Signal peptide | Native leader sequence for RSV F-protein | | | |
|---|---|---|---|---|
| | SEQ ID NO: 199 | | | |
| Antigenic unit | As described | As described | As described | As described |
| | below | below | below | below |

IV71-IV74 comprise a nucleotide sequence encoding:
a first polypeptide comprising elements/units listed in Table 7. The antigenic unit of the first polypeptide comprises RSV T cell epitopes U13, U08, U02, U09, U12, U06 and U11 from various RSV proteins (sequences provided in Table 8) which are separated from each other by GGGGSGGGGS linkers (SEQ ID NO: 29);
a GSG linker and a T2A co-expression element as listed in Table 7; and
a further polypeptide comprising the signal peptide listed in Table 7 and an antigenic unit which is different for all four DNA plasmids:
   IV71: the antigenic unit of the further polypeptide comprises the soluble PreF protein scDs-Cav1 5K61 (SEQ ID NO: 200) and a trimerization unit (C-terminal domain of T4 fibritin, amino acid residues 460-481 in SEQ ID NO: 200) which promotes trimerization of said PreF protein.
   IV72: the antigenic unit of the further polypeptide comprises the soluble PreF protein scDs-Cav1 sc11 (SEQ ID NO: 201) and a trimerization unit (C-terminal domain of T4 fibritin, amino acids 460-481 in SEQ ID NO: 201) which promotes trimerization of said PreF protein.
   IV73: the antigenic unit of the further polypeptide comprises the PreF protein PreF protein scDs-Cav1 sc11 (SEQ ID NO: 202) which comprises a transmembrane domain that promotes cell membrane localization of the protein of the protein to the cell membrane after its expression (amino acids 450-510 in SEQ ID NO: 202).
   IV74: the antigenic unit of the further polypeptide comprises the PreF protein scDs-Cav1 sc11 (SEQ ID NO: 203) which comprises a transmembrane domain that promotes cell membrane localization of the protein of the protein to the cell membrane after its expression (amino acids 452-512 in SEQ ID NO: 203).

TABLE 8

| T cell | | |
|---|---|---|
| epitope | RSV protein | Sequence |
| U13 | Phosphoprotein | MTNDRLEAMARLRNEESEKMAKDTSDEV (SEQ ID NO: 204) |
| U08 | F protein | TVSVGNTLYYV (SEQ ID NO: 205) |
| U02 | NS2 | ATFTFLVNYEMKLLHKVGSTKYKKYTEYNTKYGTFPMPIFINH |
| | | (SEQ ID NO: 206) |
| U09 | Matrix | PADLLIKELANVNILVKQISTPKGPSLRVMINSRSAVLAQM |
| | | (SEQ ID NO: 207) |
| U12 | Matrix | STYTAAVQYNVLEKDDDPASLTIWV (SEQ ID NO: 208) |
| U06 | NS1 | SLSMIKVRLQNLFDNDEVALLKITCY (SEQ ID NO: 209) |
| U11 | Nucleoprotein | AEMEQVVEVYEYAQKLGGEAGFYHILNNPKASLLSL (SEQ ID |
| | | NO: 210) |

An illustration of the first polypeptide (in the form of a dimeric protein) and further polypeptide is provided in FIG. 38.

### IV082-IV085

TABLE 9

| | | | | |
|---|---|---|---|---|
| Elements/Units | IV082 | IV083 | IV084 | IV085 |

First polypeptide

| Signal peptide | Native leader sequence for human CCL3L1 | |
|---|---|---|
| | Amino acids 1-23 of SEQ ID NO: 198 | |
| Targeting unit | Full length human CCL3L1 sequence | |
| | Amino acids 24-93 of SEQ ID NO: 198 | |
| Dimerization | Human hinge-region 1 from IgG | |
| unit | Amino acids 94-105 of SEQ ID NO: 198 | |
| | Human hinge-region 4 from IgG3 | |
| | Amino acids 106-120 of SEQ ID NO: 198 | |
| | Glycine-serine-linker | |
| | Amino acids 121-130 of SEQ ID NO: 198 | |
| | Human CH3 domain from lgG3 | |
| | Amino acids 131-237 of SEQ ID NO: 198 | |
| Unit linker | Glycine-leucine linker | |
| | Amino acids 238-242 of SEQ ID NO: 198 | |
| Antigenic unit | RSV T cell epitopes | As described below |
| | as in IV71-IV74 | |

| | |
|---|---|
| Linker | GSG |
| Co-expression | T2A |
| element | SEQ ID NO: 3 |

Further polypeptide

| Signal peptide | Native leader sequence for RSV F-protein | | | |
|---|---|---|---|---|
| | (SEQ ID NO: 199) | | | |
| Antigenic unit | As described | As described | As described | As described |
| | below | below | below | below |

IV082-IV085 comprise a nucleotide sequence encoding:
a first polypeptide comprising elements/units listed in Table 9;
   IV082 and IV083: the antigenic unit of the first polypeptide of IV082 and of IV083 is identical to that of the first polypeptide of IV71-IV74, i.e. said antigenic unit comprises RSV T cell epitopes U13, U08, U02, U09, U12, U06 and U11 from various RSV proteins (sequences provided in Table 8) which are separated from each other by GGGGSGGGGS linkers (SEQ ID NO: 29);
   IV084 and IV085: the antigenic unit of the first polypeptide of IV084 and of IV085 comprises the DS-Cav1 sc11 PreF protein with a sequence of SEQ ID NO: 201;
a GSG linker and a T2A co-expression element as listed in Table 9; and
a further polypeptide comprising the signal peptide listed in Table 9;
   IV082 and IV084: the antigenic unit of the further polypeptide of IV082 and of IV084 comprises the single chain PreF protein and ferritin as described in K. A. Swanson et al, Sci. Immunol. 5, eaba6466 (2020) (SEQ ID NO: 211);
   IV083 and IV085: the antigenic unit of the further polypeptide of IV083 and of IV085 comprises a PreF protein and ferritin as described in K. A. Swanson et al, Sci. Immunol. 5, eaba6466 (2020) (SEQ ID NO: 212).

Due to the presence of ferritin, the PreF protein (further polypeptide) oligomerizes and a ferritin nanoparticle is formed. An illustration of the first polypeptide (in the form of a dimeric protein) and the ferritin nanoparticle formed by several molecules of the further polypeptide is provided in FIG. 39.

mRNA constructs IV009 and IV010 were purchased from from TriLink BioTechnologies (San Diego, CA, USA) and combined with lipid nanoparticles (LNPs) formulations supplied by Precision Nanosystems Inc. (San Francisco, CA, USA).

IV009 (SEQ ID NO: 294) comprises the same elements/units as IV71 and IV010 (SEQ ID NO: 295) comprises the same elements/units as IV73.

IV009 and IV010 comprise a nucleotide sequence encoding:
a first polypeptide comprising elements/units listed in Table 7. The antigenic unit of the first polypeptide comprises RSV T cell epitopes U13, U08, U02, U09, U12, U06 and U11 from various RSV proteins (sequences provided in Table 8) which are separated from each other by GGGGSGGGGS linkers;
a GSG linker and a T2A co-expression element as listed in Table 7; and
a further polypeptide comprising the signal peptide listed in Table 9 and the following antigenic unit:
   IV009: the antigenic unit of the further polypeptide comprises the soluble PreF protein scDs-Cav1 5K61 (SEQ ID NO: 200) and a trimerization unit (C-terminal domain of T4 fibritin, amino acid residues 460-481 in SEQ ID NO: 200) which promotes trimerization of said PreF protein.
   IV010: the antigenic unit of the further polypeptide comprises the PreF protein scDs-Cav1 sc11 (SEQ ID NO: 201) which comprises a transmembrane domain that anchors the protein to the cell membrane after its expression (amino acid residues 450-510 in SEQ ID NO: 201).

### Example 8: In Vitro Assessment of Expression and Secretion of Proteins Encoded by IV12, IV58 and IV59 DNA Plasmids

The purpose of this study was to determine the protein expression level post transient transfection of mammalian cells with the DNA plasmids IV12, IV58 and IV59 by measuring the presence of secreted proteins in the cell supernatant by an ELISA assay using antibodies detecting the respective targeting units and the dimerization unit. In addition, Western Blot (WB) analysis was performed on supernatant samples from transfected Expi293F cells to characterize the secreted proteins.

Briefly, Expi293F cells (2×10⁶ cells/mL, 1 mL) were seeded in a 96-well culture plate. The cells were transfected with 0.64 μg/mL plasmid DNA using ExpiFectamine 293 Reagent (100014994, Thermo Fisher Sci.), and the plates were incubated on an orbital shaker (3 mm diameter, 850-900 rpm) in a humidified CO₂ cell incubator (8% CO₂, 37° C.). The plates were incubated for 72 h before the supernatant was harvested.

The protein expression levels of proteins expressed from the DNA plasmids and present in the supernatants were determined in an RSV F-protein-specific sandwich ELISA using recombinant human anti-RSV D25 antibody (capture antibody, 100 μL/well, 0.1 μg/mL, PABL-322, Creative Biolabs) and recombinant human anti-RSV Motavizumab biotinylated antibody (detection antibody, 100 μL/well, 0.1 μg/mL, TAB-709, Creative Biolabs) followed by incubation with HRP-conjugated Streptavidin (secondary HRP-conjugated reagent, 100 μL/well, S2438-250UG, Sigma-Aldrich). The amount of protein present in the various cell supernatants was determined from a calibration curve constructed using known concentrations of a purified PreF protein (scDs-Cav1, 5K61, 82007-162, Nexelis).

The results presented in FIG. 40 demonstrate that the polypeptides encoded by IV12, IV58 and IV59 were well expressed and secreted from transfected Expi293F cells.

For the WB analysis, the samples were prepared for SDS-PAGE by mixing 35 μL supernatant from transfected Expi293F cells with 12.5 μL 4× Laemmli sample buffer (Bio-Rad) with 2.5 μL DTT (Thermo Fisher Sci.) or 2.5 μL water for reducing and non-reducing condition, respectively. The samples were heated at 70° C. for 10 minutes (min) before adding 25 μL per lane to 4%-20% Criterion TGX Stain-Free precast gels (Bio-Rad). SDS-PAGE was performed in 1× Tris/Glycine/SDS running buffer (Bio-Rad) with Precision Plus Protein All Blue Prestained and Unstained protein standards (Bio-Rad). Proteins were transferred from the gel onto EtOH activated low fluorescence (LF) 0.45 μm PVDF membranes (Bio-Rad) by using the Trans-Blot Turbo semi-dry transfer system (Bio-Rad). PVDF membranes were blocked in EveryBlot buffer (Bio-Rad) for 5 min and probed with recombinant biotinylated human Motavizumab antibody (1 μg/mL, TAB-709, Creative Biolabs). The membranes were washed, incubated with Dylight 800-conjugated Streptavidin (21851, Thermo Fisher) for 1 h at room temperature, and then washed and dried (rinsed in ethanol). Images were acquired by using a ChemiDoc™ MP Imaging System (setting Dylight 650 and 800, Auto Optimal).

The WB analysis for detection of PreF protein (FIG. 41) showed that both monomeric first polypeptides and dimeric proteins comprised of 2 first polypeptides were expressed from all 3 DNA plasmids at the expected MW (monomer of 72 and dimer of 144 kDa for both IV58 and IV59, and monomer of 53 and dimer of 106 kDa for IV12). Also, for all 3 constructs, the further polypeptide was expressed at the expected MW of 28 kDa.

Taken together, the ELISA and WB data demonstrate efficient secretion and expression of the proteins encoded by IV12, IV58 and IV59, which all comprise different targeting units.

### Example 9: Assessment of T Cell Response Induced by DNA Plasmid IV12

Female, 6-week-old BALB/c mice were obtained from Janvier Labs (France). All animals were housed in the animal facility at the University of Oslo (Oslo, Norway). All animal protocols were approved by the Norwegian Food Safety Authority (Oslo, Norway). 5 mice/group were used for the testing of DNA plasmid IV12, whereas 5 mice/group were used for the negative control (PBS).

A final dose of 25 μg plasmid DNA dissolved in sterile PBS was administered by intramuscular needle injection to each tibialis anterior, followed by electroporation with AgilePulse in vivo electroporation system (BTX, USA). Two doses were administered, one on day 0 and another on day 21.

The spleens were collected on day 35 and mashed in a cell strainer to obtain a single cell suspension. The red blood cells were lysed using ACK lysing buffer. The splenocytes were pooled within the respective groups, counted using the NucleoCounter NC-202 (ChemoMetec) and re-suspended to a final concentration of 4×10⁶ cells/ml. The splenocytes were then seeded culture plates (4×10⁵ cells/well) and re-stimulated for 40-44 hours with 6 μg/mL of peptide pools 2-6 (Table 10) comprising of single peptides corresponding to predicted PreF protein T cell epitopes. The pools were designed based on the sequence of single chain (sc) 11 DS-Cav1 PreF protein from RSV A, overlapping by 10 amino acids. No-peptide-stimulation was used as negative control. The stimulated splenocytes were analyzed for IFN-γ responses using the IFN-γ FluoroSpot kit (Mabtech AB). Spot-forming cells were measured in an IRIS Fluorospot and ELISpot plate reader (Mabtech AB) and analyzed using the Apex software (Mabtech AB). Results are shown as the mean number of IFN-γ⁺ spots/10⁶ splenocytes.

TABLE 10

| Pool ID | Composition |
|---|---|
| Pool 1 | PreF A, pep 1-4: 4 peptides |
| Pool 2 | PreF A, pep 7-15: 9 peptides (Head only) |
| Pool 3 | PreF A, pep 16, 18-26: 10 peptides (Head only) |
| Pool 4 | PreF A, pep 27-29, 31-37: 10 peptides (Head only) |
| Pool 5 | PreF A, pep 38-42, 44, 46-48: 9 peptides (Head only) |
| Pool 6 | PreF A, pep 49-57: 9 peptides (Head only) |
| Pool 7 | PreF A, pep 58-62, 64-65: 7 peptides |
| Pool 8 | PreF A, pep 68-77: 10 peptides |
| Pool 9 | PreF A, pep 78-87: 10 peptides |

The results shown in FIG. 42 demonstrate that T cell responses against the predicted PreF protein T cell epitopes were detected on day 35 in spleens of mice to which 25 μg plasmid DNA (IV12) had been administered twice.

### Example 10: Assessment of Humoral Immune Response Induced in Mice by DNA Plasmid IV12

Sera from mice administered with DNA plasmid IV12 as described in Example 9 or from mice administered with PBS were collected on day 35 and tested for anti-PreF IgG antibodies binding the PreF protein.

Briefly, blood was collected from the saphenous vein of the mice, coagulated blood was centrifuged twice (1000×g, 15 min) and the serum was collected and transferred to a clean tube. The humoral immune response was evaluated in an ELISA assay detecting total IgG in the sera binding to PreF protein. ELISA plates (MaxiSorp Nunc-Immuno plates) were coated with 1 μg/mL recombinant PreF protein antigen in PBS overnight at 4° C. Plates were blocked with 4% BSA in PBS for 1 hour at RT, then incubated with serial dilutions of mouse sera and incubated for 2 h at 37° C. Plates were washed 3× and incubated with 1:50000 dilution of anti-mouse total IgG-HRP antibody (Southern Biotech) and incubated for 1 h at 37° C. After final washing, plates were developed using TMB substrate (CL07-1000, Merck). Plates were read at 450 nm wavelength within 30 min using a SPARK® Multimode Microplate Reader (Tecan). Binding antibody endpoint titers were calculated as the reciprocal of the highest dilution resulting in a signal above the cutoff. PreF protein DS_Cav1 (82007-162, Nexelis) was used as binding antigen.

The results shown in FIG. 43 demonstrate that administration of plasmid DNA (IV12) to mice induced total IgG responses against the PreF protein comprised in the antigenic unit.

### Example 11: In Vitro Assessment of Expression and Secretion of Proteins Encoded by DNA Plasmids IV71, IV72, IV73 and IV74 and Confirmation of the Presence of Membrane-Bound PreF Protein in Expi293F Cells Transfected with IV73 and IV74

The protein expression level post transient transfection of mammalian cells with DNA plasmids IV71, IV72, IV73 and IV74 was measured by quantifying the presence of secreted proteins in the cell supernatant by an ELISA assay. WB analysis was performed on supernatant and lysate samples from transfected Expi293F cells to characterize the secreted and expressed proteins, respectively.

Expi293F cells were seeded, transfected and incubated as described in Example 8, supernatants and lysates were harvested after 72 hours. The expression levels of the first polypeptide expressed from the DNA plasmids and present in the supernatants were determined in a sandwich ELISA using mouse anti human IgG CH3 domain antibody (capture antibody, 100 L/well, 2 μg/mL, MCA878G, Bio-Rad), and goat anti-human CCL3/MIP-1α biotinylated antibody (detection antibody, 100 μL/well, 0.2 μg/mL, BAF270, R&D Systems) followed by incubation with HRP-conjugated Streptavidin (secondary HRP-conjugated reagent, 100 μL/well, S2438-250UG, Sigma-Aldrich). A calibration curve was constructed using known concentrations of a purified control protein (VB4219, consisting of the targeting unit and dimerization unit of IV71-IV74 (as listed in Table 7)) and the amount of protein present in the various cell supernatants was determined from said calibration curve.

The secretion level of the further polypeptides (PreF proteins) expressed from DNA plasmids IV71-IV72 was characterized in an RSV F protein-specific sandwich ELISA of supernatant using recombinant human anti-RSV D25 antibody (capture antibody, 100 μL/well, 0.1 μg/mL, PABL-322, Creative Biolabs) and recombinant human anti-RSV Motavizumab biotinylated antibody (detection antibody, 100 μL/well, 0.1 μg/mL, TAB-709, Creative Biolabs) followed by incubation with HRP-conjugated Streptavidin (secondary HRP-conjugated reagent, 100 μL/well, S2438-250UG, Sigma-Aldrich). The amount of protein present in the various cell supernatants was determined from a calibration curve constructed using known concentrations of a PreF protein containing mutations to stabilize the prefusion conformation (scDS-Cav1, 5K61, 82007-162, Nexelis).

FIG. 44 shows that the further polypeptide encoded by IV71 and IV72 (soluble RSV PreF antigen) were well secreted. FIG. 45 shows that the first polypeptides encoded by IV71-IV74 were expressed and secreted from transfected Expi293F.

For the WB analysis, the samples were prepared for SDS-PAGE by mixing 35 μl supernatant (IV71 and IV72) or lysate (IV73 and IV74) from transfected Expi293F cells with 12.5 μl 4× Laemmli sample buffer (Bio-Rad) with 2.5 μL 1 M DTT (Thermo Fisher Sci.) for reducing condition. The recombinant PreF protein (Nexelis, 7.43 μg/mL) was diluted to a final concentration of 1 ng/μL in PBS (20 μL loaded on gel). The samples were heated at 80° C. for 10 minutes (min) before adding 25 μL or 30 μL per lane to 18-well and 12+2-well 4%-20% Criterion TGX Stain-Free precast gels (Bio-Rad), respectively. SDS-PAGE was performed in 1× Tris/Glycine/SDS running buffer (Bio-Rad) with Precision Plus Protein All Blue Prestained and Unstained protein standards (Bio-Rad). Proteins were transferred from the gel onto EtOH activated low fluorescence (LF) 0.45 μm PVDF membranes (Bio-Rad) by using the Trans-Blot Turbo semi-dry transfer system (Bio-Rad). PVDF membranes were blocked in EveryBlot buffer (Bio-Rad) for 5 min and probed with goat anti-human MIP1a detection antibody (0.2 μg/mL, AF270, R&D Systems) or recombinant biotinylated human Motavizumab antibody (1 μg/mL, TAB-709, Creative Biolabs). The membranes were washed, incubated with Dylight 800-conjugated donkey anti-goat secondary antibody (SA5-10092, Thermo Fisher) or Dylight 800-conjugated Streptavidin (21851, Thermo Fisher) for 1 h at room temperature, and then washed and dried. Images were acquired by using a ChemiDoc™ MP Imaging System (setting Dylight 650 and 800, Auto Optimal).

The WB analysis shows that the PreF protein (further polypeptide) was detected at the expected MW of around 70 kDa (FIG. 46). Also, the WB analysis for detection of the hCCL3L1 targeting unit (FIG. 47) showed that monomers (reduced/non reduced) and dimeric proteins (non-reduced) of all first polypeptides IV71-IV74 were detected at the expected MWs (monomer of 51.4 kDa and dimer of 102.8 kDa).

Cell surface expression of membrane-bound PreF antigen was assessed after transient transfection of Expi293F cells with DNA plasmids IV71-IV74 (IV71 and IV72 were included as negative controls) using flow cytometry. Expi293F cells were seeded, transfected and incubated as described in Example 8. The transfected cells were transferred to a new 96-well plate (5×10⁵ cells/well), washed once in 1×PBS (BioWest), and re-suspended in 50 μL of Fixable viability dye eFluor™ 506 diluted 1:500 in 1×PBS (65-0866-14, eBiosience). The cells were incubated for 20 min on ice in the dark. At the end of the incubation, the cells were washed with FACS buffer (1×PBS with 2% FBS and 2 mM EDTA (VWR)) and then re-suspended in 50 μL biotinylated D25 Fab antibody (recombinant anti-RSV-F protein antibody Fab antibody fragment 2.5 μg/mL, PFBL-322, Creative BioLabs) or Motavizumab antibody (recombinant anti-RSV-F protein antibody, 2.5 μg/mL, TAB-709, Creative BioLabs) and incubated for 20 min on ice in the dark. At the end of the incubation, the cells were washed with FACS buffer, re-suspended in 50 μL Streptavidin-Alexa Fluor 647 (0.625 μg/mL, 405237, BioLegend) and incubated 15 min on ice in the dark. The cells were then washed with FACS buffer and fixed in 50 μL BD Cytofix Fixation Buffer (554655, BD Biosciences) for 20 min on ice in the dark, then washed, re-suspended in FACS buffer, and stored at 4° C. until sample acquisition. The samples were acquired within a week using a BD FACSymphony™ A3 cytometer (BD Biosciences) and further analyzed using FlowJo software (version 10.9, BD Biosciences).

The results presented in FIG. 48A show that transient transfection of Expi293F with plasmids IV73 or IV74 resulted in the expression of membrane-bound F protein on Expi293F cell surfaces, which could be detected using Motavizumab antibody which recognizes the antigenic site II of the RSV F protein. Similarly, expression of membrane-bound F protein on Expi293F cell surfaces could be detected using D25 Fab antibody (FIG. 48B), which recognizes the antigenic site Ø of the RSV F protein, confirming the expression of the membrane-bound RSV F protein in a prefusion conformation.

### Example 12: Assessment of T Cell Response Induced by DNA Plasmids IV71-IV74

Female, 6-week-old BALB/c mice were obtained from Janvier Labs (France). For animal housing and approval of animal protocols see Example 8. 6 mice/group were used for the testing of the DNA plasmids, whereas 5 mice/group were used for the negative control (PBS) and 6 mice/group were used for a comparison, wherein said mice were administered with 10 μg of DS-Cav1 protein, a PreF protein with mutations stabilizing the prefusion conformation (Nexelis, 82007-162), in adjuvanted Addavax medium (InvivoGen, vac-adx-10).

DNA plasmids were administered to animals as described in Example 8. Spleens were collected, processed, pooled and counted as described in Example 8.

The splenocytes were then seeded with 4×10⁵ cells/well and re-stimulated for 40-44 hours with 6 μg/ml of PreF peptide pools 1-9 (Table 10), as described in Example 9, and peptide pools 2, 6, 8, 9, 11, 12 and 13 comprised of T cell epitopes derived from other RSV proteins (Tables 11 and 12). No-peptide-stimulation was used as negative control. The stimulated splenocytes were analyzed for IFN-γ responses using the IFN-γ FluoroSpot kit (Mabtech AB, Sweden). Spot-forming cells were measured in an IRIS Fluorospot and ELISpot plate reader (Mabtech AB) and analyzed using the Apex software (Mabtech AB). Results are shown as the mean number of IFN-γ+ spots/10⁶ splenocytes.

TABLE 11

| Pool ID | Composition (RSV protein) |
|---|---|
| RSV T cell epitope pool 02 | T pep 2A-2G: 7 peptides (NS2) |
| RSV T cell epitope pool 06 | T pep 6A-D: 4 peptides (NS1) |
| RSV T cell epitope pool 08 | T pep 8: 1 peptide (G) |
| RSV T cell epitope pool 09 | T pep 9A-G: 7 peptides (M) |
| RSV T cell epitope pool 11 | T pep 11A-F: 6 peptides (N) |
| RSV T cell epitope pool 12 | T pep 12A-C: 3 peptides (M) |
| RSV T cell epitope pool 13 | T pep 13A-D: 4 peptides (P) |

TABLE 12

| PreF T cell | |
|---|---|
| epitopes | Sequence |
| T Pep2-A | ATFTFLVNYEMKLLH (SEQ ID NO: 213) |
| T Pep2-B | LVNYEMKLLHKVGST (SEQ ID NO: 214) |
| T Pep2-C | MKLLHKVGSTKYKKY (SEQ ID NO: 215) |
| T Pep2-D | KVGSTKYKKYTEYNT (SEQ ID NO: 216) |
| T Pep2-E | KYKKYTEYNTKYGTF (SEQ ID NO: 217) |
| T Pep2-F | TEYNTKYGTFPMPIF (SEQ ID NO: 218) |
| T Pep2-G | KYGTFPMPIFINH (SEQ ID NO: 219) |
| T Pep6 | SLSMIKVRLQNLFDNDEVALLKITCY (SEQ ID |
| | NO: 220) |
| T Pep6-A | SLSMIKVRLQNLFDN (SEQ ID NO: 221) |
| T Pep6-B | KVRLQNLFDNDEVAL (SEQ ID NO: 222) |
| T Pep6-C | NLFDNDEVALLKITC (SEQ ID NO: 223) |
| T Pep6-D | DEVALLKITCY (SEQ ID NO: 224) |
| T Pep8 | TVSVGNTLYYV (SEQ ID NO: 225) |
| T Pep9-A | PADLLIKELANVNIL (SEQ ID NO: 226) |
| T Pep9-B | IKELANVNILVKQIS (SEQ ID NO: 227) |
| T Pep9-C | NVNILVKQISTPKGP (SEQ ID NO: 228) |
| T Pep9-D | VKQISTPKGPSLRVM (SEQ ID NO: 229) |
| T Pep9-E | TPKGPSLRVMINSRS (SEQ ID NO: 230) |
| T Pep9-F | SLRVMINSRSAVLAQ (SEQ ID NO: 231) |
| T Pep9-G | INSRSAVLAQM (SEQ ID NO: 232) |
| T Pep11-A | AEMEQVVEVYEYAQK (SEQ ID NO: 233) |
| T Pep11-B | VVEVYEYAQKLGGEA (SEQ ID NO: 234) |
| T Pep11-C | EYAQKLGGEAGFYHI (SEQ ID NO: 235) |
| T Pep11-D | LGGEAGFYHILNNPK (SEQ ID NO: 236) |
| T Pep11-E | GFYHILNNPKASLLS (SEQ ID NO: 237) |
| T Pep11-F | LNNPKASLLSL (SEQ ID NO: 238) |
| T Pep12 | STYTAAVQYNVLEKDDDPASLTIWV (SEQ ID |
| | NO: 239) |
| T Pep12-A | STYTAAVQYNVLEKD (SEQ ID NO: 240) |
| T Pep12-B | AVQYNVLEKDDDPAS (SEQ ID NO: 241) |
| T Pep12-C | VLEKDDDPASLTIWV (SEQ ID NO: 242) |
| T Pep13 | MTNDRLEAMARLRNEESEKMAKDTSDEV |
| | (SEQ ID NO: 243) |
| T Pep13-A | MTNDRLEAMARLRNE (SEQ ID NO: 244) |
| T Pep13-B | LEAMARLRNEESEKM (SEQ ID NO: 245) |
| T Pep13-C | RLRNEESEKMAKDTS (SEQ ID NO: 246) |
| T Pep13-D | ESEKMAKDTSDEV (SEQ ID NO: 247) |

The results shown in FIG. 49 demonstrate that T cell responses against predicted PreF T cell epitopes were detected on day 35 in spleens of mice to which 25 μg of DNA from either IV71, IV72, IV73 or IV74 had been administered twice. Furthermore, as shown in FIG. 50, these DNA plasmids induce both CD8+ and CD4+RSV specific T cells, with a higher predominance of the former.

### Example 13: Assessment of Humoral Immune Response Induced in Mice by DNA Plasmids IV71-IV74

Sera from the mice administered with DNA plasmids IV71, IV72, IV73, IV74, DS-Cav1 or PBS as described in Example 12 were collected on day 35 and tested for anti-PreF IgG antibodies binding the PreF protein. The test was carried out as described in Example 10.

The results shown in FIG. 51 demonstrate that all four DNA plasmids induced total IgG responses against the PreF protein comprised in the antigenic unit of the respective plasmids, and that humoral response increased significantly in mice administered with DNA from IV71 and IV73 (and DS-Cav1 protein).

### Example 14: Assessment of Neutralizing Antibodies Induced in Mice by DNA Plasmids IV71-IV74

To determine if the antibodies raised against IV74-IV74 in mice can neutralize viral infection in vitro, the sera collected in Example 13 were sent to and evaluated by Nexelis (Laval, Canada).

Vero cells were seeded in 96-well plates at 1.2×104 per well and incubated overnight at 5% CO₂, 37° C.). At the day of testing, the seeded Vero cells were at least 80% confluent prior to testing. Sera samples were inactivated at 56° C. for 30 min prior testing. Palivimuzab, a therapeutic monoclonal antibody against severe disease caused by RSV, was used as positive control. Tests of serum samples and positive controls were done in duplicates and each plate included in addition to positive control also a virus control and a cell control. The positive control was diluted in tubes with virus growth medium. Serum samples and positive control were loaded in round bottom 96-well plates (dilution plates) and 2-fold serially diluted in virus growth medium to a volume of 30 μl/well. RSV stocks were diluted at the pre-determined working dilution in virus growth medium and 30 μl was loaded to each well of the dilution plate. Plates were incubated for 60 minutes at 5% CO₂, 33° C. 50 μl of serum-virus mixture and of the controls were transferred from the dilution plates into cell seeded plates (test plates). The test plates were emptied and rinsed prior to this step. The test plates were then incubated for 2 h at 5% CO₂, 33° C. Serum-virus mixtures and controls were removed from the test plates and 200 μl of maintenance medium containing an overlay agent (carboxymethyl cellulose) were added to each well. The test plates were incubated for 3 days at 5% CO₂, 33° C. After incubation, cells were rinsed once with PBS and fixed with 4% paraformaldehyde solution for 15 min at room temperature. The plates were emptied and immune-stained as follows: primary antibody (anti-RSV PreF antibody): incubation 1 h at, 37° C., secondary antibody HRP (horseradish peroxidase)-conjugated: incubation 30 min at 33° C.; addition of 3,3′,5,5′-tetramethylbenzidine solution for 10 min in the dark. Viral particle forming units (PFUs) were detected by image acquisition using an automated microscope system. Viral PFUs were counted using ICARUS software and then neutralizing titers were calculated.

The results shown in FIG. 52 demonstrate that all four constructs IV71-IV74 induced neutralizing antibodies responses in mice administered with the plasmids, with levels of neutralizing antibodies induced by IV71, IV72 and IV73 being similar to that induced by the palivimuzab positive control. This indicates that the presence of the T cell epitope-containing antigenic unit in the first polypeptide does not influence the antibody response elicited against the PreF protein comprised in the antigenic unit of the further polypeptide. All four constructs induced lower levels of neutralizing antibodies than those induced by the Ds-Cav1 protein, however, it needs to be noted that 2×10 μg Ds-Cav1 protein was administered. In a recent publication by Ye Che et al, relating to the design of a highly immunogenic prefusion-stabilized F antigen for a RSV vaccine, DS-Cav1 was used immunogen in the same mouse model at a maximum dose of 2×0.25 μg/mouse, i.e. at a dose which was 40 times lower (Y. Che et al., Sci Transl Med 15(693), 2023). At that dose, similar levels of neutralizing antibodies were obtained with DS-Cav1 as those with IV71-IV74.

### Example 15: In Vitro Assessment of Expression and Secretion of Proteins Encoded by DNA Plasmids IV082, IV083, IV084 and IV085

The protein expression level post transient transfection of mammalian cells with DNA plasmids IV082, IV083, IV084 and IV085 was measured by quantifying the presence of proteins in the cell supernatant by an ELISA assay. WB analysis was performed on supernatant samples from transfected Expi293F cells to characterize the secreted proteins.

Expi293F cells were seeded, transfected and incubated as described in Example 8, supernatants and lysates were harvested after 72 hours.

The expression levels of the first polypeptides expressed from the DNA plasmids and present in the supernatants were determined in a sandwich ELISA as described in Example 11. As shown in FIGS. 53A and B, the first polypeptide could be detected in the supernatant.

The expression levels of the further polypeptides (PreF proteins) expressed from DNA plasmids IV084 and IV085 and present in the supernatants were determined in a sandwich ELISA as described in Example 11. The results shown in FIG. 54 demonstrate that the further polypeptides were very well secreted (dilution 1:500 for the darker bars and 1:1000 for the lighter bars).

For the WB analysis, the samples were prepared for SDS-PAGE by using supernatant from transfected Expi293F cells for reducing conditions as described in Example 11. The WB analysis for detection of the hCCL3L1 targeting unit comprised in the first polypeptide (FIG. 55) shows that the first polypeptide encoded by each of the 4 constructs is expressed and secreted at the expected MW (monomer of 53.4 kDa for both IV082 and IV083, and monomer of 76 kDa for both IV084 and IV085, respectively). Further, the WB analysis for detection of the PreF protein shows that the further polypeptide encoded by each of the 4 constructs is expressed and secreted at the expected MW 69.3-69.8 kDa for IV082 and IV083 (FIG. 56), however, the presence of PreF protein in the first polypeptide and further polypeptide expressed from IV084 and IV085 makes the detection of said further polypeptide difficult.

Furthermore, the native conformation of the further polypeptide nanoparticles was assessed by blue native PAGE and WB analysis of supernatants from transfected Expi293F cells. Prior to sample preparation for native PAGE, supernatants were concentrated 3.4-fold (IV082, IV083) or 6.9-fold (IV084, IV085) using Vivaspin® Turbo 4.5 kDa molecular weight cut-off (MWCO) (VS04T31, Sartorius). As a control, non-concentrated supernatants from Expi293F cells transfected with a DNA plasmid (pUMVC4a) comprising a sequence encoding only the further polypeptide of IV082/IV04 or IV03/IV85 (denoted IV080 and IV081, respectively) were included. All supernatant samples were prepared by mixing 75 μL supernatant (concentrated or non-concentrated) with 25 μL 4× NativePAGE sample buffer (Invitrogen). Then, 25 μL of sample was loaded per lane to 4-16% NativePAGE, Bis-Tris, 1.0 mm, Mini Protein Gels (Invitrogen). Blue native PAGE was performed in 1× NativePAGE Light Blue Cathode Buffer (Invitrogen) and 1× NativePAGE Anoge Buffer (Invitrogen) using the XCell SureLock Mini-Cell (Life Technologies). NativeMark Protein standard (Invitrogen) was used as marker. Proteins were run at 150 V for 240 min using the ZOOM Dual Power Supply (Invitrogen) at 4 C. Proteins were transferred from the gel onto an EtOH-activated Invitrolon 0.45 μm PVDF membrane (Invitrogen) by wet transfer in NuPAGE Transfer buffer using the XCell II Blot Module (Invitrogen). After transfer, the membrane was incubated in 8% acetic acid for 15 min to fix the proteins. The membrane was first washed in MilliQ water and air dried, then washed in EtOH and air dried (three times). After the last step, the membrane was rinsed in MilliQ water before immunoblotting. For immunoblotting, the membrane was blocked for 5 min in EveryBlot Blocking buffer and probed with recombinant biotinylated human Motavizumab antibody (1 μg/mL, TAB-709, Creative Biolabs). The membrane was washed, incubated with Poly HRP-conjugated Streptavidin (21140, Thermo Fisher) for 1 h at room temperature, and then washed and dried (rinsed in ethanol). Images were acquired by ECL detection (1705062, Bio-Rad) using a ChemiDoc™ MP Imaging System.

The results of the WB analysis for detection of the further polypeptide (PreF protein)-ferritin nanoparticles are shown in FIG. 57. The further polypeptide was expressed and secreted from all four constructs, and 24-mer ferritin-nanoparticles were formed as determined by the corresponding band at 1663.2 kDa for IV082 and IV084, and 1675.2 kDa for IV083 and IV085, respectively.

### Example 16 In Vitro Assessment of Expression and Secretion of Proteins Encoded by IV009 and IV010 mRNA Constructs

The protein expression level after transient transfection of Expi293F cells with mRNA constructs IV009 and IV010 was measured by quantifying the presence of secreted proteins in the cell supernatant by an ELISA assay. WB analysis was performed on supernatant samples from transfected Expi293F cells to characterize the secreted proteins.

Expi293F cells were seeded and incubated as described in Example 8; supernatants were harvested after 6, 24, 48 and 72 hours. No Expifectamine was used since the LNPs are able to transfect the cells without addition of a transfection agent. For transfection, 1000 ng of mRNA construct per 2×10⁶ Expi293F cells was used.

The expression levels of the first polypeptide and further polypeptides (PreF proteins) expressed from the RNA constructs and present in the supernatants were determined as described in Example 11.

FIGS. 61 and 62 show that the first (FIG. 61) and further (FIG. 62) polypeptides encoded by IV009 and IV010 were expressed and secreted from transfected Expi293F cells at good levels. As expected, the further polypeptide was not detected in the supernatant of Expi293F cells transfected with IV010, since the PreF protein in the antigenic unit of said construct comprises a transmembrane domain that anchors the protein to the cell membrane after its expression.

For the WB analysis, the samples were prepared for SDS-PAGE from supernatant from transfected Expi293F cells as described in Example 11, however, PVDF membranes were probed only with goat anti-human MIP1α detection antibody (0.2 μg/mL, AF270, R&D Systems) after blocking.

The WB analysis shows that first polypeptide was detected at the expected MW of around 53 kDa (FIG. 63). The bands' intensity increased with time after transfection, implying a greater protein quantity.

### Example 17: Assessment of T Cell Response Induced by mRNA Constructs IV009 and IV010

The assessment of T cell responses induced by mRNA constructs IBV009 and IV010 was carried out as described in Example 12, however, two doses of either 0.1 μg or 1 μg of mRNA construct were administered on days 0 and 21, and the spleens were collected on day 36.

The results shown in FIG. 64 demonstrate that T cell responses against predicted PreF T cell epitopes were detected on day 36 in spleens of mice to which either 0.1 μg or 1 μg of mRNA construct had been administered twice and that the T cell responses were dose-dependent.

### Example 18: Assessment of Humoral Immune Response Induced in Mice by mRNA Constructs IV009 and IV010

Sera from the mice administered with mRNA constructs IV009 and IV010, PBS (negative control) and DS-Cav1 protein (comparison) as described in Example 17 were collected on days 15 and 36 and tested for anti-PreF IgG antibodies binding the PreF protein. The test was carried out as described in Example 10.

The results are shown in FIG. 65. The humoral immune responses on day 36 were higher than those on day 16 within groups that were administered with the same dose, for both mRNA constructs and DS-Cav1 protein, i.e. as expected, antibody responses increased in all groups after the second administration on day 21. Similar to T cell responses, higher doses of mRNA constructs (and higher doses of DS-Cav1 protein) induced higher humoral responses against PreF protein. While administration of DS-Cav1 protein induced higher humoral responses compared to both IV009 and IV010, the difference was not statistically significant.

### REFERENCES

Alvarez-Cienfuegos, Ana et al. “Intramolecular trimerization, a novel strategy for making multispecific antibodies with controlled orientation of the antigen binding domains.” Scientific reports vol. 6 28643. 27 Jun. 2016, doi:10.1038/srep28643 Andersson, Helen A, and Michael A Barry. “Maximizing antigen targeting to the proteasome for gene-based vaccines.” Molecular therapy: the journal of the American Society of Gene Therapy vol. 10,3 (2004): 432-46. doi:10.1016/j.ymthe.2004.05.035
Asimaki, Evrydiki et al. “Fast friends—Ubiquitin-like modifiers as engineered fusion partners.” Seminars in cell & developmental biology, S1084-9521(21)00289-5. 25 Nov. 2021, doi:10.1016/j.semcdb.2021.11.013
Békés, Miklós et al. “DUB-resistant ubiquitin to survey ubiquitination switches in mammalian cells.” Cell reports vol. 5,3 (2013): 826-38. doi:10.1016/j.celrep.2013.10.008
Blount, Jessica R et al. “Expression and Regulation of Deubiquitinase-Resistant, Unanchored Ubiquitin Chains in Drosophila.” Scientific reports vol. 8,1 8513. 31 May. 2018, doi:10.1038/s41598-018-26364-x
Cousens et al.: “Tregitope: Immunomodulation Powerhouse”, Human Immunology, Volume 75, Issue 12, December 2014, Pages 1139-1146
Craig, Patricio O et al. “Polymeric Display of Proteins through High Affinity Leucine Zipper Peptide Adaptors.” Biomacromolecules vol. 13,4 (2012): 1112-21. doi:10.1021/bm201875p
Curin M, Khaitov M, Karaulov A, Namazova-Baranova L, Campana R, Garib V, Valenta R. Next-Generation of Allergen-Specific Immunotherapies: Molecular Approaches. Curr Allergy Asthma Rep. 2018 Jun. 9; 18(7):39. doi:10.1007/s11882-018-0790-x.
Curley, Stephanie M, and David Putnam. “Biological Nanoparticles in Vaccine Development.” Frontiers in bioengineering and biotechnology vol. 10 867119. 23 Mar. 2022, doi:10.3389/fbioe.2022.867119
Golding, M C, and M R W Mann. “A bidirectional promoter architecture enhances lentiviral transgenesis in embryonic and extraembryonic stem cells.” Gene therapy vol. 18,8 (2011): 817-26. doi:10.1038/gt.2011.26
Glasgow, Joel N et al. “A strategy for adenovirus vector targeting with a secreted single chain antibody.” PloS one vol. 4, 12e8355. 21 Dec. 2009, doi:10.1371/journal.pone.0008355
Grifoni, Alba et al. “SARS-COV-2 human T cell epitopes: Adaptive immune response against COVID-19.” Cell host & microbe vol. 29,7 (2021): 1076-1092. doi:10.1016/j.chom.2021.05.010
He, Kevin et al. “Compact Bidirectional Promoters for Dual-Gene Expression in a Sleeping Beauty Transposon.” International journal of molecular sciences vol. 21,23 9256. 4 Dec. 2020, doi:10.3390/ijms21239256
Houser, Katherine V et al. “Safety and immunogenicity of a ferritin nanoparticle H2 influenza vaccine in healthy adults: a phase 1 trial.” Nature medicine vol. 28,2 (2022): 383-391. doi:10.1038/s41591-021-01660-8
Kang et al.: “Low-Dose Peptide Tolerance Therapy of Lupus Generates Plasmacytoid Dendritic Cells That Cause Expansion of Autoantigen-Specific Regulatory T Cells and Contraction of Inflammatory Th17 Cells”, J Immunol Jun. 15, 2007, 178 (12) 7849-7858
Kim, Dongwook et al. “Tribody: robust self-assembled trimeric targeting ligands with high stability and significantly improved target-binding strength.” Biochemistry vol. 52,41 (2013): 7283-94. doi:10.1021/bi400716w
Joyce, M Gordon et al. “A SARS-COV-2 ferritin nanoparticle vaccine elicits protective immune responses in nonhuman primates.” Science translational medicine vol. 14,632 (2022): eabi5735. doi:10.1126/scitranslmed.abi5735
Linhart B, Valenta R. Mechanisms underlying allergy vaccination with recombinant hypoallergenic allergen derivatives. Vaccine. 2012 Jun. 19; 30(29):4328-35. doi: 10.1016/j.vaccine.2011.11.011.
Litowski, Jennifer R, and Robert S Hodges. “Designing heterodimeric two-stranded alpha-helical coiled-coils. Effects of hydrophobicity and alpha-helical propensity on protein folding, stability, and specificity.” The Journal of biological chemistry vol. 277,40 (2002): 37272-9. doi:10.1074/jbc.M204257200
López-Sagaseta, Jacinto et al. “Self-assembling protein nanoparticles in the design of vaccines.” Computational and structural biotechnology journal vol. 14 58-68. 26 Nov. 2015, doi:10.1016/j.csbj.2015.11.001
Kiyokawa, Tomohiro et al. “Selective formation of AAB- and ABC-type heterotrimeric alpha-helical coiled coils.” Chemistry (Weinheim an der Bergstrasse, Germany) vol. 10,14 (2004): 3548-54. doi:10.1002/chem.200305729
Ma, Xiancai et al. “Nanoparticle Vaccines Based on the Receptor Binding Domain (RBD) and Heptad Repeat (HR) of SARS-COV-2 Elicit Robust Protective Immune Responses.” Immunity vol. 53,6 (2020): 1315-1330.e9. doi:10.1016/j.immuni.2020.11.015
Moll, J R et al. “Designed heterodimerizing leucine zippers with a ranger of pls and stabilities up to 10(-15) M.” Protein science: a publication of the Protein Society vol. 10,3 (2001): 649-55. doi:10.1110/ps.39401
Mu, Zekun et al. “mRNA-encoded HIV-1 Env trimer ferritin nanoparticles induce monoclonal antibodies that neutralize heterologous HIV-1 isolates in mice.” Cell reports vol. 38,11 (2022): 110514. doi:10.1016/j.celrep.2022.110514
Nautiyal, S, and T Alber. “Crystal structure of a designed, thermostable, heterotrimeric coiled coil.” Protein science: a publication of the Protein Society vol. 8,1 (1999): 84-90. doi:10.1110/ps.8.1.84
Pearson, W R. “Flexible sequence similarity searching with the FASTA3 program package.” Methods in molecular biology (Clifton, N.J.) vol. 132 (2000): 185-219. doi:10.1385/1-59259-192-2:185
Picarda, E. et al: “Cross-Reactive Donor-Specific CD8+ Tregs Efficiently Prevent Transplant Rejection” Cell Reports Volume 29, Issue 13, 24 Dec. 2019, Pages 4245-4255.e6
Qu, Yiran et al. “Immunogenicity study of engineered ferritins with C- and N-terminus insertion of Epstein-Barr nuclear antigen 1 epitope.” Vaccine vol. 39,34 (2021): 4830-4841. doi:10.1016/j.vaccine.2021.07.021
Rappuoli, Rino, and Davide Serruto. “Self-Assembling Nanoparticles Usher in a New Era of Vaccine Design.” Cell vol. 176,6 (2019): 1245-1247. doi:10.1016/j.cell.2019.02.008
Rodrigues, Margarida Q et al. “Functionalizing Ferritin Nanoparticles for Vaccine Development.” Pharmaceutics vol. 13,10 1621. 5 Oct. 2021, doi:10.3390/pharmaceutics13101621
Sladitschek, Hanna L, and Pierre A Neveu. “Bidirectional Promoter Engineering for Single Cell MicroRNA Sensors in Embryonic Stem Cells.” PloS one vol. 11,5 e0155177. 6 May. 2016, doi:10.1371/journal.pone.0155177
Wai C Y, Leung N Y, Ho M H, Gershwin L J, Shu S A, Leung P S, Chu K H. Immunization with Hypoallergens of shrimp allergen tropomyosin inhibits shrimp tropomyosin specific IgE reactivity. PLoS One. 2014 Nov. 3; 9(11):e111649. doi: 10.1371/journal.pone.0111649.
Walseng, Even et al. “Soluble T cell receptors produced in human cells for targeted delivery.” PloS one vol. 10,4 e0119559. 13 Apr. 2015, doi:10.1371/journal.pone.0119559
Walls, Alexandra C et al. “Elicitation of Potent Neutralizing Antibody Responses by Designed Protein Nanoparticle Vaccines for SARS-COV-2.” Cell vol. 183,5 (2020): 1367-1382.e17. doi:10.1016/j.cell.2020.10.043
Wang, Bo et al. “Self-assembling ferritin nanoparticles coupled with linear sequences from canine distemper virus haemagglutinin protein elicit robust immune responses.” Journal of nanobiotechnology vol. 20,1 32. 10 Jan. 2022, doi:10.1186/s12951-021-01229-0
Weinberger E E, Himly M, Myschik J, Hauser M, Altmann F, Isakovic A, Scheiblhofer S, Thalhamer J, Weiss R. Generation of hypoallergenic neoglycoconjugates for dendritic cell targeted vaccination: a novel tool for specific immunotherapy. J Control Release. 2013 Jan. 28; 165(2):101-9. doi:10.1016/j.jconrel.2012.11.002.
Wichgers Schreur, Paul J et al. “Vaccine Efficacy of Self-Assembled Multimeric Protein Scaffold Particles Displaying the Glycoprotein Gn Head Domain of Rift Valley Fever Virus.” Vaccines vol. 9,3 301. 23 Mar. 2021, doi:10.3390/vaccines9030301
Zottig, Ximena et al. “Protein Supramolecular Structures: From Self-Assembly to Nanovaccine Design.” Nanomaterials (Basel, Switzerland) vol. 10,5 1008. 25 May. 2020, doi:10.3390/nano10051008
WO2004/076489 A1
WO2011/161244 A1
WO 2013/092875 A1
WO 2017/118695 A1
WO 2019/048928
WO 2020/065023A1
WO 2020/176797A1
WO 2020/221783A1
WO 2021/205027 A1
WO 2021/219897 A1
WO 2022/013277 A1
US2019/0022202A1
PCT/EP2022/057955
PCT/EP2022/061819

### Items—1

1. A vector comprising:
   (a) a first nucleic acid sequence encoding a first polypeptide, wherein the first polypeptide comprises a targeting unit that targets antigen-presenting cells, a multimerization unit, such as a dimerization unit, and an antigenic unit comprising one or more epitopes; and
   (b) one or more further nucleic acid sequences encoding one or more further polypeptides, wherein the one or more further polypeptides comprise a further antigenic unit comprising one or more further epitopes, wherein the vector allows for the co-expression of the first polypeptide and the one or more further polypeptides as separate molecules.
2. The vector according to item 1, wherein the one or more epitopes and/or the one or more further epitopes are selected from the group consisting of non-self epitopes, non-self antigens or parts thereof, disease relevant epitopes and disease relevant antigens or parts thereof.
3. The vector according to any one of the previous items, wherein the one or more epitopes and/or the one or more further epitopes are cancer associated, such as selected from the group consisting of tumor associated, tumor specific, patient-present shared, patient-present specific, neoantigens and neoepitopes.
4. The vector according to any one of the previous items, wherein the one or more epitopes and/or the one or more further epitopes are derived from one or more cancer antigens, wherein said cancer antigens are selected from the group consisting of cancer antigens associated with multiple myeloma or lymphoma, malignant melanoma, HPV induced cancers, prostate cancer, breast cancer, lung cancer, ovarian cancer, and/or liver cancer.
5. The vector according to any one of the previous items, wherein the one or more epitopes and/or the one or more further epitopes are derived from one or more pathogen, wherein the one or more pathogens are selected from the group consisting of viruses, bacteria, fungi and parasites.
6. The vector according to any one of the previous items, wherein the antigenic unit of the first polypeptide comprises one or more epitopes, wherein the one or more epitopes are comprised in one or more antigens and/or wherein the further antigenic unit of at least one of the one or more further polypeptides comprises one or more further epitopes, wherein the one or more further epitopes are comprised in one or more further antigens.
7. The vector according to any one of the previous items, wherein the further antigenic unit of at least one of the one or more further polypeptides comprises one or more full length proteins, wherein said one or more full length proteins comprises the one or more further epitopes.
8. The vector according to any one of the previous items, wherein the further antigenic unit of at least one of the one or more further polypeptides comprises an antigen or a part thereof, wherein said antigen or a part thereof comprises the one or more further epitopes.
9. The vector according to any one of the previous items, wherein the vector induces a CD4+ T cell response which is HLA independent and/or antigen independent, such as tumor-antigen independent, pathogen-antigen independent.
10. The vector according to any one of the previous items, wherein the antigenic unit of the first polypeptide and/or the further antigenic unit of at least one of the one or more further polypeptides comprise at least one universal CD4+ T cell epitope.
11. The vector according to any one of the previous items, wherein the antigenic unit of the first polypeptide and/or the further antigenic unit of the one of further polypeptides comprise at least two universal CD4+ T cell epitopes.
12. The vector according to item 11, wherein the at least two universal CD4+ T cell epitopes are different universal CD4+ T cell epitopes.
13. The vector according to any one of the previous items, wherein the antigenic unit of the first polypeptide comprises at least one CD8+ T cell epitope, and the further antigenic unit of at least one of the one or more further polypeptides comprises at least one universal CD4+ T cell epitope.
14. The vector according to any one of the previous items, wherein the further antigenic unit of at least one of the one of further polypeptides comprises a least one CD8+ T cell epitope, and the further antigenic unit of at least one other of the one or more further polypeptides comprises at least one universal CD4+ T cell epitope.
15. The vector according to any one of the previous items, wherein the further antigenic unit of at least one of the one of further polypeptides comprises a least one CD8+ T cell epitope, and at least one universal CD4+ T cell epitope.
16. The vector according to any one of items 10 to 15, wherein the universal CD4+ T cell epitope promotes activation of CD4+ T cells independently of the antigenic unit of the first polynucleotide.
17. The vector according to any one of items 10 to 16, wherein at least one universal CD4+ T cell epitope is derived from an antigen used in a vaccine, such as in a paediatric vaccine.
18. The vector according to any one of items 10 to 17, wherein at least one universal CD4+ T cell epitope is derived from an antigen of a pathogen, such as from antigens of tetanus and/or diphtheria, such as derived from tetanus and/or diphtheria toxoids.
19. The vector according to one any of items 10 to 18, wherein at least one universal CD4+ T cell epitope is derived from tetanus, such as from a tetanus toxoid.
20. The vector according to one any of items 10 to 19, wherein at least one universal CD4+ T cell epitope comprises an amino acid sequence selected from the group consisting of:

i.
(p30-SEQ ID NO: 119)
FNNFTVSFWLRVPKVSASHLE,
ii.
(p2-SEQ ID NO: 120)
ILMQYIKANSKFIGITE,

and
   iii. variants thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.
21. The vector according to one any of items 10 to 20, wherein at least one universal CD4+ T cell epitope is derived from an antigen of diphtheria, such as from a diphtheria toxoid.
22. The vector according to one any of items 10 to 21, wherein at least one universal CD4+ T cell epitope comprises an amino acid sequence selected from the group consisting of:

i.
(SEQ ID NO: 121)
QSIQLSSLMVAQAIP,

ii. variants thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.
23. The vector according to any one of items 10 to 22, wherein at least one universal CD4+ T cell epitope is non-naturally occurring.
24. The vector according to any one of items 10 to 23, wherein at least one universal CD4+ T cell epitope is a pan HLA-DR binding epitope.
25. The vector according to any one of items 10 to 24, wherein at least one universal CD4+ T cell epitope comprises an amino acid sequence selected from the group consisting of:

i.
(SEQ ID NO: 122)
AKFVAAWTLKAAA,

ii. variants thereof having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.
26. The vector according to any one of the previous items, wherein the further antigenic unit of at least one of the one of further polypeptides comprises an invariant chain-derived peptide amino acid sequence.
27. The vector according to any one of the previous items, wherein the further antigenic unit of at least one of the one of further polypeptides comprises a class II-associated invariant chain peptide (CLIP) amino acid sequence.
28. The vector according to any one of the previous items, wherein at least one of the one of further polypeptides comprises an antigen, a linker or 2A self-cleaving peptide, and the further antigenic unit comprises a class II-associated invariant chain peptide (CLIP) amino acid sequence and the at least one universal CD4+ T cell epitope, such as AKFVAAWTLKAAA (SEQ ID NO: 122).
29. The vector according to any one of the previous items, wherein the further antigenic unit of at least one of the one or more further polypeptides comprises an antigen.
30. The vector according to any one of the previous items, wherein the further antigenic unit of at least one of the one of further polypeptides comprises a furin linker amino acid sequence.
31. The vector according to any one of the previous items, wherein the further antigenic unit of at least one of the one of further polypeptides comprises an antigen, a furin linker amino acid sequence and the at least one universal CD4+ T cell epitope, such as FNNFTVSFWLRVPKVSASHLE (p30-SEQ ID NO: 119).
32. The vector according to any one of the previous items, wherein at least one of the one or more further polypeptides comprises an interaction unit.
33. The vector according to item 32, wherein the interaction unit is selected from the group consisting of a dimerization unit, a trimerization unit, and a tetramerization unit.
34. The vector according to any one of items 32 to 33, wherein the interaction unit is selected from the group consisting of a leucine zipper motif, a sequence capable of promoting oligomerization, such as a homo-trimerization domain, a heterodimerization unit, such as an heterodimerization unit comprising a coiled coil dimer-forming peptide, an oligomerization unit and a self-assembly unit.
35. The vector according to any one of the previous items, wherein the first polypeptide and at least one of the one or more further polypeptides are capable of forming a multimer, such as a dimer.
36. The vector according to any one of items 32 to 35, wherein the multimerization unit and the interaction unit are capable of forming a multimer, such as a dimer, such as a heterodimer.
37. The vector according to any one of items 35 to 36, wherein the multimer is a heterodimer.
38. The vector according to any one of items 32 to 37, wherein the multimerization unit and the interaction unit are different and are capable of forming a multimer, such as a dimer, such as a heterodimer.
39. The vector according to any one of items 34 to 37, wherein the heterodimerization unit comprises a coiled coil dimer-forming peptide.
40. The vector according to any one of the previous items, wherein the multimerization unit of the first polypeptide is a heterodimerization unit.
41. The vector according to any one of the previous items, wherein the multimerization unit of the first polypeptide is a heterodimerization unit comprising a coiled coil dimer-forming peptide.
42. The vector according to any one of items 34 to 41, wherein the coiled coil dimer is selected from the group of:
   i. a P7A:P8A coiled coil dimer;
   ii. a N7:N8 coiled coil dimer;
   iii. a N5:N6 coiled coil dimer.
43. The vector according to any one of items 34 to 42, wherein the coiled coil dimer-forming peptide is:
   i. P7A and/or P8A;
   ii. N7 and/or N8; or
   iii. N5 and/or N6.
44. The vector according to any one of items 34 to 43, wherein the coiled coil dimer-forming peptide is P7A or P8A.
45. The vector according to any one of the previous items, wherein at least one of the one or more further polypeptides comprises an interaction unit comprising a coiled coil dimer-forming peptide.
46. The vector according to any one of items 34 to 45, wherein the heterodimerization unit and the further heterodimeric dimerization form a coiled
47. The vector according to any one of items 34 to 46, wherein the heterodimerization unit of the first polypeptide and the further heterodimeric dimerization form a coiled coil dimer.
48. The vector according to any one of the previous items, wherein at least one of the one or more further polypeptides comprises an interaction unit, wherein the interaction unit is a further heterodimerization unit, and wherein the heterodimerization unit and the further heterodimerization unit form a P7A:P8A coiled coil dimer.
49. The vector according to any one of the previous items, wherein:
   i. the first polypeptide comprises a heterodimerization unit comprising a coiled coil dimer-forming peptide selected from P7A and P8A; and
   ii. at least one of the one or more further polypeptides comprises a further heterodimerization unit comprising a coiled coil dimer-forming peptide selected from P7A and P8A;
   wherein the heterodimerization unit and the further heterodimeric dimerization form a P7A:P8A coiled coil dimer.
50. The vector according to any one of items 42 to 49, wherein P7A comprises or consists of the amino acid sequence

(SEQ ID NO: 16)
YGEIAALEAKNAALKAEIAALEAKNAALKAGC.

51. The vector according to any one of items 42 to 50, wherein P8A comprises or consists of the amino acid sequence

(SEQ IS NO: 17)
YGKIAALKAENAALEAKIAALKAENAALEAGGC.

52. The vector according to any one of the previous items, wherein:
   i. the first polypeptide comprises a heterodimerization unit comprising a coiled coil dimer-forming peptide selected from N7 and N8; and
   ii. at least one of the one or more further polypeptides comprises a further heterodimerization unit comprising a coiled coil dimer-forming peptide selected from N7 and N8;
   wherein the heterodimerization unit and the further heterodimeric dimerization form a N7:N8 coiled coil dimer.
53. The vector according to any one of items 42 to 52, wherein N7 comprises or consists of the amino acid sequence

(SEQ ID NO: 18)
YEIAALEAKNAALKAEIAALEAKIAALKAGC.

54. The vector according to any one of items 42 to 53, wherein N8 comprises or consists of the amino acid sequence

(SEQ IS NO: 19)
YKIAALKAENAALEAKIAALKAEIAALEAGC.

55. The vector according to any one of the previous items, wherein:
   i. the first polypeptide comprises a heterodimerization unit comprising a coiled coil dimer-forming peptide selected from N5 and N6; and
   ii. at least one of the one or more further polypeptides comprises a further heterodimerization unit comprising a coiled coil dimer-forming peptide selected from N5 and N6;
   wherein the heterodimerization unit and the further heterodimeric dimerization form a N5:N6 coiled coil dimer.
56. The vector according to any one of items 42 to 55, wherein N5 comprises or consists of the amino acid sequence

(SEQ ID NO: 20)
YEIAALEAKIAALKAKNAALKAEIAALEAGC.

57. The vector according to any one of items 42 to 56, wherein N6 comprises or consists of the amino acid sequence

(SEQ ID NO: 21)
YKIAALKAEIAALEAENAALEAKIAALKAGC.

58. The vector according to any one of the previous items, wherein at least one of the one or more further nucleic acid sequences encodes a further polypeptide, wherein the further polypeptide comprises a further targeting unit that targets antigen-presenting cells, an interaction unit, such as a further multimerization unit, such as a further dimerization unit, and a further antigenic unit comprising one or more further epitopes, and wherein the first polypeptide and the further polypeptide are different.
59. The vector according to any one of items 32 to 58, wherein at least one further polypeptide forms a hetero-multimer, such as a heterodimer, with the first polypeptide via interaction of the multimerization unit of the first polypeptide and the interaction unit of said one further polypeptide.
60. The vector according to any of the previous items, wherein:
   i. the antigenic unit of the first polypeptide comprises one or more T cell epitopes;
   ii. the further antigenic unit of at least one of the one or more further polypeptides comprises one or more antigens, such as antigens comprising B-cell epitopes; and
   iii. the first polypeptide and at least one further polypeptide form a heterodimer.
61. The vector according to any one of the previous items, wherein:
   i. the antigenic unit of the first polypeptide comprises one or more antigens, such as antigens comprising B-cell epitopes;
   ii. the further antigenic unit of at least one of the one or more further polypeptides comprises one or more T cell epitopes; and
   iii. the first polypeptide and at least one further polypeptide form a heterodimer.
62. The vector according to any one of the previous items, wherein:
   i. the antigenic unit of the first polypeptide comprises one or more antigens, such as antigens comprising B-cell epitopes;
   ii. the further antigenic unit of at least one of the one or more further polypeptides comprises one or more antigens, such as antigens comprising B-cell epitopes; and
   iii. the first polypeptide and at least one further polypeptide form a heterodimer
   wherein the antigens comprising B-cell epitopes of the first polypeptide, and the antigens comprising B-cell epitopes of the at least one of the one or more further polypeptides are different.
63. The vector according to any one of the previous items, wherein the further antigenic unit of at least one of the one or more further polypeptides comprises an antigen which is capable of oligomerizing with at least one antigen comprised in the antigenic unit of the first polypeptide.
64. The vector according to any one of items 32 to 63, wherein the antigenic unit of at least one of the one or more further polypeptides comprises an antigen and the interaction unit of said further polypeptide is capable of oligomerizing with the antigenic unit of the first polypeptide, and/or with the interaction unit of another further polypeptides.
65. The vector according to any one of the previous items, wherein the antigenic unit of at least one of the one or more further polypeptides comprises an antigen which is capable of oligomerizing with at least one antigen comprised in the antigenic unit of the first polypeptide, and/or with at least one antigen comprised in the further antigenic unit of another further polypeptides.
66. The vector according to any one of the previous items, wherein the antigenic unit of at least one of the further polypeptides forms an oligomer, such as a dimer, such as a trimer, such as a tetramer, with at least one antigen comprised in the antigenic unit of the first polypeptide.
67. The vector according to any one of the previous items, wherein the antigenic unit of at least one of the further polypeptides forms an oligomer, such as a dimer, such as a trimer, such as a tetramer, with at least one antigen comprised in the antigenic unit of the first polypeptide and/or with at least one antigen comprised in the further antigenic unit of another further polypeptide.
68. The vector according to any one of items 32 to 67, wherein the interaction unit of at least one of the further polypeptides forms an oligomer, such as a dimer, such as a trimer, such as a tetramer, with at least one antigen comprised in the antigenic unit of the first polypeptide and/or with the interaction unit of another further polypeptide.
69. The vector according to any one of items 32 to 68, wherein the interaction unit of at least one of the one or more further polypeptides comprises a sequence that encodes an amino acid sequence that facilitates the trimerization of the antigens, such as a hetero-trimerization sequence, e.g., a heterotrimeric coiled coil pair.
70. The vector according to any one of items 32 to 69, wherein the interaction unit of at least two of the one or more further polypeptides comprise a heterotrimeric coiled coil pair.
71. The vector according to any one of items 69 to 70, wherein the heterotrimeric coiled coil pair of the antigenic unit of the first polypeptide and the heterotrimeric coiled coil pair of the interaction unit of at least one further polypeptide facilitates the oligomerization of an antigen comprised in the antigenic unit of the first polypeptide and a further antigen in the further antigenic unit of the further polypeptide.
72. The vector according to any one of items 32 to 71, wherein the antigenic unit of the first polypeptide, and/or the interaction unit of at least one of the one or more further polypeptides comprise or consists of the amino acid sequence

(SEQ ID NO: 22)
YGGIEAKIEAIEAKAEAIEAKIEAIEAKIEA.

73. The vector according to any one of items 32 to 72, wherein the antigenic unit of the first polypeptide, and/or the interaction unit of at least one of the one or more further polypeptides comprise or consists of the amino acid sequence

(SEQ ID NO: 23)
GGIEQKIEAIEWKWEAIEQKIEAIEQKIEA.

74. The vector according to any one of items 32 to 73, wherein the antigenic unit of the first polypeptide, and/or the interaction unit of at least one of the one or more further polypeptides comprise or consists of the amino acid sequence

(SEQ ID NO: 12)
AEIAAIEYEQAAIKEEIAAIKDKIAAIKEYIAAI.

75. The vector according to any one of items 32 to 74, wherein the antigenic unit of the first polypeptide, and/or the interaction unit of at least one of the one or more further polypeptides comprise or consists of the amino acid sequence

(SEQ ID NO: 13)
EKIAAIKEEQAAIEEEIQAIKEEIAAIKYLIAQI.

76. The vector according to any one of items 32 to 75, wherein the antigenic unit of the first polypeptide, and/or the interaction unit of at least one of the one or more further polypeptides comprise or consists of the amino acid sequence

(SEQ ID NO: 14)
AEIAAIKYKQAAIKNEIAAIKQEIAAIEQMIAAI.

77. The vector according to any one of items 34 to 76, wherein the self-assembly unit of at least one further polypeptide is ferritin.
78. The vector according to any one of items 34 to 77, wherein the oligomerization unit of at least one further polypeptide is sortase A.
79. The vector according to any one of items 34 to 78, wherein the self-assembly unit of at least one further polypeptide is derived from the self-forming structure component of a self-assembling molecule, such as ferritin, lumazine synthase from Aquifex aeolicus (LS), E2 from Geobacillus stearothermophilus, or 13-01-modified 2-dehydro-3-deoxy-phosphogluconate aldolase (=2-Keto-3-deoxy-6-phosphogluconate (KDPG) aldolase).
80. The vector according to any one of items 34 to 79, wherein the oligomerization unit of at least one further polypeptide is selected from the group consisting of: sortase A; lumazine synthase from Aquifex aeolicus (LS), E2 from Geobacillus stearothermophilus, and 13-01-modified 2-dehydro-3-deoxy-phosphogluconate aldolase (=2-Keto-3-deoxy-6-phosphogluconate (KDPG) aldolase).
81. The vector according to any one of the previous items, wherein at least one of the one or more further polypeptides comprises a sequence that promotes formation of nanoparticles, such as antigen-presenting nanoparticles.
82. The vector according to any one of the previous items, wherein the first polypeptide comprises a first leucine zipper motif.
83. The vector according to any one of items 32 to 80, wherein the interaction unit of at least one of the one or more further polypeptides comprise a second leucine zipper motif.
84. The vector according to item 83, wherein the first leucine zipper motif and the second leucine zipper motif are capable of forming a dimer.
85. The vector according to any one of items 83 to 84, wherein the first leucine zipper motif is at the C terminal end of the first polypeptide and the second leucine zipper motif is at the C terminal end or at the N terminal end of the one or more further polypeptides.
86. The vector according to any one of items 32 to 85, wherein an interaction unit comprising a first leucine zipper motif is at the C terminal end or at the N terminal end of one further polypeptide, and an interaction unit comprising a second leucine zipper motif is at the C terminal end or at the N terminal end of one other further polypeptide.
87. The vector according to any one of the previous items, wherein at least two of the one or more further polypeptides comprise a zipper motif, wherein the leucine zipper motif are capable of forming a dimer between the at least two further polypeptides.
88. The vector according to any one of items 34 to 87, wherein at least one leucine zipper motif comprises the amino acid sequence

(SEQ ID NO: 24)
LEIRAAFLRQRNTALRTEVAELEQEVQRLENEVSQYETRYGPLGGGK.

89. The vector according to any one of items 34 to 88, wherein at least one leucine zipper motif comprises the amino acid sequence

(SEQ ID NO: 25)
LEIEAAFLERENTALETRVAELRQRVQRLRNRVSQYRTRYGPLGGGK.

90. The vector according to any of the previous items, wherein at least one of the one or more further polypeptides comprises a further targeting unit.
91. The vector according to item 90, wherein the further targeting unit of at least one further polypeptide is a MHC II targeting unit.
92. The vector according to any one of items 90 to 91, wherein the further targeting unit of at least one further polypeptide targets antigen-presenting cells.
93. The vector according to any one of items 90 to 92, wherein the further targeting unit of at least one further polypeptide is an antibody or part thereof.
94. The vector according to any one of items 90 to 93, wherein the further targeting unit of at least one further polypeptide is a scFv or a an alpaca derived VHH, such as a VHH_{MHCII}.
95. The vector according to any one of the previous items, wherein the first nucleic acid sequence encodes a signal peptide and/or at least one of the one or more further nucleic acid sequences encodes a further signal peptide.
96. The vector according to any one of the previous items, wherein the first polypeptide comprises a signal peptide and/or at least one of the one or more further polypeptides comprises a further signal peptide.
97. The vector according to any one of the previous items, wherein at least one of the one or more further polypeptides comprises a further signal peptide.
98. The vector according to any one of items 95 to 97, wherein the further signal peptide of at least one further polypeptide is different from the signal peptide of the targeting unit of the first polypeptide.
99. The vector according to any one of items 95 to 98, wherein the signal peptide and/or the further signal peptide of at least one further polypeptide is the natural leader sequence of the protein which is the targeting unit.
100. The vector according to any one of items 95 to 99, wherein the signal peptide and/or the further signal peptide of at least one further polypeptide is selected from the group consisting of Ig VH signal peptide, human serum albumin signal peptide (SEQ ID NO: 138), human modified IgG H signal peptide (SEQ ID NO: 139), human HC H6 signal peptide (SEQ ID NO: 140), human TPA signal peptide and human CCL3L1 signal peptide.
101. The vector according to any one of items 95 to 100, wherein the targeting unit and/or the further targeting unit is human CCL3L1 and the signal peptide and/or the further signal peptide of at least one further polypeptide comprises an amino acid sequence having at least 85% sequence identity to the amino acid sequence 1-23 of SEQ ID NO: 128.
102. The vector according to any one of items 95 to 101, wherein the signal peptide and/or the further signal peptide of at least one further polypeptide consists of an amino acid sequence having at least 85% sequence identity to the amino acid sequence 1-23 of SEQ ID NO: 128.
103. The vector according to any one of items 95 to 102, wherein the signal peptide and/or the further signal peptide of at least one further polypeptide consists of the amino acid sequence 1-23 of SEQ ID NO: 128.
104. The vector according to any one of the previous items, wherein the antigenic unit of the first polypeptide comprises one or more antigens comprising one or more B-cell epitopes, such as one or more conformational B-cell epitopes, and the further antigenic unit of at least one of the one or more further polypeptides comprises one or more further antigen comprising one or more further B-cell epitopes, such as one or more further conformational B-cell epitopes.
105. The vector according to any one of the previous items, wherein at least one antigen and at least one further antigen are from different strains or serotypes, for example different strains or serotypes of a pathogen.
106. The vector according to any one of items 35 to 105, wherein the multimer is a heterodimer of a first and a further polypeptide, wherein the first polypeptide comprise a targeting unit and a heterodimerization unit, wherein the further polypeptide comprises a further targeting unit and an interaction unit, wherein the antigenic unit of the first polypeptide comprises one or more antigens comprising B-cell epitopes, wherein the further antigenic unit of the further polypeptide comprises one or more further antigens comprising B-cell epitopes, and wherein at least one antigen and at least one further antigen are derived from the same pathogen but from different strains or serotypes.
107. The vector according to any one of items 35 to 105, wherein the multimer is a heterodimer of a first polypeptide and a further polypeptide, wherein the first polypeptide comprise a first heterodimerization unit, and wherein the further polypeptide comprises a further antigenic unit comprising an antigen or a T cell epitope and a further heterodimerization unit which is different from the first heterodimerization unit.
108. The vector according to any one of items 35 to 105, wherein the multimer is a heterodimer of a first polypeptide and a further polypeptide, wherein the first polypeptide comprise an antigenic unit comprising an antigen and a first heterodimerization unit, and wherein the further polypeptide comprises a further antigenic unit comprising a further antigen and a further heterodimerization unit which is different from the first heterodimerization unit.
109. The vector according to any one of items 35 to 105, wherein the multimer is a heterodimer of a first polypeptide and a further polypeptide, wherein the first polypeptide comprise an antigenic unit comprising an antigen and a first heterodimerization unit, and wherein the further polypeptide comprises a further antigenic unit comprising at least one T cell epitope and a further heterodimerization unit which is different from the first heterodimerization unit.
110. The vector according to any one of items 35 to 106, wherein the multimer is a heterodimer of a first polypeptide and a further polypeptide, wherein the first polypeptide comprise a targeting unit and a heterodimerization unit, wherein the further polypeptide comprises a further targeting unit and a further heterodimerization unit, and wherein the antigenic unit of the first polypeptide comprises T cell epitopes of a pathogen and the further antigenic unit of the further polypeptide comprises a further antigen of the same pathogen.
111. The vector according to any one of items 35 to 107, wherein the multimer is a heterodimer of a first polypeptide and a further polypeptide, wherein the first polypeptide comprise a targeting unit and a heterodimerization unit, wherein the further polypeptide comprises a further targeting unit and a further heterodimerization unit, and wherein the antigenic unit of the first polypeptide comprises T cell epitopes of several different pathogens and the antigenic unit of the second polypeptide comprises an antigen of a pathogen, whose T cell epitopes are included in the antigenic unit of the first polypeptide.
112. The vector according to any one of the previous items, wherein the vector further encodes for one or more additional polynucleotides, wherein the one or more additional polynucleotides comprise one or more nucleic acid sequences encoding one or more immunostimulatory compounds, and wherein the vector allows for the co-expression of the first polypeptide and the one or more immunostimulatory compounds as separate molecules.
113. The vector according to any one of the previous items, wherein the vector further encodes one or more additional polynucleotides, wherein the one or more additional polynucleotides comprise one or more nucleic acid sequences encoding at least two, such as at least three, immunostimulatory compounds, wherein said immunostimulatory compounds are identical or different, preferably identical, and wherein the vector allows for the co-expression of the first polypeptide and the one or more immunostimulatory compounds as separate molecules.
114. The vector according to any one of the previous items, wherein at least one of the one or more further polypeptides comprises more than one epitope, such as 2, 3, 4, 5, 6, 7 or 8 epitopes, such as 2, 3, 4, 5, 6, 7 or 8 different epitopes.
115. The vector according to any one of the previous items, wherein at least one of the one or more further polypeptides comprises more than one antigen, such as 2, 3, 4, 5, 6, 7 or 8 antigens, such as 2, 3, 4, 5, 6, 7 or 8 different antigens.
116. The vector according to any one of the previous items, wherein at least one of the one or more further polypeptides comprises one or more full length antigens.
117. The vector according to any one of the previous items, wherein at least one of the one or more further polypeptides comprises one full length antigen.
118. The vector according to any one of the previous items, wherein the further antigen is an antigen with an amino acid sequence of at least 50, 60, 80, 100, 120, 140, 160, 180, 200, 250, 300, 350, 400, 450, 500, 550, 600, 650, 750, 800, 900, 1000, 1250, 1500, 1750, 2000, 2500, 3000 amino acids.
119. The vector according to any one of the previous items, wherein the further antigen is an antigen of a membrane protein.
120. The vector according to any one of the previous items, wherein the further antigen is is an oligomeric antigen.
121. The vector according to any one of the previous items, wherein at least one of the one or more further polypeptides comprises one or more T cell epitopes and one or more B cell epitopes.
122. The vector according to any one of the previous items, wherein at least one of the one or more further polypeptides comprises one or more T cell epitopes.
123. The vector according to any one of the previous items, wherein at least one of the one or more further polypeptides comprises one or more B cell epitope.
124. The vector according to any one of the previous items, wherein at least one of the one or more further polypeptides comprises a ubiquitin unit.
125. The vector according to item 124, wherein the ubiquitin unit comprises or consists of an amino acid sequence selected from the group consisting of:

| | |
|---|---|
| | (SEQ ID NO: 123) |
| i. | MQIFVKTLTGKTITLEVEPSDTIENVKAKIQDKEGIPPDQQRLI |
| | FAGKQLEDGRTLSDYNIQKESTLHLVLRLRGG; |
| | (SEQ ID NO: 124) |
| ii. | MQIFVKTLTGKTITLEVEPSDTIENVKAKIQDKEGIPPDQQRLI |
| | FAGKQLEDGRTLSDYNIQKESTLHLVLRLRGA; |
| | (SEQ ID NO: 125) |
| iii. | MQIFVKTLTGKTITLEVEPSDTIENVKAKIQDKEGIPPDQQRLI |
| | FAGKQLEDGRTLSDYNIQKESTLHLVLRLRGV; |
| | (SEQ ID NO: 126) |
| iv. | MQIFVKTLTGKTITLEVEPSDTIENVKAKIQDKEGIPPDQQRLI |
| | FAGKQLEDGRTLSDYNIQKESTLHLVLRPRGG; |
| | and |
| | (SEQ ID NO: 127) |
| v. | MQIFVKTLTGKTITLEVEPSDTIENVKAKIQDKEGIPPDQQRLI |
| | FAGKQLEDGRTLSDYNIQKESTLHLVLRLR. |

126. The vector according to any one of items 124 to 125, wherein:
   i. at least one of the one or more further polypeptides comprises a ubiquitin unit; and
   ii. the further antigenic unit of the at least one further polypeptide comprises a further antigen, wherein the N-terminal amino acid of the further antigen has been substituted to R compared to the natural sequence of said further antigen.
127. The vector according to any one of items 124 to 126, wherein at least one of the one or more further epitopes is fused to the ubiquitin unit.
128. The vector according to any one of items 124 to 127, wherein the ubiquitin unit is upstream of the further antigenic unit comprising one or more further epitopes.
129. The vector according to any one of items 124 to 128, wherein the further antigenic unit of the at least one further polypeptide comprises one or more T cell epitopes.
130. The vector according to any one of items 124 to 129, wherein the proteasomal degradation, and/or MHC antigen presentation of the at least one of the one or more further polypeptides is increased compared to one or more polypeptides without an ubiquitin unit but otherwise identical sequence to the at least one of the one or more further polypeptides comprising the ubiquitin unit.
131. The vector according to any one of items 124 to 130, wherein the ubiquitin unit consists of a motif that promotes the proteasomal degradation, and/or MHC antigen presentation of the one or more further polypeptides.
132 The vector according to any of the previous items, wherein:
   i. the first polypeptide comprises a first leucine zipper motif at the C terminal end of the first polypeptide;
   ii. at least one of the one or more further polypeptides comprises a second leucine zipper motif at the C terminal end or at the N terminal end; and
   iii. the further antigenic unit of the at least one further polypeptide comprises an antigen; and
   iv. the first leucine zipper motif forms a dimer with the second leucine zipper motif.
133. The vector according to item 132, wherein the first leucine zipper motif and the second leucine zipper motif are capable of forming a dimer.
134. The vector according to any one of the previous items, wherein at least one of the one or more further polypeptides comprise:
   i. a further signal peptide; and
   ii. an interaction unit.
135. The vector according to any one of the previous items, wherein the antigenic unit of the first polypeptide comprises one or more B cell epitopes.
136. The vector according to any one of the previous items, wherein the at least one further polypeptide comprises one or more further epitopes.
137. The vector according to any one of items 32 to 136, wherein:
   i. the targeting unit of the first polypeptide comprises or consists of human CCL3L1;
   ii. the antigenic unit of the first polypeptide comprises one or more B cell epitopes;
   iii. the interaction unit of the first polypeptide comprises the amino acid sequence YGGIEAKIEAIEAKAEAIEAKIEAIEAKIEA (SEQ ID NO: 22);
   iv. at least one of the one or further polypeptides comprise a further signal peptide, wherein the further signal peptide comprises or consists of the signal peptide of human CCL3L1; and
   v. the interaction unit of at least one further polypeptide comprises the amino acid sequence GGIEQKIEAIEWKWEAIEQKIEAIEQKIEA (SEQ ID NO: 23).
138. The vector according to any one of items 32 to 137, wherein:
   i. the targeting unit of the first polypeptide comprises or consists of human CCL3L1;
   ii. the antigenic unit of the first polypeptide comprises one or more B cell epitopes;
   iii. the interaction unit of the first polypeptide comprises the amino acid sequence YGGIEAKIEAIEAKAEAIEAKIEAIEAKIEA (SEQ ID NO: 22);
   iv. at least one of the one or further polypeptides comprise a further signal peptide, wherein the further signal peptide comprises or consists of the signal peptide of human CCL3L1;
   v. the further antigenic unit of the at least one further polypeptide comprises a further antigen comprising one or more epitopes; and
   vi. the interaction unit of at least one further polypeptide comprises the amino acid sequence GGIEQKIEAIEWKWEAIEQKIEAIEQKIEA (SEQ ID NO: 23).
139. The vector according to any one of items 32 to 138, wherein:
   i. the targeting unit of the first polypeptide comprises or consists of human CCL3L1;
   ii. the antigenic unit of the first polypeptide comprises an antigen comprising one or more B cell epitopes;
   iii. the interaction unit of the first polypeptide comprises the amino acid sequence YGGIEAKIEAIEAKAEAIEAKIEAIEAKIEA (SEQ ID NO: 22);
   iv. at least one of the one or further polypeptides comprise a further signal peptide, wherein the further signal peptide comprises or consists of the signal peptide of human CCL3L1;
   v. the further antigenic unit of the at least one further polypeptide comprises a further antigen, wherein said antigen and further antigen are identical; and
   vi. the interaction unit of at least one further polypeptide comprises the amino acid sequence GGIEQKIEAIEWKWEAIEQKIEAIEQKIEA (SEQ ID NO: 23).
140. The vector according to any one of items 32 to 139, wherein:
   i. the targeting unit of the first polypeptide comprises or consists of human CCL3L1;
   ii. the antigenic unit of the first polypeptide comprises an antigen comprising one or more B cell epitopes;
   iii. the interaction unit of the first polypeptide comprises the amino acid sequence YGGIEAKIEAIEAKAEAIEAKIEAIEAKIEA (SEQ ID NO: 22);
   iv. at least one of the one or further polypeptides comprise a further signal peptide, wherein the further signal peptide comprises or consists of the signal peptide of human CCL3L1;
   v. the further antigenic unit of the at least one further polypeptide comprises a further antigen, wherein said antigen and further antigen are naturally found in the same protein or protein complex or are identical; and
   vi. the interaction unit of at least one further polypeptide comprises the amino acid sequence GGIEQKIEAIEWKWEAIEQKIEAIEQKIEA (SEQ ID NO: 23).
141. The vector according to any one of items 32 to 140, wherein:
   i. the targeting unit of the first polypeptide comprises or consists of human CCL3L1;
   ii. the antigenic unit of the first polypeptide comprises one or more B cell epitopes;
   iii. the antigenic unit of the first polypeptide comprises the amino acid sequence AEIAAIEYEQAAIKEEIAAIKDKIAAIKEYIAAI (SEQ ID NO: 12);
   iv. at least one of the one or further polypeptides comprise a further signal peptide, wherein the further signal peptide comprises or consists of the signal peptide of human CCL3L1;
   v. the interaction unit of at least one other further polypeptide comprises the amino acid sequence EKIAAIKEEQAAIEEEIQAIKEEIAAIKYLIAQI (SEQ ID NO: 13); and
   vi. the interaction unit of at least one other further polypeptide comprises the amino acid sequence AEIAAIKYKQAAIKNEIAAIKQEIAAIEQMIAAI (SEQ ID NO: 14).
142. The vector according to any one of the previous items, wherein:
   i. the targeting unit of the first polypeptide comprises or consists of human CCL3L1;
   ii. the antigenic unit of the first polypeptide comprises one or more T cell epitopes, wherein the one or more T cell epitopes are CD4+ and CD8⁺ T cell epitopes, wherein the one or more T cell epitopes are separated by linkers;
   iii. the further antigenic unit of at least one of the one or more further polypeptides comprises one or more further epitopes; and
   iv. at least one of the one or more further polypeptides comprises a further targeting unit, wherein the further targeting unit is a scFv or a VHH targeting MHC II, such as a VHH_{MHCII}.
143. The vector according to any one of the previous items, wherein:
   i. the antigenic unit of the first polypeptide comprises or consists of T cell epitopes; and
   ii. the further antigenic unit of at least one the one or more further polypeptides comprises one or more full length antigens.
144. The vector according to any one of the previous items, wherein:
   i. the targeting unit of the first polypeptide comprises or consists of human CCL3L1;
   ii. the antigenic unit of the first polypeptide comprises or consists of T cell epitopes; and
   iii. the further antigenic unit of at least one of the one or more further polypeptides comprises one or more full length antigens.
145. The vector according to any one of the previous items, wherein;
   i. the targeting unit of the first polypeptide comprises or consists of human CCL3L1;
   ii. the antigenic unit of the first polypeptide comprises or consists of T cell epitopes;
   iii. the further antigenic unit of at least one of the one or more further polypeptides comprises one full length antigen.
146. The vector according to any one of the previous items, wherein;
   i. the targeting unit of the first polypeptide comprises or consists of human CCL3L1;
   ii. the antigenic unit of the first polypeptide comprises or consists of T cell epitopes; and
   iii. the further antigenic unit of at least one of the one or more further polypeptides comprises one or more further antigens.
147. The vector according to any one of the previous items, wherein:
   i. the targeting unit of the first polypeptide comprises or consists of human CCL3L1;
   ii. the antigenic unit of the first polypeptide comprises or consists of T cell epitopes; and
   iii. the further antigenic unit of at least one of the one or more further polypeptides comprises one further antigen.
148. The vector according to any one of the previous items, wherein:
   i. the antigenic unit of the first polypeptide comprises or consists of T cell epitopes;
   ii. at least one of the one or more further polypeptides comprises a further targeting unit;
   iii. the first polypeptide further comprises a heterodimerization unit, wherein the heterodimerization unit comprises a coiled coil dimer-forming peptide, such as P7A or P8A;
   iv. the at least one further polypeptide comprises a further heterodimerization unit, wherein the further heterodimerization unit comprises a coiled coil dimer-forming peptide, such as P7A or P8A; and
   v. the at least one further polypeptide further comprises one or more B cell epitopes and/or one or more antigens, such as further antigens.
149. The vector according to any one of the previous items, wherein:
   i. the antigenic unit of the first polypeptide comprises or consists of one or more B cell epitopes and/or one or more antigens;
   ii. at least one of the one or more further polypeptides comprises a further targeting unit;
   iii. the multimerization unit of the first polypeptide comprises a heterodimerization unit, wherein the heterodimerization unit comprises a coiled coil dimer-forming peptide, such as P7A or P8A;
   iv. the at least one further polypeptide comprises a further heterodimerization unit, wherein the heterodimerization unit comprises a coiled coil dimer-forming peptide, such as P7A or P8A;
   v. the further antigenic unit of the at least one further polypeptide comprises one or more B cell epitopes or one or more further epitopes, such as T cell epitopes.
150. The vector according to any one of the previous items, wherein:
   i. the antigenic unit of the first polypeptide comprises or consists of one or more antigens;
   ii. at least one of the one or more further polypeptides comprises a further targeting unit;
   iii. the multimerization unit of the first polypeptide comprises a heterodimerization unit, wherein the heterodimerization unit comprises a coiled coil dimer-forming peptide, such as P7A or P8A;
   iv. the at least one further polypeptides comprises a further heterodimerization unit, wherein the heterodimerization unit comprises a coiled coil dimer-forming peptide, such as P7A or P8A;
   v. the further antigenic unit of the at least one further polypeptides further comprises one or more further epitopes, such as T cell epitopes.
151. The vector according to any one of items 148 to 150, wherein the heterodimerization unit and the further heterodimerization unit are capable of forming a heterodimer.
152. The vector according to any one of items 124 to 152, wherein:
   i. the antigenic unit of the first polypeptide comprises or consists of B cell epitopes and/or antigens;
   ii. at least one of the one or more further polypeptides comprises a ubiquitin unit;
   iii. the ubiquitin unit is upstream from the further antigenic unit comprising one or more further epitopes, wherein the one or more further epitopes are T cell epitopes;
   iv. the ubiquitin unit consists of a nucleic acid motif that promotes secretion of the at least one further polypeptide.
153. The vector according to any of the previous items, wherein:
   i. the antigenic unit of the first polypeptide comprises or consists of an antigen or parts thereof; and
   ii. the further antigenic unit of at least one of the one or more further polypeptides comprises or consists of the amino acid sequence of FNNFTVSFWLRVPKVSASHLE (p30-SEQ ID NO: 119).
154. The vector according to any one of the previous items, wherein:
   i. the antigenic unit of the first polypeptide comprises or consists of an antigen or parts thereof; and
   ii. the further antigenic unit of at least one of the one or more further polypeptides comprises or consists of a universal CD4+ cell epitope comprising or consisting of the amino acid sequence AKFVAAWTLKAAA (SEQ ID NO: 122) and an invariant chain-derived peptide, such as class II-associated invariant chain peptide (CLIP).
155. The vector according to any one of the previous items, wherein:
   i. the targeting unit of the first polypeptide is CCL3L1;
   ii. the multimerization unit of the first polypeptide is a hinge exon h1, a hinge exon h4, a dimerization unit linker and a CH3 domain of human IgG3;
   iii. the antigenic unit of the first polypeptide comprises one antigen; and
   iv. at least one of the one or more further polypeptides comprises an interaction unit which is a self-assembly unit, such as ferritin; or said further polypeptide is capable of spontaneously forming an oligomer or multimer.
156. The vector according to any one of the previous items, wherein:
   i. the targeting unit of the first polypeptide is CCL3L1;
   ii. the multimerization unit of the first polypeptide is a hinge exon h1, a hinge exon h4, a dimerization unit linker and a CH3 domain of human IgG3;
   iii. the antigenic unit of the first polypeptide comprises one antigen;
   iv. at least one of the one or more further polypeptides comprises an interaction unit which is a self-assembly unit, such as ferritin; or said further polypeptide is capable of spontaneously forming an oligomer or multimer; and
   v. the vector encodes for an additional polynucleotide encoding an immunostimulatory compound.
157 The vector according to any one of the previous items, wherein:
   i. the targeting unit of the first polypeptide is CCL3L1;
   ii. the multimerization unit of the first polypeptide is a hinge exon h1, a hinge exon h4, a dimerization unit linker and a CH3 domain of human IgG3;
   iii. the antigenic unit of the first polypeptide comprises one antigen;
   iv. at least one of the one or more further polypeptides comprises an interaction unit which is a self-assembly unit, such as ferritin; or said further polypeptide is capable of spontaneously forming an oligomer or multimer; and
   v. the further antigenic unit of the at least one further polypeptide comprises a further antigen, wherein the further antigen is a variant of the antigen having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto.
158. The vector according to any one of the previous items, wherein:
   i. the targeting unit is CCL3L1;
   ii. the multimerization unit is a hinge exon h1, a hinge exon h4, a dimerization unit linker and a CH3 domain of human IgG3;
   iii. the antigenic unit comprises one antigen;
   iv. at least one of the one or more further polypeptides comprises an interaction unit which is a self-assembly unit, such as ferritin; or said further polypeptide is capable of spontaneously forming an oligomer or multimer;
   v. the further antigenic unit of the at least one further polypeptide comprises a further antigen, wherein the further antigen is a variant of the antigen having at least 60% sequence identity thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% sequence identity thereto; and
   vi. the vector encodes for an additional polynucleotide encoding an immunostimulatory compound.
159. The vector according to according to any one of the previous items, wherein said vector comprises one or more co-expression elements.
160. The vector according to according to any one of the previous items, wherein the one or more co-expression elements cause the transcription of the first polypeptide and the one or more further polypeptides on a single transcript and the independent translation into a separate first polypeptide and separate one or more further polypeptides.
161. The vector according to any one of items 159 to 160, wherein at least one of the one or more co-expression elements are IRES elements, or nucleic acid sequences encoding 2A self-cleaving peptides.
162. The vector according to any to any one of items 159 to 161, wherein at least one of the one or more co-expression elements is a nucleic acid sequence encoding a 2A self-cleaving peptide.
163. The vector according to item 162, wherein the 2A self-cleaving peptide is selected from the group consisting of T2A peptide, P2A peptide, E2A peptide and F2A peptide.
164. The vector according to any one of items 159 to 163, wherein at least one of the one or more co-expression elements promotes the transcription of the first polypeptide and the one or more further polypeptides as separate transcripts.
165. The vector according to any one of items 159 to 164, wherein the one or more co-expression elements are a) bidirectional promoters or b) promoters, wherein the vector comprises a separate bidirectional promoter or separate promoter for each of the nucleic acid sequences encoding the first polypeptide and the one or more further polypeptides.
166. The vector according to any one of the previous items, wherein the targeting unit of the first polypeptide comprises or consists of human CCL3L1.
167. The vector according to any one of the previous items, wherein the multimerization unit of the first polypeptide is selected from the group consisting of dimerization unit, trimerization unit, such as a collagen-derived trimerization unit, such as a human collagen-derived trimerization domain, such as human collagen derived XVIII trimerization domain or human collagen XV trimerization domain or the C-terminal domain of T4 fibritin and tetramerization unit, such as a domain derived from p53 and wherein said multimerization unit optionally comprises a hinge region, such as hinge exon h1 and hinge exon h4.
168. The vector according to any one of the previous items, wherein the vector comprises a hinge region which has the ability to form one or more covalent bonds and is preferably Ig derived.
169. The vector according to any one of the previous items, wherein the multimerization unit of the first polypeptide is a dimerization unit and said dimerization unit further comprises another domain that facilitates dimerization, preferably wherein the other domain is an immunoglobulin domain, more preferably an immunoglobulin constant domain.
170. The vector according to item 169, wherein the other domain is a carboxyterminal C domain derived from IgG, preferably from IgG3.
171. The vector according to any one of the previous items, wherein the dimerization unit of the first polypeptide further comprises a dimerization unit linker, such as glycine-serine rich linker, such as GGGSSGGGSG (SEQ ID NO: 15) and preferably wherein the dimerization unit linker connects the hinge region and the other domain that facilitates dimerization.
172. The vector according to any one of the previous items, wherein the dimerization unit of the first polypeptide comprises hinge exon h1 and hinge exon h4, a dimerization unit linker and a CH3 domain of human IgG3.
173. The vector according to any one of the previous items, wherein the first nucleic acid sequence encodes a first polypeptide which further comprises a unit liker that connects the antigenic unit to the multimerization unit, and wherein the unit linker is a non-immunogenic linker and/or flexible or rigid linker.
174. The vector according to any one of the previous items, wherein the first nucleic acid sequence encodes a first polypeptide which comprises a signal peptide, and preferably wherein at least one of the one or more further nucleic acid sequences encodes a further signal peptide.
175. The vector according to any one of the previous items, wherein the vector is a viral vector, such as an RNA viral vector or DNA viral vector or a plasmid, such as an RNA plasmid or DNA plasmid.
176. The vector according to any one of the previous items, wherein the first nucleic acid sequence and/or the one or more further nucleic acid sequences is selected from DNA sequence and RNA sequence.
177. A method of producing a vector as defined in any one of the previous items, the method comprising the following steps:
   a) transfecting cells in vitro with the vector according to any one of the previous items;
   b) culturing said cells;
   c) optionally, lysing the cells to release the vector from the cells; and
   d) collecting and optionally purifying the vector.
178. A host cell comprising a vector as defined in any one of items 1 to 176, such as a host cell selected from the group consisting of prokaryote cells, yeast cells, insect cells, higher eukaryotic cells such as cells from animals or humans.
179. A vector as defined in any one of items 1 to 176 for use as a medicament.
180. A pharmaceutical composition comprising the vector as defined in any one of items 1 to 176 and a pharmaceutically acceptable carrier or diluent.
181. The pharmaceutical composition according to item 180, wherein the composition further comprises a transfection agent.
182. The pharmaceutical composition according to any one of items 180 to 181, wherein the composition comprises said vector, e.g., said DNA plasmid, in a range of from 0.1 to 10 mg.
183. A method of treating a subject having a disease or being in need of prevention of said disease, the method comprising administering to the subject a vector as defined in any one of items 1 to 176 or a pharmaceutical composition as defined in any one of items 180 to 182.
184. The method according to item 183, wherein the vector or the pharmaceutical composition is administered in a therapeutically or prophylactically effective amount, such as administered by intradermal, intramuscular, or subcutaneous injection, or by mucosal or epithelial application, such as intranasal or oral.
185. A method of treating a subject having cancer, the method comprising administering to the subject a vector as defined in any one of items 1 to 176 or a pharmaceutical composition as defined in any one of items 180 to 182 comprising such vector.
186. The method according to item 185, wherein the vector or the pharmaceutical composition is administered in a therapeutically effective amount, such as administered by intradermal, intramuscular, or subcutaneous injection, or by mucosal or epithelial application, such as intranasal or oral.
187. A method for treating a subject having an infectious disease or being in need of prevention of an infectious disease, the method comprising administering to the subject a vector as defined in any one of items 1 to 176 or a pharmaceutical composition as defined in any one of items 180 to 182, comprising such vector.
188. The method according to item 187, wherein the vector or the pharmaceutical composition is administered in a therapeutically or prophylactically effective amount, such as administered by intradermal, intramuscular, or subcutaneous injection, or by mucosal or epithelial application, such as intranasal or oral.

### Items—2

189. A vector comprising:
   (a) a first nucleic acid sequence encoding a first polypeptide, wherein the first polypeptide comprises a targeting unit that targets antigen-presenting cells, a multimerization unit, such as a dimerization unit, and an antigenic unit comprising one or more T cell epitopes of an allergen, self-antigen or alloantigen; and
   (b) one or more further nucleic acid sequences encoding one or more further polypeptides, wherein the one or more further polypeptides comprise a further antigenic unit comprising one or more allergens, hypoallergenic allergens, self-antigens or alloantigens, derivatives thereof or parts thereof,
   wherein the vector allows for the co-expression of the first polypeptide and the one or more further polypeptides as separate molecules.
190. The vector according to item 189, wherein the further antigenic unit comprises one or more allergens, hypoallergenic allergens, self-antigens or alloantigens.
191. The vector according to any one of items 189 to 190, wherein the antigenic unit comprises one or more T cell epitopes of an allergen and the further antigenic unit comprises one or more allergens.
192. The vector according to any of items 189 to 191, wherein the antigenic unit comprises one or more T cell epitopes of an allergen and the further antigenic unit comprises one or more hypoallergenic allergens.
193. The vector according to any of the items 189 to 192, wherein the antigenic unit comprises one or more T cell epitopes of an allergen and the further antigenic unit comprises one allergen.
194. The vector according to any of items 189 to 193, wherein the antigenic unit comprises one or more T cell epitopes of an allergen and the further antigenic unit comprises one hypoallergenic allergen.
195. The vector according to any of items 189 to 194, wherein the one or more T cell epitopes and/or one or more allergens and/or one or more hypoallergenic allergens are derived from one or more allergens, wherein the allergens are selected from the group consisting of shellfish allergen, cow's milk allergen, egg allergen, fish allergen, fruit allergen, wheat allergen, peanut allergen, tree nut allergen, soy allergen, seed allergen, buckwheat allergen, celery allergen, garlic allergen, gluten allergen, oat allergen, legumes allergen, maize allergen, milk allergen, mustard allergen, nuts allergen, poultry allergen, meat allergen, rice allergen, sesame allergen, bee venom allergen, vespid allergen, latex allergen, dust mite allergen, insect allergen, mold allergen, fungal allergen, furry animal allergen, pollen allergen and drug allergen.
196. The vector according to any of the items 189 to 195, wherein the drug allergen is selected from the group consisting of Factor VIII, insulin and therapeutic monoclonal antibody.
197. The vector according to any of items 189 to 196, wherein the antigenic unit comprises one or more T cell epitopes of a self-antigen and the further antigenic unit comprises one or more self-antigens.
198. The vector according to any of items 189 to 197, wherein the antigenic unit comprises one or more T cell epitopes of a self-antigen and the further antigenic unit comprises one self-antigen.
199. The vector according to any of items 189 to198, wherein the self-antigen is selected from the group consisting of multiple sclerosis self-antigen, type 1 diabetes mellitus self-antigen, celiac disease self-antigen, rheumatoid arthritis self-antigen, chronic inflammatory demyelinating polyradiculoneuropathy self-antigen, Hashimoto's thyroiditis self-antigen, pemphigus foliaceus self-antigen, pemphigus vulgaris self-antigen, thyroid eye disease self-antigen, Grave's disease self-antigen, primary biliary cirrhosis, myasthenia gravis self-antigen, insulin-resistant diabetes self-antigen and hemolytic anemia self-antigen.
200. The vector according to any of items 189 to 199, wherein the antigenic unit comprises one or more T cell epitopes of an alloantigen and the further antigenic unit comprises one or more alloantigens.
201. The vector according to any of items 189 to 200, wherein the antigenic unit comprises one or more T cell epitopes of an alloantigen and the further antigenic unit comprises one alloantigen.
202. The vector according to any one of items 189 to 201, wherein the further antigenic unit of at least one of the one or more further polypeptides comprises one or more further epitopes, wherein the one or more further epitopes are comprised in one or more allergens, hypoallergenic allergens, self-antigens or alloantigens.
203. The vector according to any one of items 189 to 202, wherein the further antigenic unit of at least one of the one or more further polypeptides comprises one or more full length allergens, hypoallergenic allergens, self-antigens or alloantigens, wherein said one or more full length allergens, hypoallergenic allergens, self-antigens or alloantigens comprises one or more further epitopes.
204. The vector according to any one of items 189 to 203, wherein the further antigenic unit of at least one of the one or more further polypeptides comprises an allergen, hypoallergenic allergen, self-antigen or alloantigen or a part thereof, wherein said allergen, hypoallergenic allergen, self-antigen or alloantigen or a part thereof comprises one or more further epitopes.
205. The vector according to any one of items 189 to 204, wherein the targeting unit comprises or consists of a moiety that binds to a receptor selected from the group consisting of TGFβ receptor, such as TGFβR1, TGFβR2, or TGFβR3, IL10R, such as IL-10RA and IL10-RB, IL2R, IL4R, IL6R, IL11R and IL13R, IL27R, IL35R, IL37R, GM-CSFR, FLT3, CCR7, CD11b, CD11c, CD103, CD14, CD36, CD205, CD109, VISTA, MARCO, MHCII, CD83, SIGLEC, MGL/Clec10A, ASGR (ASGR1/ASGR2), CD80, CD86, Clec9A, Clec12A, Clec12B, DCIR2, Langerin, MR, DC-Sign, Treml4, Dectin-1, PDL1, PDL2, HVEM, CD163, CD32b and CD141.
206. The vector according to any one of items 189 to 205, wherein the targeting unit is selected from the group consisting of TGFβ, such as TGFβ1, TGFβ2 or TGFβ3, IL-10, IL2, IL4, IL6, IL11, IL13, IL27, IL35, IL37, GM-CSF, FLT3L, CCL19, CCL21, ICAM-1 (Intercellular Adhesion Molecule 1 also known as CD54), keratin, VSIG-3, SCGB3A2, CTLA-4, preferably the extracellular domain of CTLA-4, PD-1, preferably the extracellular domain of PD-1 and BTLA, preferably the extracellular domain of BTLA.
207. The vector according to any one of items 189 to 206, wherein the one or more further polypeptides comprise a further targeting unit.
208. The vector according to item 207, wherein the further targeting unit comprises or consist of XCL1, a XCL1 homolog or a XCL1 variant.
209. The vector according to item 207, wherein the further targeting unit comprises or consist of MHCII, a MHCII homolog or a MHCII variant.
210. The vector according to item 207, wherein the further targeting unit comprises or consists of a moiety that binds to a receptor selected from the group consisting of TGFβ receptor, such as TGFβR1, TGFβR2, or TGFβR3, IL10R, such as IL-10RA and IL10-RB, IL2R, IL4R, IL6R, IL11R and IL13R, IL27R, IL35R, IL37R, GM-CSFR, FLT3, CCR7, CD11b, CD11c, CD103, CD14, CD36, CD205, CD109, VISTA, MARCO, MHCII, CD83, SIGLEC, MGL/Clec10A, ASGR (ASGR1/ASGR2), CD80, CD86, Clec9A, Clec12A, Clec12B, DCIR2, Langerin, MR, DC-Sign, Treml4, Dectin-1, PDL1, PDL2, HVEM, CD163, CD32b and CD141.
211. The vector according to item 207, wherein the further targeting unit is selected from the group consisting of TGFβ, such as TGFβ1, TGFβ2 or TGFβ3, IL-10, IL2, IL4, IL6, IL11, IL13, IL27, IL35, IL37, GM-CSF, FLT3L, CCL19, CCL21, ICAM-1 (Intercellular Adhesion Molecule 1 also known as CD54), keratin, VSIG-3, SCGB3A2, CTLA-4, preferably the extracellular domain of CTLA-4, PD-1, preferably the extracellular domain of PD-1 and BTLA, preferably the extracellular domain of BTLA.
212. The vector according to item 207, wherein the further targeting unit has affinity for a surface molecule selected from the group consisting of CD14, CD40, TLR-2, TLR-4 and TLR-5, preferably affinity for a surface molecule selected from the group consisting of hCD14, hCD40, hTLR-2, hTLR-4 and hTLR-5.
213. The vector according to item 207, wherein the further targeting comprises or consists of an antibody-binding region, such as the antibody variable domains (VL and VH), with specificity for CD14, CD40, TLR-2, TLR-4 or TLR-5, such as anti-CD14, anti-CD40, anti-TLR-2, anti-TLR-4 or anti-TLR-5, preferably with specificity for hCD14, hCD40, hTLR-2, hTLR-4 or hTLR-5, such as anti-hCD14, anti-hCD40, anti-hTLR-2, anti-hTLR-4 or anti-hTLR-5.
214. The vector according to item 207, wherein the further targeting unit comprises or consists of chemokine human macrophage inflammatory protein alpha CCL3L1 variant.
215. The vector according to any one of items 189 to 207, wherein the one or more further polypeptides comprises a further multimerization unit, such as a further dimerization unit.
216. The vector according to item 215, wherein the further multimerization unit is a further dimerization unit.
217. The vector according to item 215, wherein the multimerization unit is a trimerization unit, such as a collagen-derived trimerization unit, such as a human collagen-derived trimerization domain, such as human collagen derived XVIII trimerization domain or human collagen XV trimerization domain.
218. The vector according to any one of items 189 to 208, wherein the antigenic unit of the first polypeptide and/or the further antigenic unit of at least one of the one or more further polypeptides comprise at least one tolerance-inducing universal CD4+ T cell epitope.
219. The vector according to any one of items 189 to 209, wherein the antigenic unit of the first polypeptide and/or the further antigenic unit of the one of further polypeptides comprise at least two tolerance-inducing universal CD4+ T cell epitopes.
220. The vector according to item 219, wherein the at least two tolerance-inducing universal CD4+ T cell epitopes are different tolerance-inducing universal CD4+ T cell epitopes.
221. The vector according to any of items 219 to 220, wherein the one or more tolerance-inducing universal CD4+ T cell epitopes are selected from the group consisting of T reg epitopes (Tregitopes), inhibitory epitopes, apitopes, dominant autoepitopes from nucleosomal histones and epitopes of peptides that share a consensus motif across individuals and species.
222. The vector according to any one of items 189 to 221, wherein the further antigenic unit of at least one of the one of further polypeptides comprises a furin linker amino acid sequence.
223. The vector according to any one of items 189 to 222, wherein at least one of the one or more further polypeptides comprises an interaction unit.
224. The vector according to item 223, wherein the interaction unit is selected from the group consisting of a leucine zipper motif, a sequence capable of promoting oligomerization, such as a homo-trimerization domain, a heterodimerization unit, such as an heterodimerization unit comprising a coiled coil dimer-forming peptide and an oligomerization unit.
225. The vector according to any one of items 189 to 224, wherein the first polypeptide and at least one of the one or more further polypeptides are capable of forming a multimer, such as a dimer.
226. The vector according to any one of items 189 to 225, wherein the multimerization unit and the interaction unit are capable of forming a multimer, such as a dimer, such as a heterodimer.
227. The vector according to any one of items 189 to 226, wherein the multimer is a heterodimer.
228. The vector according to any one of items 189 to 227, wherein the multimerization unit and the interaction unit are different and are capable of forming a multimer, such as a dimer, such as a heterodimer.
229. The vector according to any one of items 189 to 228, wherein the heterodimerization unit comprises a coiled coil dimer-forming peptide.
230. The vector according to any one of items 189 to 229, wherein the multimerization unit of the first polypeptide is a heterodimerization unit.
231. The vector according to any one of items 189 to 230, wherein the multimerization unit of the first polypeptide is a heterodimerization unit comprising a coiled coil dimer-forming peptide.
232. The vector according to any one of items 224 to 231, wherein the coiled coil dimer is selected from the group of:
   i. a P7A:P8A coiled coil dimer;
   ii. a N7:N8 coiled coil dimer;
   iii. a N5:N6 coiled coil dimer.
233. The vector according to any one of items 224 to 232, wherein the coiled coil dimer-forming peptide is:
   i. P7A and/or P8A;
   ii. N7 and/or N8; or
   iii. N5 and/or N6.
234. The vector according to any one of items 224 to 233, wherein the coiled coil dimer-forming peptide is P7A and/or P8A.
235. The vector according to any one of items 189 to 234, wherein at least one of the one or more further polypeptides comprises an interaction unit comprising a coiled coil dimer-forming peptide.
236. The vector according to any one of items 189 to 235, wherein the heterodimerization unit and the further heterodimeric dimerization form a coiled 237. The vector according to any one of items 189 to 236, wherein the heterodimerization unit of the first polypeptide and the further heterodimeric dimerization form a coiled coil dimer.
238. The vector according to any one of items 189 to 237, wherein at least one of the one or more further polypeptides comprises an interaction unit, wherein the interaction unit is a further heterodimerization unit, and wherein the heterodimerization unit and the further heterodimerization unit form a P7A:P8A coiled coil dimer.
239. The vector according to any one of items 189 to 238, wherein:
   i. the first polypeptide comprises a heterodimerization unit comprising a coiled coil dimer-forming peptide selected from P7A and P8A; and
   ii. at least one of the one or more further polypeptides comprises a further heterodimerization unit comprising a coiled coil dimer-forming peptide selected from P7A and P8A;
wherein the heterodimerization unit and the further heterodimeric dimerization form a P7A:P8A coiled coil dimer.
240. The vector according to any one of items 189 to 239, wherein P7A comprises or consists of the amino acid sequence

(SEQ ID NO: 16)
YGEIAALEAKNAALKAEIAALEAKNAALKAGC.

241. The vector according to any one of items 189 to 240, wherein P8A comprises or consists of the amino acid sequence

(SEQ IS NO: 17)
YGKIAALKAENAALEAKIAALKAENAALEAGGC.

242. The vector according to any one of items 189 to 241, wherein:
   i. the first polypeptide comprises a heterodimerization unit comprising a coiled coil dimer-forming peptide selected from N7 and N8; and
   ii. at least one of the one or more further polypeptides comprises a further heterodimerization unit comprising a coiled coil dimer-forming peptide selected from N7 and N8;
wherein the heterodimerization unit and the further heterodimeric dimerization form a N7:N8 coiled coil dimer.
243. The vector according to any one of items 189 to 242, wherein N7 comprises or consists of the amino acid sequence

(SEQ ID NO: 18)
YEIAALEAKNAALKAEIAALEAKIAALKAGC.

244. The vector according to any one of items 189 to 243, wherein N8 comprises or consists of the amino acid sequence

(SEQ IS NO: 19)
YKIAALKAENAALEAKIAALKAEIAALEAGC.

245. The vector according to any one of items 189 to 244, wherein:
   i. the first polypeptide comprises a heterodimerization unit comprising a coiled coil dimer-forming peptide selected from N5 and N6; and
   ii. at least one of the one or more further polypeptides comprises a further heterodimerization unit comprising a coiled coil dimer-forming peptide selected from N5 and N6;
wherein the heterodimerization unit and the further heterodimeric dimerization form a N5:N6 coiled coil dimer.
246. The vector according to any one of items 232 to 245, wherein N5 comprises or consists of the amino acid sequence

(SEQ ID NO: 20)
YEIAALEAKIAALKAKNAALKAEIAALEAGC.

247. The vector according to any one of items 232 to 246, wherein N6 comprises or consists of the amino acid sequence

(SEQ ID NO: 21)
YKIAALKAEIAALEAENAALEAKIAALKAGC.

248. The vector according to any one of items 232 to 247, wherein at least one further polypeptide forms a hetero-multimer, such as a heterodimer, with the first polypeptide via interaction of the multimerization unit of the first polypeptide and the interaction unit of said one further polypeptide.
249. The vector according to any of items 189 to 248, wherein:
   i. the antigenic unit of the first polypeptide comprises one or more T-cell epitopes of an allergen;
   ii. the further antigenic unit of at least one of the one or more further polypeptides comprises one or more allergens or hypoallergenic allergens; and
   iii. the first polypeptide and at least one further polypeptide form a heterodimer.
250. The vector according to any one of items 223 to 249, wherein the interaction unit of at least one of the one or more further polypeptides comprises a sequence that encodes an amino acid sequence that facilitates the trimerization of the antigens, such as a hetero-trimerization sequence, e.g., a heterotrimeric coiled coil trimer.
251. The vector according to any one of items 222 to 250, wherein the self-assembly unit of at least one further polypeptide is ferritin.
252. The vector according to any one of items 222 to 251, wherein the oligomerization unit of at least one further polypeptide is sortase A.
253. The vector according to any one of items 222 to 252, wherein the self-assembly unit of at least one further polypeptide is derived from the self-forming structure component of a self-assembling molecule, such as ferritin, lumazine synthase from Aquifex aeolicus (LS), E2 from Geobacillus stearothermophilus, or 13-01-modified 2-dehydro-3-deoxy-phosphogluconate aldolase (=2-Keto-3-deoxy-6-phosphogluconate (KDPG) aldolase)
254. The vector according to any one of items 222 to 253, wherein the oligomerization unit of at least one further polypeptide is selected from the group consisting of: sortase A; lumazine synthase from Aquifex aeolicus (LS), E2 from Geobacillus stearothermophilus, and 13-01-modified 2-dehydro-3-deoxy-phosphogluconate aldolase (=2-Keto-3-deoxy-6-phosphogluconate (KDPG) aldolase).
255. The vector according to any one of items 189 to 254, wherein the first polypeptide comprises a first leucine zipper motif.
256. The vector according to any one of the items 189 to 255, wherein the one or more further polypeptides comprise a second leucine zipper motif.
257. The vector according to item 256, wherein the first leucine zipper motif and the second leucine zipper motif are capable of forming a dimer.
258. The vector according to any one of items 256 to 257, wherein the first leucine zipper motif is at the C terminal end of the first polypeptide and the second leucine zipper motif is at the C terminal end or at the N terminal end of the one or more further polypeptides.
259. The vector according to any one of items 223 to 258, wherein an interaction unit comprising a first leucine zipper motif is at the C terminal end or at the N terminal end of one further polypeptide, and an interaction unit comprising a second leucine zipper motif is at the C terminal end or at the N terminal end of one other further polypeptide.
260. The vector according to any one of items 189 to 259, wherein at least two of the one or more further polypeptides comprise a zipper motif, wherein the leucine zipper motifs are capable of forming a dimer between the at least two further polypeptides.
261. The vector according to any one of items 224 to 260, wherein at least one leucine zipper motif comprises the amino acid sequence

(SEQ ID NO: 24)
LEIRAAFLRQRNTALRTEVAELEQEVQRLENEVSQYETRYGPLGGGK.

262. The vector according to any one of items 224 to 261, wherein at least one leucine zipper motif comprises the amino acid sequence

(SEQ ID NO: 25)
LEIEAAFLERENTALETRVAELRQRVQRLRNRVSQYRTRYGPLGGGK.

263. The vector according to any one of items 189 to 262, wherein the first nucleic acid sequence encodes a signal peptide and/or at least one of the one or more further nucleic acid sequences encodes a further signal peptide.
264. The vector according to any one of items 189 to 263, wherein the first polypeptide comprises a signal peptide and/or at least one of the one or more further polypeptides comprises a further signal peptide.
265. The vector according to any one of items 189 to 264, wherein at least one of the one or more further polypeptides comprises a further signal peptide.
266. The vector according to any one of items 263 to 265, wherein the further signal peptide of at least one further polypeptide is different from the signal peptide of the targeting unit of the first polypeptide.
267. The vector according to any one of items 263 to 266, wherein the signal peptide and/or the further signal peptide of at least one further polypeptide is the natural leader sequence of the protein which is the targeting unit.
268. The vector according to any one of items 263 to 267, wherein the signal peptide and/or the further signal peptide of at least one further polypeptide is selected from the group consisting of Ig VH signal peptide, human serum albumin signal peptide (SEQ ID NO: 138), human modified IgG H signal peptide (SEQ ID NO: 139), human HC H6 signal peptide (SEQ ID NO: 140), human TPA signal peptide and human CCL3L1 signal peptide.
269. The vector according to any one of items 263 to 268, wherein the further targeting unit is human CCL3L1 and the signal peptide and/or the further signal peptide of at least one further polypeptide comprises an amino acid sequence having at least 85% sequence identity to the amino acid sequence 1-23 of SEQ ID NO: 128.
270. The vector according to any one of items 263 to 269, wherein the signal peptide and/or the further signal peptide of at least one further polypeptide consists of an amino acid sequence having at least 85% sequence identity to the amino acid sequence 1-23 of SEQ ID NO: 128.
271. The vector according to any one of items 263 to 270, wherein the signal peptide and/or the further signal peptide of at least one further polypeptide consists of the amino acid sequence 1-23 of SEQ ID NO: 128.
272. The vector according to any one of items 189 to 271, wherein the further antigenic unit of at least one of the one or more further polypeptides comprises one or more allergens, hypoallergenic allergens, self-antigens or alloantigens comprising one or more B-cell epitopes, such as one or more conformational B-cell epitopes.
273. The vector according to any one of items 189 to 272, wherein the first polypeptide comprises more than one T cell epitope of an allergen, self-antigen or alloantigen, such as 2, 3, 4, 5, 6, 7 or 8 T cell epitopes, such as 2, 3, 4, 5, 6, 7 or 8 different T cell epitopes.
274. The vector according to any one of items 189 to 273, wherein at least one of the one or more further polypeptides comprises more than one epitope, such as 2, 3, 4, 5, 6, 7 or 8 epitopes, such as 2, 3, 4, 5, 6, 7 or 8 different epitopes.
275. The vector according to any one of items 189 to 274, wherein at least one of the one or more further polypeptides comprises more than one allergen, hypoallergenic allergen, self-antigen or alloantigen, such as 2, 3, 4, 5, 6, 7 or 8 antigens, such as 2, 3, 4, 5, 6, 7 or 8 allergens, hypoallergenic allergens, self-antigens or alloantigens.
276. The vector according to any one of items 189 to 275, wherein at least one of the one or more further polypeptides comprises one or more full length antigens.
277. The vector according to any one of items 189 to 276, wherein at least one of the one or more further polypeptides comprises one full length allergen, hypoallergenic allergen, self-antigen or alloantigen.
278. The vector according to any one of items 189 to 277, wherein the allergen, hypoallergenic allergen, self-antigen or alloantigen has an amino acid sequence of at least 20, 50, 60, 80, 100, 120, 140, 160, 180, 200, 250, 300, 350, 400, 450, 500, 550, 600, 650, 750, 800, 900, 1000, 1250, 1500, 1750, 2000, 2500, 3000 amino acids.
279. The vector according to any one of items 189 to 278, wherein the allergen, hypoallergenic allergen, self-antigen or alloantigen comprises one or more T cell epitopes and one or more B cell epitopes.
280. The vector according to any one of items 189 to 279, wherein the allergen, hypoallergenic allergen, self-antigen or alloantigen comprises one or more T cell epitopes.
281. The vector according to any one of items 189 to 280, wherein the allergen, hypoallergenic allergen, self-antigen or alloantigen comprises one or more B cell epitope.
282. The vector according to any one of items 189 to 281, wherein said vector comprises one or more co-expression elements.
283. The vector according to any one of items 189 to 282, wherein at least one co-expression element causes the transcription of the first polypeptide and the one or more further polypeptides on a single transcript and the independent translation into a separate first polypeptide and separate one or more further polypeptides.
284. The vector according to any one of items 282 to 283, wherein at least one of the one or more co-expression elements are IRES elements, or nucleic acid sequences encoding 2A self-cleaving peptides.
285. The vector according to any one of items 282 to 284, wherein at least one of the one or more co-expression elements is a nucleic acid sequence encoding a 2A self-cleaving peptide.
286. The vector according to item 287, wherein the 2A self-cleaving peptide is selected from the group consisting of T2A peptide, P2A peptide, E2A peptide and F2A peptide.
287. The vector according to any one of items 282 to 286, wherein at least one of the one or more co-expression elements promotes the transcription of the first polypeptide and the one or more further polypeptides as separate transcripts.
288. The vector according to any one of items 282 to 287, wherein the one or more co-expression elements are a) bidirectional promoters or b) promoters, wherein the vector comprises a separate bidirectional promoter or separate promoter for each of the nucleic acid sequences encoding the first polypeptide and the one or more further polypeptides.
289. The vector according to any one of items 189 to 288, wherein the multimerization unit of the first polypeptide is selected from the group consisting of dimerization unit, trimerization unit, such as a collagen-derived trimerization unit, such as a human collagen-derived trimerization domain, such as human collagen derived XVIII trimerization domain or human collagen XV trimerization domain or the C-terminal domain of T4 fibritin and tetramerization unit, such as a domain derived from p53 and wherein said multimerization unit optionally comprises a hinge region, such as hinge exon h1 and hinge exon h4.
290. The vector according to any one of items 189 to 289, wherein the multimerization unit and/or further multimerization unit is a dimerization unit and said dimerization unit further comprises another domain that facilitates dimerization, preferably wherein the other domain is an immunoglobulin domain, more preferably an immunoglobulin constant domain.
291. The vector according to item 290, wherein the other domain is a carboxyterminal C domain derived from IgG, preferably from IgG3.
292. The vector according to any one of items 189 to 291, wherein the dimerization unit and/or further dimerization unit comprises a dimerization unit linker, such as glycine-serine rich linker, such as GGGSSGGGSG (SEQ ID NO: 15) and preferably wherein the dimerization unit linker connects the hinge region and the other domain that facilitates dimerization.
293. The vector according to any one of items 189 to 292, wherein the dimerization unit and/or further dimerization unit comprises hinge exon h1 and hinge exon h4, a dimerization unit linker and a CH3 domain of human IgG3.
294. The vector according to any one of items 189 to 293, wherein the first nucleic acid sequence encodes a first polypeptide which further comprises a unit liker that connects the antigenic unit to the multimerization unit, and wherein the unit linker is a non-immunogenic linker and/or flexible or rigid linker.
295. The vector according to any one of items 189 to 294, wherein the first nucleic acid sequence encodes a first polypeptide which comprises a signal peptide, and preferably wherein at least one of the one or more further nucleic acid sequences encodes a further signal peptide.
296. The vector according to any one of items 189 to 295, wherein the vector is a viral vector, such as an RNA viral vector or DNA viral vector or a plasmid, such as an RNA plasmid or DNA plasmid 297. The vector according to any one of items 189 to 296, wherein the first nucleic acid sequence and/or the one or more further nucleic acid sequences is selected from DNA sequence and RNA sequence.
298. The vector according to any one of items 189 to 297, wherein the vector further encodes one or more additional polynucleotides, wherein the one or more additional polynucleotides comprise one or more nucleic acid sequences encoding one or more immunoinhibitory compounds and wherein the vector allows for the co-expression of the first polypeptide and the one or more immunoinhibitory compounds as separate molecules.
299. The vector according to item 298, wherein the one or more immunoinhibitory compounds is an inhibitory checkpoint molecule, preferably selected from the group consisting of: CTLA-4 (SEQ ID NO: 248), PD-1 (SEQ ID NO: 273), BLTA, TIM-3, IL-10 (SEQ ID NO: 260), TGFβ1 (SEQ ID NO: 249), TGFβ2 (SEQ ID NO: 250), TGFβ3 (SEQ ID NO: 251), IL-27, IL-2, IL-37 and IL-35.
300. The vector according to any one of items 298 to 299, wherein the one or more immunoinhibitory compounds is an inhibitor of the cGAS-STING pathway, such as Vaccinia E5 (SEQ ID NO: 292).
301. The vector according to any one of the preceding items, wherein the vectors comprises or consists of one or more of:
   CCpG;
   CpGG;
   CpGCpGCpG;
   CpG repeats.
302. The vector according to any one of the preceding items, wherein the vector has been modified to reduce the number of CpG-S motifs such as CpGC, CpGG, ApACpGGTpT, ApACpGCTpT, GpACpGGTpT, GpACpGCTpT GpTCpGGTpT, and GpTCpGCTpT.
303. A method of producing a vector as defined in any one of items 189 to 302, the method comprising the following steps:
   a) transfecting cells in vitro with the vector according to any one of the previous items;
   b) culturing said cells;
   c) optionally, lysing the cells to release the vector from the cells; and
   d) collecting and optionally purifying the vector.
304. A host cell comprising a vector as defined in any one of items 189 to 303, such as a host cell selected from the group consisting of prokaryote cells, yeast cells, insect cells, higher eukaryotic cells such as cells from animals or humans.
305. A vector as defined in any one of items 189 to 303 for use as a medicament.
306. A pharmaceutical composition comprising the vector as defined in any one of items 189 to 303 and a pharmaceutically acceptable carrier or diluent.
307. The pharmaceutical composition according to item 306, wherein the composition further comprises a transfection agent.
308. The pharmaceutical composition according to any one of items 306 to 307, wherein the composition comprises said vector, e.g., said DNA plasmid, in a range of from 0.1 to 10 mg.
309. A method of treating a subject having a disease selected from autoimmune diseases, allergic diseases and graft rejection or being in need of prevention of said disease, the method comprising administering to the subject a vector as defined in any one of items 189 to 303 or a pharmaceutical composition as defined in any one of items 306 to 308.
310. The method according to item 309, wherein the vector or the pharmaceutical composition is administered in a therapeutically or prophylactically effective amount, such as administered by intradermal, intramuscular, or subcutaneous injection, or by mucosal or epithelial application, such as intranasal or oral.
311. A vector as defined in any one of items 189 to 303 or a pharmaceutical composition as defined in any one of items 306 to 308 for use in the prophylactic or therapeutic treatment of autoimmune diseases, allergic diseases or graft rejection.
312. A method of treating a subject having an allergic disease or being in need of prevention of said allergic disease, the method comprising administering to the subject a vector as defined in any one of items 189 to 303 or a pharmaceutical composition as defined in any one of items 306 to 308.
313. The method according to item 312, wherein the vector or the pharmaceutical composition is administered in a therapeutically or prophylactically effective amount, such as administered by intradermal, intramuscular, or subcutaneous injection, or by mucosal or epithelial application, such as intranasal or oral.
314. A vector as defined in any one of items 189 to 303 or a pharmaceutical composition as defined in any one of items 306 to 308 for use in the prophylactic or therapeutic treatment an allergic disease.

1. A vector comprising:
(a) a first nucleic acid sequence encoding a first polypeptide, wherein the first polypeptide comprises a targeting unit that targets antigen-presenting cells, a multimerization unit, such as a dimerization unit, and an antigenic unit comprising one or more epitopes; and
(b) one or more further nucleic acid sequences encoding one or more further polypeptides, wherein the one or more further polypeptides comprise a further antigenic unit comprising one or more further epitopes,
wherein the vector allows for the co-expression of the first polypeptide and the one or more further polypeptides as separate molecules.

2. The vector according to claim 1, wherein the one or more epitopes and/or the one or more further epitopes are selected from the group consisting of non-self epitopes, non-self antigens or parts thereof, disease relevant epitopes and disease relevant antigens or parts thereof.

3. The vector according to any one of the previous claims, wherein the one or more epitopes and/or the one or more further epitopes are derived from one or more pathogen, optionally wherein the one or more pathogens are selected from the group consisting of viruses, bacteria, fungi and parasites.

4. The vector according to claim 1, wherein the one or more epitopes are one or more T cell epitopes of an allergen, self-antigen or alloantigen.

5. The vector according to any one of claim 1 or 4, wherein the further antigenic unit comprises one or more allergens, hypoallergenic allergens, self-antigens or alloantigens.

6. The vector according to any one of the preceding claims, wherein the further antigenic unit of at least one of the one or more further polypeptides comprises one or more full length proteins, wherein said one or more full length proteins comprises the one or more further epitopes.

7. The vector according to any one of the previous claims, wherein the further antigenic unit of at least one of the one or more further polypeptides comprises an antigen or a part thereof, wherein said antigen or a part thereof comprises the one or more further epitopes.

8. The vector according to any one of the previous claims, wherein the antigenic unit of the first polypeptide and/or the further antigenic unit of at least one of the one or more further polypeptides comprise at least one universal CD4+ T cell epitope, such as at least two universal CD4+ T cell epitopes.

9. The vector according to any one of the previous claims, wherein the further antigenic unit of at least one of the one or more further polypeptides comprises an antigen and/or a furin linker amino acid sequence.

10. The vector according to any one of the previous claims, wherein the antigenic unit comprises one or more T cell epitopes from an antigen, such as an antigen from a pathogen, and at least one of the one or more further antigenic unit comprises a further antigen, such as an antigen from a pathogen, such as a full length antigen from a pathogen.

11. The vector according to any one of the previous claims, wherein the antigenic unit comprises an antigen, such as an antigen from a pathogen, and at least one of the one or more further antigenic unit comprises a further antigen which is identical to the first antigen.

12. The vector according to any one of the previous claims, wherein the antigen and at least one of the one or more further antigens can form a multimer, such as a dimer or a trimer.

13. The vector according to any one of the previous claims, wherein the antigenic unit comprises one or more T cell epitopes from an antigen, such as an antigen from a pathogen, and at least one of the one or more further antigenic unit comprises a further antigen which is a membrane bound antigen, such as a membrane bound antigen from a pathogen.

14. The vector according to any one of the previous claims, wherein at least one of the one or more further polypeptides comprises an interaction unit, preferably wherein the interaction unit is selected from the group consisting of a dimerization unit, a trimerization unit, and a tetramerization unit.

15. The vector according to claim 14, wherein the interaction unit is selected from the group consisting of a leucine zipper motif, a sequence capable of promoting oligomerization, such as a homo-trimerization domain, a heterodimerization unit, such as a heterodimerization unit comprising or consisting of a coiled coil dimer-forming peptide, an oligomerization unit and a self-assembly unit.

16. The vector according to any one of the previous claims, wherein the first polypeptide comprises a sequence which interacts with the interaction unit, such as a first leucine zipper motif, if the interaction unit is a second leucine zipper motif or a first coiled coil dimer-forming peptide, if the interaction unit is a second coil dimer-forming peptide.

17. The vector according to any one of the previous claims, wherein the antigenic unit comprises one or more T cell epitopes from an antigen, such as an antigen from a pathogen, and at least one of the one or more further polypeptides comprises an interaction unit which is an oligomerization unit, such a homotrimerization unit, such as a T4-phage fibritin trimerization domain or ferritin, and a further antigenic unit that comprises a further antigen, such as one or more further antigens from a pathogen.

18. The vector according to any one of the previous claims, wherein the multimerization unit of the first polypeptide and the interaction unit of at least one of the one or more further polypeptides are capable of forming a first polypeptide/further polypeptide multimer, such as a dimer, such as a heterodimer, optionally wherein the multimerization unit and the interaction unit are different.

19. The vector according to any one of the previous claims, wherein more than one further polypeptide comprises an interaction unit and said interaction units are capable of forming a multimer, such as a homo- or heteromultimer of said further polypeptides.

20. The vector according to any one of the previous claims, wherein the interaction unit of at least one of the one or more further polypeptides comprises a sequence that facilitates the trimerization of the antigens comprised in the antigenic unit of the first polypeptide and the antigens comprised in the further antigenic unit of the at least one further polypeptide, such as a hetero-trimerization sequence, e.g., a coiled coil peptide.

21. The vector according to any one of the previous claims, wherein the vector encodes at least 3 further polypeptides comprising an interaction unit that comprises a sequence that facilitates the trimerization of the antigens comprised in the further antigenic units of said further polypeptides, such as a heterotrimerization sequence, e.g., a coiled coil peptide or a homotrimerization unit, such as a T4-phage fibritin trimerization domain.

22. The vector according to any one of the previous claims, wherein at least one of the one or more further polypeptide comprises an interaction unit which is a self-assembly unit, such as ferritin.

23. The vector according to any one of the previous claims, wherein at least one of the one or more further polypeptide comprises an interaction unit which is an oligomerization unit, such as sortase A.

24. The vector according to any one of the previous claims, wherein at least one of the one or more further polypeptide comprises an interaction unit which is a self-assembly unit derived from the self-forming structure component of a self-assembling molecule, such as lumazine synthase from Aquifex aeolicus (LS), E2 from Geobacillus stearothermophilus, or 13-01-modified 2-dehydro-3-deoxy-phosphogluconate aldolase (=2-Keto-3-deoxy-6-phosphogluconate (KDPG) aldolase) and/or wherein the oligomerization unit of at least one further polypeptide is selected from the group consisting of: sortase A; lumazine synthase from Aquifex aeolicus (LS), E2 from Geobacillus stearothermophilus, and 13-01-modified 2-dehydro-3-deoxy-phosphogluconate aldolase (=2-Keto-3-deoxy-6-phosphogluconate (KDPG) aldolase).

25. The vector according to any one of the previous claims, wherein the first polypeptide comprises a first leucine zipper motif and wherein the interaction unit of at least one of the one or more further polypeptides comprises a second leucine zipper motif, and wherein the first and second leucine zipper motifs are capable of forming a dimer.

26. The vector according to claim 25, wherein the first leucine zipper motif is at the C terminal end of the first polypeptide and the second leucine zipper motif is at the C terminal end or at the N terminal end of the one or more further polypeptides.

27. The vector according to any one of the previous claims, wherein at least two of the one or more further polypeptides comprise an interaction unit which is a leucine zipper motif, wherein the leucine zipper motifs are capable of forming a dimer between the at least two further polypeptides.

28. The vector according to any one of the previous claims, wherein at least one of the one or more further polypeptides comprises a further targeting unit.

29. The vector according to any one of the previous claims, wherein the first nucleic acid sequence encodes a signal peptide and/or at least one of the one or more further nucleic acid sequences encodes a further signal peptide, optionally wherein the further signal peptide is different from the signal peptide.

30. The vector according to claim 29, wherein the signal peptide and/or the further signal peptide is the natural leader sequence of the protein which is the targeting unit.

31. The vector according to any one of the preceding claims, wherein the targeting unit and/or the further targeting unit comprises or consists of a moiety that interacts with a surface molecule selected from the group consisting of CD14, CD40, CLEC9A, chemokine receptors, such as CCR1, CCR3, CCR4, CCR5, CCR6, CCR7, CCR8 and XCR1, and Toll-like receptors (TLRs), such as TLR-2, TLR-4 and TLR-5.

32. The vector according to any one of the preceding claims, wherein the targeting unit and/or the further targeting unit comprises or consists of an antibody-binding region, such as antibody variable domains (VL and VH) with specificity for MHC/HLA, CD14, CD40, CLEC9A or Toll-like receptors; or a synthetic or natural ligand, such as soluble CD40 ligand (CD40L), natural ligands like chemokines, preferably CCL5, CCL3, CCL4, CCL19, CCL21, XCL1 or XCL2), bacterial antigens like for example flagellin.

33. The vector according to any one of claims 29 to 34, wherein the targeting unit and/or the further targeting unit comprises or consists of human CCL3L1 and the signal peptide and/or the further signal peptide comprises an amino acid sequence having at least 85% sequence identity to the amino acid sequence 1-23 of SEQ ID NO: 128.

34. The vector according to any one of the preceding claims, wherein the targeting unit and/or the further targeting comprises or consists of a moiety that binds to a receptor selected from the group consisting of TGFβ receptor, such as TGFβR1, TGFβR2, or TGFβR3, IL10R, such as IL-10RA and IL10-RB, IL2R, IL4R, IL6R, IL11R and IL13R, IL27R, IL35R, IL37R, GM-CSFR, FLT3, CCR7, CD11b, CD11c, CD103, CD14, CD36, CD205, CD109, VISTA, MARCO, MHCII, CD83, SIGLEC, MGL/Clec10A, ASGR (ASGR1/ASGR2), CD80, CD86, Clec9A, Clec12A, Clec12B, DCIR2, Langerin, MR, DC-Sign, Treml4, Dectin-1, PDL1, PDL2, HVEM, CD163, CD32b and CD141.

35. The vector according to any one of the preceding claims, wherein at least one of the one or more further polypeptides comprises one or more full length antigens.

36. The vector according to according to any one of the previous claims, wherein said vector comprises one or more co-expression elements causing the transcription of the first polypeptide and of the one or more further polypeptides on a single transcript, and the independent translation into a separate first polypeptide and separate one or more further polypeptides, preferably wherein at least one of the one or more co-expression elements are IRES elements, or nucleic acid sequences encoding 2A self-cleaving peptides.

37. The vector according to claim 36, wherein the 2A self-cleaving peptide is selected from the group consisting of T2A peptide, P2A peptide, E2A peptide and F2A peptide.

38. The vector according to any one of claims 36 to 37, wherein the one or more co-expression elements are a) bidirectional promoters or b) promoters, wherein the vector comprises a separate bidirectional promoter or separate promoter for each of the nucleic acid sequences encoding the first polypeptide and the one or more further polypeptides.

39. The vector according to any one of the preceding claims, wherein the multimerization unit of the first polypeptide is selected from the group consisting of dimerization unit, trimerization unit, such as a collagen-derived trimerization unit, such as a human collagen-derived trimerization domain, such as human collagen derived XVIII trimerization domain or human collagen XV trimerization domain or the C-terminal domain of T4 fibritin and tetramerization unit, such as a domain derived from p53 and wherein said multimerization unit optionally comprises a hinge region, such as hinge exon h1 and hinge exon h4.

40. The vector according to any one of the previous claims, wherein the multimerization unit of the first polypeptide is a dimerization unit and said dimerization unit further comprises another domain that facilitates dimerization, preferably wherein the other domain is an immunoglobulin domain, more preferably an immunoglobulin constant domain, such as a carboxyterminal C domain derived from IgG, preferably from IgG3.

41. The vector according to any one of the previous claims, wherein the dimerization unit of the first polypeptide further comprises a dimerization unit linker, such as glycine-serine rich linker, such as GGGSSGGGSG (SEQ ID NO: 15), preferably wherein the dimerization unit linker connects the hinge region and the other domain that facilitates dimerization.

42. The vector according to any one of the previous claims, wherein the dimerization unit of the first polypeptide comprises hinge exon h1 and hinge exon h4, a dimerization unit linker and a CH3 domain of human IgG3.

43. The vector according to any one of the previous claims, wherein the first nucleic acid sequence encodes a first polypeptide which further comprises a unit liker that connects the antigenic unit to the multimerization unit, and wherein the unit linker is a non-immunogenic linker and/or flexible or rigid linker.

44. The vector according to any one of the preceding claims, comprising one or more additional nucleic acid sequences encoding one or more immunostimulatory compounds.

45. The vector according to any one of the preceding claims, comprising one or more additional nucleic acid sequences encoding one or more immunoinhibitory compounds.

46. A method of producing a vector as defined in any one of the previous claims, the method comprising the following steps:
a) transfecting cells in vitro with the vector according to any one of the previous claims;
b) culturing said cells;
c) optionally, lysing the cells to release the vector from the cells; and
d) collecting and optionally purifying the vector.

47. A host cell comprising a vector as defined in any one of claims 1 to 45, such as a host cell selected from the group consisting of prokaryote cells, yeast cells, insect cells, higher eukaryotic cells such as cells from animals or humans.

48. A vector as defined in any one of claims 1 to 45 for use as a medicament.

49. A pharmaceutical composition comprising the vector as defined in any one of claims 1 to 45 and a pharmaceutically acceptable carrier or diluent and optionally a transfection agent.

50. The pharmaceutical composition according to claim 49, wherein the composition comprises said vector in a range of from 0.1 to 10 mg.

51. The pharmaceutical composition according to any one of claims 49 to 50 for use as a medicament.

52. The pharmaceutical composition according to any one of claims 49 to 51 or the vector according to any one of claims 1 to 45, for use in a method of treatment or prophylaxis of a disease or disorder in a subject in need thereof,
the method comprising administering to the subject a vector as defined in any one of claims 1 to 45 or a pharmaceutical composition as defined in any one of claims 49 to 50,
preferably wherein the vector or the pharmaceutical composition is administered in a therapeutically or prophylactically effective amount, such as is administered by intradermal, intramuscular, or subcutaneous injection, or by mucosal or epithelial application, such as intranasal or oral.

53. The pharmaceutical composition or the vector for the use according to claim 52, wherein the disease is cancer.

54. The pharmaceutical composition or the vector for the use according to claim 52, wherein the disease is an infectious disease.

55. The pharmaceutical composition or the vector for the use according to claim 52, wherein the disease is an auto-immune disease, an allergic disease, or graft rejection.


SEQUENCE LISTING (297 sequences)

SEQ ID NO: 1 - protein, 8 aa, synthetic construct
DXEXNPGP

SEQ ID NO: 2 - protein, 8 aa, synthetic construct
DVEENPGP

SEQ ID NO: 3 - protein, 18 aa, synthetic construct
EGRGSLLTCGDVEENPGP

SEQ ID NO: 4 - protein, 19 aa, synthetic construct
ATNFSLLKQAGDVEENPGP

SEQ ID NO: 5 - protein, 8 aa, synthetic construct
DVESNPGP

SEQ ID NO: 6 - protein, 20 aa, synthetic construct
QCTNYALLKLAGDVESNPGP

SEQ ID NO: 7 - protein, 22 aa, synthetic construct
VKQTLNFDLLKLAGDVESNPGP

SEQ ID NO: 8 - protein, 21 aa, synthetic construct
GSGEGRGSLLTCGDVEENPGP

SEQ ID NO: 9 - protein, 22 aa, synthetic construct
GSGATNFSLLKQAGDVEENPGP

SEQ ID NO: 10 - protein, 23 aa, synthetic construct
GSGQCTNYALLKLAGDVESNPGP

SEQ ID NO: 11 - protein, 25 aa, synthetic construct
GSGVKQTLNFDLLKLAGDVESNPGP

SEQ ID NO: 12 - protein, 34 aa, synthetic construct
AEIAAIEYEQAAIKEEIAAIKDKIAAIKEYIAAI

SEQ ID NO: 13 - protein, 34 aa, synthetic construct
EKIAAIKEEQAAIEEEIQAIKEEIAAIKYLIAQI

SEQ ID NO: 14 - protein, 34 aa, synthetic construct
AEIAAIKYKQAAIKNEIAAIKQEIAAIEQMIAAI

SEQ ID NO: 15 - protein, 10 aa, synthetic construct
GGGSSGGGSG

SEQ ID NO: 16 - protein, 32 aa, synthetic construct
YGEIAALEAKNAALKAEIAALEAKNAALKAGC

SEQ ID NO: 17 - protein, 33 aa, synthetic construct
YGKIAALKAENAALEAKIAALKAENAALEAGGC

SEQ ID NO: 18 - protein, 31 aa, synthetic construct
YEIAALEAKNAALKAEIAALEAKIAALKAGC

SEQ ID NO: 19 - protein, 31 aa, synthetic construct
YKIAALKAENAALEAKIAALKAEIAALEAGC

SEQ ID NO: 20 - protein, 31 aa, synthetic construct
YEIAALEAKIAALKAKNAALKAEIAALEAGC

SEQ ID NO: 21 - protein, 31 aa, synthetic construct
YKIAALKAEIAALEAENAALEAKIAALKAGC

SEQ ID NO: 22 - protein, 31 aa, synthetic construct
YGGIEAKIEAIEAKAEAIEAKIEAIEAKIEA

SEQ ID NO: 23 - protein, 30 aa, synthetic construct
GGIEQKIEAIEWKWEAIEQKIEAIEQKIEA

SEQ ID NO: 24 - protein, 47 aa, synthetic construct
LEIRAAFLRQRNTALRTEVAELEQEVQRLENEVSQYETRYGPLGGGK

SEQ ID NO: 25 - protein, 47 aa, synthetic construct
LEIEAAFLERENTALETRVAELRQRVQRLRNRVSQYRTRYGPLGGGK

SEQ ID NO: 26 - protein, 5 aa, synthetic construct
GLGGL

SEQ ID NO: 27 - protein, 5 aa, synthetic construct
GLSGL

SEQ ID NO: 28 - protein, 5 aa, synthetic construct
GGGGS

SEQ ID NO: 29 - protein, 10 aa, synthetic construct
GGGGSGGGGS

SEQ ID NO: 30 - protein, 5 aa, synthetic construct
  REPEAT 1..5: note = the peptide can be repeated m times, wherein m is an integer from 1 to 5, e.g., 1, 2, 3, 4, or 5.
GGGGS

SEQ ID NO: 31 - protein, 5 aa, synthetic construct
EAAAK

SEQ ID NO: 32 - protein, 5 aa, synthetic construct
  REPEAT 1..5: note = the peptide can be repeated m times, wherein m is an integer from 1 to 5, e.g., 1, 2, 3, 4, or 5.
EAAAK

SEQ ID NO: 33 - protein, 7 aa, synthetic construct
  REPEAT 1..5: note = the peptide can be repeated m times, wherein m is an integer from 1 to 5, e.g., 1, 2, 3, 4, or 5.
EAAAKGS

SEQ ID NO: 34 - protein, 6 aa, synthetic construct
  REPEAT 1..4: note = the peptide can be repeated m times, wherein m is an integer from 1 to 5, e.g., 1, 2, 3, 4, or 5.
EAAKGS

SEQ ID NO: 35 - protein, 17 aa, synthetic construct
GPSRLEEELRRRLTEPG

SEQ ID NO: 36 - protein, 12 aa, synthetic construct
HEYGAEALERAG

SEQ ID NO: 37 - protein, 5 aa, synthetic construct
GGGSS

SEQ ID NO: 38 - protein, 5 aa, synthetic construct
GGGSG

SEQ ID NO: 39 - protein, 5 aa, synthetic construct
GGSGG

SEQ ID NO: 40 - protein, 6 aa, synthetic construct
SGSSGS

SEQ ID NO: 41 - protein, 5 aa, synthetic construct
  REPEAT 1..5: note = the peptide can be repeated m times, wherein m is an integer from 1 to 5, e.g., 1, 2, 3, 4, or 5.
GGGSS

SEQ ID NO: 42 - protein, 5 aa, synthetic construct
  REPEAT 1..5: note = the peptide can be repeated m times, wherein m is an integer from 1 to 5, e.g., 1, 2, 3, 4, or 5.
GGSGG

SEQ ID NO: 43 - protein, 5 aa, synthetic construct
  REPEAT 1..5: note = the peptide can be repeated m times, wherein m is an integer from 1 to 5, e.g., 1, 2, 3, 4, or 5.
GGGSG

SEQ ID NO: 44 - protein, 6 aa, synthetic construct
SGSSGS

SEQ ID NO: 45 - protein, 5 aa, synthetic construct
LGGGS

SEQ ID NO: 46 - protein, 5 aa, synthetic construct
GLGGS

SEQ ID NO: 47 - protein, 5 aa, synthetic construct
GGLGS

SEQ ID NO: 48 - protein, 5 aa, synthetic construct
GGGLS

SEQ ID NO: 49 - protein, 5 aa, synthetic construct
GGGGL

SEQ ID NO: 50 - protein, 5 aa, synthetic construct
LGGSG

SEQ ID NO: 51 - protein, 5 aa, synthetic construct
GLGSG

SEQ ID NO: 52 - protein, 5 aa, synthetic construct
GGLSG

SEQ ID NO: 53 - protein, 5 aa, synthetic construct
GGGLG

SEQ ID NO: 54 - protein, 5 aa, synthetic construct
GGGSL

SEQ ID NO: 55 - protein, 5 aa, synthetic construct
LGGSS

SEQ ID NO: 56 - protein, 5 aa, synthetic construct
GLGSS

SEQ ID NO: 57 - protein, 5 aa, synthetic construct
GGLSS

SEQ ID NO: 58 - protein, 5 aa, synthetic construct
LGLGS

SEQ ID NO: 59 - protein, 5 aa, synthetic construct
GLGLS

SEQ ID NO: 60 - protein, 5 aa, synthetic construct
GLLGS

SEQ ID NO: 61 - protein, 5 aa, synthetic construct
LGGLS

SEQ ID NO: 62 - protein, 5 aa, synthetic construct
LGLSG

SEQ ID NO: 63 - protein, 5 aa, synthetic construct
GLLSG

SEQ ID NO: 64 - protein, 5 aa, synthetic construct
GGLSL

SEQ ID NO: 65 - protein, 5 aa, synthetic construct
GGLLG

SEQ ID NO: 66 - protein, 5 aa, synthetic construct
GLGSL

SEQ ID NO: 67 - protein, 5 aa, synthetic construct
LGLSS

SEQ ID NO: 68 - protein, 5 aa, synthetic construct
GGLLS

SEQ ID NO: 69 - protein, 10 aa, synthetic construct
LGGGSGGGGS

SEQ ID NO: 70 - protein, 10 aa, synthetic construct
GLGGSGGGGS

SEQ ID NO: 71 - protein, 10 aa, synthetic construct
GGLGSGGGGS

SEQ ID NO: 72 - protein, 10 aa, synthetic construct
GGGLSGGGGS

SEQ ID NO: 73 - protein, 10 aa, synthetic construct
GGGGLGGGGS

SEQ ID NO: 74 - protein, 10 aa, synthetic construct
LGGSGGGGSG

SEQ ID NO: 75 - protein, 10 aa, synthetic construct
GLGSGGGGSG

SEQ ID NO: 76 - protein, 10 aa, synthetic construct
GGLSGGGGSG

SEQ ID NO: 77 - protein, 10 aa, synthetic construct
GGGLGGGGSG

SEQ ID NO: 78 - protein, 10 aa, synthetic construct
GGGSLGGGSG

SEQ ID NO: 79 - protein, 10 aa, synthetic construct
LGGSSGGGSS

SEQ ID NO: 80 - protein, 10 aa, synthetic construct
GLGSSGGGSS

SEQ ID NO: 81 - protein, 10 aa, synthetic construct
GGLSSGGGSS

SEQ ID NO: 82 - protein, 10 aa, synthetic construct
GGGLSGGGSS

SEQ ID NO: 83 - protein, 10 aa, synthetic construct
GGGSLGGGSS

SEQ ID NO: 84 - protein, 10 aa, synthetic construct
LGGGSLGGGS

SEQ ID NO: 85 - protein, 10 aa, synthetic construct
GLGGSGLGGS

SEQ ID NO: 86 - protein, 10 aa, synthetic construct
GGLGSGGLGS

SEQ ID NO: 87 - protein, 10 aa, synthetic construct
GGGLSGGGLS

SEQ ID NO: 88 - protein, 10 aa, synthetic construct
GGGGLGGGGL

SEQ ID NO: 89 - protein, 10 aa, synthetic construct
LGGSGLGGSG

SEQ ID NO: 90 - protein, 10 aa, synthetic construct
GLGSGGLGSG

SEQ ID NO: 91 - protein, 10 aa, synthetic construct
GGLSGGGLSG

SEQ ID NO: 92 - protein, 10 aa, synthetic construct
GGGLGGGGLG

SEQ ID NO: 93 - protein, 10 aa, synthetic construct
GGGSLGGGSL

SEQ ID NO: 94 - protein, 10 aa, synthetic construct
LGGSSLGGSS

SEQ ID NO: 95 - protein, 10 aa, synthetic construct
GLGSSGLGSS

SEQ ID NO: 96 - protein, 10 aa, synthetic construct
GGLSSGGLSS

SEQ ID NO: 97 - protein, 6 aa, synthetic construct
GSGGGA

SEQ ID NO: 98 - protein, 12 aa, synthetic construct
GSGGGAGSGGGA

SEQ ID NO: 99 - protein, 18 aa, synthetic construct
GSGGGAGSGGGAGSGGGA

SEQ ID NO: 100 - protein, 24 aa, synthetic construct
GSGGGAGSGGGAGSGGGAGSGGGA

SEQ ID NO: 101 - protein, 10 aa, synthetic construct
GENLYFQSGG

SEQ ID NO: 102 - protein, 10 aa, synthetic construct
SGGGSSGGGS

SEQ ID NO: 103 - protein, 10 aa, synthetic construct
SSGGGSSGGG

SEQ ID NO: 104 - protein, 10 aa, synthetic construct
GGSGGGGSGG

SEQ ID NO: 105 - protein, 10 aa, synthetic construct
GSGSGSGSGS

SEQ ID NO: 106 - protein, 6 aa, synthetic construct
GGGSSS

SEQ ID NO: 107 - protein, 15 aa, synthetic construct
GGGSSGGGSSGGGSS

SEQ ID NO: 108 - protein, 8 aa, synthetic construct
GLGGLAAA

SEQ ID NO: 109 - protein, 12 aa, synthetic construct
KPEPKPAPAPKP

SEQ ID NO: 110 - protein, 12 aa, synthetic construct
AEAAAKEAAAKA

SEQ ID NO: 111 - protein, 15 aa, synthetic construct
PSRLEEELRRRLTEP

SEQ ID NO: 112 - protein, 8 aa, synthetic construct
SACYCELS

SEQ ID NO: 113 - protein, 16 aa, synthetic construct
TQKSLSLSPGKGLGGL

SEQ ID NO: 114 - protein, 13 aa, synthetic construct
SLSLSPGKGLGGL

SEQ ID NO: 115 - protein, 36 aa, synthetic construct
GGSAGGSGSGSSGGSSGASGTGTAGGTGSGSGTGSG

SEQ ID NO: 116 - protein, 36 aa, synthetic construct
GGSGGGSEGGGSEGGGSEGGGSEGGGSEGGGSGGGS

SEQ ID NO: 117 - protein, 12 aa, synthetic construct
ELKTPLGDTTHT

SEQ ID NO: 118 - protein, 15 aa, synthetic construct
EPKSCDTPPPCPRCP

SEQ ID NO: 119 - protein, 21 aa, synthetic construct
FNNFTVSFWLRVPKVSASHLE

SEQ ID NO: 120 - protein, 17 aa, synthetic construct
ILMQYIKANSKFIGITE

SEQ ID NO: 121 - protein, 15 aa, synthetic construct
QSIQLSSLMVAQAIP

SEQ ID NO: 122 - protein, 13 aa, synthetic construct
AKFVAAWTLKAAA

SEQ ID NO: 123 - protein, 76 aa, synthetic construct [sequence omitted]

SEQ ID NO: 124 - protein, 76 aa, synthetic construct
MQIFVKTLTGKTITLEVEPSDTIENVKAKIQDKEGIPPDQQRLIFAGKQLEDGRTLSDYNIQKESTLHLVLRLRGA

SEQ ID NO: 125 - protein, 76 aa, synthetic construct
MQIFVKTLTGKTITLEVEPSDTIENVKAKIQDKEGIPPDQQRLIFAGKQLEDGRTLSDYNIQKESTLHLVLRLRGV

SEQ ID NO: 126 - protein, 76 aa, synthetic construct
MQIFVKTLTGKTITLEVEPSDTIENVKAKIQDKEGIPPDQQRLIFAGKQLEDGRTLSDYNIQKESTLHLVLRPRGG

SEQ ID NO: 127 - protein, 74 aa, synthetic construct
MQIFVKTLTGKTITLEVEPSDTIENVKAKIQDKEGIPPDQQRLIFAGKQLEDGRTLSDYNIQKESTLHLVLRLR

SEQ ID NO: 128 - protein, 237 aa, synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 129 - DNA, 210 bp, synthetic construct
  source 1..210: mol_type = other DNA
gcaccacttgctgctgacacgccgaccgcctgctgcttcagctacacctcccgacagattccacagaatttcatagctgactactttgagacgagcagccagtgctccaagcccagtgtcatcttcctaaccaagagaggccggcaggtctgtgctgaccccagtgaggagtgggtccagaaatacgtcagtgacctggagctgagtgcc

SEQ ID NO: 130 - protein, 19 aa, synthetic construct
MNFGLRLIFLVLTLKGVQC

SEQ ID NO: 131 - protein, 22 aa, synthetic construct
MDAMKRGLCCVLLLCGAVFVSP

SEQ ID NO: 132 - protein, 17 aa, synthetic construct
MWLQSLLLLGTVACSIS

SEQ ID NO: 133 - protein, 23 aa, synthetic construct
MKVSAAALAVILIATALCAPASA

SEQ ID NO: 134 - protein, 22 aa, synthetic construct
MCPARSLLLVATLVLLDHLSLA

SEQ ID NO: 135 - protein, 22 aa, synthetic construct
MCHQQLVISWFSLVFLASPLVA

SEQ ID NO: 136 - protein, 24 aa, synthetic construct
MRSSPGNMERIVICLMVIFLGTLV

SEQ ID NO: 137 - DNA, 69 bp, synthetic construct
  source 1..69: mol_type = other DNA
atgcaggtctccactgctgcccttgccgtcctcctctgcaccatggctctctgcaaccaggtcctctct

SEQ ID NO: 138 - protein, 18 aa, synthetic construct
MKWVTFISLLFLFSSAYS

SEQ ID NO: 139 - protein, 20 aa, synthetic construct
MEAPAQLLFLLLLWLPSSRA

SEQ ID NO: 140 - protein, 19 aa, synthetic construct
MEFGLSWLFLVAILKGVQC

SEQ ID NO: 141 - DNA, 180 bp, synthetic construct
  source 1..180: mol_type = other DNA
gctgggcaggtgaggatctgggccacataccagaccatgctggacaagatccgggaggtgccggagggctggctcatctttgtggccgagagggaagagctctatgtacgcgttagaaatggcttccggaaggtgctgctggaggcccggacagccctcccgagaggcacgggcaatgag

SEQ ID NO: 142 - protein, 27 aa, synthetic construct
GYIPEAPRDGQAYVRKDGEWVLLSTFL

SEQ ID NO: 143 - DNA, 117 bp, synthetic construct
  source 1..117: mol_type = other DNA
aagcctctggacggagagtatttcactctccagatccggggccccgaaaggttcgaaatgttccgggagcttaacgaggccttggagctgaaagacgcacaggccggaaaggaaccg

SEQ ID NO: 144 - DNA, 333 bp, synthetic construct
  source 1..333: mol_type = other DNA
gccgaactcccgcccaaggtgtccgtgttcgtccctccccgcgatgggttcttcggcaatccacgaaaatccaaactgatttgtcaggccaccggcttctccccccgacagatccaggtgagttggctacgagagggtaaacaggtggggagcggagtgaccactgaccaggtgcaggccgaggccaaggaaagcggacccacaacatacaaagtgacaagcactctgacgattaaggagtcagactggctcggccaatccatgtttacatgccgggttgatcacagagggttgaccttccaacagaacgcatccagtatgtgcgttccagat

SEQ ID NO: 145 - DNA, 81 bp, synthetic construct
  source 1..81: mol_type = other DNA
gagctcaaaaccccacttggtgacacaactcacacagagcccaaatcttgtgacacacctcccccgtgcccaaggtgccca

SEQ ID NO: 146 - DNA, 321 bp, synthetic construct
  source 1..321: mol_type = other DNA
ggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 147 - DNA, 432 bp, synthetic construct
  source 1..432: mol_type = other DNA
gagctcaaaaccccacttggtgacacaactcacacagagcccaaatcttgtgacacacctcccccgtgcccaaggtgcccaggcggtggaagcagcggaggtggaagtggaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaa

SEQ ID NO: 148 - protein, 524 aa, synthetic construct
DTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLV

SEQ ID NO: 149 - protein, 9 aa, synthetic construct
CTELKLSDY

SEQ ID NO: 150 - protein, 9 aa, synthetic construct
NLVPMVATV

SEQ ID NO: 151 - protein, 9 aa, synthetic construct
KLVANNTRL

SEQ ID NO: 152 - protein, 68 aa, synthetic construct
SPYSSDTTPCCFAYIARPLPRAHIKEYFYTSGKCSNPAVVFVTRKNRQVCANPEKKWVREYINSLEMS

SEQ ID NO: 153 - DNA, 36 bp, synthetic construct
  source 1..36: mol_type = other DNA
tggcccaggttccacagcagcgtgttccacacccac

SEQ ID NO: 154 - DNA, 324 bp, synthetic construct
  source 1..324: mol_type = other DNA
atggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctcctgaccagccctgggtggatcgcatcatccgaagactgaagaagtcttctgccaagaacaaaggcaacagcaccagaaggagccctgtgtct

SEQ ID NO: 155 - protein, 43 aa, synthetic construct
LEIRAAFLRQRNTALRTEVAELEQEVQRLENEVSQYETRYGPL

SEQ ID NO: 156 - protein, 23 aa, synthetic construct
MQVSTAALAVLLCTMALCNQVLS

SEQ ID NO: 157 - protein, 70 aa, synthetic construct
APLAADTPTACCFSYTSRQIPQNFIADYFETSSQCSKPSVIFLTKRGRQVCADPSEEWVQKYVSDLELSA

SEQ ID NO: 158 - protein, 107 aa, synthetic construct
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 159 - protein, 15 aa, synthetic construct
RSFIEDLLFNKVTLA

SEQ ID NO: 160 - protein, 25 aa, synthetic construct
MTYRRLISMMGFKMNYQVNGYPNMF

SEQ ID NO: 161 - protein, 15 aa, synthetic construct
LMIERFVSLAIDAYP

SEQ ID NO: 162 - protein, 15 aa, synthetic construct
RAMPNMLRIMASLVL

SEQ ID NO: 163 - protein, 15 aa, synthetic construct
MVYMPASWVMRIMTW

SEQ ID NO: 164 - protein, 17 aa, synthetic construct
FLNRFTTTLNDFNLVAM

SEQ ID NO: 165 - protein, 18 aa, synthetic construct
SSVELKHFFFAQDGNAAI

SEQ ID NO: 166 - protein, 25 aa, synthetic construct
HFAIGLALYYPSARIVYTACSHAAV

SEQ ID NO: 167 - protein, 15 aa, synthetic construct
YFIKGLNNLNRGMVL

SEQ ID NO: 168 - protein, 15 aa, synthetic construct
YLNTLTLAVPYNMRV

SEQ ID NO: 169 - protein, 16 aa, synthetic construct
AQFAPSASAFFGMSRI

SEQ ID NO: 170 - protein, 15 aa, synthetic construct
EIVDTVSALVYDNKL

SEQ ID NO: 171 - protein, 28 aa, synthetic construct
SSGDATTAYANSVFNICQAVTANVNALL

SEQ ID NO: 172 - protein, 17 aa, synthetic construct
HVISTSHKLVLSVNPYV

SEQ ID NO: 173 - protein, 24 aa, synthetic construct
MLSDTLKNLSDRVVFVLWAHGFEL

SEQ ID NO: 174 - protein, 20 aa, synthetic construct
TANPKTPKYKFVRIQPGQTF

SEQ ID NO: 175 - protein, 18 aa, synthetic construct
ASIKNFKSVLYYQNNVFM

SEQ ID NO: 176 - protein, 16 aa, synthetic construct
FVNEFYAYLRKHFSMM

SEQ ID NO: 177 - protein, 15 aa, synthetic construct
RVWTLMNVLTLVYKV

SEQ ID NO: 178 - protein, 15 aa, synthetic construct
FAYANRNRFLYIIKL

SEQ ID NO: 179 - protein, 107 aa, synthetic construct
GGSSGGKFGGSTTAPSAQLEKELQALEKENAQLEWELQALEKELAQGGGSGGLTKFGGSTTAPSAQLEKELQALEKENAQLEWELQALEKELAQGGGSGGLTGLSGL

SEQ ID NO: 180 - protein, 108 aa, synthetic construct
GGSSGGKFGGSTTAPSAQLKKKLQALKKKNAQLKWKLQALKKKLAQGGGSGGLTKFGGSTTAPSAQLKKKLQALKKKNAQLKWKLQALKKKLAQGGGSSGGLTGLSGL

SEQ ID NO: 181 - protein, 31 aa, synthetic construct
GEIAALEAKNAALKAEIAALEAKNAALKAGC

SEQ ID NO: 182 - protein, 32 aa, synthetic construct
GKIAALKAENAALEAKIAALKAENAALEAGGC

SEQ ID NO: 183 - protein, 31 aa, synthetic construct
GEIAALEAKNAALKAEIAALEAKIAALKAGY

SEQ ID NO: 184 - protein, 32 aa, synthetic construct
YGKIAALKAENAALEAKIAALKAEIAALEAGY

SEQ ID NO: 185 - protein, 30 aa, synthetic construct
GEIAALEAKIAALKAKNAALKAEIAALEAG

SEQ ID NO: 186 - protein, 30 aa, synthetic construct
GKIAALKAEIAALEAENAALEAKIAALKAG

SEQ ID NO: 187 - protein, 245 aa, synthetic construct
DIVLTQSPASLAVSLGQRATISCRASKSVSTSGYSYMHWYQQKPGQPPKLLIYLTSNLESGVPARFSGSGSGTDFTLNIHPVEEEDAATYYCQHSRELPWTFGGGTKLEIKGGGGSGGGGSGGGGSQVQLQQSGPDLVKPGASVTISCKASGYAFSSSWMSWLKQRPGKGLEWIGWIFPRDGDTNYNGKFKGKATLTADKSSSTAYMQLSSLTSEDSAVYFCARRGDYHYGMDYWGQGTSVTVSS

SEQ ID NO: 188 - protein, 43 aa, synthetic construct
NITNLCPFGEVFNATRFASVYAWNRKRISNCVADYSVLYNSAS

SEQ ID NO: 189 - protein, 43 aa, synthetic construct
CVADYSVLYNSASFSTFKCYGVSPTKLNDLCFTNVYADSFVIR

SEQ ID NO: 190 - protein, 43 aa, synthetic construct
CFTNVYADSFVIRGDEVRQIAPGQTGKIADYNYKLPDDFTGCV

SEQ ID NO: 191 - protein, 43 aa, synthetic construct
YNYKLPDDFTGCVIAWNSNNLDSKVGGNYNYLYRLFRKSNLKP

SEQ ID NO: 192 - protein, 46 aa, synthetic construct
YLYRLFRKSNLKPFERDISTEIYQAGSTPCNGVEGFNCYFPLQSYG

SEQ ID NO: 193 - protein, 39 aa, synthetic construct
GFNCYFPLQSYGFQPTNGVGYQPYRVVVLSFELLHAPAT

SEQ ID NO: 194 - protein, 33 aa, Synthetic construct
GSYGGIEAKIEAIEAKAEAIEAKIEAIEAKIEA

SEQ ID NO: 195 - protein, 5 aa, synthetic construct
GGGGS

SEQ ID NO: 196 - protein, 219 aa, Respiratory Syncytial Virus
SAIASGVAVCKVLHLEGEVNKIKSALLSTNKAVVSLSNGVSVLTFKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGGPGGWYTSVITIELSNIKENKCNGTDAKVKLIKQELDKYKNAVTELQLLMQSTPATN

SEQ ID NO: 197 - protein, 35 aa, Synthetic construct
GSYGGIEQKIEAIEWKWEAIEQKIEAIEQKIEAGS

SEQ ID NO: 198 - protein, 242 aa, Homo sapiens
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGL

SEQ ID NO: 199 - protein, 24 aa, Respiratory Syncytial Virus
MELLIKANAITTILTAVTFCFASG

SEQ ID NO: 200 - protein, 481 aa, synthetic construct
QNITEEFYQSTCSAVSKGYLGALRTGWYTSVITIELSNIKENKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLSAIGGYIPEAPRDGQAYVRKDGEWVLLSTFLG

SEQ ID NO: 201 - protein, 483 aa, synthetic construct
QNITEEFYQSTCSAVSKGYLGALRTGWYTSVITIELSNIKENKCNGTDAKVKLIKQELDKYKNAVTDLQLLMQSTPATNNGSGSAIASGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGQSLYVKGEPIINFYDPLVFPSDEFDASISQVNEKINQSLAFIRKSDELLSAIGGYIPEAPRDGQAYVRKDGEWVLLSTFLG

SEQ ID NO: 202 - protein, 510 aa, synthetic construct
QNITEEFYQSTCSAVSKGYLGALRTGWYTSVITIELSNIKENKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLHNVNVGKSTTNIMITTIIIVIIVILLLLIAVGLFLYCKARSTPVTLSKDQLSGINNIAFSN

SEQ ID NO: 203 - protein, 512 aa, synthetic construct
QNITEEFYQSTCSAVSKGYLGALRTGWYTSVITIELSNIKENKCNGTDAKVKLIKQELDKYKNAVTDLQLLMQSTPATNNGSGSAIASGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGQSLYVKGEPIINFYDPLVFPSDEFDASISQVNEKINQSLAFIRKSDELLHNVNVGKSTTNIMITTIIIVIIVILLLLIAVGLFLYCKARSTPVTLSKDQLSGINNIAFSN

SEQ ID NO: 204 - protein, 28 aa, Respiratory Syncytial Virus
MTNDRLEAMARLRNEESEKMAKDTSDEV

SEQ ID NO: 205 - protein, 11 aa, Respiratory Syncytial Virus
TVSVGNTLYYV

SEQ ID NO: 206 - protein, 43 aa, Respiratory Syncytial Virus
ATFTFLVNYEMKLLHKVGSTKYKKYTEYNTKYGTFPMPIFINH

SEQ ID NO: 207 - protein, 41 aa, synthetic construct
PADLLIKELANVNILVKQISTPKGPSLRVMINSRSAVLAQM

SEQ ID NO: 208 - protein, 25 aa, Respiratory Syncytial Virus
STYTAAVQYNVLEKDDDPASLTIWV

SEQ ID NO: 209 - protein, 26 aa, Respiratory Syncytial Virus
SLSMIKVRLQNLFDNDEVALLKITCY

SEQ ID NO: 210 - protein, 36 aa, Respiratory Syncytial Virus
AEMEQVVEVYEYAQKLGGEAGFYHILNNPKASLLSL

SEQ ID NO: 211 - protein, 624 aa, synthetic construct
QNITEEFYQSTCSAVSKGYLSALRTGWYTSVITIELSNIKENKCNGTDAKVKLIKQELDKYKNAVTELQLLMGSGNVGLGGAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSGGSSGSSGGSDIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKCKDHATFNFLQWYVAEQHEEEVLFKDILDKIELIGNENHGLYLADQYVKGIAKSRKSGS

SEQ ID NO: 212 - protein, 626 aa, synthetic construct
QNITEEFYQSTCSAVSKGYLSALRTGWYTSVITIELSNIKENKCNGTDAKVKLIKQELDKYKNAVTELQLLMGSGNVGLGGAIASGVAVSKVLHLEGEVNKIKSALLSTNKAVVSLSNGVSVLTFKVLDLKNYIDKQLLPILNKQSCSISNPETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKNGSNICLTRTDRGWYCDNAGNVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFINKSDELLSGSGSESQVRQQFSKDIEKLLNEQVNKEMQSSNLYMSMSSWSYTHSLDGAGLFLFDHAAEEYEHAKKLIIFLNENNVPVQLTSISAPEHCFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 213 - protein, 15 aa, synthetic construct
ATFTFLVNYEMKLLH

SEQ ID NO: 214 - protein, 15 aa, synthetic construct
LVNYEMKLLHKVGST

SEQ ID NO: 215 - protein, 15 aa, synthetic construct
MKLLHKVGSTKYKKY

SEQ ID NO: 216 - protein, 15 aa, synthetic construct
KVGSTKYKKYTEYNT

SEQ ID NO: 217 - protein, 15 aa, synthetic construct
KYKKYTEYNTKYGTF

SEQ ID NO: 218 - protein, 15 aa, synthetic construct
TEYNTKYGTFPMPIF

SEQ ID NO: 219 - protein, 13 aa, synthetic construct
KYGTFPMPIFINH

SEQ ID NO: 220 - protein, 26 aa, synthetic construct
SLSMIKVRLQNLFDNDEVALLKITCY

SEQ ID NO: 221 - protein, 15 aa, synthetic construct
SLSMIKVRLQNLFDN

SEQ ID NO: 222 - protein, 15 aa, synthetic construct
KVRLQNLFDNDEVAL

SEQ ID NO: 223 - protein, 15 aa, synthetic construct
NLFDNDEVALLKITC

SEQ ID NO: 224 - protein, 11 aa, synthetic construct
DEVALLKITCY

SEQ ID NO: 225 - protein, 11 aa, synthetic construct
TVSVGNTLYYV

SEQ ID NO: 226 - protein, 15 aa, synthetic construct
PADLLIKELANVNIL

SEQ ID NO: 227 - protein, 15 aa, synthetic construct
IKELANVNILVKQIS

SEQ ID NO: 228 - protein, 15 aa, synthetic construct
NVNILVKQISTPKGP

SEQ ID NO: 229 - protein, 15 aa, synthetic construct
VKQISTPKGPSLRVM

SEQ ID NO: 230 - protein, 15 aa, synthetic construct
TPKGPSLRVMINSRS

SEQ ID NO: 231 - protein, 15 aa, synthetic construct
SLRVMINSRSAVLAQ

SEQ ID NO: 232 - protein, 11 aa, synthetic construct
INSRSAVLAQM

SEQ ID NO: 233 - protein, 15 aa, synthetic construct
AEMEQVVEVYEYAQK

SEQ ID NO: 234 - protein, 15 aa, synthetic construct
VVEVYEYAQKLGGEA

SEQ ID NO: 235 - protein, 15 aa, synthetic construct
EYAQKLGGEAGFYHI

SEQ ID NO: 236 - protein, 15 aa, synthetic construct
LGGEAGFYHILNNPK

SEQ ID NO: 237 - protein, 15 aa, synthetic construct
GFYHILNNPKASLLS

SEQ ID NO: 238 - protein, 11 aa, synthetic construct
LNNPKASLLSL

SEQ ID NO: 239 - protein, 25 aa, synthetic construct
STYTAAVQYNVLEKDDDPASLTIWV

SEQ ID NO: 240 - protein, 15 aa, synthetic construct
STYTAAVQYNVLEKD

SEQ ID NO: 241 - protein, 15 aa, Synthetic construct
AVQYNVLEKDDDPAS

SEQ ID NO: 242 - protein, 15 aa, synthetic construct
VLEKDDDPASLTIWV

SEQ ID NO: 243 - protein, 28 aa, synthetic construct
MTNDRLEAMARLRNEESEKMAKDTSDEV

SEQ ID NO: 244 - protein, 15 aa, synthetic construct
MTNDRLEAMARLRNE

SEQ ID NO: 245 - protein, 15 aa, synthetic construct
LEAMARLRNEESEKM

SEQ ID NO: 246 - protein, 15 aa, synthetic construct
RLRNEESEKMAKDTS

SEQ ID NO: 247 - protein, 13 aa, synthetic construct
ESEKMAKDTSDEV

SEQ ID NO: 248 - protein, 126 aa, Mus musculus
EAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSD

SEQ ID NO: 249 - protein, 112 aa, Mus musculus
ALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 250 - protein, 442 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSETVCPVVTTPSGSVGSLCSRQSQVLCGYLDAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 251 - protein, 309 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFR

SEQ ID NO: 252 - DNA, 462 bp, synthetic construct
  source 1..462: mol_type = other DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 253 - protein, 390 aa, synthetic construct
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 254 - protein, 442 aa, synthetic construct; identical to SEQ ID NO: 250

SEQ ID NO: 255 - protein, 309 aa, synthetic construct; identical to SEQ ID NO: 251

SEQ ID NO: 256 - DNA, 1173 bp, synthetic construct
  source 1..1173: mol_type = other DNA
atgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 257 - DNA, 1320 bp, synthetic construct
  source 1..1320: mol_type = other DNA
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaactgtctgcccagttgttacaacaccctctggctcagtgggcagcttgtgctccagacagtcccaggtgctctgtgggtaccttgatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaa

SEQ ID NO: 258 - DNA, 930 bp, synthetic construct
  source 1..930: mol_type = other DNA
atgaagatgcacttgcaaagggctctggtggtcctggccctgctgaactttgccacggtcagcctctctctgtccacttgcaccaccttggacttcggccacatcaagaagaagagggtggaagccattaggggacagatcttgagcaagctcaggctcaccagcccccctgagccaacggtgatgacccacgtcccctatcaggtcctggccctttacaacagcacccgggagctgctggaggagatgcatggggagagggaggaaggctgcacccaggaaaacaccgagtcggaatactatgccaaagaaatccataaattcgacatgatccaggggctggcggagcacaacgaactggctgtctgccctaaaggaattacctccaaggttttccgcttcaatgtgtcctcagtggagaaaaatagaaccaacctattccgagcagaattccgggtcttgcgggtgcccaaccccagctctaagcggaatgagcagaggatcgagctcttccagatccttcggccagatgagcacattgccaaacagcgctatatcggtggcaagaatctgcccacacggggcactgccgagtggctgtcctttgatgtcactgacactgtgcgtgagtggctgttgagaagagagtccaacttaggtctagaaatcagcattcactgtccatgtcacacctttcagcccaatggagatatcctggaaaacattcacgaggtgatggaaatcaaattcaaaggcgtggacaatgaggatgaccatggccgtggagatctggggcgcctcaagaagcagaaggatcaccacaaccctcatctaatcctcatgatgattcccccacaccggctcgacaacccgggccaggggggtcagaggaagaagcgggctttggacaccaattactgcttccggtga

SEQ ID NO: 259 - protein, 361 aa, synthetic construct
LSTCKTIDMELVKRKRIEAIRGQILSKLRLASPPSQGEVPPGPLPEAVLALYNSTRDRVAGESADPEPEPEADYYAKEVTRVLMVDRNNAIYEKTKDISHSIYMFFNTSDIREAVPEPPLLSRAELRLQRLKSSVEQHVELYQKYSNNSWRYLGNRLLTPTDTPEWLSFDVTGVVRQWLNQGDGIQGFRFSAHCSCDSKDNKLHVEINGISPKRRGDLGTIHDMNRPFLLLMATPLERAQHLHSSRHRRALDTNYCFSSTEKNCCVRQLYIDFRKDLGWKWIHEPKGYHANFCLGPCPYIWSLDTQYSKVLALYNQHNPGASASPCCVPQALEPLPIVYYVGRKPKVEQLSNMIVRSCKCS

SEQ ID NO: 260 - protein, 178 aa, synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 261 - DNA, 537 bp, synthetic construct
  source 1..537: mol_type = other DNA
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 262 - protein, 160 aa, synthetic construct
SRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 263 - protein, 93 aa, synthetic construct
MKLVTIFLLVTISLCSYSATAFLINKVPLPVDKLAPLPLDNILPFMDPLKLLLKTLGISVEHLVEGLRKCVNELGPEASEAVKKLLEALSHLV

SEQ ID NO: 264 - DNA, 282 bp, synthetic construct
  source 1..282: mol_type = other DNA
atgaagctggtaactatcttcctgctggtgaccatcagcctttgtagttactctgctactgccttcctcatcaacaaagtgccccttcctgttgacaagttggcacctttacctctggacaacattcttccctttatggatccattaaagcttcttctgaaaactctgggcatttctgttgagcaccttgtggaggggctaaggaagtgtgtaaatgagctgggaccagaggcttctgaagctgtgaagaaactgctggaggcgctatcacacttggtgtga

SEQ ID NO: 265 - protein, 70 aa, synthetic construct
LLINRLPVVDKLPVPLDDIIPSFDPLKMLLKTLGISVEHLVTGLKKCVDELGPEASEAVKKLLEALSHLV

SEQ ID NO: 266 - protein, 241 aa, synthetic construct
MTSQRSPLAPLLLLSLHGVAASLEVSESPGSIQVARGQPAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIG

SEQ ID NO: 267 - DNA, 723 bp, synthetic construct
  source 1..723: mol_type = other DNA
atgacttctcagcgttcccctctggcgcctttgctgctcctctctctgcacggtgttgcagcatccctggaagtgtcagagagccctgggagtatccaggtggcccggggtcagccagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattgga

SEQ ID NO: 268 - protein, 218 aa, synthetic construct
LEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPQPRSV

SEQ ID NO: 269 - protein, 246 aa, synthetic construct
DIQMTQSPSFLSTSLGNSITITCHASQNIKGWLAWYQQKSGNAPQLLIYKASSLQSGVPSRFSGSGSGTDYIFTISNLQPEDIATYYCQHYQSFPWTFGGGTKLELKGGGGSGGGGSGGGGSEVKLLESGGGLVQPGGSLRLSCAASGFTFNDFYMNWIRQPPGQAPEWLGVIRNKGNGYTTEVNTSVKGRFTISRDNTQNILYLQMNSLRAEDTAIYYCARGGPYYYSGDDAPYWGQGVMVTVSS

SEQ ID NO: 270 - protein, 161 aa, synthetic construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSD

SEQ ID NO: 271 - DNA, 483 bp, synthetic construct
  source 1..483: mol_type = other DNA
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgac

SEQ ID NO: 272 - protein, 126 aa, synthetic construct; identical to SEQ ID NO: 248

SEQ ID NO: 273 - protein, 170 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 274 - DNA, 510 bp, synthetic construct
  source 1..510: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgccttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtg

SEQ ID NO: 275 - protein, 145 aa, synthetic construct
LEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGM

SEQ ID NO: 276 - DNA, 204 bp, synthetic construct
  source 1..204: mol_type = other DNA
atggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccaca

SEQ ID NO: 277 - DNA, 36 bp, synthetic construct; identical to SEQ ID NO: 153
  source 1..36: mol_type = other DNA

SEQ ID NO: 278 - protein, 18 aa, synthetic construct
MNFGLRLIFLVLTLKGVQ

SEQ ID NO: 279 - protein, 18 aa, synthetic construct
MPGSALLCCLLLLTGMRI

SEQ ID NO: 280 - protein, 21 aa, synthetic construct
MKLVSIFLLVTIGICGYSATA

SEQ ID NO: 281 - protein, 22 aa, synthetic construct
MTRRRSAPASWLLVSLLGVATS

SEQ ID NO: 282 - protein, 35 aa, synthetic construct
MACLGLRRYKAQLQLPSRTWPFVALLTLLFIPVFS

SEQ ID NO: 283 - protein, 29 aa, synthetic construct
MPPSGLRLLPLLLPLPWLLVLTPGRPAAG

SEQ ID NO: 284 - protein, 24 aa, synthetic construct
MWVRQVPWSFTWAVLQLSWQSGWL

SEQ ID NO: 285 - protein, 13 aa, synthetic construct
HCLGKWLGHPDKF

SEQ ID NO: 286 - protein, 29 aa, synthetic construct
AHSLERVCHCLGKWLGHPDKFVGITYALT

SEQ ID NO: 287 - protein, 14 aa, synthetic construct
NTWTTCQSIAFPSK

SEQ ID NO: 288 - protein, 30 aa, synthetic construct
AVPVYIYFNTWTTCQSIAFPSKTSASIGSL

SEQ ID NO: 289 - protein, 21 aa, synthetic construct
VHFFKNIVTPRTPPPSQGKGR

SEQ ID NO: 290 - protein, 36 aa, synthetic construct
RTQDENPVVHFFKNIVTPRTPPPSQGKGRGLSLSRF

SEQ ID NO: 291 - protein, 8 aa, synthetic construct
SIINFEKL

SEQ ID NO: 292 - protein, 341 aa, unidentified
MLILTKVNIYMLIIVLWLYGYNFIISESQCPMINDDSFTLKRKYQIDSAESTIKMDKKRTKFQNRAKMVKEINQTIRAAQTHYETLKLGYIKFKRMIRTTTLEDIAPSIPNNQKTYKLFSDISAIGKASRNPSKMVYALLLYMFPNLFGDDHRFIRYRMHPMSKIKHKIFSPFKLNLIRILVEERFYNNECRSNKWRIIGTQVDKMLIAESDKYTIDARYNLKPMYRIKGKSEEDTLFIKQMVEQCVTSQELVEKVLKILFRDLFKSGEYKAYRYDDDVENGFIGLDTLKLNIVHDIVEPCMPVRRPVAKILCKEMVNKYFENPLHIIGKNLQECIDFVSE

SEQ ID NO: 293 - DNA, 1579 bp, unidentified
  source 1..1579: mol_type = other DNA
ggtgccgctagtacccttaatcatatcacatagtgttttatattccaaatttgtggcaatagacggtttatttctatacgatagtttgtttctggaatcctttgagtattctataccaatattattctttgattcgaatttagtttcttcgatattagattttgtattacctatattcttgatgtagtactttgatgatttttccatggcccattctattaagtcttccaagttggcatcatccacatattgtgatagtaattctcggatatcagtagcggttaccgccattgatgtttgttcattggatgagtaactactaatgtatacattttccatttataacacttatgtattaactttgttcatttatattttttcattattatgttgatattaacaaaagtgaatatatatatgttaataattgtattgtggttatacggccacaattttataatgagtgaaagtcagtgtccgatgatcaatgacgatagctttactctgaaaagaaagtatcaaatcgatagtgcggagtcaacaataaaaatggataagaagaggataaagtttcagaatagagccaaaatggtaaaagaaataaatcagacaataagagtagcacaaactcattacgagacattgaaactaggatacataaaatttaagagaatgattaggactactactctagaagatatagcaccatctattccaaataatcagaaaacttataaactattctcggacatttcagccatcggcaaagcatcacagaatccgagtaagatggtatatgctctgctgctttacatgtttcccaatttgtttggagatgaccatagattcattcgttatagaatgcatccaatgagtaaaatcaaacacaagatcttctctcctttcaaacttaatcttattagaatattagtggaagaaagattctataataatgaatgcagatctaataaatggagaataattggaacacaagttgataaaatgttgatagctgaatctgataaatatacaatagatgcaaggtatcgcctaagacctatatatagaatcaagggaaaatctgaagaagataccctctttatcaaacagatggtagaacaatgtgtgacatcccaggaattggtggaaaaagtgttgaagatactgtttagagatttgttcaagagtggagaatacaaagcgtacagatacgatgatgatgtagaaaatggatttattggattggatacactaaaattaaacattgttcatgatatagttgaaccatgtatgcctgttcgtaggccagtggctaagatactgtgtaaagaaatggtaaataaatactttgagaatccgctacatattattggtaagaatcttcaagagtgcattgactttgttagtgaataggcatttcatctttctccaatactaattcaaattgttaaattaataatggatagtataaatagttattagttataagatagtaaaaataattattagaataagagtgtagtatcatagataactctcttctataaaaatggattttattcgtagaaagtatcttata

SEQ ID NO: 294 - protein, 1039 aa, synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMTNDRLEAMARLRNEESEKMAKDTSDEVGGGGSGGGGSTVSVGNTLYYVGGGGSGGGGSATFTFLVNYEMKLLHKVGSTKYKKYTEYNTKYGTFPMPIFINHGGGGSGGGGSPADLLIKELANVNILVKQISTPKGPSLRVMINSRSAVLAQMGGGGSGGGGSSTYTAAVQYNVLEKDDDPASLTIWVGGGGSGGGGSSLSMIKVRLQNLFDNDEVALLKITCYGGGGSGGGGSAEMEQVVEVYEYAQKLGGEAGFYHILNNPKASLLSLGSGEGRGSLLTCGDVEENPGPMELLILKANAITTILTAVTFCFASGQNITEEFYQSTCSAVSKGYLGALRTGWYTSVITIELSNIKENKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLSAIGGYIPEAPRDGQAYVRKDGEWVLLSTFLG

SEQ ID NO: 295 - protein, 1068 aa, synthetic construct
MQVSTAALAVLLCTMALCNQVLSAPLAADTPTACCFSYTSRQIPQNFIADYFETSSQCSKPSVIFLTKRGRQVCADPSEEWVQKYVSDLELSAELKTPLGDTTHTEPKSCDTPPPCPRCPGGGSSGGGSGGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGKGLGGLMTNDRLEAMARLRNEESEKMAKDTSDEVGGGGSGGGGSTVSVGNTLYYVGGGGSGGGGSATFTFLVNYEMKLLHKVGSTKYKKYTEYNTKYGTFPMPIFINHGGGGSGGGGSPADLLIKELANVNILVKQISTPKGPSLRVMINSRSAVLAQMGGGGSGGGGSSTYTAAVQYNVLEKDDDPASLTIWVGGGGSGGGGSSLSMIKVRLQNLFDNDEVALLKITCYGGGGSGGGGSAEMEQVVEVYEYAQKLGGEAGFYHILNNPKASLLSLGSGEGRGSLLTCGDVEENPGPMELLILKANAITTILTAVTFCFASGQNITEEFYQSTCSAVSKGYLGALRTGWYTSVITIELSNIKENKCNGTDAKVKLIKQELDKYKNAVTDLQLLMQSTPATGSGSAICSGVAVCKVLHLEGEVNKIKSALLSTNKAVVSLSNGVSVLTFKVLDLKNYIDKQLLPILNKQSCSIPNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSRTLPSEVNLCNVDIFNPKYDCKIMTSKTDVSSSVITSLGAIVSCYGKTKCTASNKNRGIIKTFSNGCDYVSNKGVDTVSVGNTLYCVNKQEGQSLYVKGEPIINFYDPLVFPSDEFDASISQVNEKINQSLAFIRKSDELLHNVNVGKSTTNIMITTIIIVIIVILLLLIAVGLFLYCKARSTPVTLSKDQLSGINNIAFSN

SEQ ID NO: 296 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 297 - protein, 237 aa, synthetic construct
DTVLTQSPALAVSPGERVTISCRASDSVSTLMHWYQQKPGQQPKLLIYLASHLESGVPARFSGSGSGTDFTLTIDPVEADDTATYYCQQSWNDPWTFGGGTKLELKGGGGSGGGGSGGGGSEVQLVESDGGLVQPGRSLKLPCAASGFTFSDYYMAWVRQAPTKGLEWVASISYDGSSTYYRDSVKGRFTISRDNAKSTLYLQMDSLRSEDTATYYCGRHSSYFDYWGQGVMVTVSS